SEQUENCE LISTING (3 sequences)

SEQ ID NO: 1 - DNA, 23 bp, Artificial Sequence
  Synthetic Polynucleotide
gccacggtggggtttctaaactt

SEQ ID NO: 2 - DNA, 24 bp, Artificial Sequence
  Synthetic Polynucleotide
gccccagtggtcttacatgcacat

SEQ ID NO: 3 - DNA, 29 bp, Artificial Sequence
  Synthetic Polynucleotide
tgcaccagacccgggctcaggtactccga

(19) United States
(12) Patent Application Publication
(10) Pub. No.: US 2023/0140290 A1
(43) Pub. Date: May 4, 2023

(54) SALICYLAMIDE DERIVATIVES AND RELATED METHODS OF MAKING

(71) Applicants: THE BOARD OF REGENTS OF THE UNIVERSITY OF TEXAS SY, Austin, TX (US); UNIVERSITY OF SEVILLE, Sevilla (ES); SERVICIO ANDALUZ DE SALUD, Sevilla (ES)

(72) Inventors: Jia ZHOU, Galveston, TX (US); Jimin XU, Galveston, TX (US); Javier SANCHEZ-CESPEDES, Sevilla (ES); Judith BERASTEGUI-CABRERA, Sevilla (ES); Maria Eugenia PACHON-IBANEZ, Sevilla (ES); Jeronimo PACHON-DIAZ, Sevilla (ES)

(21) Appl. No.: 17/795,223

(22) PCT Filed: Jan. 26, 2021
(86) PCT No.: PCT/US2021/015072

§ 371 Date: Jul. 25, 2022

Related U.S. Application Data
(60) Provisional application No. 62/966,004, filed on Jan. 26, 2020.
(60) Provisional application No. 63/079,118, filed on Sep. 16, 2020.

(51) Int. Cl. C07F 9/6561; C07F 9/6512; C07C 233/81; C07D 307/85; C07C 233/76; C07C 225/22

(52) U.S. Cl. CPC C07F 9/65616; C07F 9/6512; C07C 233/81; C07D 307/85; C07C 233/76; C07C 225/22

(57) ABSTRACT

Certain embodiments describe antiviral compounds and related methods of using such compounds.

This application claims benefit of priority of U.S. Provisional Patent Application No. 62/966,004 filed Jan. 26, 2020 and U.S. Provisional Patent Application No. 63/079,118 filed Sep. 16, 2020 which are hereby incorporated by reference in their entirety.

The instant application contains a Sequence Listing which has been submitted in ASCII format and is hereby incorporated by reference in its entirety. Said ASCII copy, created on Jan. 22, 2021, is named UTSG.P0390WO_Sequence_Listing_ST25.txt and is 931 bytes in size.

### BACKGROUND

Human adenoviruses (HAdVs) are non-enveloped viruses with an icosahedral capsid which contains a linear and double stranded DNA genome of approximately 36 kb in size that encodes about 35 genes (Robinson et al., Sci. Rep. 2013, 3, 1812). Currently, HAdVs consist of at least 70 different serotypes divided into seven species (HAdV A-G) that belong to the Mastadenovirus genus of the family Adenoviridae; most of these species spread globally with predominant types differing geographically (Hage et al., J. Gen. Virol. 2015, 96, 2734-2742; Echavarria, Clin. Microbiol. Rev. 2008, 21, 704-715; Sandkovsky et al., Curr. Infect. Dis. Rep. 2014, 16, 416). HAdVs can cause a wide variety of clinical diseases including acute upper respiratory disease, conjunctivitis, gastroenteritis, hepatitis, myocarditis, and pneumonia. Primary infections occur in young children with virus transmitted via multiple routes (respiratory, fecal-oral, direct conjunctival inoculation, environmentally, etc.), and are typically self-limiting and rarely associated with severe clinical symptoms in immunocompetent individuals (Echavarria, Clin. Microbiol. Rev. 2008, 21, 704-715; Lion, Clin. Microbiol. Rev. 2014, 27, 441-462). However, HAdV infections can cause significant morbidity and mortality in immunocompromised patients, such as solid-organ transplant (SOT) or allogenic hematopoietic stem cell transplant (allo-HSCT) patients, AIDS patients, and patients with genetic immunodeficiencies (Lion, Clin. Microbiol. Rev. 2014, 27, 441-462; Ison, Clin. Infect. Dis. 2006, 43, 331-339; Abbas et al., Int. J. Infect. Dis. 2017, 62, 86-93; Kojaoghlanian et al., Rev. Med. Virol. 2003, 13, 155-171; Chakrabarti et al., Blood. 2002, 100, 1619-1627)

Pediatric allo-HSCT patients are particularly at risk of HAdV infections with frequencies ranging from 5% to 47%; the reported mortality rates are up to 26% for them with symptomatic infection and as high as 80% for them with disseminated disease (Sandkovsky et al., Curr. Infect. Dis. Rep. 2014, 16, 416; Lion, Clin. Microbiol. Rev. 2014, 27, 441-462; Ison, Clin. Infect. Dis. 2006, 43, 331-339; Sedlicek et al., Biol. Blood Marrow Transplant. 2019, 25, 810-818). Despite the significant clinical impact, there remains no specifically approved antiviral therapy for HAdV infection to date. Several broad-spectrum antiviral drugs are used off-label to treat HAdV infections in immunocompromised patients, being the most common the acyclic nucleoside phosphonates (Toth et al., Antiviral Res. 2018, 153, 1-9; Martínez-Aguado et al., Drug Discov. Today. 2015, 20, 1235-1242; Wold et al., FEMS Microbiol. Rev. 2019, 43, 380-388; Lenaerts et al., Rev. Med. Virol. 2008, 18, 357-374). Cidofovir (FIG. 1), an acyclic nucleoside phosphonate cytosine analogue, is the most frequently used drug to treat HAdV infections. Cidofovir was approved to treat cytomegalovirus (CMV) retinitis and can inhibit viral DNA replication acting as a chain terminator (Chamberlain et al., Antimicrob. Agents Chemother. 2019, 63, e01925-18). Cidofovir displays broad antiviral activities against all HAdV species, but it has low oral bioavailability, long plasma half-life, and significant nephrotoxicity (Wold et al., FEMS Microbiol. Rev. 2019, 43, 380-388; Lenaerts et al., Rev. Med. Virol. 2008, 18, 357-374; Lenaerts and Naesens, Antiviral Res. 2006, 71, 172-180; Yusuf et al., Transplantation. 2006, 81, 1398-1404; Florescu et al., Pediatr. Infect. Dis. J. 2015, 34, 47-51; Lugthart et al., Biol. Blood Marrow Transplant. 2015, 21, 293-299). Considering its proven efficacy, a lipid-linked derivative of cidofovir, named brincidofovir (BCV, previously named CMX001, 3-hexadecyloxy-1-propanol-cidofovir), was developed with improved antiviral potency, safety, bioavailability and pharmacokinetics (Hostetler, Antiviral Res. 2009, 82, 84-98; Tippin et al., Ther. Drug Monit. 2016, 38, 777-786; Florescu et al., Biol. Blood Marrow Transplant. 2012, 18, 731-738; Florescu and Keck, Expert Rev. Anti Infect. Ther. 2014, 12, 1171-1178; Hartline et al., J. Infect. Dis. 2005, 191, 396-399). BCV was proven to be effective in eliminating disseminated HAdV infection in the Syrian Hamster model which is permissive for HAdV-5 replication (Toth et al., Proc. Natl. Acad. Sci. U.S.A. 2008, 105, 7293-7297). More excitingly, the good results from the phase II and III trials (NCT01231344 and NCT02087306) confirm the antiviral activity of BCV against adenoviruses and support the continued development of BCV as the first therapeutic option for HAdV infection (Grimley et al., Biol. Blood Marrow Transplant. 2015, 21, 108-109; Prasad et al., Biol. Blood Marrow Transplant. 2017, 23, 57-58; Grimley et al., Biol. Blood Marrow Transplant. 2017, 23, 512-521; Chittick et al., Antiviral Res. 2017, 143, 269-277). However, BCV administration caused gastrointestinal disturbances, primarily diarrhea in some patients; and a new administration strategy is needed to avoid this adverse effect (Grimley et al., Biol. Blood Marrow Transplant. 2017, 23, 512-521). USC-087 (3), an N-alkyl tyrosinamide phosphonate ester prodrug of HPMPA, the adenine analog of cidofovir, was highly effective against multiple HAdV types in cell culture (Toth et al., Antiviral Res. 2018, 153, 1-9). Promisingly, USC-087 protected Syrian hamsters against lethal challenge with HAdV-5 or -6 even when administered starting at 4 days post challenge. Besides the nucleoside analogues reported in recent years and described above, a few non-nucleoside analogues have also been identified as anti-HAdV agents (Kang et al., Biorg. Med. Chem. Lett. 2016, 26, 5182-5186; Andersson et al., Antimicrob. Agents Chemother. 2010, 54, 3871-3877; Oberg et al., J. Med. Chem. 2012, 55, 3170-3181; Sanchez-Cespedes et al., Antiviral Res. 2014, 108, 65-73; Sanchez-Cespedes et al., J. Med. Chem. 2016, 59, 5432-5448; Marrugal-Lorenzo et al., Sci. Rep. 2019, 9, 17; Marrugal-Lorenzo et al., Antiviral Res. 2018, 159, 77-83; Hamdy and El-Senousy, Acta Pol. Pharm. 2013, 70, 99-110; Liu et al., Virus Res. 2013, 172, 9-14; Mohamed et al., Arch. Pharm. 2015, 348, 194-205; Grosso et al., J. Virol. 2017, 91, e01623-16). Benzoic acid analogue (4) was discovered by high-throughput screening (HTS) and subsequent optimization (Andersson et al., Antimicrob. Agents Chemother. 2010, 54, 3871-3877; Oberg et al., J. Med. Chem. 2012, 55, 3170-3181). It inhibited HAdV-5 replication with an EC₅₀ of 0.58 μM and exhibited low cell toxicity. The current investigators reported a series of inhibitors of HAdV infection including compounds 5-7. Piperazine derivative 5 significantly inhibited HAdV and CMV infections in different phases of their life cycle, with little or no cytotoxicity (Sanchez-Cespedes et al., Antiviral Res. 2014, 108, 65-73; Sanchez-Cespedes et al., J. Med. Chem. 2016, 59, 5432-5448). Niclosamide (6) is an FDA-approved anthelminthic drug used in humans to treat tapeworm infections, involving uncoupling of oxidative phosphorylation. Accumulated studies indicated that niclosamide can modulate multiple signaling pathways and biological processes (Li et al., J. Insect Physiol. 2014, 70, 8-14; Chen et al., Cell. Signal. 2018, 41, 89-96; Fonseca et al., J. Biol. Chem. 2012, 287, 17530-17545; Ren et al., ACS Med. Chem. Lett. 2010, 1, 454-459; Chen et al., ACS Med. Chem. Lett. 2013, 4, 180-185; Xu et al., Biorg. Med. Chem. Lett. 2019, 29, 1399-1402), and has great antiviral potential against various viruses including Zika virus (Xu et al., Nat. Med. 2016, 22, 1101-1107; Li et al., Cell Res. 2017, 27, 1046-1064), coronavirus (Wu et al., Antimicrob. Agents Chemother. 2004, 48, 2693-2696) hepatitis C virus (Stachulski et al., J. Med. Chem. 2011, 54, 8670-8680), Ebola Virus (Madrid et al., ACS Infect. Dis. 2015, 1, 317-326), HIV (Fan et al., Tuberculosis. 2019, 116, 28-33), Japanese encephalitis virus (Fang et al., PLoS One. 2013, 8, e78425), human rhinoviruses or influenza virus (Jurgeit et al., PLoS Pathog. 2012, 8, e1002976), etc. The investigators first found that niclosamide significantly inhibited HAdV infection at low micromolar concentration (IC₅₀=0.60 μM). However, it showed moderate cytotoxicity (CC₅₀=22.9 μM) and a relatively narrow safety window with a low selectivity index (SI=38.2) (Marrugal-Lorenzo et al., Sci. Rep. 2019, 9, 17). Mifepristone (7), a commercially available synthetic steroid drug, showed potent in vitro anti-HAdV activity by interfering with HAdV genome accessibility into the nucleus (Marrugal-Lorenzo et al., Antiviral Res. 2018, 159, 77-83).

While significant progress has been made in anti-HAdV drug discovery, there is still an urgent need to develop highly effective antiviral agents lacking major adverse effects. As part of ongoing antiviral drug discovery and development program (Fan et al., Tuberculosis. 2019, 116, 28-33; Xu et al., J. Med. Chem. 2019, 62, 7941-7960; Niu et al., J. Clin. Invest. 2019, 129, 3361-3373; Ye et al., ACS Infect. Dis. 2016, 2, 382-392), it is herein reported continued structure-activity relationship (SAR) optimization studies of niclosamide and its salicylamide derivatives, aiming to acquire an alternative for the treatment of HAdV infections with increased activity and low toxicity.

### SUMMARY

The effective treatment of adenovirus (HAdV) infections in immunocompromised patients still poses great challenges. Certain embodiments described herein describe continued efforts to optimize a series of salicylamide derivatives as potent inhibitors of HAdV infection.

Certain embodiments are directed to compounds of Formula I:

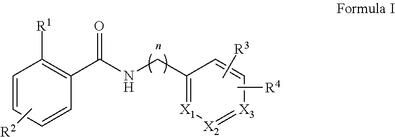

wherein: R¹ is chosen from OH, —OR⁵, —NHSO₂R⁵ or —NHCOR⁵, wherein R⁵ is a straight chained, or branched alkyl; R¹ can be ester prodrugs formed from OH; R² is chosen from H, halogen, CN, NO₂, amino, and alkyl; R³ and/or R⁴ are independently chosen from H, halogen, CN, NO₂, CF₃, various mono- or di-substituted amino groups, or R³ and R⁴ taken together with other atoms to form 5-membered or 6-membered fused ring; X₁, X₂, X₃ are independently chosen from CH and N; and n is 0, 1, or 2 (0-2).

Certain embodiments are directed to compounds of Formula Ia:

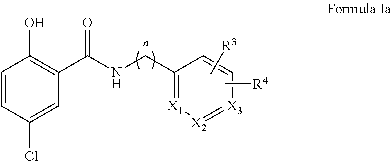

wherein R³ and/or R⁴ are independently chosen from H, halogen, CN, NO₂, CF₃, various mono- or di-substituted amino groups, or R³ and R⁴ taken together with other atoms to form 5-membered or 6-membered fused ring; X₁, X₂, X₃ are independently chosen from CH and N; and n is 0, 1, or 2 (0-2).

Certain embodiments are directed to compounds of Formula Ib:

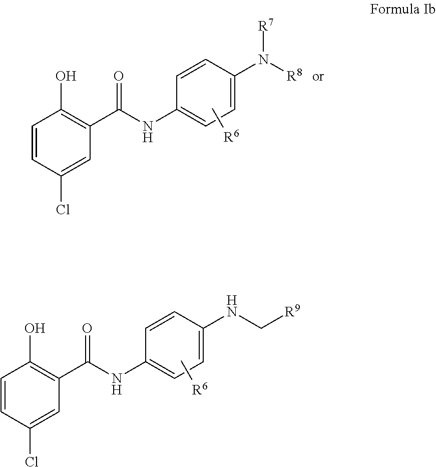

wherein R⁶ is chosen from H, halogen; R⁷ and/or R⁸ are independently chosen from H, alkyl, aryl, arylalkyl, cycloalkyl, alkoxy, heteroalkyl, hydroxyalkyl, or R⁷ and R⁸ taken together with other atoms to form 5-membered or 6-membered fused ring; R⁹ is chosen from various unsubstituted or substituted aryl or heteroaryl.

Certain embodiments are directed to compounds of Formula II:

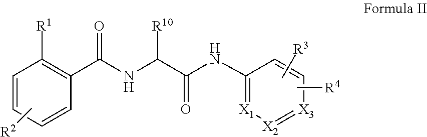

wherein, R¹ is chosen from OH, —OR⁵, —NHSO₂R⁵ or —NHCOR⁵ wherein R⁵ is a straight chained, or branched alkyl; R¹ can be ester prodrugs formed from OH; R² is chosen from H, halogen, CN, NO₂, amino, or alkyl; R³ and/or R⁴ are independently chosen from H, halogen, CN, NO₂, CF₃, various mono- or di-substituted amino groups, or R³, R⁴ taken together with other atoms to form 5-membered or 6-membered fused ring; R¹⁰ is chosen from H, alkyl, cycloalkyl, heteroalkyl, aryl, heteroaryl, arylalkyl, or heteroarylalkyl, wherein the heteroalkyl includes an ester bond, an amide bond, a carbamate, sulfur, or oxygen; aryl; X₁, X₂, X₃ are independently chosen from CH and N.

Certain embodiments are directed to compounds of Formula III:

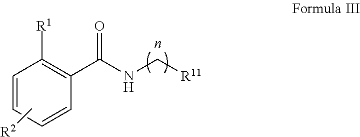

wherein, R¹ is chosen from OH, —OR⁵, —NHSO₂R₅ or —NHCOR₅, wherein R⁵ is a straight chained, or branched alkyl; R¹ can be ester prodrugs formed from OH; R² is chosen from H, halogen, CN, NO₂, amino and alkyl; R¹¹ is chosen from alkyl, heteroalkyl, cycloalkyl, or heterocycle, wherein the heteroalkyl and/or the heterocycle include an ester bond, an amide bond, a carbamate or oxygen atom; and n is 0, 1, 2, 3, 4, 5, or 6 (n is 0-6).

Certain embodiments are directed to 5-Chloro-N-(2-fluoro-4-nitrophenyl)-2-hydroxybenzamide (11), 5-Chloro-2-hydroxy-N-(4-nitrophenyl)benzamide (13), 5-Chloro-N-(2-chloro-4-(trifluoromethyl)phenyl)-2-hydroxybenzamide (14), 5-Chloro-N-(3-fluoro-5-(trifluoromethyl)phenyl)-2-hydroxybenzamide (17), N-(3-Fluoro-5-(trifluoromethyl)phenyl)-2-hydroxybenzamide (18), N-(3-Fluoro-5-(trifluoromethyl)phenyl)-2-hydroxy-5-methylbenzamide (19), 4-Chloro-N-(3-fluoro-5-(trifluoromethyl)phenyl)-2-hydroxybenzamide (20), 5-Chloro-N-(2,4-dichlorobenzyl)-2-hydroxybenzamide (32), 5-Chloro-N-(3-fluoro-4-(trifluoromethyl)benzyl)-2-hydroxybenzamide (36), 5-Chloro-N-(2-fluoro-4-(trifluoromethyl)benzyl)-2-hydroxybenzamide (37), (S)-5-Chloro-N-(1-((2-chloro-4-nitrophenyl)amino)-3-methyl-1-oxobutan-2-yl)-2-hydroxybenzamide (58), (R)-5-Chloro-N-(1-((2-chloro-4-nitrophenyl)amino)-1-oxo-3-phenylpropan-2-yl)-2-hydroxybenzamide (60), 5-Chloro-N-((2S,3R)-1-((2-chloro-4-nitrophenyl)amino)-3-methyl-1-oxopentan-2-yl)-2-hydroxybenzamide (62), (S)-tert-Butyl 4-(5-chloro-2-hydroxybenzamido)-5-((2-chloro-4-nitrophenyl)amino)-5-oxopentanoate (64), (S)—N-(1-((3,5-Bis(trifluoromethyl)phenyl)amino)-3-methyl-1-oxobutan-2-yl)-5-chloro-2-hydroxybenzamide (65), (S)-5-Chloro-N-(1-((3-fluoro-5-(trifluoromethyl)phenyl)amino)-3-methyl-1-oxobutan-2-yl)-2-hydroxybenzamide (67), and (S)-5-Chloro-N-(1-((2-chloro-4-nitrophenyl)amino)-3-methyl-1-oxobutan-2-yl)-2-(methylsulfonamido)benzamide (70).

Certain embodiments are directed to N-(3-Fluoro-5-(trifluoromethyl)phenyl)-2-hydroxybenzamide (18), N-(3-Fluoro-5-(trifluoromethyl)phenyl)-2-hydroxy-5-methylbenzamide (19), 4-Chloro-N-(3-fluoro-5-(trifluoromethyl)phenyl)-2-hydroxybenzamide (20), 5-Chloro-N-(2,4-dichlorobenzyl)-2-hydroxybenzamide (32), 5-Chloro-N-(3-fluoro-4-(trifluoromethyl)benzyl)-2-hydroxybenzamide (36), 5-Chloro-N-(2-fluoro-4-(trifluoromethyl)benzyl)-2-hydroxybenzamide (37), (S)-5-Chloro-N-(1-((2-chloro-4-nitrophenyl)amino)-3-methyl-1-oxobutan-2-yl)-2-hydroxybenzamide (58), (R)-5-Chloro-N-(1-((2-chloro-4-nitrophenyl)amino)-1-oxo-3-phenylpropan-2-yl)-2-hydroxybenzamide (60), 5-Chloro-N-((2S,3R)-1-((2-chloro-4-nitrophenyl)amino)-3-methyl-1-oxopentan-2-yl)-2-hydroxybenzamide (62), (S)-tert-Butyl 4-(5-chloro-2-hydroxybenzamido)-5-((2-chloro-4-nitrophenyl)amino)-5-oxopentanoate (64), (S)—N-(1-((3,5-Bis(trifluoromethyl)phenyl)amino)-3-methyl-1-oxobutan-2-yl)-5-chloro-2-hydroxybenzamide (65), (S)-5-Chloro-N-(1-((3-fluoro-5-(trifluoromethyl)phenyl)amino)-3-methyl-1-oxobutan-2-yl)-2-hydroxybenzamide (67), and (S)-5-Chloro-N-(1-((2-chloro-4-nitrophenyl)amino)-3-methyl-1-oxobutan-2-yl)-2-(methylsulfonamido)benzamide (70).

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

Throughout this application, the term “about” is used to indicate that a value includes the inherent variation of error for the device, the method being employed to determine the value, or the variation that exists among the study subjects.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “derivative” refers to a compound that is chemically modified to form a derivative or variant compound wherein one or more atom or substituent is added or replaces an atom or substituent of the parent compound while maintaining the general structure of the parent compound.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “prevention,” or any variation of these terms, when used in the claims and/or the specification includes any measurable decrease or complete inhibition to achieve a desired result.

The use of the term “or” in the claims is used to mean “and/or” unless explicitly indicated to refer to alternatives only or the alternatives are mutually exclusive, although the disclosure supports a definition that refers to only alternatives and “and/or.”

The term “treatment” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. Treatment may include prophylaxis which refers to preventing a disease or condition or preventing the occurrence of symptoms of such a disease or condition, in a patient. As used herein, the term “patient” refers to a mammal, including a human.

Combination therapies comprise the administration of a compound of the present invention or a pharmaceutically acceptable salt thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The use of the word “a” or “an” when used in conjunction with the term “comprising” in the claims and/or the specification may mean “one,” but it is also consistent with the meaning of “one or more,” “at least one,” and “one or more than one.”

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Representative nucleoside and non-nucleoside inhibitors of adenovirus infection.

FIG. 2. Effect of selected compounds on HAdV infection at different time points. The concentrations selected were depending on their CC₅₀: 10 μM for derivatives 14, 17, 58, 60 and 70; 5 μM for derivatives 11, 13, 20 and 62; and 1 μM for 6. Line chart represent means±SD of duplicate samples.

FIG. 3. Percentage of nuclear-associated HAdV genome of selected compounds. Concentration of these compounds for the assay was selected based on CC₅₀: 11 was tested at 2 μM, 6, 13, 20 and 62 at 5 μM, 14 at 10 μM, 60 at 20 μM and 17, 58 and 70 at 50 μM. Bars represent means±SD of triplicate samples. *p≤0.05, **p≤0.01, ***p≤0.001, ****p≤0.0001.

FIG. 4. Effect of selected compounds on HAdV DNA replication. Concentration of these compounds for the assay was selected based on CC₅₀: compound 11 was tested at 2 M, compounds 6, 13, 20 and 62 at 5 μM, 14 at 10 μM, 60 at 20 μM and 17, 58 and 70 at 50 μM. Bars represent means±SD of triplicate samples. *p≤0.05, **p≤0.01, ***p≤0.001, ****p≤0.0001.

FIGS. 5A-B. (FIG. 5A) Percentage of nuclear-associated HAdV genome of selected compounds. Concentration of niclosamide was 5 μM and 10-fold IC₅₀ concentration obtained in the plaque assay for each compound. Bars represent means±SD of triplicate samples. ***p≤0.001, ****p≤0.0001. (FIG. 5B) Percentage of nuclear-associated HAdV genome of compound 161. Concentration of niclosamide was 5 μM and 10-fold IC₅₀ concentration obtained in the plaque assay for compound 161. Bars represent means±SD of triplicate samples. ****p≤0.0001.

FIGS. 6A-B. (FIG. 6A) Effect of selected compounds on HAdV DNA replication. Concentration of these compounds for the assay was selected based on CC₅₀: niclosamide and 6 were tested at 5 μM, 15 at 2.7 μM, 29 at 11.1 μM, 40 at 7.8 μM, 43 at 12.7 μM, 46 at 9.0 μM, 47 at 12.0 μM and 54 at 9.3 μM. *p≤0.05, **p≤0.01, ***p≤0.001, ****p≤0.0001. (FIG. 6B) Effect of compound 161 on HAdV DNA replication. Concentration of niclosamide was 5 μM and 10-fold IC₅₀ concentration obtained in the plaque assay for compound 161. Bars represent means±SD of triplicate samples. ***p≤0.001, ****p≤0.0001.

FIG. 7. Time-dependence curve assay. Effect of selected compounds on HAdV infection at different time points at concentrations 10-fold the IC₅₀ concentration obtained in the plaque assay of each compound. Line chart represent mean±SD of duplicate samples.

FIG. 8. Effect of selected compounds on HCMV DNA replication. The concentration of these compounds in this assay were selected based on their CC₅₀: niclosamide and 6 were tested at 5 μM, 15 at 2.7 μM, 29 at 11.1 μM, 40 at 7.8 μM, 43 at 12.7 μM, 46 at 9.0 μM, 47 at 12.0 μM and 54 at 9.3 μM. **p≤0.01, ***p≤0.001.

FIG. 9. Percentage of body weight gains for hamsters treated with compounds 1, 2, 3 and 15. Dosing of the compounds started at Day 0. Symbols represent the group mean, while the error bars signify the standard deviation.

FIG. 10. Effect of compound 161 on HAdV infection at different time points. The concentrations used were 5 μM for niclosamide and 0.78 μM for derivative 161. Line chart represent mean±SD of duplicate samples.

FIG. 11. Activity of compound 161 on HAdV-mediated endosomolysis in a α-sarcin assay. The concentrations used were 5 μM for niclosamide and 0.78 μM for derivative 14. L The results represent means±SD of triplicate assays.

FIG. 12. Compound 161 stabilizes HAdV capsid. The concentration used was 50 μM. Data are representative of three experiments.

FIG. 13. Effect of compound 161 on HAdV CMV replication. Concentration of niclosamide was 5 μM and 10-fold IC₅₀ concentration obtained in the plaque assay for compound 161. Bars represent means±SD of triplicate samples. ***p≤0.001.

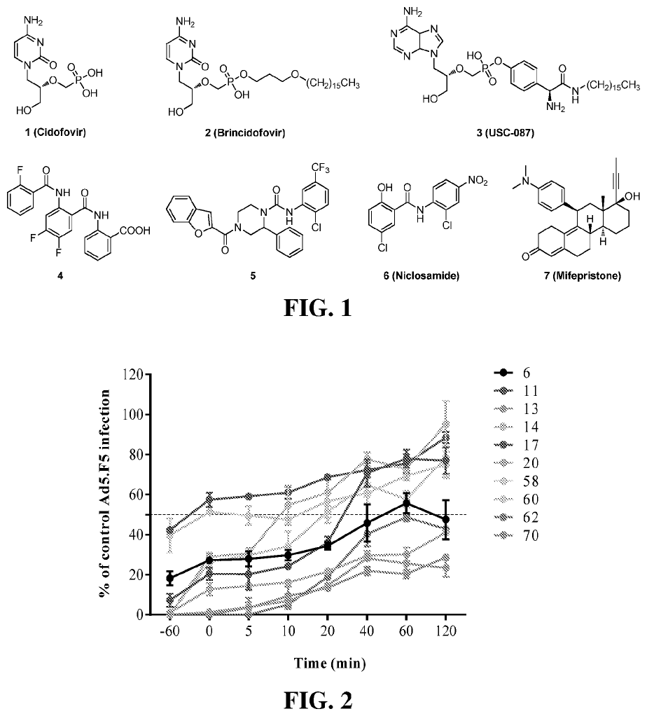

### DESCRIPTION

Described herein is a series of salicylamide derivatives that inhibit HAdV infection. For instance, compounds 11, 13, 14, 17, 18, 19, 20, 32, 36, 37, 58, 60, 62, 64, 65, 67, and 70 (as described below) showed significantly improved anti-HAdV activities with nanomolar to submicromolar IC₅₀ values and high selectivity indexes (SI>100), indicating better safety windows, compared to the lead compound niclosamide. Mechanistic assays suggest that compounds 13, 62, and 70 exert their activities in the HAdV entry pathway, while compounds 14 and 60 likely target the HAdV DNA replication, and 11, 17, 20, and 58 inhibit later steps after DNA replication. Given the broad antiviral activity profile of niclosamide, these derivatives may offer therapeutic potential for other viral infections such as Zika, Ebola or hepatitis C virus.

Certain derivatives described herein are derived from a parent salicylanilide (2-Hydroxy-N-phenylbenzamide).

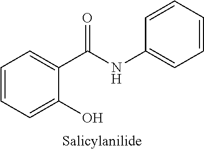 [SMILES: O=C(Nc1ccccc1)c1ccccc1O]

Chemistry. The general synthesis of salicylanilide derivatives is summarized in Scheme 1. Commercially available substituted 2-methoxybenzoic acids 8a-d were coupled with various anilines 9a-i in the presence of PCl₃, followed by demethylation with BBr₃, to afford the corresponding salicylanulides 10-20. R²-R⁴ groups are defined in Table 1. Derivative 21 was accessed under the same coupling conditions by condensation of 3-chlorobenzoic acid 8e with 3-fluoro-5-(trifluoromethyl)aniline 9d.

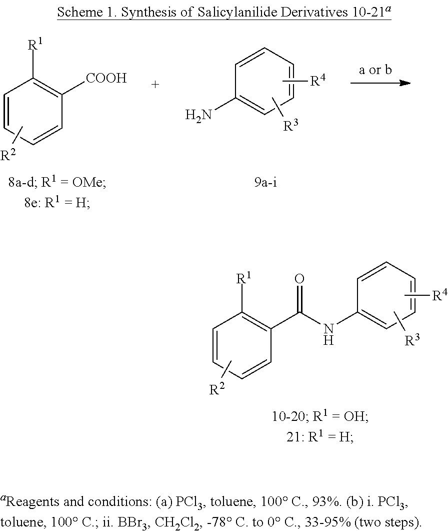

As described in Scheme 2, condensation of 5-chloro-2-methoxybenzoic acid 8a with different substituted benzylamine 22a-p was followed by demethylation using BBr₃ to give salicylamide derivatives 30-45. Direct coupling of 5-chlorosalicylic acid 24 with substituted benzylamine 25a-e afforded salicylamide derivatives 46-50. R¹-R⁴ groups are defined in Table 2. Derivative 51 was synthesized by EDCI-mediated condensation of acid 8a and cumylamine 26 and subsequent demethylation with BBr₃. Substitution of methyl 5-chloro-2-hydroxybenzoate 27 with 4-fluorophenethylamine 28 in methanol directly provided derivative 52.

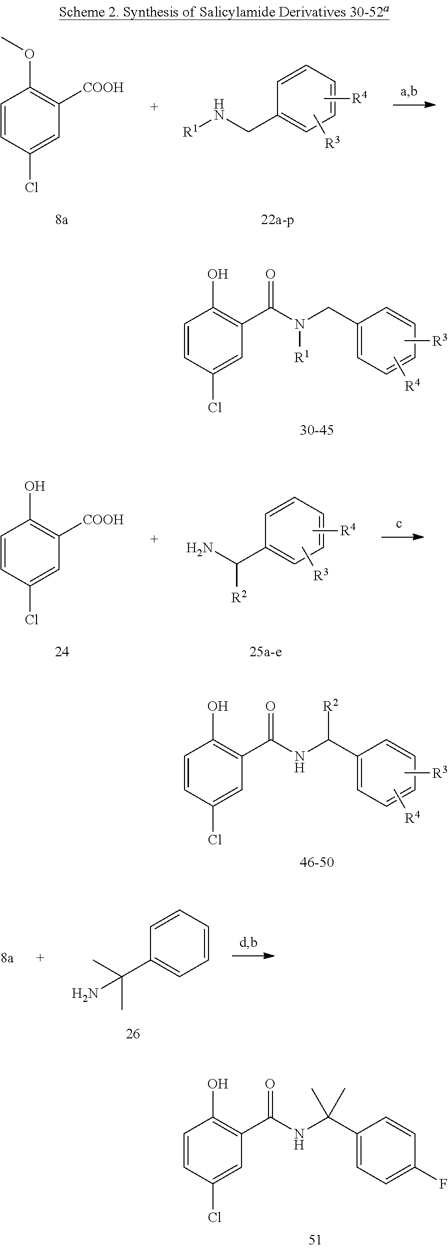

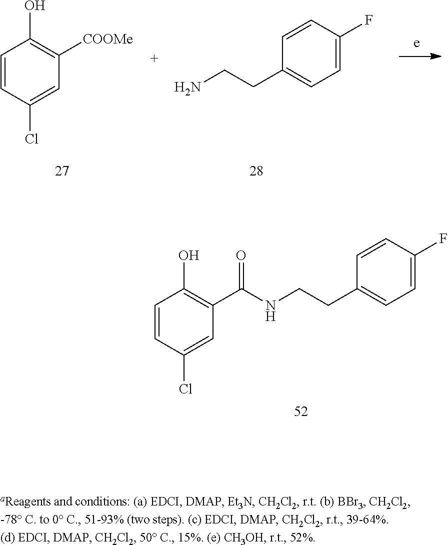 [SMILES: COC(=O)c1cc(Cl)ccc1O ; NCCc1ccc(F)cc1 ; O=C(NCCc1ccc(F)cc1)c1cc(Cl)ccc1O]

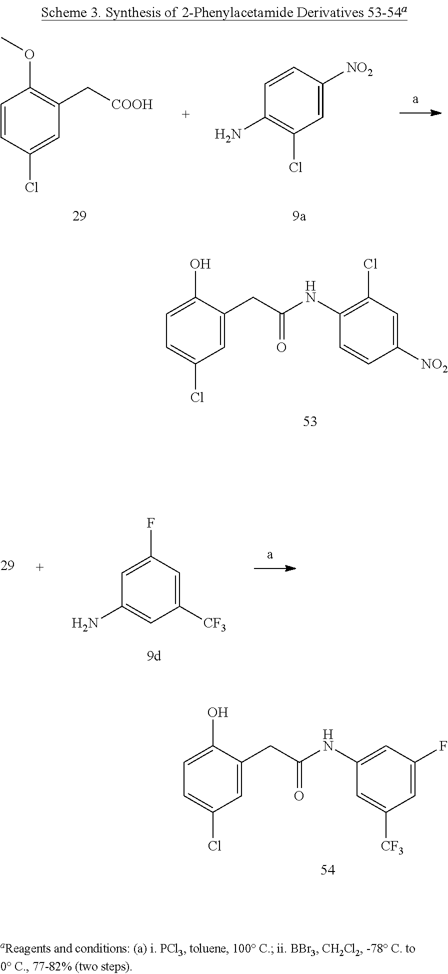 [SMILES: COc1ccc(Cl)cc1CC(=O)O ; Nc1ccc([N+](=O)[O-])cc1Cl ; O=C(Cc1cc(Cl)ccc1O)Nc1ccc([N+](=O)[O-])cc1Cl ; Nc1cc(F)cc(C(F)(F)F)c1 ; O=C(Cc1cc(Cl)ccc1O)Nc1cc(F)cc(C(F)(F)F)c1]

As shown in Scheme 3, 2-phenylacetamide derivatives 53-54 were prepared in a similar manner to that described above for compounds 10-20. Condensation of (5-chloro-2-methoxyphenyl)acetic acid 29 with anilines 9a or 9d was followed by demethylation to afford compound 53 and 54, respectively.

Compounds 56-72 were prepared according to the general synthesis outlined in Scheme 4. Various anilines 9a-e were coupled with different Fmoc-protected amino acids in the presence of PCl₃, followed by piperidine deprotection, to give the amino intermediates 55a-n. The subsequent condensation of amino intermediates 55a-h, j-n with acid 8a was followed by demethylation to afford salicylamide derivatives 56-63 and 65-69. The amino intermediate 55i with tert-butoxycarbonyl moiety was condensed with 5-chlorosalicylic acid 24 to provide compound 64. Derivatives 70-72 were accessed by the coupling reaction between amino 55d and 5-chloro-2-(methylsulfonamido)benzoic acid 23. R¹-R⁵ groups are defined in Table 3.

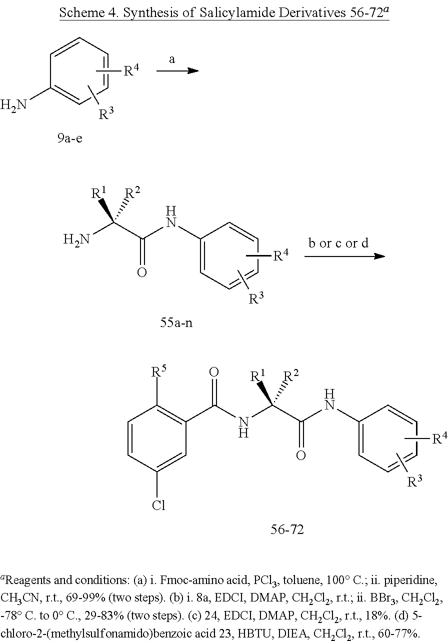

In Vitro Evaluation of Human Adenovirus Inhibition. All newly synthesized compounds were first screened in plaque assay at the concentration of 10 μM, quantifying their abilities to inhibit HAdV plaque formation. The active compounds screened out (inhibition >90%) were further evaluated to characterize their antiviral activity (IC₅₀) in plaque assay and their cytotoxicity (CC₅₀ values). Starting from niclosamide, a simple exploration was performed on the effect generated in its antiviral activity by the replacement of the substituents on its two phenyl rings. As shown in Table 1, moving the 2′-Cl group on the aniline ring to the 3′-position (10) maintained the same level of potency (IC₅₀=1.00 μM) and cytotoxicity (CC₅₀=15.1 μM), in comparison with the parent compound (6). Interestingly, substitution of 2′-Cl with 2′-F (11) resulted in a 11-fold increase in potency (IC₅₀=0.05 μM) and an improved selectivity index (SI=218.2), while it also displayed increased cytotoxicity (CC₅₀=10.9 μM). In contrast to compound 11, moving the 4′-NO₂ group to 5′-position (12) led to a slight loss of activity (IC₅₀=0.31 μM). Nevertheless, it still showed the same level of potency compared to niclosamide. Removal of the 2′-Cl group of niclosamide yielded compound 13 with enhanced potency (IC₅₀=0.11 μLM) and selectivity index (SI=246.6). Excitingly, compound 14 with 2′-Cl-4′-CF₃-aniline moiety showed significantly increased potency (IC₅₀=0.08 μM) and decreased cytotoxicity (CC₅₀=35.0 μM), resulting in a high selectivity index (SI=437.5). Compounds 15 with 3′,4′-difluoro substitution and 16 with 3′,5′-bis(trifluoromethyl) substitution exhibited potent anti-HAdV activities with IC₅₀ values of 0.27 μM and 0.06 μM, respectively. These two compounds also showed cytotoxicity of CC₅₀=8.0 μM and 4.1 μM, respectively; and consequently no obvious improvement on their selectivity index was observed (SI=29.6 and 68.3, respectively). Remarkably, compound 17 with 3′-F-5′-CF₃-aniline moiety possessed improved potency against HAdV (IC₅₀=0.18 μM), meanwhile it showed significantly decreased cytotoxicity (CC₅₀=120.0 μM), with a high selectivity index (SI=666.7). These explorations described above indicated that the introduction of fluoro and trifluoromethyl groups at the proper positions on the aniline ring was beneficial for antiviral potency.

Considering its potent activity and low cytotoxicity, the effect of the substitution on the salicylic ring was explored while retaining the 3′-F-5′-CF₃-aniline moiety of compound 17. Removal of the 5-Cl group (18) or replacement of the 5-Cl group with 5-methyl group (19) led to a slight loss of potency (IC₅₀=1.07 μM and 0.65 μM, respectively) and increased cytotoxicity (CC₅₀=16.3 μM and 15.2 μM, respectively), compared to compound 17. Moving the 5-Cl group to 4-position (20) maintained the same level of potency (IC₅₀=0.11 μM), with increased cytotoxicity (CC₅₀=20.6 μM) and decreased selectivity index (SI=186.8). However, removal of the 2-OH group resulted in a significant loss of potency (IC₅₀=5.6 μM). These results suggested the 2-phenolic hydroxyl group at the salicylic part can benefit salicylanilide derivatives and maintain their anti-HAdV activities.

TABLE 1
Inhibition of HAdV in Plaque Assay, Cytotoxicity and
Selectivity Index for Compounds 10-21

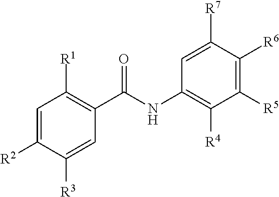

| | | | | | | | | Plaque Assay^{a} | | | |
|---|---|---|---|---|---|---|---|---|---|---|---|
| | | | | | | | | (%) | | | Selectivity |
| | | | | | | | | Inhibition | | | index |
| Compd | R¹ | R² | R³ | R⁴ | R⁵ | R⁶ | R⁷ | (10 μM) | IC₅₀ (μM) | CC₅₀ (μM)^{b} | (SI)^{c} |
| 6 | OH | H | Cl | Cl | H | NO₂ | H | 100 ± 0 | 0.6 ± 0.05 | 22.9 ± 9.8 | 38.2 |
| 10 | OH | H | Cl | H | Cl | NO₂ | H | 100 ± 0 | 1.00 ± 0.37 | 15.1 ± 0.5 | 15.1 |
| 11 | OH | H | Cl | F | H | NO₂ | H | 98.7 ± 1.9 | 0.05 ± 0.01 | 10.9 ± 0.5 | 218.2 |
| 12 | OH | H | Cl | F | H | H | NO₂ | 100 ± 0 | 0.31 ± 0.10 | 8.1 ± 3.2 | 26.2 |
| 13 | OH | H | Cl | H | H | NO₂ | H | 98.0 ± 2.8 | 0.11 ± 0.01 | 27.1 ± 0.1 | 246.6 |
| 14 | OH | H | Cl | Cl | H | CF₃ | H | 100 ± 0 | 0.08 ± 0.00 | 35.0 ± 3.7 | 437.5 |
| 15 | OH | H | Cl | H | F | F | H | 100 ± 0 | 0.27 ± 0.02 | 8.0 ± 0.8 | 29.6 |
| 16 | OH | H | Cl | H | CF₃ | H | CF₃ | 100 ± 0 | 0.06 ± 0.00 | 4.1 ± 1.8 | 68.3 |
| 17 | OH | H | Cl | H | F | H | CF₃ | 100 ± 0 | 0.18 ± 0.01 | 120.0 ± 33.6 | 666.7 |
| 18 | OH | H | H | H | F | H | CF₃ | 98.7 ± 1.9 | 1.07 ± 0.09 | 16.3 ± 8.6 | 15.2 |
| 19 | OH | H | Me | H | F | H | CF₃ | 98.0 ± 2.8 | 0.65 ± 0.43 | 15.2 ± 11.0 | 23.3 |
| 20 | OH | Cl | H | H | F | H | CF₃ | 96.8 ± 1.9 | 0.11 ± 0.05 | 20.6 ± 1.0 | 186.8 |
| 21 | H | H | Cl | H | F | H | CF₃ | 99.3 ± 0.9 | 5.61 ± 0.37 | 38.6 ± 1.7 | 6.9 |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
The results represent means ± SD of triplicate samples from three independent experiments.

TABLE 2
Inhibition of HAdV in Plaque Assay, Cytotoxicity and
Selectivity Index for Compounds 30-54

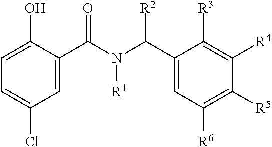

| | | | | | | | Plaque Assay^{a} | | | |
|---|---|---|---|---|---|---|---|---|---|---|
| | | | | | | | (%) | | | Selectivity |
| | | | | | | | Inhibition | | | index |
| Compd | R¹ | R² | R³ | R⁴ | R⁵ | R⁶ | (10 μM) | IC₅₀ (μM) | CC₅₀ (μM)^{b} | (SI)^{c} |
| 30 | H | H | Cl | H | NO₂ | H | 100 ± 0 | 1.40 ± 0.11 | 51.4 ± 3.7 | 5.5 |
| 31 | H | H | Cl | H | F | H | 97.1 ± 4.1 | 2.56 ± 0.06 | 30.9 ± 10.2 | 12.1 |
| 32 | H | H | Cl | H | Cl | H | 99.2 ± 0.4 | 1.73 ± 0.51 | 30.9 ± 13.6 | 17.9 |
| 33 | H | H | F | H | F | H | 74.7 ± 6.9 | NT^{d} | NT | NT |
| 34 | H | H | H | F | F | H | 37.2 ± 23.4 | NT | NT | NT |
| 35 | H | H | H | F | H | CF₃ | 98.1 ± 1.4 | 2.40 ± 0.05 | 36.3 ± 7.9 | 15.1 |
| 36 | H | H | H | F | CF₃ | H | 99.0 ± 1.5 | 1.40 ± 0.02 | 22.3 ± 7.2 | 15.9 |
| 37 | H | H | F | H | CF₃ | H | 98.8 ± 0.8 | 1.32 ± 0.04 | 45.6 ± 7.3 | 34.6 |
| 38 | H | H | H | H | Cl | H | 97.4 ± 3.7 | 3.57 ± 1.43 | 27.1 ± 1.1 | 7.6 |
| 39 | H | H | H | Cl | H | H | 72.0 ± 1.5 | NT | NT | NT |
| 40 | H | H | H | H | F | H | 72.0 ± 3.0 | NT | NT | NT |
| 41 | Me | H | H | H | F | H | 0.0 ± 0.0 | NT | NT | NT |
| 42 | H | H | H | H | CF₃ | H | 99.6 ± 0.2 | 2.59 ± 0.42 | 23.9 ± 9.2 | 9.2 |
| 43 | H | H | H | H | NO₂ | H | 99.5 ± 0.7 | 6.56 ± 0.47 | 175.0 ± 43.9 | 26.7 |
| 44 | H | H | H | H | CH₃ | H | 96.6 ± 4.0 | 8.12 ± 2.16 | 87.7 ± 4.7 | 10.8 |
| 45 | H | H | H | H | H | H | 12.7 ± 2.9 | NT | NT | NT |
| 46 | H | (R)-Me | H | H | H | H | 100 ± 0 | NT | 84.6 ± 20.5 | NT |
| 47 | H | (S)-Me | H | H | H | H | 100 ± 0 | 2.54 ± 0.1 | 199.3 ± 8.7 | 78.5 |
| 48 | H | (R)-Me | H | H | Cl | H | 100 ± 0 | NT | 31.1 ± 3.2 | NT |
| 49 | H | (R)-Me | H | H | F | H | 100 ± 0 | 3.02 ± 0.07 | >200 | 66.2 |
| 50 | H | (R)-Me | H | H | OMe | H | 33.5 ± 14.8 | NT | NT | NT |

| | | | | | |
|---|---|---|---|---|---|
| 51 | 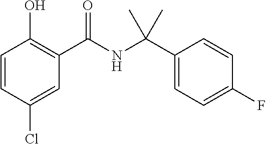 [SMILES: CC(C)(NC(=O)c1cc(Cl)ccc1O)c1ccc(F)cc1] | 100 ± 0 | 3.46 ± 0.88 | >200 | 57.8 |
| 52 | 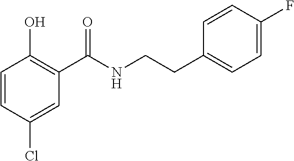 [SMILES: O=C(NCCc1ccc(F)cc1)c1cc(Cl)ccc1O] | 98.7 ± 1.8 | 3.94 ± 1.38 | 154.1 ± 13.1 | 39.1 |
| 53 | 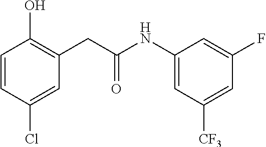 [SMILES: O=C(Cc1cc(Cl)ccc1O)Nc1cc(F)cc(C(F)(F)F)c1] | 91.0 ± 3.6 | 4.92 ± 0.01 | 23.7 ± 1.5 | 4.8 |
| 54 | 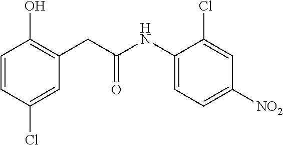 [SMILES: O=C(Cc1cc(Cl)ccc1O)Nc1ccc([N+](=O)[O-])cc1Cl] | 0.0 ± 0.0 | NT | NT | NT |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
^{d}NT: not tested.
The results represent means ± SD of triplicate samples from three independent experiments.

Next, attention was focused on the amide linker region. In order to improve the flexibility of salicylanilide derivatives, insertion of one or two additional carbon atoms between two aromatic rings was attempted, and compounds 30-54 were designed, synthesized, and evaluated (Table 2). First, the aniline moiety was replaced with various substituted benzylamines to yield a series of N-benzylsalicylamide derivatives (30-51), and most of these compounds showed potency at the concentration of 10 μM, with IC₅₀ values ranging from 1 to 10 μM. In general, disubstituted benzylamine derivatives (30-32 and 35-37) were more potent than monosubstituted benzylamine derivatives (38-40 and 42-44). Unexpectedly, difluoro substitution (33 and 34) reduced the potency, especially 3,4-difluoro substituted derivative 34 with no inhibition against HAdV at 10 μM, and inconsistent with the trend observed before. Methylation of amide (41) resulted in a complete loss of potency (0% at 10 μM) in contrast to 40. Compound 45 with unsubstituted benzylamine moiety displayed no inhibitory activity against HAdV up to 10 μM. Introduction of one or two methyl groups at the benzylic site produced compounds 46-51, and all these compounds except compound 50 with (R)-4-methoxy-α-methylbenzylamine moiety fully inhibited HAdV plaque formation at the concentration of 10 μM. Interestingly, compounds 47 with (S)-α-methylbenzylamine moiety, 49 with (R)-4-fluoro-α-methylbenzylamine moiety and 51 with α, α-dimethyl-4-fluorobenzylamine showed similar potency (IC₅₀=2.54 μM, 3.02 μM and 3.46 μM, respectively) and very low cytotoxicity (CC₅₀=199.3 μM, >200 μM and >200 μM, respectively), along with similar selectivity index as well (SI=78.5, 57.8 and 39.1, respectively). Insertion of two carbon atoms between the nitrogen atom of the amide and the aromatic ring (52) also retained the potency with an IC₅₀ of 3.94 μM, while it showed low cytotoxicity (CC₅₀=154.1 μM) and a similar selectivity index (SI=39.1) compared to niclosamide. Inserting one carbon between the amide carbonyl and the aromatic ring yielded two derivatives 53 and 54 with entirely different inhibitory activities against HAdV. Intriguingly, compound 53 with 3′-F-5′-CF₃-aniline moiety exhibited potency with an IC₅₀ of 4.92 μM, while 54 with 2′-Cl-4′-NO₂-aniline moiety showed no inhibitory activity against HAdV at 10 μM. Due to the weak potency, no more optimization efforts on this series of compounds were further pursued.

TABLE 3
Inhibition of HAdV in Plaque Assay, Cytotoxicity and
Selectivity Index for Compounds 56-72

| | | 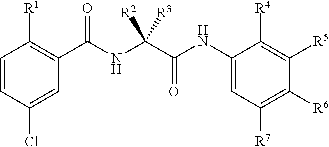 | | | | | | | | | |
|---|---|---|---|---|---|---|---|---|---|---|---|
| | | | | | | | | Plaque Assay^{a} | | | |
| | | | | | | | | (%) | | | Selectivity |
| | | | | | | | | Inhibition | | | index |
| Compd | R¹ | R² | R³ | R⁴ | R⁵ | R⁶ | R⁷ | (10 μM) | IC₅₀ (μM) | CC₅₀ (μM)^{b} | (SI)^{c} |
| 56 | OH | H | H | Cl | H | NO₂ | H | 100 ± 0 | 2.43 ± 0.59 | 129.2 ± 34.7 | 53.2 |
| 57 | OH | Me | H | Cl | H | NO₂ | H | 97.4 ± 2.7 | 3.85 ± 0.04 | 100.1 ± 13.3 | 26.0 |
| 58 | OH | iso-Pr | H | Cl | H | NO₂ | H | 100 ± 0 | 0.45 ± 0.06 | 200.0 ± 1.9 | 444.4 |
| 59 | OH | Bn | H | Cl | H | NO₂ | H | 100 ± 0 | NT | 15.2 ± 2.1 | NT |
| 60 | OH | H | Bn | Cl | H | NO₂ | H | 100 ± 0 | 0.30 ± 0.00 | 77.9 ± 10.2 | 259.7 |
| 61 | OH | iso-Bu | H | Cl | H | NO₂ | H | 17.4 ± 12.3 | NT | NT | NT |
| 62 | OH | sec-Bu | H | Cl | H | NO₂ | H | 99.0 ± 0.3 | 0.16 ± 0.00 | 20.2 ± 6.9 | 127.6 |
| 63 | OH | 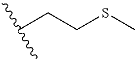 [SMILES: *CCSC] | H | Cl | H | NO₂ | H | 34.8 ± 16.9 | NT | NT | NT |
| 64 | OH | 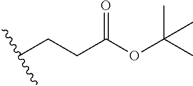 [SMILES: *CCC(=O)OC(C)(C)C] | H | Cl | H | NO₂ | H | 97.3 ± 0.3 | 0.67 ± 0.11 | 22.9 ± 5.2 | 34.2 |
| 65 | OH | iso-Pr | H | H | CF₃ | H | CF₃ | 99.5 ± 0.7 | 0.40 ± 0.09 | 30.9 ± 0.3 | 77.4 |
| 66 | OH | iso-Pr | H | Cl | H | CF₃ | H | 24.8 ± 12.3 | NT | NT | NT |
| 67 | OH | iso-Pr | H | H | F | H | CF₃ | 95.8 ± 5.9 | 0.66 ± 0.03 | 43.0 ± 14.4 | 65.2 |
| 68 | OH | iso-Pr | H | H | H | F | F | 98.3 ± 2.4 | 2.96 ± 0.50 | 95.0 ± 18.7 | 32.1 |
| 69 | OH | Bn | H | H | CF₃ | H | CF₃ | 99.0 ± 1.4 | 0.40 ± 0.02 | 17.9 ± 1.1 | 44.9 |
| 70 | 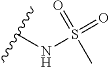 [SMILES: *NS(C)(=O)=O] | iso-Pr | H | Cl | H | NO₂ | H | 99.1 ± 1.2 | 0.68 ± 0.08 | 174.5 ± 12.1 | 256.0 |
| 71 | 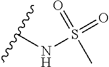 [SMILES: *NS(C)(=O)=O] | Bn | H | Cl | H | NO₂ | H | 88.9 ± 15.7 | NT | NT | NT |
| 72 | 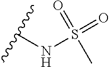 [SMILES: *NS(C)(=O)=O] | H | H | Cl | H | NO₂ | H | 99.6 ± 5.5 | 9.65 ± 0.01 | 141.1 ± 13.5 | 14.6 |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
dNT: not tested.
The results represent means ± SD of triplicate samples from three independent experiments.

Amino acid linkers were widely used to tune the flexibility and improve the pharmacokinetic profiles for those compounds with two aromatic rings linked by a simple amide bond (Xu et al., Org. Biomol. Chem. 2014, 12, 3721-3734). To explore the effect of introducing various α-amino acid linkers, compounds 56-72 were prepared and evaluated as shown in Table 3. Introducing different substitution on the linker moiety yielded compounds 56-64 while retaining the salicylic and aniline moieties of niclosamide. Use of an unsubstituted glycine linker (56) reduced the potency (IC₅₀=2.43 μM) as well as cytotoxicity (CC₅₀=129.2 μM) compared to niclosamide. The linker with (S)-methyl group (57) was similar in potency (IC₅₀=3.85 μM) and cytotoxicity (CC₅₀=100.1 μM) to compound 56. Encouragingly, compound 58 with (S)-isopropyl substitution displayed potent activity (IC₅₀=0.45 μM) similar to niclosamide, with decreased cytotoxicity (CC₅₀=200 μM) and significantly improved selectivity index (SI=444.4). Compound 60 with (R)-benzyl substitution also showed improved potency (IC₅₀=0.30 μM) and diminished cytotoxicity (CC₅₀=77.9 μM), with a high SI value of 259.7. However, its enantiomer 59 with (S)-benzyl substitution exhibited increased cytotoxicity (CC₅₀=15.2 μM). Linkers with (S)-isobutyl (61) and (S)-2-(methylthio)ethyl (63) displayed no significant inhibitory activity at the concentration of 10 μM, while compounds 62 with (S)-sec-butyl and 64 with (S)-3-(tert-butoxy)-3-oxopropyl maintained the same level of activity (IC₅₀=0.16 μM and 0.67 μM, respectively) and cytotoxicity (CC₅₀=20.2 μM and 22.9 μM, respectively) compared to niclosamide, with SI values of 127.6 and 34.2, respectively. Considering the potent activity and low cytotoxicity of compound 58, next the effect of the substitution on the aniline ring was investigated, using L-valine as a linker. Compounds 65 with 3′,5′-bis(trifluoromethyl) substitution and 67 with 3′-fluoro-5′-trifluoromethyl substitution retained similar potency (IC₅₀=0.40 μM and 0.66 μM, respectively) to compound 58, with increased cytotoxicity (CC₅₀=30.9 μM and 43.0 μM, respectively). Interestingly, 2′-chloro-4′-trifluoromethyl substitution (66) led to a complete loss of potency at 10 μM, while compounds 68 with 3′,4′-difluoro substitution retained some activity (IC₅₀=2.96 μM). Compared to compound 65, use of L-phenylalanine linker (69) maintained the same level of potency (IC₅₀=0.40 μM), with a slight increase in cytotoxicity (CC₅₀=17.9 μM). Substitution of 2-phenolic hydroxyl at the salicylic part with 2-methylsulfonamido yielded compounds 70-72 with different amino acid linkers. Of these, compound 70 with a L-valine linker displayed similar potency (IC₅₀=0.68 μM) and lower cytotoxicity (CC₅₀=174.5 μM) to compound 58, with a high selectivity index (SI=256.0). For the comparison using the same in vitro assay protocols, the antiviral activity and safety of these niclosamide derivatives demonstrated to be significantly better than those of cidofovir (IC₅₀=24.06±5.9 μM; CC₅₀=50.06±9.8 μM; SI=7.5), the drug of current choice for the treatment of HAdV infections.

Those compounds with high selectivity index (SI>100) were selected for further evaluation in entry assay, using human A549 epithelial cells infected with HAdV-GFP in presence of the candidate compound and incubated for 48 h. As shown in Table 4, most of the selected molecules inhibited expression of the HAdV-GFP transgene in a significant way (>90%) like niclosamide (6). Compounds 58 and 60 showed lower percentage of inhibition, 81.8% and 68.6%, respectively, but they were selected based on their low IC₅₀ in the plaque assay (0.45 μM and 0.30 μM, respectively). As for their IC₅₀ in entry assay, it was observed that derivatives whose percentage of inhibition was higher than 90% also showed a low IC₅₀ value, ranging between 0.2 μM and 2.8 μM. Compounds 58 and 60 were the only ones whose IC₅₀ values were higher than this range (11.20 μM and 9.70 μM, respectively). These results provided us with preliminary information regarding their potential mechanisms of inhibition since the effect of these compounds in a step between HAdV attachment to its cellular receptors and HAdV DNA import into the cell nucleus would result in decreased GFP expression.

TABLE 4
Inhibition of HAdV in Entry Assay for Selected
Compounds with High Selectivity Index

| | (%) Inhibition Entry | IC₅₀ Entry | CC₅₀ |
|---|---|---|---|
| Compound | Assay (50 μM)^{a} | Assay (μM) | (μM) |
| 6 | 100.0 ± 0.0 | 1.22 ± 0.44 | 22.9 ± 9.8 |
| 11 | 92.4 ± 0.2 | 1.57 ± 0.97 | 10.9 ± 0.5 |
| 13 | 100.0 ± 0.0 | 2.71 ± 0.07 | 27.1 ± 0.1 |
| 14 | 100.0 ± 0.0 | 1.67 ± 0.25 | 35.0 ± 3.7 |
| 17 | 100.0 ± 0.0 | 2.23 ± 0.58 | 120.0 ± 30.6 |
| 20 | 100.0 ± 0.0 | 0.26 ± 0.09 | 20.6 ± 1.0 |
| 58 | 81.8 ± 8.6 | 11.20 ± 3.60 | 200.0 ± 1.9 |
| 60 | 68.6 ± 10.7 | 9.70 ± 3.80 | 77.9 ± 10.2 |
| 62 | 98.2 ± 0.5 | 2.50 ± 0.22 | 20.2 ± 6.9 |
| 70 | 99.1 ± 1.2 | 1.38 ± 0.10 | 174.5 ± 12.1 |

^{a}Percentage of control HAdV5-GFP inhibition at 50 μM and inhibitory concentration 50% (IC₅₀) at high MOI in an entry assay using the A549 cell line.
^{b}Cell cytotoxic concentration 50% (CC₅₀) using A549 cell line.

The next step was evaluation of the effect of these selected compounds on virus replication using a virus burst assay which measures the production of virus particles. In this assay, A549 cells were infected with the HAdV-5 wild-type virus, and the TCID₅₀ values of an infection in the presence and absence of the selected compounds were calculated. As summarized in Table 5, the overall reductions in virus yield varied from as low as 1.8-fold for compound 11 to as high as 989-fold for compound 17.

TABLE 5
Virus Yield Reduction for Selected Compounds
with High Selectivity Index

| | Virus Yield Reduction |
|---|---|
| Compound | (fold)^{a} |
| 6 | 82 ± 35 |
| 11 | 1.8 ± 0.3 |
| 13 | 137 ± 71 |
| 14 | 175 ± 33 |
| 17 | 989 ± 361 |
| 20 | 385 ± 273 |
| 58 | 213 ± 78 |
| 60 | 528 ± 100 |
| 62 | 10.0 ± 3.8 |
| 70 | 1.0 ± 0.4 |

^{a}Fold-reduction in virus yield as the ratio of particles produced in the presence of DMSO divided by the yield in the presence of each selected compound at the concentration based on CC₅₀ (11 was tested at 2 μM, 6, 13, 20 and 62 at 5 μM, 14 at 10 μM, 60 at 20 μM and 17, 58 and 70 at 50 μM). Virus Yield Reduction assay used A549 cell line and the MOI of HAdV was 100 vp/cell. The results represent means ± SD of triplicate samples from three independent experiments.

To gain further mechanistic understanding for inhibition the time dependence of these analogues addition on their ability to inhibit HAdV infection was measured as an alternative step toward identifying the specific stage of HAdV replicative cycle that was inhibited by these compounds. HAdV has been shown to be internalized within 5 min after binding, to escape the endosome after 15 min, and to attach to the nuclear pore complex after 35-45 min (Greber et al., Cell. 1993, 75, 477-486). It was observed that all compounds exhibited a time-dependent decrease in inhibitory activity although with different patterns (FIG. 2). Compounds 13, 20, 62 and 70 inhibited infection in more than a 50% when they were added either at the beginning of the 60 min incubation at 4° C. (−60 min) or at 120 min post-infection (p.i.). Compounds 11 and 14 showed abrupt decreases in their antiviral activity at early time points. Compound 14 lost 20% of inhibition when added between 5 and 10 min p.i. while compound 11 lost 34% of inhibition when added between 20 and 40 p.i. In contrast, compound 60 showed a constant decrease from the beginning of the 60 min incubation at 4° C. losing the 50% inhibition when added after 20 min p.i. and compounds 17 and 58 showed a little bit more but also constant decrease in inhibition from the beginning of the 60 min incubation at 4° C. showing less than 50% inhibition after the addition at the moment of the infection (0 min) (FIG. 2). Collectively, the results indicated that these analogues were inhibiting an early step in virus entry occurring after cell attachment and also that the observed inhibition of HAdV infection were due to an effect on virus entry and not to an indirect effect on GFP expression.

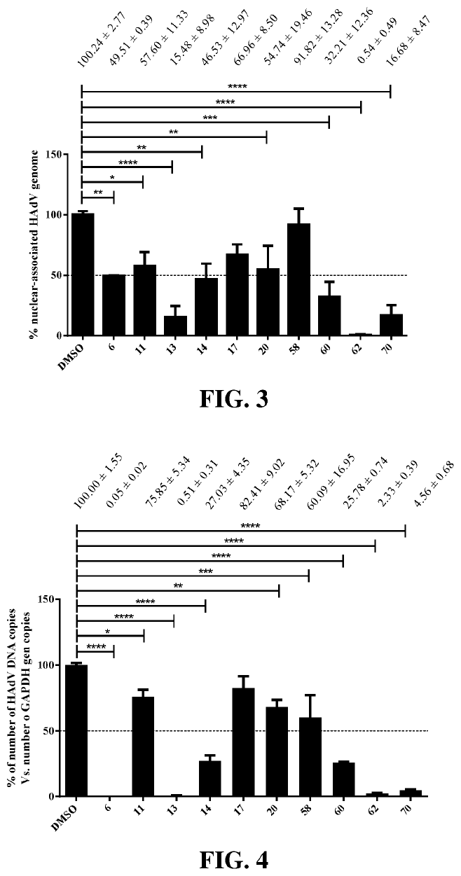

Impact on HAdV Entry. The HAdV cell entry pathway is a coordinated multi-stage process in which following attachment and internalization of the HAdV particle, the exposure of protein VI provokes endosome lysis and subsequent endosomal escape of virions into cytoplasm. Then, the partially uncoated HAdV capsid is translocated along microtubules towards the nuclear pore complex where further disassembly occurs and the HAdV genome is finally delivered into the cell nucleus. If a compound blocks any step of the HAdV entry, this inhibitory effect will be reflected in the number of HAdV genomes that reach the host nucleus after a synchronized infection. To further validate whether these compounds could inhibit any of the steps in the entry pathway, an assay was performed to quantitatively measure the HAdV genome accessibility to the nucleus. As shown in FIG. 3, cells treated with compounds 13, 62 and 70 showed significant (p≤0.0001) reductions in the amount of HAdV genomes isolated from the nucleus versus those treated with DMSO at 45 min post infection, consistent with the corresponding IC₅₀ values obtained from the entry assay. Compounds 11, 14, 20 and 60 partially inhibited the accessibility of HAdV genomes to the nucleus while compounds 17 and 58 showed no significant differences in the amount of nuclear-associated HAdV genomes versus the control group. These results indicated that compounds 13, 62 and 70 exerted their main antiviral activity in the early steps of the HAdV replication cycle while compounds 17 and 58 inhibited later steps after the HAdV genome is imported into the nucleus.

Impact on HAdV Replication. To evaluate HAdV DNA replication efficiency quantitative real-time PCR (qPCR) was performed in the presence of these selected compounds in a 24 h assay to avoid the influence of subsequent rounds of infection occurring 32-36 h post infection. qPCR was used to quantify the newly synthesized HAdV DNA copies in a single round of infection as a measure of DNA replication efficiency. As shown in FIG. 4, compounds 13, 62 and 70 significantly inhibited HAdV-5 DNA replication by more than 90% in the same way as that of niclosamide, while compounds 11, 17, 20 and 58 showed low inhibition on DNA replication when compared to a control treated with the same concentration of DMSO. Compounds 14 and 60 which did not show a significant inhibition in the nuclear-associate genomes assay exhibited a significant (p≤0.0001) inhibitory activity, indicating that they may be targeting the HAdV DNA replication process.

In summary, compounds 13, 62 and 70 caused a significant decrease in HAdV DNA replication, and these activities were possibly attributed to their potent inhibitory effects during the HAdV entry pathway. Compounds 17 and 58 did not show obvious inhibition against either during HAdV entry or HAdV DNA replication, indicating that their mechanism of HAdV inhibition may be related to later steps of the HAdV infection cycle, such as the viral protein maturation, viral particles assembly, or release processes. Compounds 14 and 60 showed moderate potency against HAdV-5 DNA replication, meanwhile partially blocking the accessibility of HAdV genomes to the nucleus. Thus, it is hard to conclude whether these two compounds had direct impacts on HAdV replication by interfering with a protein involved in this process or impacting transcription of the immediate early genes, which is essential for subsequent DNA replication. Despite their partial inhibition on the accessibility of HAdV genomes to the nucleus, compounds 11 and 20 did not display significant effects on HAdV-5 DNA replication. Based on the current data, it is still unable to determine at what stage compounds 11 and 20 were acting, and more studies are needed to elucidate their exact mechanisms of action.

In conclusion, a series of salicylamide derivatives was optimized to identify potent inhibitors of HAdV infection. Of these, nine compounds (11, 13, 14, 17, 20, 58, 60, 62 and 70) showed improved anti-HAdV activities with nanomolar to submicromolar IC₅₀ values and high selectivity indexes (SI>100), indicating better safety windows, compared to niclosamide. Moreover, our preliminary mechanistic assays demonstrate that compounds 13, 62 and 70 exert their activities in the HAdV entry pathway, while compounds 11, 17 (JMX0312), 20 and 58 (JMX0281) possibly inhibit later steps after DNA replication. With respect to compounds 14 and 60, they seem to be targeting the HAdV DNA replication process although more studies are imperative to elucidate their specific mechanisms of action. Given the broad antiviral activities of niclosamide, these derivatives also offer great therapeutic potential to be developed for other viral infections such as Zika, Ebola, HIV, and hepatitis C virus. The efficacy and safety of these compounds can be assessed in the animal models of viral infections.

The general synthesis of new derivatives with chemical optimizations on the amino group is outlined in Scheme 5. Reduction of the nitro group of niclosamide 73 with zinc dust provided amine 76, which was then converted into acetamide 77 via acetylation and hydrolysis. Reductive amination of amine 76 with various aldehyde or ketone afforded a series of substituted N-(4-amino-2-chlorophenyl)-5-chloro-2-hydroxybenzamide analogues 78-86, 88-92, 94-114, 116, 117,

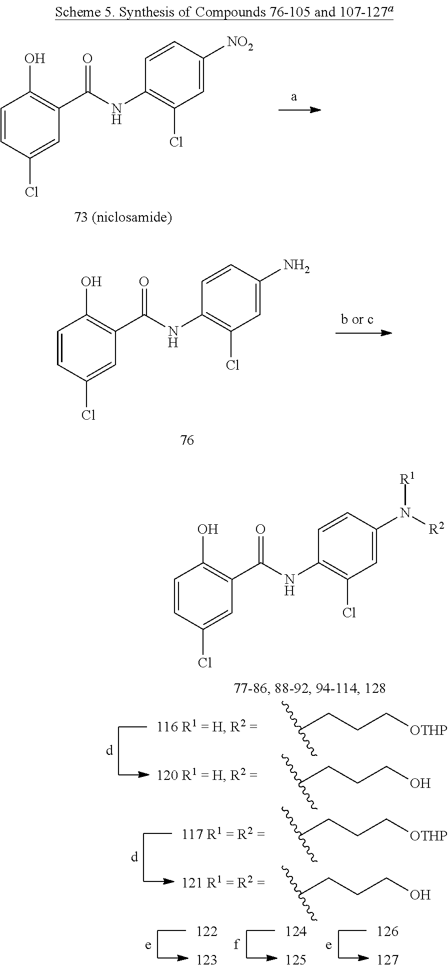

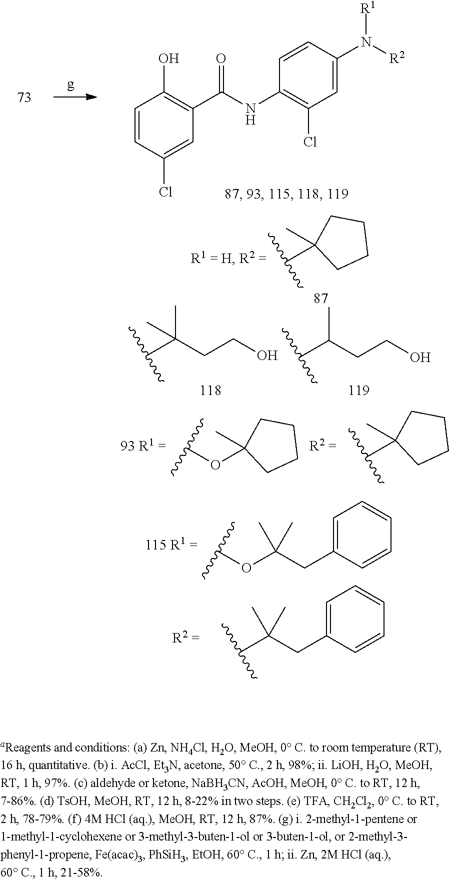

122, 124, 126 and 128. Alcohol analogues 120 and 121 were accessed from tetrahydropyran (THP) ethers 116 and 117, respectively, via acidic depyranylation. Boc deprotection of 122, 124 and 126 under acidic conditions afforded derivatives 123, 125 and 127, respectively. Direct hydroamination of niclosamide with different olefins provided the corresponding amines 87, 118 and 119 while N,O-alkylated adducts 93 and 115 were afforded as by-products without undergoing further N—O bond reduction under the same conditions (Gui et al. Science. 2015, 348, 886-891).

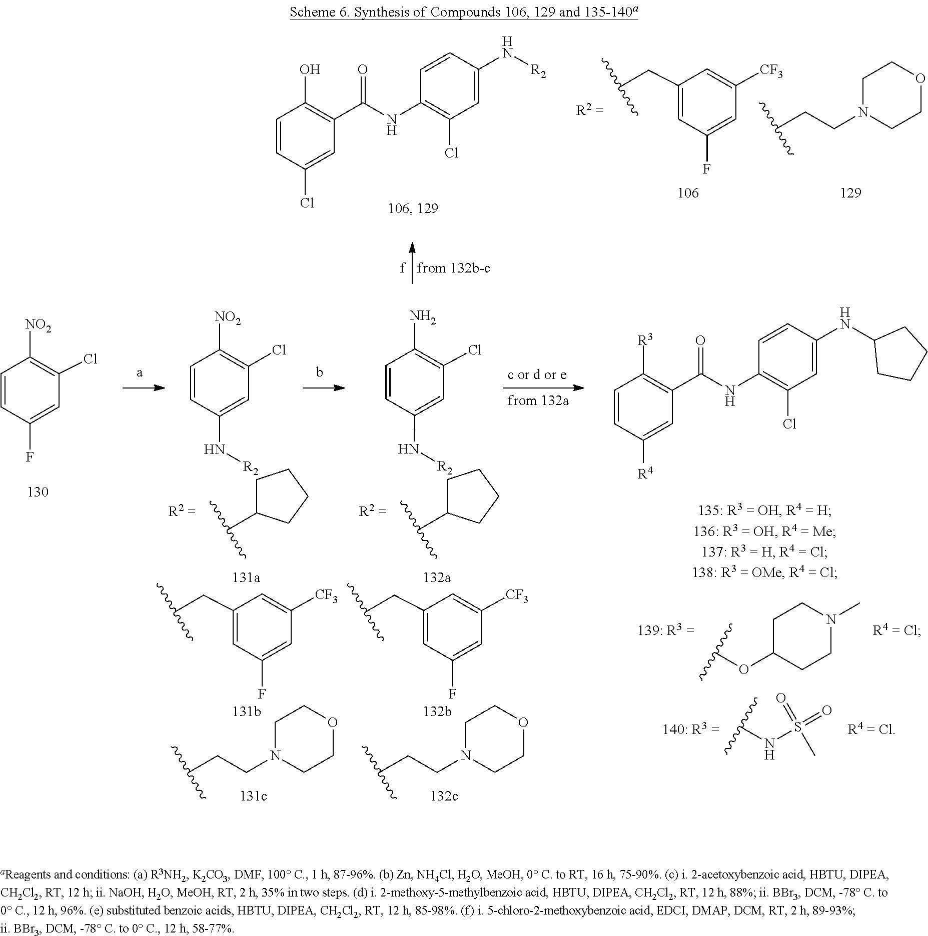

As shown in Scheme 6, substitution of 2-chloro-4-fluoro-1-nitrobenzene with the corresponding amines gave the intermediates 131a-c, which were then reduced to generate amines 132a-c. With intermediates 132a-c in hand, the desired compounds were readily synthesized by a series of condensations and deprotections. To this end, condensation of 132a with 2-acetoxybenzoic acid was followed by hydrolysis to afford compound 135. Compound 136 was accessed under the same coupling conditions, by condensation of 132a with 2-methoxy-5-methylbenzoic acid, followed by the removal of methyl group. Direct coupling of 132a with different substituted benzoic acids afforded derivatives 137-140. 5-Chloro-2-methoxybenzoic acid was condensed with 132b-c followed by demethylation with BBr₃ to provide compounds 106 and 129, respectively.

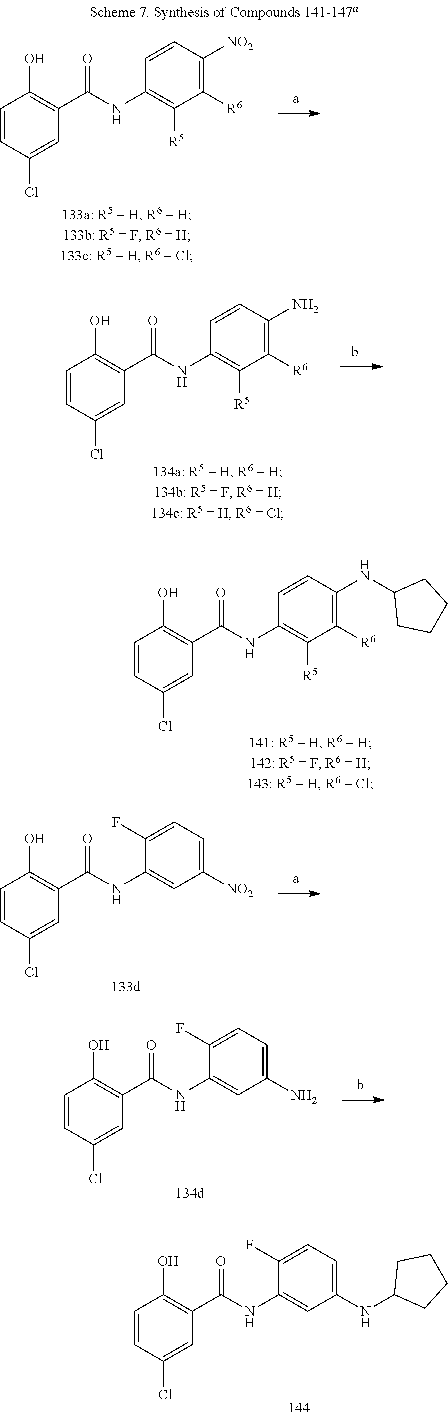

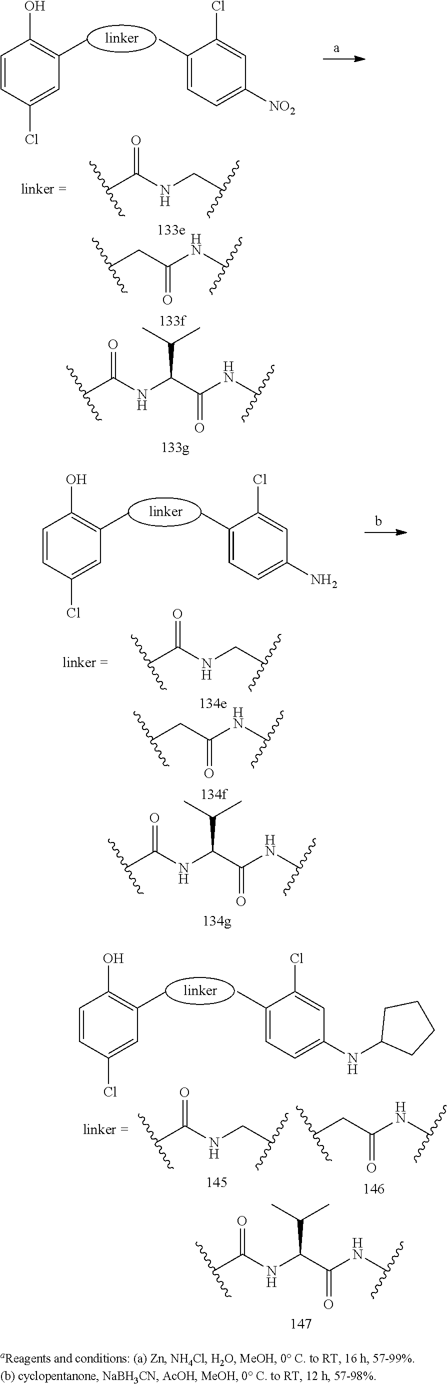

Compounds 141-147 were prepared according to the general synthesis outlined in Scheme 3. Known nitro derivatives 133a-g were reduced to afford anilines 134a-g, which were then converted into compounds 141-147 via reductive amination with cyclopentanone (Xu et al., J. Med. Chem. 2020, 63, 3142-3160).

In Vitro Evaluation of Human Adenovirus Inhibition. All newly synthesized compounds were first screened in plaque assay at the concentration of 10 μM, quantified as the percentage of HAdV plaque formation inhibition, and the active compounds (inhibition >90%) were further evaluated to characterize their anti-viral activity (IC₅₀) in plaque assay and their cytotoxicity (CC₅₀ values). Based on the potent anti-HAdV activity of N-cyclopentyl substituted derivative 78, we first investigated the effect of different alkyl substitutions on the amino moiety (Table 6). Excitingly, N-alkylated (79-88), N,N-alkylated (89-92) and N,O-alkylated (93) derivatives were all active at the concentration of 10 AM, with full inhibition against HAdV plaque formation. In contrast to lead compound 78, N-ethyl derivative 79 and N-n-propyl derivative 80 showed decreased potency (IC₅₀=2.18 μM and 5.04 μM, respectively) while compounds 81 with isopropyl and 82 with isobutyl maintained the same level of potency (IC₅₀=0.88 μM and 0.89 μM, respectively). Long-length branched alkyl groups (83 and 84) were also well tolerated, and compound 83 with octan-2-yl moiety showed very potent anti-HAdV activity with an IC₅₀ value of 0.32 μM. Nevertheless, these derivatives also displayed increased cytotoxicity (CC₅₀=11.0˜60.0 μM) with no improved selectivity index (SI=4.5˜73.4) compared to lead compound 78. Introduction of cycloalkyl groups produced compounds 85-88, and N-cyclopropylmethyl substitution (85), N-cyclopentylmethyl substitution (86) and N-cyclohexyl substitution (88) displayed slightly decreased activity (IC₅₀=1.38 μM, 1.24 μM and 1.34 μM, respectively) and increased cytotoxicity with different degrees (CC₅₀=14.4 μM, 21.5 μM and 68.3 μM, respectively) as compared with N-cyclopentyl substitution (78). Remarkably, compound 87 with 1-methylcyclopentyl moiety possessed improved potency against HAdV (IC₅₀=0.27 μM); meanwhile, it exhibited significantly decreased cytotoxicity (CC₅₀=156.8 μM) with a very high selectivity index (SI=580.7). N,N-Dialkyl substituted derivatives (89-92) displayed submicromolar to low micromolar potency against.

TABLE 6
Inhibition of HAdV in Plaque Assays, Cytotoxicity and
Selectivity Index for Compounds 76-93

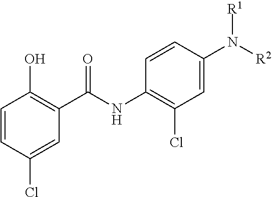

| | | | Plaque Assay^{a} | | | Selectivity |
|---|---|---|---|---|---|---|
| | | | Inhibition | | | index |
| Compd | R¹ | R² | (%, 10 μM) | IC₅₀ (μM) | CC₅₀ (μM)^{b} | (SI)^{c} |
| 73 | NA | | 100 ± 0 | 0.6 ± 0.05 | 22.9 ± 9.8 | 38.2 |
| 76 | H | H | 0 ± 0 | NT^{d} | NT | NT |
| 77 | H | 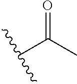 [SMILES: *C(C)=O] | 0 ± 0 | NT | NT | NT |
| 78 | H | 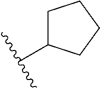 [SMILES: *C1CCCC1] | 100 ± 0 | 0.5 ± 0.1 | 92.9 ± 7.1 | 185.8 |
| 79 | H | 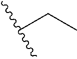 [SMILES: *CC] | 100 ± 0 | 2.18 ± 0.23 | 30.2 ± 4.1 | 13.9 |
| 80 | H | 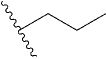 [SMILES: *CCC] | 100 ± 0 | 5.04 ± 0.01 | 22.5 ± 10.6 | 4.5 |
| 81 | H | 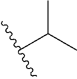 [SMILES: *C(C)C] | 100 ± 0 | 0.88 ± 0.41 | 11.0 ± 1.4 | 12.5 |
| 82 | H | 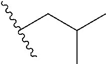 [SMILES: *CC(C)C] | 100 ± 0 | 0.89 ± 0.20 | 60.0 ± 15.0 | 67.4 |
| 83 | H | 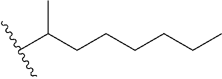 [SMILES: *C(C)CCCCCC] | 100 ± 0 | 0.32 ± 0.00 | 23.5 ± 3.0 | 73.4 |
| 84 | H | 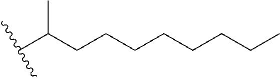 [SMILES: *C(C)CCCCCCCC] | 100 ± 0 | 1.21 ± 0.02 | 21.9 ± 8.9 | 18.1 |
| 85 | H | 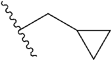 [SMILES: *CC1CC1] | 100 ± 0 | 1.38 ± 0.19 | 14.4 ± 9.8 | 10.4 |
| 86 | H | 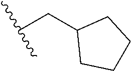 [SMILES: *CC1CCCC1] | 100 ± 0 | 1.24 ± 0.01 | 21.5 ± 5.0 | 17.3 |
| 87 | H | 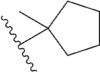 [SMILES: *C1(C)CCCC1] | 100 ± 0 | 0.27 ± 0.16 | 156.8 ± 11.0 | 580.7 |
| 88 | H | 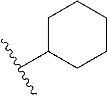 [SMILES: *C1CCCCC1] | 99.5 ± 0.7 | 1.34 ± 0.05 | 68.3 ± 1.9 | 51.1 |
| 89 | 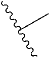 | 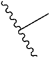 | 100 ± 0 | 1.92 ± 0.23 | 85.1 ± 11.9 | 44.3 |
| 90 | 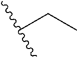 [SMILES: *CC] | 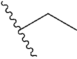 [SMILES: *CC] | 100 ± 0 | 0.86 ± 0.19 | 55.5 ± 10.2 | 64.5 |
| 91 | 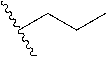 [SMILES: *CCC] | 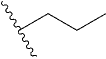 [SMILES: *CCC] | 100 ± 0 | 0.67 ± 0.03 | 10.7 ± 2.1 | 16.0 |
| 92 | 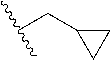 [SMILES: *CC1CC1] | 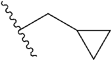 [SMILES: *CC1CC1] | 100 ± 0 | 0.62 ± 0.01 | 12.8 ± 4.2 | 20.7 |
| 93 | 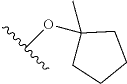 [SMILES: *OC1(C)CCCC1] | 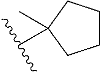 [SMILES: *C1(C)CCCC1] | 100 ± 0 | 0.74 ± 0.13 | 18.0 ± 2.7 | 24.3 |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
^{d}NT: not tested.
The results represent means ± SD of triplicate samples from three independent experiments.

HAdV (IC₅₀=0.62˜1.92 μM). When we increased the length of alkyl, compounds 89-91 showed an increasing trend in both potency and cytotoxicity. Additionally, N,N-dialkylated derivatives 80-92 were more potent than their corresponding N-alkylated derivatives 79, 80 and 85, respectively. Interestingly, N,O-dialkylated derivative 93 retained similar potency (IC₅₀=0.74 μM) and cytotoxicity (CC₅₀=18.0 μM) to those of N,N-dialkylated derivatives 90-92. These results indicated that branched alkyl groups are beneficial for potency, and N,N-dialkyl substitution is superior to its corresponding N-alkyl substitution.

TABLE 7
Inhibition of HAdV in Plaque Assays, Cytotoxicity and
Selectivity Index for Compounds 94-115

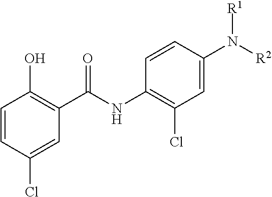

| | | | Plaque Assay^{a} | | | Selectivity |
|---|---|---|---|---|---|---|
| | | | Inhibition | | | index |
| Compd | R¹ | R² | (%, 10 μM) | IC₅₀ (μM) | CC₅₀ (μM)^{b} | (SI)^{c} |
| 94 | H | 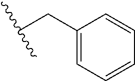 [SMILES: *Cc1ccccc1] | 100 ± 0 | 2.52 ± 0.01 | 19.7 ± 2.8 | 7.8 |
| 95 | H | 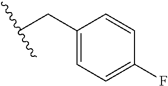 [SMILES: *Cc1ccc(F)cc1] | 100 ± 0 | 3.40 ± 0.54 | 54.4 ± 12.3 | 16.0 |
| 96 | H | 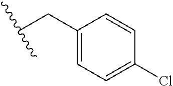 [SMILES: *Cc1ccc(Cl)cc1] | 100 ± 0 | 1.20 ± 0.01 | 59.7 ± 0.3 | 49.7 |
| 97 | H | 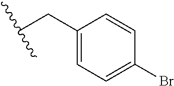 [SMILES: *Cc1ccc(Br)cc1] | 100 ± 0 | 1.84 ± 0.10 | 40.9 ± 2.2 | 22.2 |
| 98 | H | 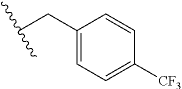 [SMILES: *Cc1ccc(C(F)(F)F)cc1] | 99.6 ± 0.6 | 1.43 ± 0.29 | 62.7 ± 6.1 | 43.8 |
| 99 | H | 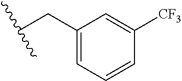 [SMILES: *Cc1cccc(C(F)(F)F)c1] | 100 ± 0 | 0.88 ± 0.40 | 13.3 ± 6.9 | 15.2 |
| 100 | H | 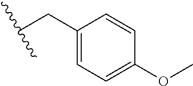 [SMILES: *Cc1ccc(OC)cc1] | 100 ± 0 | 3.61 ± 0.40 | 80.7 ± 8.2 | 22.4 |
| 101 | H | 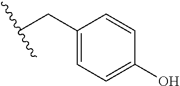 [SMILES: *Cc1ccc(O)cc1] | 80.7 ± 9.1 | 1.11 ± 0.16 | 115.6 ± 2.5 | 104.1 |
| 102 | H | 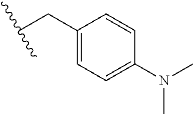 [SMILES: *Cc1ccc(N(C)C)cc1] | 100 ± 0 | 2.73 ± 0.26 | 24.5 ± 3.5 | 9.0 |
| 103 | H | 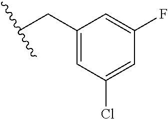 [SMILES: *Cc1cc(F)cc(Cl)c1] | 98.8 ± 0.6 | 1.28 ± 0.02 | 11.1 ± 0.7 | 8.7 |
| 104 | H | 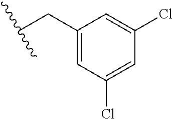 [SMILES: *Cc1cc(Cl)cc(Cl)c1] | 100 ± 0 | 1.43 ± 0.17 | 13.3 ± 7.6 | 9.3 |
| 105 | H | 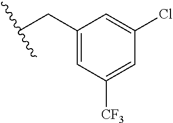 [SMILES: *Cc1cc(Cl)cc(C(F)(F)F)c1] | 99.3 ± 1.0 | 1.79 ± 0.16 | 30.9 ± 0.8 | 17.3 |
| 106 | H | 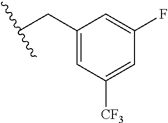 [SMILES: *Cc1cc(F)cc(C(F)(F)F)c1] | 100 ± 0 | 1.16 ± 0.13 | 30.9 ± 4.8 | 26.7 |
| 107 | H | 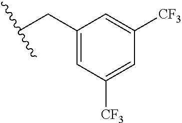 [SMILES: *Cc1cc(C(F)(F)F)cc(C(F)(F)F)c1] | 100 ± 0 | 1.12 ± 0.49 | 92.8 ± 12.8 | 82.9 |
| 108 | H | 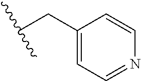 [SMILES: *Cc1ccncc1] | 100 ± 0 | 2.66 ±0.17 | 65.3 ± 5.7 | 24.6 |
| 109 | H | 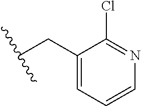 [SMILES: *Cc1cccnc1Cl] | 100 ± 0 | 4.78 ±0.01 | 100.5 ± 29.8 | 21.0 |
| 110 | H | 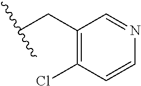 [SMILES: *Cc1cnccc1Cl] | 100 ± 0 | 1.66 ± 0.42 | 125.7 ± 25.1 | 75.7 |
| 111 | H | 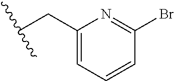 [SMILES: *Cc1cccc(Br)n1] | 100 ± 0 | 2.26 ± 0.05 | 92.3 ± 9.5 | 40.8 |
| 112 | H | 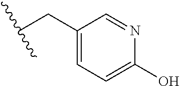 [SMILES: *Cc1ccc(O)nc1] | 100 ± 0 | 0.78 ± 0.02 | 98.5 ± 0.0 | 126.3 |
| 113 | H | 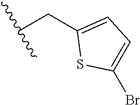 [SMILES: *Cc1ccc(Br)s1] | 100 ± 0 | 2.58 ± 0.29 | 20.7 ± 4.6 | 8.0 |
| 114 | H | 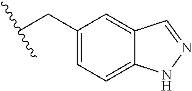 [SMILES: *Cc1ccc2[nH]ncc2c1] | 98.0 ± 0.7 | 4.65 ± 0.41 | 45.3 ± 0.4 | 9.7 |
| 115 | 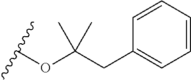 [SMILES: *OC(C)(C)Cc1ccccc1] | 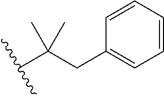 [SMILES: *C(C)(C)Cc1ccccc1] | 100 ± 0 | 1.27 ± 0.01 | 164.7 ± 3.6 | 129.7 |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
The results represent means ± SD of triplicate samples from three independent experiments.

Next, various aryl groups were introduced, and compounds 94-115 were prepared and evaluated as shown in Table 7. The substituents on the benzyl moiety (94-107) seem not to be critical for potency with IC₅₀ values ranging from 0.88 to 3.61 μM, and halogen (95-97), trifluoromethyl (98, 99), methoxyl (100), hydroxyl (101) and dimethylamino (102) were all well tolerated. Hydroxy derivatives 101 displayed potent anti-HAdV activity (IC₅₀=1.11 μM) and low cytotoxicity (CC₅₀=115.6 μM), resulting in a high selectivity index (SI=104.1). Intriguingly, disubstituted derivatives (103-107) showed similar potency with low micromolar EC₅₀ values against HAdV and varied cytotoxicity. Among these compounds, bis(trifluoromethyl) substituted derivative 107 exhibited a relatively high selectivity index (SI=82.9) due to its low cytotoxicity (CC₅₀=92.8 μM). Compounds 108-111 with pyridine moieties were all active (IC₅₀=1.66˜4.78 μM) along with relatively low cytotoxicity (CC₅₀=65.3˜125.7 μM). Notably, compound 112 with pyridone moiety showed submicromolar potency (IC₅₀=0.78 μM) meanwhile displaying low cytotoxicity (CC₅₀=98.5 μM) as well as a high selectivity index (SI=126.3). Substitution with thiophene (113) and indazole (114) moieties retained anti-HAdV activities (IC₅₀=2.58 μM and 4.65 μM, respectively). N,O-Dialkylated derivative 115 displayed similar potency (IC₅₀=1.27 μM) to its analogue 93, with decreased cytotoxicity (CC₅₀=164.7 μM) and increased selectivity index (SI=129.7).

TABLE 8
Inhibition of HAdV in Plaque Assays, Cytotoxicity and
Selectivity Index for Compounds 118-129

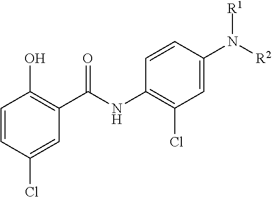

| | | | Plaque Assay^{a} | | | Selectivity |
|---|---|---|---|---|---|---|
| | | | Inhibition | | | index |
| Compd | R¹ | R² | (%, 10 μM) | IC₅₀ (μM) | CC₅₀ (μM)^{b} | (SI)^{c} |
| 118 | H | 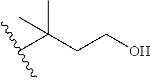 [SMILES: *C(C)(C)CCO] | 92.3 ± 0.0 | 0.90 ± 0.70 | 169.5 ± 4.2 | 188.3 |
| 119 | H | 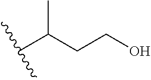 [SMILES: *C(C)CCO] | 96.9 ± 2.4 | 1.20 ± 0.40 | 200.0 ± 0.0 | 166.7 |
| 120 | H | 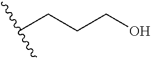 [SMILES: *CCCO] | 36.0 ± 4.2 | NT^{d} | 105.5 ± 0.0 | NT |
| 121 | 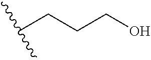 [SMILES: *CCCO] | 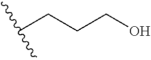 [SMILES: *CCCO] | 35.0 ± 7.1 | NT | 114.0 ± 9.2 | NT |
| 122 | H | 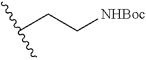 [SMILES: *CCNC(=O)OC(C)(C)C] | 100 ± 0 | 2.78 ± 0.30 | 25.0 ± 5.7 | 9.0 |
| 123 | H | 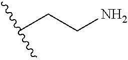 [SMILES: *CCN] | 19.4 ± 12.2 | NT | NT | NT |
| 124 | H | 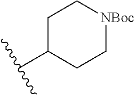 [SMILES: *C1CCN(C(=O)OC(C)(C)C)CC1] | 88.9 ± 9.0 | 1.21 ± 0.02 | 100.0 ± 1.8 | 82.6 |
| 125 | H | 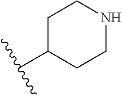 [SMILES: *C1CCNCC1] | 39.7 ± 13.5 | NT | NT | NT |
| 126 | H | 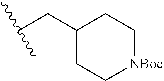 [SMILES: *CC1CCN(C(=O)OC(C)(C)C)CC1] | 97.8 ± 1.4 | 0.93 ± 0.42 | 95.0 ± 17.2 | 102.2 |
| 127 | H | 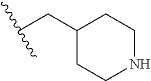 [SMILES: *CC1CCNCC1] | 20.6 ± 10.3 | NT | NT | NT |
| 128 | H | 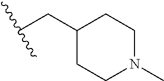 [SMILES: *CC1CCN(C)CC1] | 0.0 ± 0.0 | NT | NT | NT |
| 129 | H | 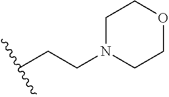 [SMILES: *CCN1CCOCC1] | 1.4 ± 12.3 | NT | NT | NT |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
^{d}NT: not tested.
The results represent means ± SD of triplicate samples from three independent experiments.

To introduce aqueous functional groups, compounds 118-129 were designed, synthesized, and evaluated (Table 8). Interestingly, branched hydroxyalkyl derivatives 118 and 119 showed potent anti-HAdV activity (IC₅₀=0.90 μM and 1.20 μM, respectively) while straight chained hydroxyalkyl analogues 120 and 121 were inactive at the concentration of 10 μM. This series of derivatives also exhibited low cytotoxicity (CC₅₀=105.5˜200.0 μM), and consequently the active molecules 118 and 119 possessed high selectivity index (SI=188.3 and 166.7, respectively). The aminoalkyl groups (123, 125 and 127-129) were unfavorable for potency, not active up to 10 μM, while their N-Boc protected derivatives 122, 124 and 126 maintained micromolar anti-viral activities with IC₅₀ values of 2.78 μM, 1.21 μM and 0.93 μM, respectively. Of these, compounds 124 and 126 showed low cytotoxicity (CC₅₀=100.0 μM and 95.0 μM, respectively) and relatively high selectivity index values (SI=82.6 and 102.2, respectively).

TABLE 9
Inhibition of HAdV in Plaque Assays, Cytotoxicity and
Selectivity Index for Compounds 135-147

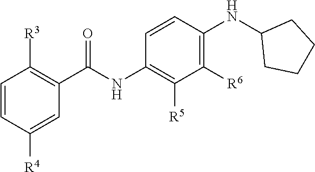

| | | | | | Plaque Assay^{a} | | | Selectivity |
|---|---|---|---|---|---|---|---|---|
| | | | | | Inhibition | | | index |
| Compd | R³ | R⁴ | R⁵ | R⁶ | (%, 10 μM) | IC₅₀ (μM) | CC₅₀ (μM)^{b} | (SI)^{c} |
| 135 | OH | H | Cl | H | 77.5 ± 2.0 | NT^{d} | NT | NT |
| 136 | OH | Me | Cl | H | 99.6 ± 0.5 | 2.72 ± 0.19 | 30.9 ± 1.6 | 11.4 |
| 137 | H | Cl | Cl | H | 82.9 ± 14.9 | NT | NT | NT |
| 138 | 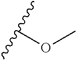 [SMILES: *OC] | Cl | Cl | H | 78.9 ± 18.6 | NT | NT | NT |
| 139 | 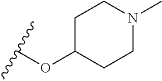 [SMILES: *OC1CCN(C)CC1] | Cl | Cl | H | 0.0 ± 0.0 | NT | NT | NT |
| 140 | 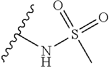 [SMILES: *NS(C)(=O)=O] | Cl | Cl | H | 99.8 ± 0.4 | 1.72 ± 0.46 | 35.4 ± 2.7 | 20.6 |
| 141 | OH | Cl | H | H | 99.8 ± 0.3 | 2.60 ± 0.03 | 10.0 ± 2.2 | 3.9 |
| 142 | OH | Cl | F | H | 98.7 ± 0.4 | 1.04 ± 0.17 | 51.0 ± 13.3 | 49.1 |
| 143 | OH | Cl | H | Cl | 100 ± 0 | 0.84 ± 0.28 | 16.9 ± 0.1 | 20.1 |

| | | | | | |
|---|---|---|---|---|---|
| 144 | 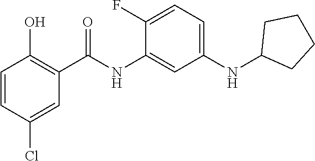 [SMILES: O=C(Nc1cc(NC2CCCC2)ccc1F)c1cc(Cl)ccc1O] | 99.8 ± 0.4 | 1.34 ± 0.05 | 44.0 ± 12.8 | 32.8 |
| 145 | 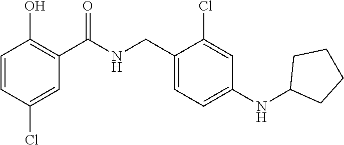 [SMILES: O=C(NCc1ccc(NC2CCCC2)cc1Cl)c1cc(Cl)ccc1O] | 98.5 ± 0.7 | 7.97 ± 0.43 | 28.3 ± 2.5 | 3.6 |
| 146 | 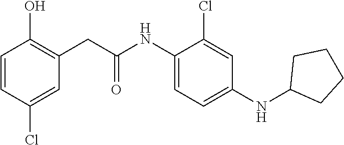 [SMILES: O=C(Cc1cc(Cl)ccc1O)Nc1ccc(NC2CCCC2)cc1Cl] | 85.8 ± 5.8 | NT | NT | NT |
| 147 | 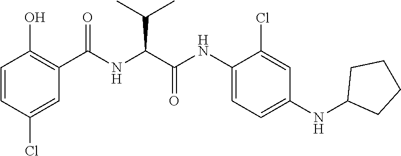 [SMILES: CC(C)[C@H](NC(=O)c1cc(Cl)ccc1O)C(=O)Nc1ccc(NC2CCCC2)cc1Cl] | 99.5 ± 0.7 | 3.83 ± 0.42 | 110.6 ± 8.4 | 28.9 |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀).
^{c}Selectivity index value was determined as the ratio of CC₅₀ to IC₅₀ in a plaque assay for each compound.
^{d}NT: not tested.
The results represent means ± SD of triplicate samples from three independent experiments.

Considering its potent anti-viral activity, we made a quick investigation on the effect of substituents on other positions of this scaffold while retaining the cyclopentylamino moiety of lead compound 78. As listed in Table 9, removal of the 5-Cl group on the salicylic ring (135) led to a significant loss of potency with inhibition of 77.5% at 10 μM while replacing it with 5-methyl group (136) retained some activity (IC₅₀=2.72 μM). Modifications of 2-phenolic hydroxyl yielded compounds 137-140, and all these changes diminished the anti-HAdV activities at different levels. Removal (137) or methylation (138) of the 2-OH group resulted in a decrease in activity with inhibition of 82.9% and 78.9% at 10 M, respectively, while substitution with 4-O-(1-methylpiperidinyl) (139) led to a complete loss of potency. Unexpectedly, replacing 2-OH with 2-methylsulfonamido (140) maintained low micromolar anti-HAdV activity (IC₅₀=1.72 μM). Next, we turned our attention to the aniline moiety. Removal of 2′-Cl on the aniline ring (141) resulted in a slight loss of potency (IC₅₀=2.60 μM) while substitution of 2′-Cl with 2′-F (142) or moving 2′-Cl to 3′-position (143) maintained the same level of potency (IC₅₀=1.04 μM and 0.84 μM, respectively) as compared with lead compound 78. In contrast to compound 142, moving the 4′-cyclopentylamino group to 5′-position produced compound 143 with a similar potency (IC₅₀=1.34 μM). Increasing the length of the linker generated compounds 145-147, and these modifications retained some activity with micromolar IC₅₀ values. Unfortunately, no compounds with high selectivity index were discovered during the modifications at other sites of the scaffold due to either moderate to high cytotoxicity or relatively low anti-viral activity.

Next eight compounds with high selectivity index (SI>100) were selected and examined their effect on viral replication in a virus burst assay by measuring the production of infectious virus particle. In this assay, A549 cells were infected with HAdV-5 wild-type virus and we calculated the TCID₅₀ of an infection in the presence and absence of the anti-HAdV compounds. As listed in Table 10, these selected derivatives showed overall virus yield reduction with varied folds, ranging from 9.3-fold for compound 119 to 232-fold for compound 126.

TABLE 10
Virus Yield Reduction for Selected Compounds
with High Selectivity Index

| | Virus Yield Reduction |
|---|---|
| Compound | (fold)^{a} |
| 73 | 82 ± 35 |
| 78 | 17.1 ± 3.1 |
| 87 | 52.6 ± 6.2 |
| 101 | 17.4 ± 14.2 |
| 112 | 26.6 ± 5.0 |
| 115 | 21.5 ± 0.0 |
| 118 | 15.7 ± 6.8 |
| 119 | 9.3 ± 2.2 |
| 126 | 232 ± 85 |

^{a}Fold-reduction in virus yield as the ratio of particles produced in the presence of DMSO divided by the yield in the presence of each selected compound at 10-fold IC₅₀ concentration obtained in the plaque assay for each compound. Virus Yield Reduction assay used A549 cell line and the MOI of HAdV was 100 vp/cell. The results represent means ± SD of triplicate samples from three independent experiments.

Impact on HAdV Entry. To gain a mechanistic understanding of their HAdV inhibition, selected derivatives were evaluated in entry assays, in which human A549 epithelial cells were infected with HAdV-GFP in the presence of the selected compound and incubated for 48 h. As summarized in Table 11, compounds 87, 101 and 119 showed significant inhibition against HAdV-GFP transgene expression with percentages of 85.4%, 83.3% and 88.6% at 50 μM, respectively, and IC₅₀ values ranging from 15.2 to 30.8 μM, while derivatives 78, 112, and 118 displayed no obvious inhibitory activity (IC₅₀>50 μM) in entry assay. As for compounds 115 and 126, lower percentage of inhibition (59.1% and 67.6%, respectively) at 50 μM was observed, with IC₅₀ values of 34.1 μM and <3.13 μM, respectively. However, these results could not provide us conclusive indication of their potential mechanism of action, as any interference at a step between HAdV attachment to its cellular receptors and HAdV genome import into the cell nucleus would eventually lead to a decrease in GFP expression.

TABLE 11
Inhibition of HAdV in Entry Assay for Selected
Compounds with High Selectivity Index

| | (%) Inhibition Entry | IC₅₀ Entry | CC₅₀ |
|---|---|---|---|
| Compound | Assay (50 μM)^{a} | Assay (μM) | (μM)^{b} |
| 73 | 100 ± 0 | 1.22 ± 0.44 | 22.9 ± 9.8 |
| 78 | 27.5 ± 3.8 | NT^{c} | 92.9 ± 7.1 |
| 87 | 85.4 ± 31.4 | 15.2 ± 5.1 | 156.8 ± 11.0 |
| 101 | 83.3 ± 16.4 | 25.0 ± 6.9 | 115.6 ± 2.5 |
| 112 | 51.7 ± 9.8 | >50.00 | 98.5 ± 0.0 |
| 115 | 59.1 ± 10.1 | 34.1 ± 6.9 | 164.7 ± 3.6 |
| 118 | 42.1 ± 3.0 | NT | 169.5 ± 4.2 |
| 119 | 88.6 ± 2.9 | 30.8 ± 4.5 | 200.0 ± 0.0 |
| 126 | 67.6 ± 15.9 | <3.13 | 95.0 ± 17.2 |

^{a}Percentage of control HAdV5-GFP inhibition at 50 μM and inhibitory concentration 50% (IC₅₀) at high MOI in an entry assay using the A549 cell line.
^{b}Cell cytotoxic concentration 50% (CC₅₀) using A549 cell line.
^{c}NT: not tested.

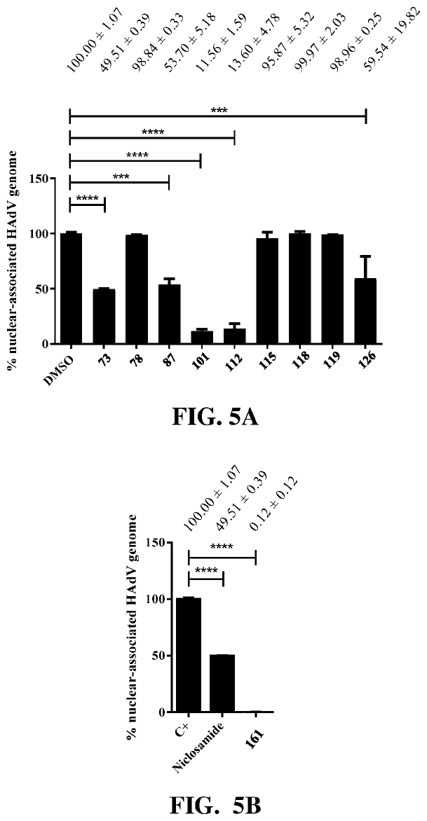

To further clarify whether these compounds was able to block any of the steps in the entry pathway, an assay was performed to quantitatively measure the HAdV genome accessibility to the nucleus. As shown in FIG. 5, at 45 min post-infection, cells treated with compounds 101 and 112 displayed significant reductions in the amount of nuclear-associated HAdV genomes versus those treated with DMSO while compounds 87 and 126 partially inhibited the accessibility of HAdV DNA to the nucleus. Compounds 78, 115, 118 and 119 exhibited no significant differences in the amount of nuclear-associated HAdV genome versus the control group. These results suggested that compounds 101 and 112 acted in the early steps of the HAdV replication cycle, while compounds 78, 115, 118 and 119 possibly suppressed later steps after the HAdV genome was imported into the nucleus.

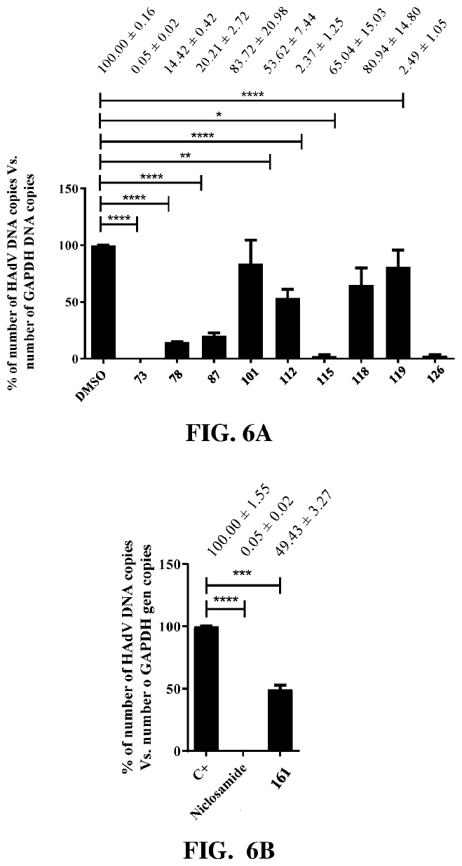

Impact on HAdV Replication. The next step was to evaluate the HAdV DNA replication efficacy by quantitative real-time PCR (qPCR) in the presence of these selected compounds. To avoid the influence of newly generated viral particles from subsequent rounds of infection occurring 32-36 h post infection, HAdV DNA was extracted at 24 h post-infection and thus quantified in a single round of infection as a measure DNA replication efficiency. As shown in FIG. 6, compounds 115 and 126 significantly inhibited HAdV-5 DNA replication by more than 95%, while moderate inhibition was observed for compounds 78 and 87. Compounds 101, 112, 118 and 119 displayed low inhibition against HAdV-5 DNA replication when compared to the control treated with the same concentration of DMSO. Taken together, compounds 78 and 115 caused a significant decrease in HAdV DNA replication while displaying no obvious effect on HAdV entry, indicating that these two compounds possibly target the HAdV DNA replication process. Compounds 118 and 119 exhibited no obvious inhibitory effect on either HAdV entry or HAdV DNA replication, likely suppressing later steps of HAdV life cycle such as the viral particle assembly, maturation, or release processes. Compounds 87 and 126, which partially blocked the accessibility of HAdV genomes to the nucleus, showed potent inhibition against HAdV DNA replication. However, based on our current data, it is still hard to tell whether these compounds had direct impact on HAdV replication. Interestingly, although compounds 101 and 112 significantly suppressed the accessibility of HAdV DNA to the nucleus, they showed a low decrease in HAdV DNA replication. Further investigation is deemed necessary to clarify this phenomenon and figure out their exact mechanisms of action.

Time of Addition Assay. Next, we measured the ability of these analogues to inhibit HAdV infection depending on their time of addition as an alternative way to confirm our previous results. All compounds exhibited a time-dependent decrease in inhibitory activity, showing different patterns (FIG. 7). Compounds 78 and 101 showed an inhibition of HAdV infection ≥50% when they were added either at the beginning of the 60 min incubation at 4° C. (−60 min) or at 120 min post infection (p.i.), just like niclosamide. Compounds 112, 118, and 119 showed a sudden decrease in inhibition from the beginning, showing less than 50% inhibition after the addition at the moment of the infection (0 min). In the same way but a little later, compounds 87 and 115 showed a decrease in their antiviral activity with inhibitions below 50% when they were added at 5 min p.i. In all these cases these results would suggest a very early target step during HAdV internalization after binding to the cell. Compound 126 loses its anti-HAdV activity when added between 10 and 20 min p.i., suggesting a target involved in the HAdV scape from the endosome or the microtubular transport of the partially uncoated particle to the nucleus of the cell.

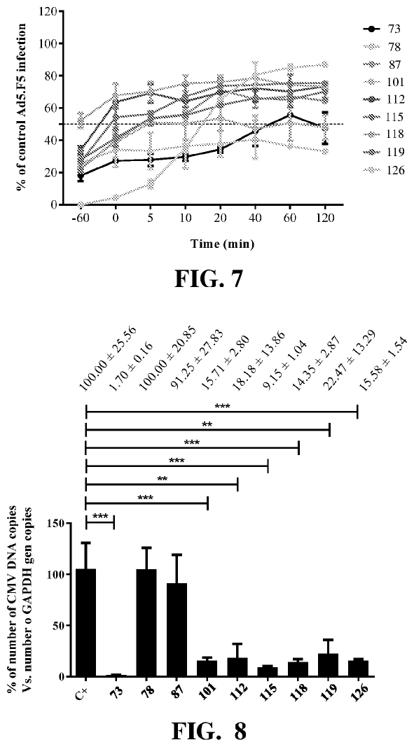

Impact on HCMV Replication. The selected compounds were further tested to evaluate their potential broad antiviral effects on human cytomegalovirus (HCMV) quantifying the total DNA 72 h after the infection of HFF cells in a single round of infection as a measure DNA replication efficiency. The addition of derivatives 73, 101, 115, 118 and 126 generated significant reductions (p≤0.001) in the quantification of total HCMV DNA showing a decrease from 84% to 98% compared to the DMSO control (FIG. 8). Compounds 112 and 119 also decreased the number of HCMV DNA copies (p≤0.001) although the percentages of reduction were lower (82% and 78%, respectively). Compounds 78 and 87 showed no significant inhibition of HCMV DNA replication indicating a high antiviral specificity against HAdV.

Synergistic Activity of Selected Derivatives. Since derivatives 17 (JMX0312) and 58 (JMX0281) were found to be targeting later steps after HAdV DNA replication and compound 87 seems to be addressing early stages during HAdV entry in the host cell, we decided to evaluate the effect of their combination on their anti-HAdV activity. We hypothesized that since these compounds are targeting different processes during HAdV replicative cycle their combination would result in an improved antiviral activity blocking HAdV infection at lower concentrations due to their synergistic effect. To assess this hypothesis, we conducted a combination study based on the Chou-Talalay method for drug combination using the CalcuSyn software (Chou, and Talalay, Adv. Enzyme Regul. 1984, 22, 27-55; Matthews et al., PLoS One. 2017, 12, e0173303) using those compounds with lower IC₅₀ in the plaque assay and higher SI, compound 87 from this work and compounds 17 (JMX0312), 58 (JMX0281) from our first library of derivatives. The constant ratio for each combination was selected based on the IC₅₀ values for each drug. The data for all the combinations showed good conformity with the mass action law principle (r=0.78-0.95) (Table 12). The combination of these three compounds was classified as strong synergism for the combination between compounds 17 and 58 at ED₉₀ to slight synergism for the combination of 87 and 58 at ED₇₅. At ED₅₀, the combination of 17 and 58 (ratio 1:2.5) were classified as synergistic (ED₅₀=0.68) and the combination of 17 with 87 (ratio 1:1.5) showed slight synergism (ED₅₀=0.88). However, the combination of compounds 87 and 58 (ratio 1:1.7) resulted in a clear antagonistic effect (ED₅₀=1.81).

TABLE 12
CalcuSyn results for the different combinations of compounds 17, 58 and 87^{a}

| Combination | | IC₅₀ | IC₅₀ | Combinatory index (CI) values in Plaque assay | | | |
|---|---|---|---|---|---|---|---|
| (Ratio) | Derivative | Single | combination | ED₅₀ | ED₇₅ | ED₉₀ | r |
| 17 & 58 | 17 | 0.18 ± 0.01 | 0.10 ± 0.01 | 0.68 | 0.47 | 0.11 | 0.92 |
| (1:2.5) | 58 | 0.45 ± 0.06 | 0.25 ± 0.02 | | | | |
| 87 & 58 | 87 | 0.27 ± 0.16 | 0.30 ± 0.02 | 1.81 | 0.91 | 0.46 | 0.95 |
| (1:1.7) | 58 | 0.45 ± 0.06 | 0.50 ± 0.04 | | | | |
| 17 & 87 | 17 | 0.18 ± 0.01 | 0.09 ± 0.00 | 0.88 | 0.63 | 0.48 | 0.78 |
| (1:1.5) | 87 | 0.27 ± 0.16 | 0.14 ± 0.00 | | | | |

^{a}The combinatory index values are shown for the combinations at the IC₅₀, IC₇₅, and IC₉₀ levels of inhibition. The r value for each combination is also reported to indicate the correlation coefficient of the data to the mass-action law.

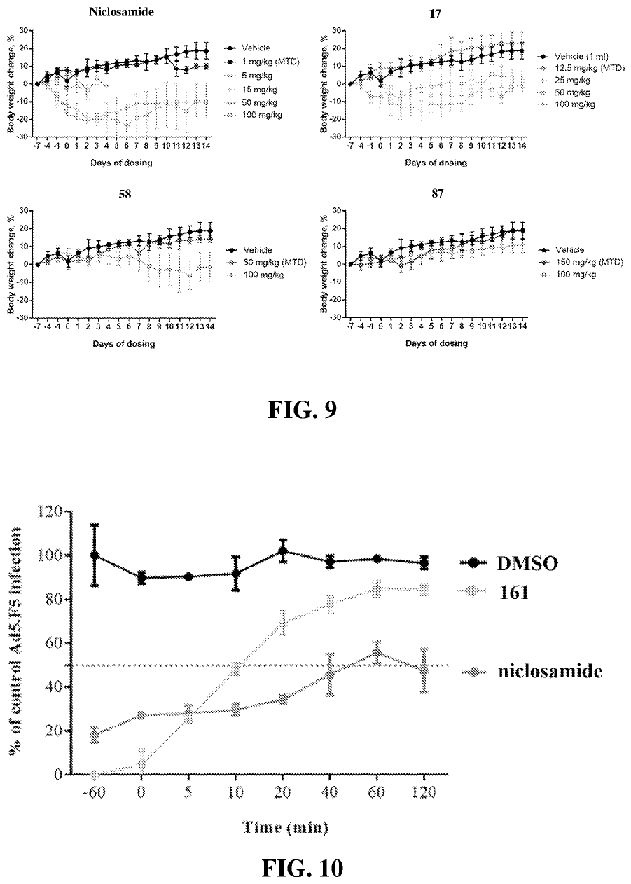

Maximum Tolerated Dose (MTD). To assist the selection of the best candidates for further in vivo efficacy evaluation, the MTD values of compound 87, which showed the best SI in this library of compounds, together with derivatives 17 and 58, were tested in the immunosuppress Syrian hamster model. As shown in FIG. 9, compound 87 presented the safest MTD at 150 mg/kg, followed by derivatives 58 and 17 with MTD of 50 and 12.5 mg/kg, respectively. Niclosamide showed high toxicity at doses ≥5 mg/kg, and finally an MTD of 1 mg/kg that prevented the loss of weight of the animals was determined. These results were consistent with the trend in the cytotoxicity for each of the molecules and showed a wide therapeutic window for the further in vivo evaluation of their efficacy in the treatment of HAdV infection, especially for derivatives 58 and 87.

A series of novel substituted N-(4-amino-2-chlorophenyl)-5-chloro-2-hydroxybenzamide analogues were discovered and identified as potent HAdV inhibitors. Compounds 78 (NY0624), 87 (JMX0510-2), 101 (JMX0452), 112 (JMX0464), 115 (JMX0508), 118 (NY0611), 119 (NY0621) and 126 (JMX0461) exhibited significantly high selectivity indexes (SI>100) while maintaining submicromolar to low micromolar potency against HAdV compared to niclosamide. The preliminary mechanistic studies indicated that compounds 78 and 115 possibly target the HAdV DNA replication process, while compounds 118 and 119 suppress later steps of HAdV life cycle. With respect to compounds 87, 101, 112 and 126, it is hard to conclude what stage they function at and further investigation is imperative to clarify their exact mechanisms of action. Notably, among all the derivatives tested in our two rounds of optimization, compounds 17, 58 and 87 showed the highest anti-HAdV activity (IC₅₀=0.18, 0.45 and 0.27 μM, respectively), significantly decreased cytotoxicity (CC₅₀=120.0, 200.0 and 156.8 μM, respectively) and very low in vivo toxicity (MTD=12.5, 50 and 150 mg/kg, respectively) as compared with niclosamide (IC₅₀=0.6 μM, CC₅₀=22.9 μM and MTD=1 mg/kg). Thus, compounds 17, 58 and 87 were selected for further in vivo characterization to evaluate first their pharmacokinetics profiles and subsequently their efficacy for the treatment of HAdV infection in the immunosuppressed Syrian hamster model. The findings on such extensive in vivo studies will be reported in due course.

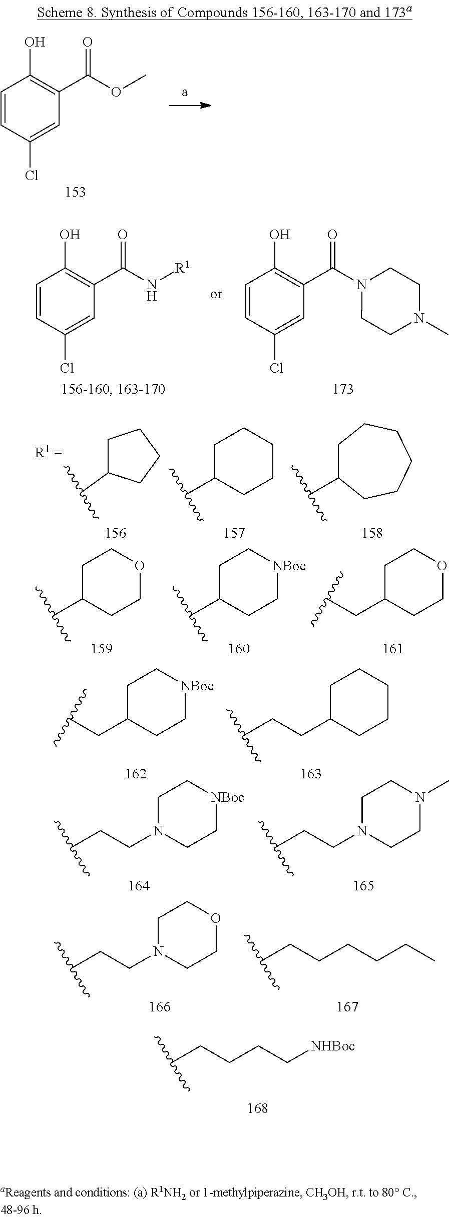

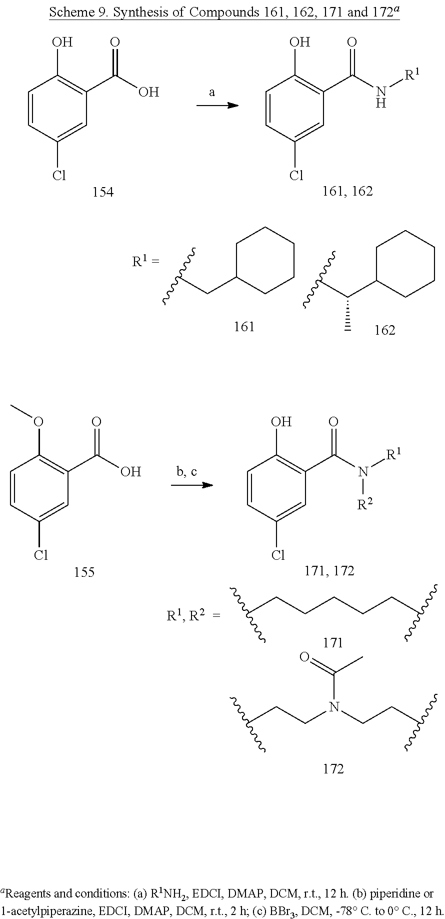

In vitro evaluation of human adenovirus inhibition. The newly synthesized compounds were first evaluated in plaque assay by measuring HAdV plaque-formation inhibition. A shown in Table 13, the aniline moiety of niclosamide was substituted with simple cycloalkanamines (156-160). When the size of cycloalkyl groups increased, the corresponding compounds 156-158 showed an increased trend in potency with inhibition of 22.6%, 57.6% and 99.6% at 10 μM, respectively. Compound 158 with N-cycloheptyl substitution displayed micromolar potency (EC₅₀=4.7 μM) and similar cytotoxicity (CC₅₀=30.9 μM) to niclosamide. The tetrahydropyran derivative 159 was completely inactive at 10 M while compound 160 with N-Boc-piperidinyl moiety inhibited HAdV plaque-formation with a percentage of 52.2%. Inserting one carbon atom as a linker between the amide and cycloalkyl produced compounds 161-164. Excitingly, in contrast to niclosamide, N-cyclohexylmethyl substituted derivative 161 exhibited similar potency (EC₅₀=0.78 μM) and significantly decreased cytotoxicity (CC₅₀=91.2 μM), resulting in an increased selectivity index (SI=116.9). Introducing (S)-methyl on the methylene linker (162) retained the same level of potency (EC₅₀=0.51 μM) and significantly diminished the cytotoxicity (CC₅₀=23.0 M) as compared with derivative 161. Compound 163 with tetrahydropyran moiety showed no anti-HAdV activity while N-Boc protected derivative 164 are active with inhibition of 88.5% against HAdV plaque formation at 10 μM. Interestingly, N-cyclohexylethyl derivative 165 suffered a slight loss of potency (EC₅₀=1.0 μM) meanwhile displaying decreased cytotoxicity (CC₅₀=66.3 μM) and improved selectivity index (SI=64.3) compared to niclosamide. Unfortunately, piperazine analogues 166, 167 and morpholine analogue 168 with two carbon length linkers all showed no obvious inhibitory activity against HAdV up to 10 μM. Intriguingly, derivative 168 with linear hexylamine substitution also maintained single digit micromolar potency with an EC₅₀ value of 3.73 μM. As expected, N-Boc protected derivative 170 was also active against HAdV with inhibition of 74.0% at 10 μM. Circular secondary amine moieties were not tolerated as shown by the activity data for compounds 171-173.

TABLE 13
Inhibition of HAdV in Plaque Assay, Cytotoxicity and
Selectivity Index for Compounds 156-173

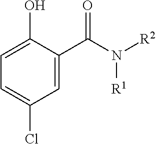

| | | | Plaque Assay (10 μM)^{a} | | | Selectivity |
|---|---|---|---|---|---|---|
| | | | (%) | | | index |
| Compd | R¹ | R² | Inhibition | IC₅₀ (μM) | CC₅₀ (μM)^{b} | (SI)^{c} |
| niclosamide | NA | | 100 ± 0 | 0.6 ± 0.05 | 22.9 ± 9.8 | 38.2 |
| 156 (JMX0725) | H | 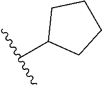 [SMILES: *C1CCCC1] | 22.6 ± 7.4 | NT^{d} | NT | NT |
| 157 (JMX0724) | H | 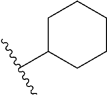 [SMILES: *C1CCCCC1] | 57.6 ± 0.2 | NT | NT | NT |
| 158 (JMX0726) | H | 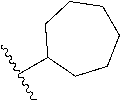 [SMILES: *C1CCCCCC1] | 99.6 ± 0.2 | 4.7 ± 0.1 | 30.9 ± 6.3 | 6.6 |
| 159 (JMX0722) | H | 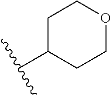 [SMILES: *C1CCOCC1] | 0.0 ± 0.0 | NT | NT | NT |
| 160 (JMX0723) | H | 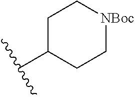 [SMILES: *C1CCN(C(=O)OC(C)(C)C)CC1] | 52.2 ± 7.1 | NT | NT | NT |
| 161 (JMX0493) | H | 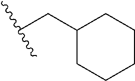 [SMILES: *CC1CCCCC1] | 100 ± 0 | 0.78 ± 0.01 | 91.2 ± 18.4 | 116.9 |
| 162 (JMX0494) | H | 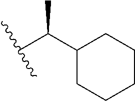 [SMILES: *[C@@H](C)C1CCCCC1] | 100 ± 0 | 0.51 ± 0.14 | 23.0 ± 1.3 | 45.1 |
| 163 (JMX0720) | H | 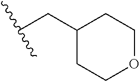 [SMILES: *CC1CCOCC1] | 0.0 ± 0.0 | NT | NT | NT |
| 164 (JMX0719) | H | 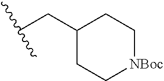 [SMILES: *CC1CCN(C(=O)OC(C)(C)C)CC1] | 88.5 ± 1.9 | NT | NT | NT |
| 165 (JMX0718) | H | 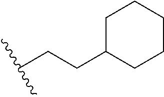 [SMILES: *CCC1CCCCC1] | 97.8 ± 1.7 | 1.0 ± 0.1 | 66.3 ± 7.9 | 64.3 |
| 166 (JMX0721) | H | 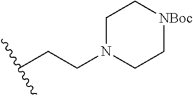 [SMILES: *CCN1CCN(C(=O)OC(C)(C)C)CC1] | 22.2 ± 13.2 | NT | NT | NT |
| 167 (JMX0881) | H | 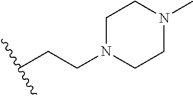 [SMILES: *CCN1CCN(C)CC1] | 0.00 ± 0.00 | NT | NT | NT |
| 168 (JMX0867) | H | 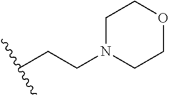 [SMILES: *CCN1CCOCC1] | 0.00 ± 0.00 | NT | NT | NT |
| 169 (JMX0947) | H | 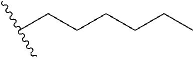 [SMILES: *CCCCCC] | 98.5 ± 2.1 | 3.73 ± 0.64 | 23.8 ± 3.1 | 6.4 |
| 170 (JMX0946) | H | 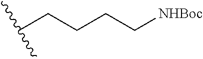 [SMILES: *CCCCNC(=O)OC(C)(C)C] | 74.0 ± 5.7 | NT | NT | NT |

| | | | | | |
|---|---|---|---|---|---|
| 171 (JMX0963) | 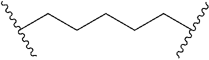 [SMILES: *CCCCC*] | 0.00 ± 0.00 | NT | NT | NT |
| 172 (JMX0873) | 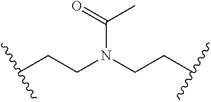 [SMILES: *CCN(CC*)C(C)=O] | 0.00 ± 0.00 | NT | NT | NT |
| 173 (JMX0965) | 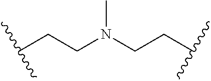 [SMILES: *CCN(C)CC*] | 0.00 ± 0.00 | NT | NT | NT |

^{a}Percentage of control HAdV5-GFP inhibition at 10 μM and inhibitory concentration 50% (IC₅₀) at low MOI in a plaque assay using the 293β5 cell line.
^{b}Cytotoxic concentration 50% (CC₅₀) using A549 cell line.
^{c}Selectivity index value was determined as the ratio of CC₅₀ to IC₅₀ in a plaque assay for each compound.
^{d}NT: not tested.
The results represent means ± SD of triplicate samples from three independent experiments.

The anti-HAdV effect of compound 161 were evaluated with high selectivity index (SI>100) using a virus burst assay which measures the ability to suppress the production of new virus particles. As shown in Table 14, treatment with compound 161 resulted in a 208-fold overall reduction in virus yield.

TABLE 14
Virus Yield Reduction for derivative 161

| | Virus Yield Reduction |
|---|---|
| Compd | (fold)^{a} |
| niclosamide | 82 ± 35 |
| 161 | 208 ± 108.1 |
| (JMX0493) | |

^{a}Fold-reduction in virus yield as the ratio of particles produced in the presence of DMSO divided by the yield in the presence of niclosamide at 5 μM and derivative 161 at 10-fold IC₅₀ concentration obtained in the plaque assay. Virus Yield Reduction assay used A549 cell line and the MOI of HAdV was 100 vp/cell. The results represent means ± SD of triplicate samples from three independent experiments.

Impact of compound 161 on HAdV entry. To investigate the mechanism of action presented by this series of derivatives, we first evaluated the representative derivative 161 in entry assay. Briefly, in this assay we used human A549 epithelial cells infected with HAdV-GFP in the presence of compound 161 and incubated for 48 h. As shown in Table 15, compound 161 inhibited the expression of the HAdV-GFP transgene in dose-dependent manner with an EC₅₀ value of 12.3 μM.

TABLE 15
Inhibition of HAdV in Entry Assay for derivative 161

| | Entry Assay (50 μM)^{a} | | |
|---|---|---|---|
| | (%) | IC₅₀ | CC₅₀ |
| Compd | Inhibition | (μM) | (μM)^{b} |
| niclosamide | 100 ± 0 | 1.22 ± 0.44 | 22.9 ± 9.8 |
| 161 | 98.6 ± 14.8 | 12.3 ± 4.8 | 91.2 ± 18.4 |
| (JMX0493) | | | |

^{a}Percentage of control HAdV5-GFP inhibition at 50 μM and inhibitory concentration 50% (IC₅₀) at high MOI in a entry assay using the A549 cell line.
^{b}Cytotoxic concentration 50% (CC₅₀) using A549 cell line.

Once escaping from the endosome, the partially uncoated HAdV capsid is transported to the cell nucleus where further disassembly occurs and HAdV genome is released. Reasonably, any inhibitory effect on that process will be reflected in the number of HAdV genomes that reach the host nucleus after a synchronized infection. To figure out if compound 161 could block some of the steps between the entry and the arrival to the nucleus, we performed an assay to quantify the HAdV genome accessibility to the nucleus. As shown in FIG. 5B, compound 161 inhibited more than 99% the accessibility of HAdV DNA to the nucleus and showed a higher inhibitory effect versus the control group and niclosamide at 45 min post-infection (p≤0.0001).

Impact of compound 161 on HAdV replication. A quantitative real-time PCR (qPCR) was carried out in a 24 h. assay in the presence of compound 161 to evaluate HAdV DNA replication efficiency. With this assay, we could quantify the new synthesized HAdV DNA copies as a measure of it. As shown in FIG. 6B, compound 161 at a concentration of 7.8 μM, inhibited HAdV-5 DNA replication by 50% showing a significant inhibitory activity in qPCR assay (p≤0.001) when compared to a control treated with the same concentration of DMSO while it showed lower inhibition on the process when it was compared to niclosamide.

This result indicated that the compound may be targeting the HAdV DNA replication process.

In summary, compound 161 caused a significant inhibition in HAdV DNA replication and it could be possible to its potent inhibitory effects during HAdV entry pathway, which was supported by the percentage of nuclear HAdV genome found in the cell after 45 min of infection.

Synergistic activity of the selected compounds. Synergistic effect of compound 161 with compound 149 (JMX0312), 150 (JMX0510-2) and 151 (JMX0281).

Time of Addition Assay. With the aim to go in depth in the mechanism of inhibition, we measured the time dependence of compound 161 addition on their ability to inhibit HAdV infection as an alternative step toward identifying the specific stage of HAdV replicative cycle that was inhibited by these compounds. HAdV has been shown to be internalized within 5 min after binding, to escape the endosome after 15 min, and to attach to the nuclear pore complex after 35-45 min. According to that, activity against HAdV of compound 161 exhibited a time-dependent decrease (FIG. 10), specially at early time points when we observed an enormous decrease in its inhibitory activity, from 95.3% at 0 min post-infection (p.i) to 30.7% at 20 min p.i. That results indicated that this compound inhibited an early step in the virus cycle, after cell attachment and before the entry into the cell nucleus.

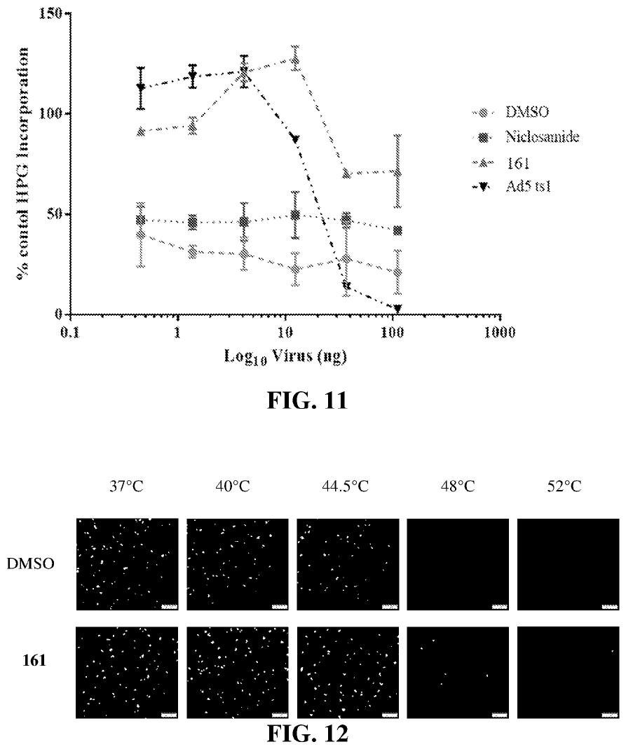

Upon attachment to its cellular receptors, HAdV particles are internalized by endocytosis into the cells and viral particles undergo partial disassembly inside the early endosomes, resulting in the release of protein VI from the interior of the capsid which plays a key role in HAdV escape from the endosome (Wiethoff et al., J. Virol. 2005, 79, 1992-2000). To evaluate the potential influence of compound 161 in preventing the HAdV escape from the endosome, we used the α-sarcin co-delivery assay as a measurement of the ability of this compound to interfere with virus-mediated endosome lysis. Since the α-sarcin alone is unable to penetrate the cell, in this assay the successful virus-mediated lysis of the endosome would result in ribotoxin-mediated inhibition of cellular protein synthesis. Increasing concentrations of HAdV in the presence or absence of 161 were mixed with α-sarcin and incubated with A549 cells. After metabolic labeling with methionine L-homopropargylglycine (HPG), cell samples were assayed for HPG incorporation into cellular proteins. As shown in FIG. 11, cells treated with either the vehicle (DMSO) or niclosamide showed rates of HPG incorporation under 50% compared to a control without α-sarcin, independent of the virus concentration used. Interestingly, 161 showed a similar behavior as the entry-defective ts1 mutant HAdV that contains a mutation in the protease gene and fails to penetrate cell endosomes (Rancourt et al., Virology. 1995, 209, 167-173). Compound 161 prevented HAdV-mediated endosome lysis at concentrations of the virus below 12 ng similar to the effects observed by the ts1 mutant. This result, together with our time of addition assay, showing a decrease of the inhibitory effect after 15-20 min p.i, suggests that the mechanism for inhibition of this compound is likely related to the blockage of HAdV escape from the endosome.

The studies described above delineate the step of infection that is targeted by 161; however, the precise molecular mechanism involved in its antiviral activity remained to be established. One alternative mode of action was that 161 could block HAdV-mediated lysis of the endosome by stabilizing the virus capsid, thereby preventing uncoating. Indeed, human defensins and neutralizing antibodies have been previously shown to act through this mechanism (Smith et al., Cell Host Microbe, 2008, 3, 11-19). To determine whether 161 impacts HAdV uncoating, we used a thermostability assay that mimics virus disassembly in the endosome, which was previously described by Wiethoff et al., (Wiethoff et al., J. Virol. 2005, 79, 1992-2000), with a few modifications. In this assay, temperatures above 48° C. promote selective removal of the virus vertex region. HAdV-5 was incubated with or without 50 μM concentrations of 161 at temperatures from 37° C. to 52° C. and then added to cells to evaluate the viability of the viruses. Viruses incubated with either 161 or DMSO at 37° C., 40° C. and 44.5° C. were largely intact, showing similar rates of infection measured by the number of cells expressing GFP (FIG. 12). Upon heating HAdV-5 to 48° C. or above, in those non-treated with compound 161, there was no GFP expression 24 h post-infection. However, in wells with the addition of 161, significant levels of GFP expression were observed for those viruses heated to 48° C. and some residual expression remained at 52° C. These findings suggest that 161 could prevent HAdV endosomal escape by stabilizing the viral capsid and subsequently inhibiting endosomal membrane lysis (Luisoni et al., Cell Host Microbe, 2015, 18, 75-85).

Synergistic Activity of Selected Compounds. In a previous report we found 3 compounds with high activity against HAdV, so it is contemplated that the double and triple combination with compound 161 should significantly increase their antiviral activity. To evaluate, a combination study was conducted based on the Chou-Talalay method for drug combination using the CalcuSyn software. The constant ration for each combination was selected based on the IC₅₀ values for each drug. The data for all the combinations showed good conformity with the mass action law principle (r=0.87-0.95) (Table 16). Two out of three double combinations showed a synergistic effect, concretely, JMX0312 and 161 (ratio 1:4.3) showed strong synergism (ED₅₀=0.26) and JMX0281 and 161 (ratio 1:1.73) were classified as synergistic (ED₅₀=0.40), however, JMX510-2 and 161 (ratio 1:2.9) showed only an additive effect (ED₅₀=1.09). Regarding triple combinations, one of them (JMX0312 & JMX0510-2 & 161; ratio 1:1.5:4.3) had a synergistic effect with an ED₅₀ value of 0.33, on the other side, the another triple combination (JMX0510-2 & JMX0281 & 161; ratio 1:1.7) showed a clear antagonistic effect (ED₅₀=2.01), probably due to compound interactions. It is worth noting that in a previous report, the combinatory index also increased when JMX0510-2 was added to the mix, reaching additive or antagonistic values.

TABLE 16
IC₅₀ values in a plaque assay for each derivative and its combination.
Combination index value was calculated with CalcuSyn software.

| | Combinatory index (CI) values in Plaque assay | | | |
|---|---|---|---|---|
| Combination (Ratio) | ED₅₀ | ED₇₅ | ED₉₀ | r |
| JMX0281 & JMX0493 | 0.40 | 0.27 | 0.19 | 0.94 |
| (1:1.73) | | | | |
| JMX0312 & JMX0493 | 0.26 | 0.38 | 0.56 | 0.87 |
| (1:4.3) | | | | |
| JMX0510-2 & JMX0493 | 1.09 | 1.08 | 1.14 | 0.92 |
| (1:2.9) | | | | |
| JMX0510-2 & JMX0281 | 2.01 | 1.17 | 0.72 | 0.95 |
| & JMX0493 (1:1.7:2.9) | | | | |
| JMX0312 & JMX0510-2 | 0.33 | 0.26 | 0.22 | 0.94 |
| & JMX0493 (1:1.5:4.3) | | | | |

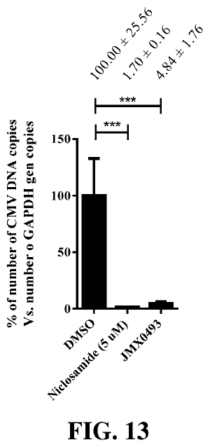

Impact of Compound 161 on HCMV Replication. The broad antiviral activity of the selected compound was tested on HCMV in a DNA replication assay. To avoid the influence of newly generated viral particles from subsequent rounds of infection, CMV DNA was extracted at 72 h post-infection of HFF cells and thus quantified in a single round of infection as a measure DNA replication efficiency. The addition of derivative 161 generated significant reductions (p≤0.001) in the quantification of total HCMV DNA showing a 95% decrease compared to the DMSO control as we showed in FIG. 13.

To conclude, a set of salicylamide derivatives was optimized to identify potent inhibitors of HAdV infections. According to our results, compound 14 showed a similar inhibition effect on HAdV to niclosamide with comparable IC₅₀ values (both of them were under micromolar IC₅₀ values) but a higher selectivity index was found for compound 14 (SI>100). In addition, compound 14 was also able to reduce the virus yield 2.5-fold more than niclosamide. Therefore, with a similar HAdV inhibition, compound 14 is safer and more effective with virus yield reduction than niclosamide. As for its mechanism of action and unlike niclosamide, compound 14 showed a strong inhibition of HAdV infection blocking the scape of the viral particles from the endosome and their subsequent migration to the nuclear membrane.

### I. Chemical Definitions

Various chemical definitions related to such compounds are provided as follows.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

As used herein, the term “water soluble” means that the compound dissolves in water at least to the extent of 0.010 mole/liter or is classified as soluble according to literature precedence.

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”)

The term “monosaccharide” refers to a cyclized monomer unit based on a compound having a chemical structure H(CHOH)ₙC(═O)(CHOH)ₘH wherein n+m is 4 or 5. Thus, monosaccharides include, but are not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

An “isomer” of a first compound is a separate compound in which each molecule contains the same constituent atoms as the first compound, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

It is contemplated that any embodiment discussed in this specification can be implemented with respect to any method or composition of the invention, and vice versa. Furthermore, compositions of the invention can be used to achieve methods of the invention.

### II. Method of Therapy

The term “therapy” is intended to encompass any form of treatment, prevention or diagnosis, and includes treatments to both cure and prevent disease. Thus, treatment of a healthy animal or subject is to be considered as therapy. Therapy also covers the alleviation of symptoms, in addition to curative treatments for a disease. All embodiments described herein apply equally to a method of therapy according to the present invention. The therapy according to the present invention may comprise alleviating one or more clinical symptoms of a viral infection.

The present invention provides a composition comprising a compound as described herein for use as an anti-viral medicament. Also provided is the use of a composition comprising at least one anti-viral compound for the manufacture of a medicament for the therapy of a viral infection and/or infection by a virus in an animal or subject. All embodiments described herein apply equally to such uses according to the present invention. In particular embodiments, the compounds are envisaged for use in a method of therapy comprising the reduction of viral load. In further particular embodiments, the compounds are envisaged for use in the reduction of clinical symptoms of the infection.

The present invention provides a pharmaceutical composition comprising at least one anti-viral compound for use according the invention and a pharmaceutically acceptable carrier, vehicle, diluent or excipient.

The compounds of the present invention may be formulated for oral or parenteral use in a conventional manner using known pharmaceutical carriers and excipients, and they may be presented in unit dosage form or in multiple dose containers. The compositions may be in the form of tablets, capsules, solutions, suspensions or emulsions. These compounds may also be formulated as suppositories utilizing conventional suppository bases such as cocoa butter or other fatty materials. The compounds may, if desired, be administered in combination with other antiviral compounds or treatments.

In one embodiment, the composition for use according to the invention may be administered via a route selected from the group consisting of: oral, parenteral, intravenous, intramuscular, subcutaneous, intranasal, intrapulmonary, intraperitoneal, intradermal, intrathecal and epidural.

In a preferred embodiment, the route of administration is oral, intravenous or intramuscular. In a particularly preferred embodiment, the route of administration is intramuscular, for example injectable intramuscular.

The present invention further provides formulations of the compounds of the present invention, which are particularly suited for the therapeutic use envisaged. The compounds of the invention may be formulated with conventional carriers and excipients, which will be selected in accordance with ordinary practice. Tablets may contain excipients, glidants, fillers, binders and the like. Aqueous formulations may be prepared in sterile form, and when intended for delivery by other than oral administration generally will be isotonic. Formulations optionally contain excipients such as those set forth in the “Handbook of Pharmaceutical Excipients” (1986) and include sodium hydroxide, ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e., the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol, benzyl alcohol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-step procedure, with the selected carrier material and, where appropriate, the other additives such as surface-active agents may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgents or emulsifiers, to be used in the pharmaceutical compositions of the present invention are non-ionic, cationic and/or anionic materials having good emulsifying, dispersing and/or wetting properties. A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd Ed. (Hanser Verlag, Vienna, 1981) and “Encyclopaedia of Surfactants”, (Chemical Publishing Co., New York, 1981).

While it is possible for the active ingredients to be administered alone it is preferable to present them as pharmaceutical formulations. The formulations for pharmaceutical use of the present invention comprise at least one active ingredient, as above described, together with one or more pharmaceutical acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral or parenteral (including subcutaneous, intraperitoneal, intramuscular, intravenous, intradermal, intrathecal and epidural) administration.

The formulations may conveniently be presented in unit dosage form and may be prepared by any of the methods well known in the art of pharmacy. In particular embodiments, as indicated above, the compounds of the present invention are provided as oral or injectable formulations.

The optimal dosage regimen for the treatment of an infected subject may be achieved when the compound according to the invention is administered at least once weekly, with a total dose of 10 to 1000 mg/kg. Such a regimen can ensure reduction of the viral load and/or reduction of clinical symptoms. Thus, a further aspect of the present invention provides the compounds of the present invention, for use in the treatment methods of the present invention, wherein the compound is administered at least once weekly, with a total dose of 10 to 1000 mg/kg. In certain embodiments, the compound is administered via oral route. In certain embodiments, the compound is administered via subcutaneous injections. In one embodiment, the at least one compound of the present invention is provided at a total dose of 10-1000 mg/kg, during 1 to 6 weeks. In another embodiment, a compound of the invention may be administered to a subject infected with a virus (e.g., Adenovirus) at a dosage of from 0.1 to 5 mg/kg every 24-120 hours, for example every 48-96 hours, for example every 72 hours, for a period of 1 to 8 weeks. In addition, when provided in unit dosage forms, the compositions may contain from about 0.1 to about 100 mg/kg/dose of the active anti-viral ingredient. The dosage of the compounds of the invention is dependent on such factors as the weight and age of the subject, as well as the particular nature and severity of the disease, and within the discretion of the physician or practitioner. The dosage for treatment may vary depending on the frequency and route of administration.

A dosage can be divided into one, two or more doses in a suitable form to be administered at one, two or more times throughout a given time period. The compositions of the invention can be administered for prophylactic or therapeutic treatments. In prophylactic applications, compositions can be administered to a subject with a clinically determined predisposition or increased susceptibility to development of a viral infection or related disease. Compositions of the invention can be administered to the subject in an amount sufficient to delay, reduce, or preferably prevent the onset of clinical disease or infection. In therapeutic applications, compositions are administered to a subject already suffering from disease or infection in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or infection. A therapeutically effective amount of a compound may not be required to cure a disease or infection but will provide a treatment for a disease or infection such that the onset of the disease or condition is delayed, hindered, or prevented, or the disease or infection symptoms are ameliorated, or the term of the disease or infection is changed or, for example, is less severe or recovery is accelerated. Amounts effective for this use may depend on the severity of the disease or infection and the weight and general state of the subject, but generally range from about 0.5 mg to about 3000 mg of the agent or agents per dose per subject. Suitable regimes for initial administration and booster administrations may be typified by an initial administration followed by repeated doses at one or more hourly, daily, weekly, or monthly intervals by a subsequent administration. The total effective amount of a compound or compounds present in the compositions of the invention can be administered to a subject as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more compounds present within the compositions of the invention and used in the methods of this invention applied to animals (e.g., humans or equines) can be determined by the ordinarily-skilled artisan with consideration of individual differences in age, weight, and the condition of the animal. The compositions of the invention are administered to a subject in an effective amount, which is an amount that produces a desirable result in a treated subject (e.g. the slowing or remission of infection). Therapeutically effective amounts can be determined empirically by those of skill in the art.

The subject may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. A subject may also receive an agent of the composition in the range of 0.1 to 3,000 mg per dose once every two or three weeks.

The compositions of the present invention may be used in combination with either conventional methods of treatment or therapy or may be used separately from conventional methods of treatment or therapy.

When the compositions of this invention are administered in combination therapies with other agents, they may be administered sequentially or concurrently to a subject.

### VII. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Salicylamide Derivatives

General Chemistry Information. All commercially available starting materials and solvents were reagent grade and used without further purification. Reactions were performed under a nitrogen atmosphere in dry glassware with magnetic stirring. Preparative column chromatography was performed using silica gel 60, particle size 0.063-0.200 mm (70-230 mesh, flash). Analytical TLC was carried out employing silica gel 60 F254 plates (Merck, Darmstadt). Visualization of the developed chromatograms was performed with detection by UV (254 nm). NMR spectra were recorded on a Brucker-300 (¹H, 600 and 300 MHz; ¹³C, 150 and 75 MHz) spectrometer. ¹H and ¹³C NMR spectra were recorded with TMS as an internal reference. Chemical shifts were expressed in ppm, and J values were given in Hz. High-resolution mass spectra (HRMS) were obtained from Thermo Fisher LTQ Orbitrap Elite mass spectrometer. Parameters include the following: Nano ESI spray voltage was 1.8 kV; Capillary temperature was 275° C. and the resolution was 60,000; Ionization was achieved by positive mode. Melting points were measured on a Thermo Scientific Electrothermal Digital Melting Point Apparatus and uncorrected. Purities of final compounds were established by analytical HPLC, which was carried out on a Shimadzu HPLC system (model: CBM-20A LC-20AD SPD-20A UV/VIS). HPLC analysis conditions: Waters μBondapak C18 (300×3.9 mm); flow rate 0.5 mL/min; UV detection at 270 and 254 nm; linear gradient from 10% acetonitrile in water to 100% acetonitrile in water in 20 min followed by 30 min of the last-named solvent (0.1% TFA was added into both acetonitrile and water). All biologically evaluated compounds were >95% pure.

5-Chloro-N-(3-chloro-4-nitrophenyl)-2-hydroxybenzamide (10). To a solution of 5-chloro-2-methoxybenzoic acid (281 mg, 1.5 mmol) and 3-chloro-4-nitroaniline (200 mg, 1.2 mmol) in 20 mL of toluene was added PCl₃ (286 mg, 2.1 mmol) at r.t. The resulting mixture was stirred at 100° C. for 6 h and concentrated. Then MeOH (20 mL) and H₂O (5 mL) was added successively, and the mixture was stirred at r.t. for 20 min. The amide intermediate was isolated by filtration as a pale yellow solid. The amide intermediate was dissolved in 40 mL of DCM, and BBr₃ (5.0 mL, 5.0 mmol, 1 M in DCM) was added dropwise at 0° C. The mixture was stirred at 0° C. for 2 h until the reaction was completed monitored by TLC. Then the mixture was diluted with DCM, washed with H₂O, and concentrated. The residue was purified by recrystallization (MeOH/H₂O) to afford compound 10 as a pale solid (310 mg, 95% in two steps). HPLC purity 99.1% (t_{R}=19.10 min). ¹H NMR (300 MHz, DMSO-d₆) δ 11.39 (s, 1H), 10.82 (s, 1H), 8.20-8.14 (m, 2H), 7.86 (dd, J=9.0, 2.4 Hz, 1H), 7.80 (d, J=2.7 Hz, 1H), 7.48 (dd, J=9.0, 2.7 Hz, 1H), 7.04 (d, J=8.7 Hz, 1H). ¹³C NMR (75 MHz, DMSO-d₆) δ 165.1, 155.8, 143.3, 142.0, 133.1, 128.8, 127.4, 126.6, 122.8, 121.6, 120.8, 118.9 (2C). HRMS (ESI) calcd for C₁₃H₉Cl₂N₂O₄, 326.9939 (M+H)⁺; found, 326.9931.

5-Chloro-N-(2-fluoro-4-nitrophenyl)-2-hydroxybenzamide (11). Compound 11 (318 mg, 80% in two steps) was prepared by a procedure similar to that used to prepare compound 10. The title compound was obtained as a grey solid. HPLC purity 99.3% (t_{R}=18.54 min). ¹H NMR (300 MHz, DMSO-d₆) δ 12.34 (s, 1H), 11.1 (d, J=2.1 Hz, 1H), 8.66 (t, J=8.4 Hz, 1H), 8.28-8.13 (m, 2H), 7.92 (d, J=2.7 Hz, 1H), 7.51 (dd, J=8.7, 2.7 Hz, 1H), 7.06 (d, J=8.7 Hz, 1H). ¹³C NMR (75 MHz, DMSO-d₆) δ 162.7, 155.2, 151.1 (d, J=245.6 Hz), 142.4 (d, J=8.8 Hz), 133.9, 133.0 (d, J=10.3 Hz), 129.8, 123.7, 121.1 (d, J=2.9 Hz), 120.8 (d, J=0.8 Hz), 119.5, 119.2, 111.3 (d, J=24.5 Hz). HRMS (ESI) calcd for C₁₃H₉ClFN₂O₄, 311.0235 (M+H)⁺; found, 311.0226.

5-Chloro-N-(2-fluoro-5-nitrophenyl)-2-hydroxybenzamide (12). Compound 12 (370 mg, 93% in two steps) was prepared by a procedure similar to that used to prepare compound 10. The title compound was obtained as a pale solid. HPLC purity 99.7% (t_{R}=18.27 min). ¹H NMR (300 MHz, DMSO-d₆) δ 12.25 (s, 1H), 10.94 (s, 1H), 9.24 (dd, J=6.6, 3.0 Hz, 1H), 8.11-8.03 (m, 1H), 7.93 (d, J=3.0 Hz, 1H), 7.62 (t, J=9.6 Hz, 1H), 7.50 (dd, J=8.7, 2.7 Hz, 1H), 7.05 (d, J=8.7 Hz, 1H). ¹³C NMR (75 MHz, DMSO-d₆) δ 162.9, 155.9 (d, J=252.6 Hz), 155.3, 143.8 (d, J=2.5 Hz), 133.7, 129.7, 127.2 (d, J=12.4 Hz), 123.6, 120.4 (d, J=9.7 Hz), 119.4, 119.1, 116.9 (d, J=3.5 Hz), 116.3 (d, J=22.1 Hz). HRMS (ESI) calcd for C₃H₉ClFN₂O₄, 311.0235 (M+H)⁺; found, 311.0229.

5-Chloro-2-hydroxy-N-(4-nitrophenyl)benzamide (13). Compound 13 (400 mg, 63% in two steps) was prepared by a procedure similar to that used to prepare compound 10. The title compound was obtained as a yellow solid. HPLC purity 99.4% (t_{R}=18.19 min). ¹H NMR (300 MHz, DMSO-d₆) δ 11.45 (s, 1H), 10.82 (s, 1H), 8.31-8.24 (m, 2H), 8.03-7.95 (m, 2H), 7.83 (d, J=2.7 Hz, 1H), 7.48 (dd, J=8.7, 2.7 Hz, 1H), 7.04 (d, J=9.0 Hz, 1H). ¹³C NMR (75 MHz, DMSO-d₆) δ 165.0, 155.8, 144.5, 142.7, 133.0, 128.8, 124.9 (2C), 122.8, 120.9, 120.0 (2C), 118.9. HRMS (ESI) calcd for C₁₃H₁₀ClN₂O₄, 293.0329 (M+H)⁺; found, 293.0318.

5-Chloro-N-(2-chloro-4-(trifluoromethyl)phenyl)-2-hydroxybenzamide (14). Compound 14 (120 mg, 42% in two steps) was prepared by a procedure similar to that used to prepare compound 10. The title compound was obtained as a white solid. HPLC purity 98.7% (t_{R}=21.02 min). ¹H NMR (300 MHz, CDCl₃) δ 11.42 (s, 1H), 8.59 (d, J=8.4 Hz, 2H), 7.74 (d, J=1.8 Hz, 1H), 7.62 (dd, J=8.7, 1.8 Hz, 1H), 7.53 (d, J=2.4 Hz, 1H), 7.45 (dd, J=9.0, 2.4 Hz, 1H), 7.02 (d, J=9.0 Hz, 1H). ¹³C NMR (75 MHz, CDCl3) δ 167.3, 160.6, 136.7, 135.5, 127.8 (q, J=33.6 Hz), 126.6 (q, J=3.9 Hz), 125.3 (q, J=3.8 Hz), 125.3, 124.4, 123.9, 123.3 (q, J=270.5 Hz), 122.0, 120.9, 115.4. HRMS (ESI) calcd for C₁₄H₉Cl₂F₃NO₂, 349.9962 (M+H)⁺; found, 349.9950.

5-Chloro-N-(3,4-difluorophenyl)-2-hydroxybenzamide (15). Compound 15 (120 mg, 50% in two steps) was prepared by a procedure similar to that used to prepare compound 10. The title compound was obtained as a grey solid. HPLC purity 99.9% (t_{R}=19.46 min). ¹H NMR (300 MHz, DMSO-d₆) δ 11.60 (s, 1H), 10.50 (s, 1H), 7.94-7.82 (m, 2H), 7.51-7.37 (m, 3H), 7.02 (d, J=8.7 Hz, 1H). ¹³C NMR (75 MHz, DMSO-d₆) δ 164.9, 156.4, 148.9 (dd, J=242.0, 13.1 Hz), 145.9 (dd, J=241.2, 12.7 Hz), 135.1 (dd, J=9.0, 3.0 Hz), 133.0, 128.4, 122.7, 119.9, 119.0, 117.4 (d, J=17.3 Hz), 117.1 (dd, J=6.0, 3.4 Hz), 109.8 (d, J=21.4 Hz). HRMS (ESI) calcd for C₁₃H₉ClF₂NO₂, 284.0290 (M+H)⁺; found, 284.0282.

N-(3,5-Bis(trifluoromethyl)phenyl)-5-chloro-2-hydroxybenzamide (16). Compound 16 (105 mg, 55% in two steps) was prepared by a procedure similar to that used to prepare compound 10. The title compound was obtained as a light yellow solid. HPLC purity 99.5% (t_{R}=21.53 min). ¹H NMR (300 MHz, CD₃OD) δ 8.26 (s, 2H), 7.89 (d, J=2.7 Hz, 1H), 7.62 (s, 1H), 7.29 (dd, J=9.0, 2.7 Hz, 1H), 6.87 (d, J=8.7 Hz, 1H). ¹³C NMR (75 MHz, CD₃OD) δ 167.4, 158.7, 141.2, 135.0, 133.2 (q, J=33.2 Hz, 2C), 129.8, 125.5, 124.6 (q, J=270.3 Hz, 2C), 121.5 (q, J=3.2 Hz, 2C), 120.0, 119.1, 118.2 (hept, J=3.8 Hz). HRMS (ESI) calcd for C₁₅H₉ClF₆NO₂, 384.0226 (M+H)⁺; found, 384.0219.

5-Chloro-N-(3-fluoro-5-(trifluoromethyl)phenyl)-2-hydroxybenzamide (17). Compound 17 (100 mg, 33% in two steps) was prepared by a procedure similar to that used to prepare compound 10. The title compound was obtained as a white solid. HPLC purity 96.7% (t_{R}=20.86 min). ¹H NMR (300 MHz, CDCl₃) δ 11.42 (s, 1H), 8.04 (s, 1H), 7.79 (dt, J=10.2, 2.1 Hz, 1H), 7.58 (s, 1H), 7.52 (d, J=2.4 Hz, 1H), 7.43 (dd, J=8.7, 2.4 Hz, 1H), 7.18 (d, J=7.8 Hz, 1H), 7.02 (d, J=8.7 Hz, 1H). ¹³C NMR (75 MHz, DMSO-d₆) δ 165.2, 162.0 (d, J=242.8 Hz), 156.2, 141.0 (d, J=11.3 Hz), 133.1, 131.0 (qd, J=32.4, 9.8 Hz), 128.6, 123.3 (q, J=270.6 Hz), 122.7, 120.4, 119.0, 112.9 (quint, J=3.4 Hz), 110.9 (d, J=26.1 Hz), 107.8 (dq, J=28.7, 3.8 Hz). HRMS (ESI) calcd for C₁₄H₉ClF₄NO₂, 334.0258 (M+H)⁺; found, 334.0259.

N-(3-Fluoro-5-(trifluoromethyl)phenyl)-2-hydroxybenzamide (18). Compound 18 (165 mg, 82% in two steps) was prepared by a procedure similar to that used to prepare compound 10. The title compound was obtained as a pale yellow solid. HPLC purity 99.3% (t_{R}=18.99 min). ¹H NMR (300 MHz, CD₃OD+CDCl₃) δ 7.96-7.89 (m, 1H), 7.84-7.70 (m, 2H), 7.40-7.31 (m, 1H), 7.05 (d, J=8.4 Hz, 1H), 6.96-6.87 (m, 2H). ¹³C NMR (75 MHz, CD₃OD+CDCl3) δ 168.2, 163.4 (d, J=244.5 Hz), 159.5, 141.4 (d, J=11.1 Hz), 135.0, 133.1 (qd, J=33.0, 9.3 Hz), 129.7, 124.0 (qd, J=270.5, 3.5 Hz), 120.3, 118.0, 113.8 (m), 117.2, 111.8 (d, J=26.0 Hz), 108.5 (dq, J=24.9, 3.8 Hz). HRMS (ESI) calcd for C₁₄H₁₀F₄NO₂, 300.0648 (M+H)⁺; found, 300.0640.

N-(3-Fluoro-5-(trifluoromethyl)phenyl)-2-hydroxy-5-methylbenzamide (19). Compound 19 (210 mg, 92% in two steps) was prepared by a procedure similar to that used to prepare compound 10. The title compound was obtained as a white solid. HPLC purity 98.4% (t_{R}=19.72 min). ¹H NMR (300 MHz, CDCl₃) δ 11.28 (s, 1H), 8.09 (s, 1H), 7.82 (dt, J=10.2, 2.1 Hz, 1H), 7.56 (s, 1H), 7.33-7.27 (m, 2H), 7.15 (d, J=7.8 Hz, 1H), 6.98-6.93 (m, 1H), 2.35 (s, 3H). ¹³C NMR (75 MHz, CD₃OD+CDCl₃) δ 168.1, 163.3 (d, J=244.4 Hz), 157.1, 141.3 (d, J=11.0 Hz), 135.7, 133.0 (qd, J=33.2, 9.3 Hz), 129.6, 129.6, 124.0 (qd, J=270.4, 3.5 Hz), 117.7, 116.7, 113.7 (quint, J=3.7 Hz), 111.7 (d, J=26.5 Hz), 108.3 (dq, J=25.0, 3.8 Hz), 20.5. HRMS (ESI) calcd for C₁₅H₁₂F₄NO₂, 314.0804 (M+H)⁺; found, 314.0797.

4-Chloro-N-(3-fluoro-5-(trifluoromethyl)phenyl)-2-hydroxybenzamide (20). Compound 20 (320 mg, 89% in two steps) was prepared by a procedure similar to that used to prepare compound 10. The title compound was obtained as a yellow solid. HPLC purity 99.9% (t_{R}=20.02 min). ¹H NMR (300 MHz, DMSO-d₆) δ 11.70 (s, 1H), 10.67 (s, 1H), 7.99 (s, 1H), 7.93 (dt, J=11.1, 1.8 Hz, 1H), 7.88-7.81 (m, 1H), 7.40 (d, J=8.4 Hz, 1H), 7.07-7.01 (m, 2H). ¹³C NMR (75 MHz, DMSO-d₆) δ 165.6, 162.0 (d, J=242.7 Hz), 158.1, 141.0 (d, J=11.3 Hz), 137.5, 131.1, 130.0 (qd, J=25.0, 9.9 Hz), 123.3 (qd, J=270.9, 3.5 Hz), 119.4, 118.0, 116.7, 112.8 (m), 110.8 (d, J=26.0 Hz), 107.7 (dq, J=25.0, 3.6 Hz). HRMS (ESI) calcd for C₁₄H₉ClF₄NO₂, 334.0258 (M+H)⁺; found, 334.0251.

3-Chloro-N-(3-fluoro-5-(trifluoromethyl)phenyl)benzamide (21). To a solution of 3-chlorobenzoic acid (100 mg, 0.64 mmol) and 3-fluoro-5-(trifluoromethyl)aniline (137 mg, 0.77 mmol) in toluene (15 mL) was added PCl₃ (132 mg, 0.96 mmol). The resulting mixture was stirred at 100° C. for 24 h, and then concentrated. The residue was purified by column chromatography (Hex/EtOAc=5/1 to 3/1) to afford compound 21 as a white solid (190 mg, 93%). HPLC purity 99.9% (t_{R}=19.73 min). ¹H NMR (300 MHz, CDCl₃) δ 8.83 (s, 1H), 7.80-7.70 (m, 2H), 7.66 (dt, J=7.8, 1.2 Hz, 1H), 7.59 (s, 1H), 7.50-7.40 (m, 1H), 7.31 (t, J=7.8 Hz, 1H), 7.12-7.02 (m, 1H). ¹³C NMR (75 MHz, CDCl₃) δ 165.6, 162.7 (d, J=246.4 Hz), 139.9 (d, J=11.0 Hz), 135.7, 135.1, 132.8 (qd, J=33.4, 9.3 Hz), 132.5, 130.2, 127.5, 125.4. 123.1 (qd, J=270.8, 3.3 Hz), 113.0 (quint, J=3.7 Hz), 111.2 (d, J=26.1 Hz), 109.0 (dq, J=24.6, 3.6 Hz). HRMS (ESI) calcd for C₁₄H₉ClF₄NO, 318.0309 (M+H)⁺; found, 318.0305.

5-Chloro-N-(2-chloro-4-nitrobenzyl)-2-hydroxybenzamide (30). To a solution of 5-chloro-2-methoxybenzoic acid (378 mg, 2.0 mmol), (2-chloro-4-nitrophenyl)methanamine (270 mg, 1.5 mmol) and DMAP (35 mg, 0.29 mmol) in DCM (20 mL) was added Et₃N (202 mg, 2.0 mmol) and EDCI (556 mg, 2.9 mmol) successively at 0° C. The resulting mixture was stirred at r.t. for 12 h and concentrated. The residue was purified by column chromatography (Hex/EtOAc=6/1 to 3/1) to afford the amide intermediate as a yellow solid. The amide intermediate was dissolved in DCM (30 mL), and then BBr₃ (4.4 mL, 4.4 mmol, 1 M in DCM) was added dropwise at 0° C. The mixture was stirred at 0° C. for 4 h until the reaction was completed monitored by TLC. The mixture was diluted with DCM, washed with H₂O and concentrated. The residue was purified by recrystallization (MeOH/H₂O) to afford compound 30 as a brown solid (285 mg, 57% in two steps). HPLC purity 99.7% (t_{R}=18.66 min). ¹H NMR (300 MHz, CDCl₃) δ 11.85 (s, 1H), 8.28 (d, J=2.3 Hz, 1H), 8.12 (dd, J=8.5, 2.3 Hz, 1H), 7.63 (d, J=8.5 Hz, 1H), 7.41-7.31 (m, 2H), 7.00-6.90 (m, 1H), 6.85 (s, 1H), 4.79 (d, J=6.1 Hz, 2H). ¹³C NMR (75 MHz, CDCl₃) δ 169.3, 160.3, 148.0, 141.9, 134.8, 134.5, 130.7, 125.2, 125.0, 123.8, 122.3, 120.5, 114.7, 41.6. HRMS (ESI) calcd for C₁₄H₁₁C₂N₂O₄, 341.0096 (M+H)⁺; found, 341.0091.

5-Chloro-N-(2-chloro-4-fluorobenzyl)-2-hydroxybenzamide (31). Compound 31 (257 mg, 73% in two steps) was prepared by a procedure similar to that used to prepare compound 30. The title compound was obtained as a yellow solid. HPLC purity 99.9% (t_{R}=18.82 min). ¹H NMR (300 MHz, CDCl₃) δ 12.05 (s, 1H), 7.44 (dd, J=8.4, 6.0 Hz, 1H), 7.37-7.28 (m, 2H), 7.17 (dd, J=8.4, 2.4 Hz, 1H), 7.04-6.90 (m, 2H), 6.66 (s, 1H), 4.68 (d, J=5.7 Hz, 2H). ¹³C NMR (75 MHz, CDCl₃+CD₃OD) δ 168.3, 161.8 (d, J=247.7 Hz), 158.2, 134.0 (d, J=10.4 Hz), 133.5 (2C), 131.2 (d, J=3.1 Hz), 130.6 (d, J=8.8 Hz), 127.3, 123.9, 119.0, 116.8 (d, J=24.8 Hz), 114.0 (d, J=20.9 Hz), 40.7. HRMS (ESI) calcd for C₁₄H₁₁Cl₂FNO₂, 314.0151 (M+H)⁺; found, 314.0143.

5-Chloro-N-(2,4-dichlorobenzyl)-2-hydroxybenzamide (32). Compound 32 (310 mg, 72% in two steps) was prepared by a procedure similar to that used to prepare compound 30. The title compound was obtained as a yellow solid. HPLC purity 99.4% (t_{R}=19.63 min). ¹H NMR (300 MHz, CDCl₃) δ 12.01 (s, 1H), 7.45-7.21 (m, 5H), 6.93 (d, J=8.7 Hz, 1H), 6.69 (s, 1H), 4.67 (d, J=5.7 Hz, 2H). ¹³C NMR (75 MHz, CDCl₃) δ 169.0, 160.3, 134.8, 134.6, 134.5, 133.4, 131.5, 129.8, 127.7, 125.1, 123.6, 120.4, 115.0, 41.4. HRMS (ESI) calcd for C₁₄H₁₁Cl₃NO₂, 329.9855 (M+H)⁺; found, 329.9844.

5-Chloro-N-(2,4-difluorobenzyl)-2-hydroxybenzamide (33). Compound 33 (207 mg, 53% in two steps) was prepared by a procedure similar to that used to prepare compound 30. The title compound was obtained as a yellow solid. HPLC purity 99.1% (t_{R}=18.27 min). ¹H NMR (300 MHz, CDCl₃) δ 12.06 (s, 1H), 7.46-7.29 (m, 3H), 6.99-6.79 (m, 3H), 6.58 (s, 1H), 4.63 (d, J=6.0 Hz, 2H). ¹³C NMR (75 MHz, CDCl₃) δ 169.0, 162.9 (dd, J=248.2, 11.9 Hz), 161.3 (dd, J=247.4, 12.0 Hz), 160.4, 134.5, 131.7 (dd, J=9.7, 5.7 Hz), 125.1, 123.6, 120.4, 120.4 (dd, J=14.7, 4.1 Hz), 115.1, 111.8 (dd, J=21.0, 3.7 Hz), 104.3 (t, J=25.3 Hz), 37.5 (d, J=3.2 Hz). HRMS (ESI) calcd for C₁₄H₁₁ClF₂NO₂, 298.0446 (M+H)⁺; found, 298.0439.

5-Chloro-N-(3,4-difluorobenzyl)-2-hydroxybenzamide (34). Compound 34 (288 mg, 80% in two steps) was prepared by a procedure similar to that used to prepare compound 30. The title compound was obtained as a grey solid. HPLC purity 99.2% (t_{R}=18.33 min). ¹H NMR (300 MHz, CDCl₃) δ 12.03 (s, 1H), 7.40-7.30 (m, 2H), 7.22-7.04 (m, 3H), 6.96 (d, J=8.7 Hz, 1H), 6.55 (s, 1H), 4.58 (d, J=5.7 Hz, 2H). ¹³C NMR (75 MHz, CD₃OD+CDCl₃) δ 168.7, 158.6, 150.7 (dd, J=246.3, 12.7 Hz), 150.3 (dd, J=245.5, 12.6 Hz), 136.0 (dd, J=5.1, 4.0 Hz), 133.9, 128.0, 124.4, 124.0 (dd, J=6.3, 3.7 Hz), 119.4, 117.6 (d, J=17.3 Hz), 117.3, 116.9 (d, J=17.6 Hz), 42.7. HRMS (ESI) calcd for C₁₄H₁₁ClF₂NO₂, 298.0446 (M+H)⁺; found, 298.0437.

5-Chloro-N-(3-fluoro-5-(trifluoromethyl)benzyl)-2-hydroxybenzamide (35). Compound 35 (198 mg, 66% in two steps) was prepared by a procedure similar to that used to prepare compound 30. The title compound was obtained as a grey solid. HPLC purity 99.5% (t_{R}=19.04 min). ¹H NMR (300 MHz, CDCl₃) δ 11.97 (s, 1H), 7.45-7.29 (m, 3H), 7.23 (d, J=8.4 Hz, 2H), 7.04 (s, 1H), 6.91 (d, J=9.0 Hz, 1H), 4.65 (d, J=5.4 Hz, 2H)¹³C NMR (75 MHz, CDCl₃) δ 169.2, 162.8 (d, J=248.5 Hz), 160.0, 141.4 (d, J=7.2 Hz), 134.7, 133.2 (qd, J=33.2, 8.2 Hz), 125.4, 123.9, 123.2 (qd, J=270.8, 2.9 Hz), 120.3 (m), 120.3, 118.4 (d, J=22.3 Hz), 114.9, 112.4 (dq, J=24.4, 3.7 Hz), 43.0. HRMS (ESI) calcd for C₁₅H₁₁ClF₄NO₂, 348.0414 (M+H)⁺; found, 348.0407.

5-Chloro-N-(3-fluoro-4-(trifluoromethyl)benzyl)-2-hydroxybenzamide (36). Compound 36 (215 mg, 51% in two steps) was prepared by a procedure similar to that used to prepare compound 30. The title compound was obtained as an off-white solid. HPLC purity 97.6% (t_{R}=19.17 min). ¹H NMR (300 MHz, CDCl₃) δ 11.93 (s, 1H), 7.61 (t, J=7.5 Hz, 1H), 7.41-7.32 (m, 2H), 7.25-7.16 (m, 2H), 6.97 (d, J=8.7 Hz, 1H), 6.64 (s, 1H), 4.68 (d, J=6.0 Hz, 2H). ¹³C NMR (75 MHz, CDCl₃+CD₃OD) δ 168.3, 159.7 (qd, J=254.3, 2.3 Hz), 158.1, 145.6 (d, J=7.4 Hz), 133.5, 127.4, 127.1 (dq, J=4.6, 1.7 Hz), 123.9, 122.8 (d, J=3.5 Hz), 122.5 (q, J=270.2 Hz), 118.9, 116.9 (dq, J=32.8, 12.5 Hz), 116.5, 115.5 (q, J=21.1 Hz), 42.3. HRMS (ESI) calcd for C₁₅H₁₁ClF₄NO₂, 348.0414 (M+H)⁺; found, 348.0406.

5-Chloro-N-(2-fluoro-4-(trifluoromethyl)benzyl)-2-hydroxybenzamide (37). Compound 37 (153 mg, 50% in two steps) was prepared by a procedure similar to that used to prepare compound 30. The title compound was obtained as a yellow solid. HPLC purity 98.7% (t_{R}=19.23 min). ¹H NMR (300 MHz, CDCl₃) δ 11.97 (s, 1H), 7.54 (t, J=7.5 Hz, 1H), 7.45-7.30 (m, 4H), 6.93 (d, J=9.6 Hz, 1H), 6.77 (s, 1H), 4.71 (d, J=5.7 Hz, 2H). ¹³C NMR (75 MHz, CD₃OD+CDCl₃) δ 168.9, 161.0 (d, J=247.1 Hz), 158.6, 134.1, 131.9 (qd, J=33.2, 8.0 Hz), 130.8 (d, J=4.5 Hz), 130.3 (d, J=14.9 Hz), 128.5, 124.6, 123.9 (qd, J=270.0, 2.6 Hz), 121.6 (quint, J=3.8 Hz), 119.4, 117.6, 113.1 (dq, J=24.9, 3.8 Hz), 37.5 (d, J=4.4 Hz). HRMS (ESI) calcd for C₁₅H₁₁ClF₄NO₂, 348.0414 (M+H)⁺; found, 348.0408.

5-Chloro-N-(4-chlorobenzyl)-2-hydroxybenzamide (38). Compound 38 (300 mg, 82% in two steps) was prepared by a procedure similar to that used to prepare compound 30. The title compound was obtained as a grey solid. HPLC purity 96.1% (t_{R}=18.79 min). ¹H NMR (300 MHz, CDCl₃) δ 12.11 (s, 1H), 7.37-7.23 (m, 6H), 6.94 (d, J=9.6 Hz, 1H), 6.60 (s, 1H), 4.58 (d, J=5.7 Hz, 2H). ¹³C NMR (75 MHz, CDCl₃) δ 169.0, 160.3, 135.8, 134.5, 134.0, 129.4 (2C), 129.2 (2C), 125.1, 123.6, 120.4, 115.1, 43.3. HRMS (ESI) calcd for C₁₄H₁₂Cl₂NO₂, 296.0245 (M+H)⁺; found, 296.0239.

5-Chloro-N-(3-chlorobenzyl)-2-hydroxybenzamide (39). Compound 39 (290 mg, 68% in two steps) was prepared by a procedure similar to that used to prepare compound 30. The title compound was obtained as a yellow solid. HPLC purity 98.8% (t_{R}=18.79 min). ¹H NMR (300 MHz, CDCl₃) δ 12.10 (s, 1H), 7.38-7.19 (m, 6H), 6.95 (d, J=9.0 Hz, 1H), 6.57 (s, 1H), 4.60 (d, J=5.7 Hz, 2H). ¹³C NMR (75 MHz, CDCl₃) δ 169.0, 160.4, 139.3, 135.0, 134.5, 130.4, 128.3, 128.2, 126.2, 125.1, 123.6, 120.4, 115.0, 43.4. HRMS (ESI) calcd for C₁₄H₁₂Cl₂NO₂, 296.0245 (M+H)⁺; found, 296.0237.

5-Chloro-N-(4-fluorobenzyl)-2-hydroxybenzamide (40). Compound 40 (260 mg, 51% in two steps) was prepared by a procedure similar to that used to prepare compound 30. The title compound was obtained as an off-white solid. HPLC purity 99.9% (t_{R}=18.00 min). ¹H NMR (300 MHz, CDCl₃) δ 12.15 (s, 1H), 7.38-7.27 (m, 4H), 7.05 (t, J=8.7 Hz, 2H), 6.94 (d, J=8.7 Hz, 1H), 6.55 (s, 1H), 4.59 (d, J=5.4 Hz, 2H). ¹³C NMR (75 MHz, CDCl₃) δ 168.9, 162.6 (d, J=245.1 Hz), 160.3, 134.4, 133.1 (d, J=3.2 Hz), 129.9 (d, J=8.2 Hz, 2C), 125.1, 123.6, 120.4, 116.0 (d, J=21.5 Hz, 2C), 115.1, 43.3. HRMS (ESI) calcd for C₁₄H₁₂ClFNO₂, 280.0541 (M+H)⁺; found, 280.0533.

5-Chloro-N-(4-fluorobenzyl)-2-hydroxy-N-methylbenzamide (41). Compound 41 (340 mg, 93% in two steps) was prepared by a procedure similar to that used to prepare compound 30. The title compound was obtained as a yellow solid. HPLC purity 95.0% (t_{R}=16.29 min). ¹H NMR (300 MHz, CDCl₃) δ 9.71 (s, 1H), 7.32-7.22 (m, 4H), 7.07 (t, J=8.7 Hz, 2H), 6.96 (d, J=8.4 Hz, 1H), 4.69 (s, 2H), 3.06 (s, 3H). ¹³C NMR (75 MHz, CDCl₃) δ 170.9, 162.6 (d, J=245.1 Hz), 157.8, 132.8, 131.9 (d, J=3.2 Hz), 129.6 (d, J=7.5 Hz, 2C), 127.8, 123.5, 119.7, 118.5, 116.0 (d, J=21.5 Hz, 2C), 52.6, 36.7. HRMS (ESI) calcd for C₁₅H₁₄ClFNO₂, 294.0697 (M+H)⁺; found, 294.0689.

5-Chloro-2-hydroxy-N-(4-(trifluoromethyl)benzyl)benzamide (42). Compound 42 (220 mg, 65% in two steps) was prepared by a procedure similar to that used to prepare compound 30. The title compound was obtained as an off-white solid. HPLC purity 99.8% (t_{R}=19.04 min). ¹H NMR (300 MHz, CDCl₃) δ 12.05 (s, 1H), 7.61 (d, J=7.8 Hz, 2H), 7.44 (d, J=7.9 Hz, 2H), 7.39-7.30 (m, 2H), 6.94 (d, J=9.0 Hz, 1H), 6.73 (s, 1H), 4.67 (d, J=5.4 Hz, 2H). ¹³C NMR (75 MHz, CDCl3) δ 169.1, 160.3, 141.4, 134.6, 130.4 (q, J=32.3 Hz), 128.2 (2C), 126.0 (q, J=3.8 Hz, 2C), 125.2, 124.1 (q, J=270.4 Hz), 123.7, 120.4, 115.0, 43.4. HRMS (ESI) calcd for C₁₅H₁₂ClF₃NO₂, 330.0509 (M+H)⁺; found, 330.0501.

5-Chloro-2-hydroxy-N-(4-nitrobenzyl)benzamide (43). Compound 43 (260 mg, 69% in two steps) was prepared by a procedure similar to that used to prepare compound 30. The title compound was obtained as a yellow solid. HPLC purity 99.8% (t_{R}=17.81 min). ¹H NMR (300 MHz, DMSO-d₆) δ 12.25 (s, 1H), 9.46 (s, 1H), 8.21 (d, J=8.7 Hz, 2H), 7.96 (d, J=2.4 Hz, 1H), 7.60 (d, J=8.7 Hz, 2H), 7.46 (dd, J=9.0, 2.7 Hz, 1H), 6.97 (d, J=8.7 Hz, 1H), 4.64 (d, J=6.0 Hz, 2H). ¹³C NMR (75 MHz, DMSO-d₆) δ 167.4, 158.2, 146.8, 146.5, 133.3, 128.3 (2C), 127.6, 123.5 (2C), 122.5, 119.3, 117.0, 42.1. HRMS (ESI) calcd for C₁₄H₁₂ClN₂O₄, 307.0486 (M+H)⁺; found, 307.0478.

5-Chloro-2-hydroxy-N-(4-methylbenzyl)benzamide (44). Compound 44 (330 mg, 85% in two steps) was prepared by a procedure similar to that used to prepare compound 30. The title compound was obtained as an off-white solid. HPLC purity 99.3% (t_{R}=18.65 min). ¹H NMR (300 MHz, CDCl₃) δ 12.24 (s, 1H), 7.38-7.16 (m, 6H), 6.95 (d, J=8.7 Hz, 1H), 6.47 (s, 1H), 4.58 (d, J=5.4 Hz, 2H), 2.37 (s, 3H). ¹³C NMR (75 MHz, CDCl₃) δ 168.8, 160.4, 138.0, 134.3, 134.1, 129.8 (2C), 128.2 (2C), 125.1, 123.5, 120.3, 115.3, 43.8, 21.3. HRMS (ESI) calcd for C₁₅H₁₅ClNO₂, 276.0791 (M+H)⁺; found, 276.0784.

N-Benzyl-5-chloro-2-hydroxybenzamide (45). Compound 45 (35 mg, 55% in two steps) was prepared by a procedure similar to that used to prepare compound 30. The title compound was obtained as an off-white solid. HPLC purity 99.9% (t_{R}=17.91 min). ¹H NMR (300 MHz, CDCl₃) δ 12.23 (s, 1H), 7.45-7.29 (m, 7H), 6.94 (d, J=9.3 Hz, 1H), 6.56 (s, 1H), 4.62 (d, J=5.7 Hz, 2H). ¹³C NMR (75 MHz, CDCl₃) δ 168.9, 160.3, 137.2, 134.3, 129.1 (2C), 128.2, 128.1 (2C), 125.1, 123.5, 120.3, 115.2, 44.0. HRMS (ESI) calcd for C₁₄H₁₃ClNO₂, 262.0635 (M+H)⁺; found, 262.0628.

(R)-5-Chloro-2-hydroxy-N-(1-phenylethyl)benzamide (46). To a solution of (R)-1-phenylethanamine (150 mg, 1.2 mmol), 5-chlorosalicylic acid (214 mg, 1.2 mmol) and DMAP (30 mg, 0.25 mmol) in 30 mL of DCM was added EDCI (475 mg, 2.5 mmol) at 0° C. The resulting mixture was stirred at r.t. for 24 h and then concentrated. The residue was purified by column chromatography (Hex/EtOAc=10/1) to afford compound 46 as an off-white solid (200 mg, 55%). HPLC purity 99.1% (t_{R}=18.46 min). ¹H NMR (300 MHz, CDCl3) δ 12.25 (s, 1H), 7.43-7.26 (m, 7H), 6.90 (d, J=9.0 Hz, 1H), 6.74 (d, J=7.2 Hz, 1H), 5.29 (p, J=6.9 Hz, 1H), 1.60 (d, J=6.9 Hz, 3H). ¹³C NMR (75 MHz, CDCl₃) δ 168.2, 160.0, 142.3, 134.1, 128.9 (2C), 127.8, 126.2 (2C), 125.3, 123.4, 120.1, 115.4, 49.4, 21.6. HRMS (ESI) calcd for C₁₅H₁₅ClNO₂, 276.0791 (M+H)⁺; found, 276.0790.

(S)-5-Chloro-2-hydroxy-N-(1-phenylethyl)benzamide (47). Compound 47 (108 mg, 39%) was prepared by a procedure similar to that used to prepare compound 46. The title compound was obtained as a white solid. HPLC purity 97.1% (t_{R}=19.75 min). ¹H NMR (600 MHz, CDCl₃) δ 12.22 (s, 1H), 7.28-7.48 (m, 7H), 6.93 (d, 1H, J=9.0 Hz), 6.55 (d, 1H, J=6.6 Hz), 5.29-5.34 (m, 1H), 1.64 (d, 3H, J=7.2 Hz). ¹³C NMR (150 MHz, CDCl₃) δ 168.2, 160.2, 142.3, 134.2, 129.0, 129.0, 127.9, 126.3, 126.3, 125.2, 123.4, 120.2, 115.4, 49.5, 21.8. HRMS (ESI) calcd for C₁₅H₁₅ClNO₂, 276.0791 (M+H)⁺; found, 276.0790.

(R)-5-Chloro-N-(1-(4-chlorophenyl)ethyl)-2-hydroxybenzamide (48). Compound 48 (140 mg, 40%) was prepared by a procedure similar to that used to prepare compound 46. The title compound was obtained as a pale yellow solid. HPLC purity 96.4% (t_{R}=19.30 min). ¹H NMR (300 MHz, CDCl₃) δ 12.11 (s, 1H), 7.40 (d, J=2.4 Hz, 1H), 7.34-7.24 (m, 5H), 6.89 (d, J=9.0 Hz, 1H), 6.75 (d, J=7.2 Hz, 1H), 5.22 (p, J=7.2 Hz, 1H), 1.56 (d, J=6.9 Hz, 3H). ¹³C NMR (75 MHz, CDCl₃) δ 168.2, 160.0, 140.9, 134.2, 133.5, 129.0 (2C), 127.6 (2C), 125.4, 123.5, 120.1, 115.2, 48.9, 21.6. HRMS (ESI) calcd for C₁₅H₁₄Cl₂NO₂, 310.0402 (M+H)⁺; found, 310.0399.

(R)-5-Chloro-N-(1-(4-fluorophenyl)ethyl)-2-hydroxybenzamide (49). Compound 49 (270 mg, 64%) was prepared by a procedure similar to that used to prepare compound 46. The title compound was obtained as a yellow solid. HPLC purity 99.1% (t_{R}=18.62 min). ¹H NMR (300 MHz, CDCl₃) δ 12.15 (s, 1H), 7.48-7.42 (m, 1H), 7.36-7.26 (m, 3H), 7.06-6.92 (m, 3H), 6.87 (d, J=9.0 Hz, 1H), 5.24 (p, J=7.2 Hz, 1H), 1.56 (d, J=7.2 Hz, 3H). ¹³C NMR (75 MHz, CDCl₃) δ 168.1, 162.6 (d, J=169.5 Hz), 159.7, 138.2 (d, J=2.9 Hz), 134.1, 127.9 (d, J=8.1 Hz, 2C), 125.6, 123.5, 119.9, 115.6 (d, J=21.3 Hz, 2C), 115.4, 48.7, 21.6. HRMS (ESI) calcd for C₁₅H₁₄ClFNO₂, 294.0697 (M+H)⁺; found, 294.0693.

(R)-5-Chloro-2-hydroxy-N-(1-(4-methoxyphenyl)ethyl)benzamide (50). Compound 50 (220 mg, 54%) was prepared by a procedure similar to that used to prepare compound 46. The title compound was obtained as a pale yellow solid. HPLC purity 95.2% (t_{R}=18.42 min). ¹H NMR (300 MHz, CDCl₃) δ 12.30 (s, 1H), 7.38 (d, J=2.1 Hz, 1H), 7.32-7.26 (m, 3H), 6.92-6.84 (m, 3H), 6.73 (d, J=7.2 Hz, 1H), 5.23 (p, J=6.9 Hz, 1H), 3.78 (s, 3H), 1.58 (d, J=6.9 Hz, 3H). ¹³C NMR (75 MHz, CDCl₃) δ 168.1, 160.0, 159.1, 134.3, 134.0, 127.5 (2C), 125.3, 123.3, 120.0, 115.4, 114.2 (2C), 55.3, 48.8, 21.4. HRMS (ESI) calcd for C₁₆H₁₇ClNO₃, 306.0897 (M+H)⁺; found, 306.0892.

5-Chloro-N-(2-(4-fluorophenyl)propan-2-yl)-2-hydroxybenzamide (51). To a solution of 1-(4-fluorophenyl)-1-methylethylamine (180 mg, 1.2 mmol), 5-chloro-2-methoxybenzoic acid (219 mg, 1.2 mmol) and DMAP (28 mg, 0.23 mmol) in 20 mL of DCM was added EDCI (336 mg, 1.8 mmol). The resulting mixture was stirred at 50° C. for 48 h and then concentrated. The residue was purified by column chromatography (Hex/EtOAc=10/1) to afford the amide intermediate 5-chloro-N-(2-(4-fluorophenyl)propan-2-yl)-2-methoxybenzamide (57 mg, 15%) as colorless oil. ¹H NMR (300 MHz, CDCl₃) δ 8.32-8.18 (m, 1H), 8.09 (d, J=3.0 Hz, 1H), 7.45-7.33 (m, 3H), 7.05-6.95 (m, 2H), 6.91 (d, J=8.7 Hz, 1H), 3.97 (s, 3H), 1.77 (s, 6H). ¹³C NMR (75 MHz, CDCl3) δ 162.6, 161.5 (d, J=243.0 Hz), 155.9, 142.9 (d, J=3.2 Hz), 132.3, 132.0, 126.9, 126.6 (d, J=7.9 Hz, 2C), 123.8, 115.1 (d, J=21.1 Hz, 2C), 112.9, 56.5, 55.6, 29.5 (2C).

To a solution of the amide intermediate 5-chloro-N-(2-(4-fluorophenyl)propan-2-yl)-2-methoxybenzamide (57 mg, 0.18 mmol) in 15 mL of DCM was added BBr₃ (0.35 mL, 0.35 mmol, 1M in DCM) dropwise at 0° C. The resulting mixture was stirred at r.t. for 1 h, and then the mixture was poured into 20 mL of ice water. The organic phase was separated and the aqueous phase was extracted with DCM (60 mL). The organic phases were combined, dried (Na₂SO₄) and concentrated. The residue was purified by column chromatography (Hex/EtOAc=10/1) to afford compound 51 (50 mg, 92%) as an off-white solid. HPLC purity 99.0% (t_{R}=18.91 min). ¹H NMR (300 MHz, CDCl₃) δ 12.03 (s, 1H), 7.45-7.29 (m, 4H), 7.10-6.99 (m, 2H), 6.91 (d, J=8.7 Hz, 1H), 6.49 (s, 1H), 1.80 (s, 6H). ¹³C NMR (75 MHz, CDCl₃) δ 168.4, 161.8 (d, J=244.1 Hz), 160.4, 141.7 (d, J=3.2 Hz), 134.2, 126.4 (d, J=8.0 Hz, 2C), 125.1, 123.3, 120.5, 115.7, 115.5 (d, J=21.2 Hz, 2C), 56.5, 29.6 (2C). HRMS (ESI) calcd for C₁₆H₁₆ClFNO₂, 308.0854 (M+H)⁺; found, 308.0852.

5-Chloro-N-(4-fluorophenethyl)-2-hydroxybenzamide (52). Methyl 5-chloro-2-hydroxybenzoate (100 mg, 0.54 mmol) was dissolved in 10 mL of methanol followed by addition of 2-(4-fluorophenyl)ethanamine (224 mg, 1.6 mmol). The resulting mixture was stirred at r.t. for 48 h, and then concentrated. The residue was purified by preparative TLC to afford compound 52 as a yellow solid (123 mg, 52%). HPLC purity 99.6% (t_{R}=18.46 min). ¹H NMR (300 MHz, CDCl₃) δ 12.20 (s, 1H), 7.32 (dd, J=8.7, 2.1 Hz, 1H), 7.23-7.14 (m, 3H), 7.02 (t, J=8.7 Hz, 2H), 6.92 (d, J=8.7 Hz, 1H), 6.35 (s, 1H), 3.67 (q, J=6.6 Hz, 2H), 2.91 (t, J=6.9 Hz, 2H). ¹³C NMR (75 MHz, CDCl₃) δ 169.1, 162.0 (d, J=243.5 Hz), 160.2, 134.3, 134.1 (d, J=3.2 Hz), 130.3 (d, J=7.8 Hz, 2C), 125.0, 123.5, 120.3, 115.8 (d, J=21.2 Hz, 2C), 115.3, 41.2, 34.8. HRMS (ESI) calcd for C₁₅H₁₄ClFNO₂, 294.0697 (M+H)⁺; found, 294.0691.

2-(5-Chloro-2-hydroxyphenyl)-N-(3-fluoro-5-(trifluoromethyl)phenyl)acetamide (53). Compound 53 (300 mg, 82% in two steps) was prepared by a procedure similar to that used to prepare compound 10 starting from 2-(5-chloro-2-methoxyphenyl)acetic acid and 3-fluoro-5-(trifluoromethyl)aniline. The title compound was obtained as a yellow solid. HPLC purity 99.5% (t_{R}=18.13 min). ¹H NMR (300 MHz, CD₃OD) δ 7.77-7.65 (m, 2H), 7.16 (d, J=2.4 Hz, 1H), 7.12-7.02 (m, 2H), 6.76 (d, J=8.4 Hz, 1H), 3.67 (s, 2H). ¹³C NMR (75 MHz, CD₃OD) δ 172.6, 164.1 (d, J=243.8 Hz), 155.6, 142.7 (d, J=11.0 Hz), 133.6 (qd, J=33.1, 9.4 Hz), 131.9, 129.1, 125.0, 124.8, 124.7 (qd, J=270.0, 3.5 Hz), 117.2, 113.1 (d, J=3.7 Hz), 111.0 (d, J=26.2 Hz), 108.3 (dq, J=25.1, 3.8 Hz), 39.4. HRMS (ESI) calcd for C₁₅H₁₁ClF₄NO₂, 348.0414 (M+H)⁺; found, 348.0408.

2-(5-Chloro-2-hydroxyphenyl)-N-(2-chloro-4-nitrophenyl)acetamide (54). Compound 54 (277 mg, 77% in two steps) was prepared by a procedure similar to that used to prepare compound 10 starting from 2-(5-chloro-2-methoxyphenyl)acetic acid and 2-chloro-4-nitroaniline. The title compound was obtained as a yellow solid. HPLC purity 98.4% (t_{R}=17.79 min). ¹H NMR (300 MHz, DMSO-d₆) δ 10.05 (s, 1H), 9.78 (s, 1H), 8.338-8.28 (m, 2H), 8.21 (dd, J=9.0, 2.4 Hz, 1H), 7.27 (d, J=2.7 Hz, 1H), 7.15 (dd, J=8.7, 2.4 Hz, 1H), 6.85 (d, J=8.7 Hz, 1H), 3.80 (s, 2H). ¹³C NMR (75 MHz, CDCl₃+CD₃OD) δ 170.4, 153.1, 142.8, 140.9, 130.7, 128.9, 124.8, 124.7, 123.3, 122.5, 122.2, 120.4, 116.3, 40.3. HRMS (ESI) calcd for C₁₄H₁₁Cl₂N₂O₄, 341.0096 (M+H)⁺; found, 341.0086.

5-Chloro-N-(2-((2-chloro-4-nitrophenyl)amino)-2-oxoethyl)-2-hydroxybenzamide (56). To a solution of 2-chloro-4-nitroanilin (1.0 g, 5.8 mmol) and Fmoc-Gly-OH (2.24 g, 7.5 mmol) in 100 mL of toluene was added PCl₃ (1.0 g, 7.5 mmol). The mixture was stirred at 80° C. for 1 h. The mixture was diluted with 200 mL of EtOAc, washed with water (70 mL), dried (Na₂SO₄) and concentrated to give the intermediate. The intermediate was dissolved in 200 mL of CH₃CN, and piperidine (1.3 g, 15.1 mmol) was added. The mixture was stirred at r.t. for 24 h and then concentrated. The residue was purified by column chromatography (DCM/MeOH) to give 2-amino-N-(2-chloro-4-nitrophenyl)acetamide 55a as a yellow solid (800 mg, 69% in two steps). ¹H NMR (300 MHz, DMSO-d₆) δ 8.67 (d, J=9.3 Hz, 1H), 8.42 (d, J=2.7 Hz, 1H), 8.27 (dd, J=9.0, 2.7 Hz, 1H), 5.33 (s, 2H), 3.37 (s, 2H).

To a solution of 2-amino-N-(2-chloro-4-nitrophenyl)acetamide 55a (173 mg, 0.75 mmol), 5-chloro-2-methoxybenzoic acid (168 mg, 0.90 mmol) and DMAP (22 mg, 0.18 mmol) in 30 mL of DCM was added EDCI (345 mg, 1.8 mmol) at 0° C. The resulting mixture was stirred at r.t. for 12 h. The solvent was evaporated and 30 mL of MeOH was added. The resulting mixture was stirred at r.t. for 20 min. The intermediate 5-chloro-N-(2-((2-chloro-4-nitrophenyl)amino)-2-oxoethyl)-2-methoxybenzamide (120 mg, 40%) was isolated by filtration as an off-white solid. ¹H NMR (300 MHz, DMSO-d6) δ 9.93 (s, 1H), 8.79 (t, J=5.7 Hz, 1H), 8.38 (d, J=2.4 Hz, 1H), 8.32 (d, J=9.0 Hz, 1H), 8.23 (dd, J=9.3, 2.4 Hz, 1H), 7.81 (d, J=2.7 Hz, 1H), 7.58 (dd, J=9.0, 2.7 Hz, 1H), 7.24 (d, J=9.0 Hz, 1H), 4.29 (d, J=5.7 Hz, 2H), 3.32 (s, 3H). ¹³C NMR (75 MHz, DMSO-d6) δ 168.8, 163.9, 156.2, 143.2, 140.8, 132.3, 130.0, 125.0, 124.5, 123.9, 123.3, 123.2, 123.0, 114.4, 109.1, 56.5, 44.0.

To a solution of the intermediate 5-chloro-N-(2-((2-chloro-4-nitrophenyl)amino)-2-oxoethyl)-2-methoxybenzamide (100 mg, 0.25 mmol) in 20 mL of DCM was added BBr₃ (1.3 mL, 1.3 mmol, 1M in DCM) dropwise at 0° C. The resulting mixture was stirred at 0° C. for 12 h until the reaction was completed monitored by TLC. Then the mixture was poured into 20 mL of ice water, extracted with EtOAc (2×60 mL), washed with water (30 mL) and brine (30 mL), dried over Na₂SO₄ and concentrated. The residue was purified by column chromatography (Hex/EtOAc=3/1 to 1/1) to afford compound 56 (70 mg, 72%) as a white solid. HPLC purity 99.8% (t_{R}=18.39 min). ¹H NMR (300 MHz, DMSO-d6) δ 12.08 (s, 1H), 10.01 (s, 1H), 9.24 (t, J=5.4 Hz, 1H), 8.38 (d, J=2.4 Hz, 1H), 8.28 (d, J=9.0 Hz, 1H), 8.22 (dd, J=9.0, 2.4 Hz, 1H), 7.95 (d, J=2.7 Hz, 1H), 7.46 (dd, J=8.7, 2.7 Hz, 1H), 6.99 (d, J=8.7 Hz, 1H), 4.31 (d, J=5.4 Hz, 2H). ¹³C NMR (75 MHz, DMSO-d6) δ 168.5, 166.9, 157.5, 143.3, 140.8, 133.3, 128.2, 125.0, 124.3, 123.4, 123.2, 122.6, 119.2, 117.5, 43.5. HRMS (ESI) calcd for C₁₅H₁₂Cl₂N₃O₅, 384.0154 (M+H)⁺; found, 384.0148.

(S)-5-Chloro-N-(1-((2-chloro-4-nitrophenyl)amino)-1-oxopropan-2-yl)-2-hydroxybenzamide (57). Compound 57 was prepared by a procedure similar to that used to prepare compound 56 starting from 2-chloro-4-nitroanilin, Fmoc-L-Ala-OH and 5-chloro-2-methoxybenzoic acid. The corresponding intermediate (S)-2-amino-N-(2-chloro-4-nitrophenyl)propanamide 55b was obtained as a yellow solid (1.0 g, 71% in two steps). ¹H NMR (300 MHz, CDCl3) δ 10.70 (s, 1H), 8.73 (d, J=9.0 Hz, 1H), 8.26 (d, J=2.4 Hz, 1H), 8.13 (dd, J=9.0, 2.4 Hz, 1H), 3.70 (q, J=6.9 Hz, 1H), 1.72 (s, 2H), 1.47 (d, J=6.9 Hz, 3H). ¹³C NMR (75 MHz, CDCl₃) δ 174.7, 142.9, 140.7, 124.9, 123.6, 123.0, 119.6, 51.7, 21.5.

Compound 57 was obtained as a yellow solid (160 mg, 82% in two steps). HPLC purity 95.2% (t_{R}=18.20 min). ¹H NMR (300 MHz, CDCl₃) δ 11.70 (s, 1H), 8.89 (s, 1H), 8.62 (d, J=9.3 Hz, 1H), 8.29 (d, J=1.5 Hz, 1H), 8.16 (d, J=9.01 Hz, 1H), 7.45-7.32 (m, 2H), 6.99-6.85 (m, 2H), 4.97-4.85 (m, 1H), 1.65 (d, J=6.6 Hz, 3H). ¹³C NMR (75 MHz, CDCl₃) δ 170.8, 169.4, 159.8, 143.3, 140.1, 134.8, 125.9, 124.9, 123.9, 123.4, 123.3, 120.8, 120.1, 114.4, 50.4, 16.8. HRMS (ESI) calcd for C₁₆H₁₄Cl₂N₃O₅, 398.0311 (M+H)⁺; found, 398.0303.

(S)-5-Chloro-N-(1-((2-chloro-4-nitrophenyl)amino)-3-methyl-1-oxobutan-2-yl)-2-hydroxybenzamide (58). Compound 58 was prepared by a procedure similar to that used to prepare compound 56 starting from 2-chloro-4-nitroanilin, Fmoc-L-Val-OH and 5-chloro-2-methoxybenzoic acid. The corresponding intermediate (S)-2-amino-N-(2-chloro-4-nitrophenyl)-3-methylbutanamide 55c was obtained as a yellow solid (1.45 g, 92% in two steps). ¹H NMR (300 MHz, CDCl3) δ 10.72 (s, 1H), 8.71 (d, J=9.1 Hz, 1H), 8.23 (d, J=2.4 Hz, 1H), 8.10 (dd, J=9.1, 2.4 Hz, 1H), 3.45 (d, J=3.6 Hz, 1H), 2.52-2.35 (m, 1H), 1.62 (s, 2H), 1.04 (d, J=6.9 Hz, 3H), 0.86 (d, J=6.9 Hz, 3H). ¹³C NMR (75 MHz, CDCl₃) δ 173.7, 142.7, 140.5, 124.8, 123.5, 122.9, 119.5, 60.9, 30.8, 19.7, 16.0.

Compound 58 was obtained as a pale yellow solid (170 mg, 74% in two steps). HPLC purity 98.9% (t_{R}=19.85 min). ¹H NMR (300 MHz, CDCl₃) δ 11.76 (s, 1H), 8.65 (d, J=9.3 Hz, 1H), 8.45 (s, 1H), 8.33 (d, J=2.4 Hz, 1H), 8.19 (dd, J=9.3, 2.4 Hz, 1H), 7.44 (d, J=2.4 Hz, 1H), 7.39 (dd, J=9.0, 2.4 Hz, 1H), 6.97 (d, J=8.7 Hz, 1H), 6.90 (d, J=7.5 Hz, 1H), 4.61 (dd, J=8.1, 7.2 Hz, 1H), 2.46-2.32 (m, 1H), 1.16-1.10 (m, 6H). ¹³C NMR (75 MHz, CDCl₃) δ 169.5, 169.4, 160.4, 143.7, 139.7, 135.1, 125.3, 125.0, 124.0, 123.7, 123.1, 120.8, 120.6, 114.6, 60.0, 30.8, 19.5, 18.6. HRMS (ESI) calcd for C₁₈H₁₈Cl₂N₃O₅, 426.0624 (M+H)⁺; found, 426.0619.

(S)-5-chloro-N-(1-((2-chloro-4-nitrophenyl)amino)-1-oxo-3-phenylpropan-2-yl)-2-hydroxybenzamide (59). Compound 59 was prepared by a procedure similar to that used to prepare compound 56 starting from 2-chloro-4-nitroanilin, Fmoc-L-Phe-OH and 5-chloro-2-methoxybenzoic acid. The corresponding intermediate (S)-2-amino-N-(2-chloro-4-nitrophenyl)-3-phenylpropanamide 55d was afforded as a yellow solid (1.75 g, 94% in two steps). ¹H NMR (300 MHz, CDCl₃) δ 10.66 (s, 1H), 8.79 (d, J=9.0 Hz, 1H), 8.29 (d, J=2.4 Hz, 1H), 8.17 (dd, J=9.3, 2.7 Hz, 1H), 7.39-7.20 (m, 5H), 3.83 (dd, J=9.0, 2.7 Hz, 1H), 3.40 (dd, J=13.8, 3.9 Hz, 1H), 2.83 (dd, J=13.8, 9.6 Hz, 1H), 1.61 (s, 2H). ¹³C NMR (75 MHz, CDCl₃) δ 173.5, 143.0, 140.5, 137.2, 129.4 (2C), 129.1 (2C), 127.3, 124.9, 123.7, 123.1, 119.7, 57.3, 40.6.

Compound 59 was obtained as a brown solid (100 mg, 83% in two steps). HPLC purity 99.2% (t_{R}=20.48 min). ¹H NMR (300 MHz, CDCl₃) δ 11.71 (s, 1H), 8.62 (d, J=9.0 Hz, 1H), 8.24 (d, J=2.4 Hz, 2H), 8.16 (dd, J=9.0, 2.45 Hz, 1H), 7.41-7.27 (m, 7H), 7.00-6.92 (m, 2H), 5.09-4.93 (m, 1H), 3.40 (dd, J=13.8, 6.0 Hz, 1H), 3.23 (dd, J=13.8, 8.4 Hz, 1H). ¹³C NMR (75 MHz, CDCl₃) δ 169.5, 169.2, 160.2, 143.6, 139.6, 135.5, 135.0, 129.4 (2C), 129.2 (2C), 128.0, 125.5, 123.9, 123.6, 123.0, 120.6, 120.4, 118.2, 114.5, 56.0, 37.9. HRMS (ESI) calcd for C₂₂H₁₈Cl₂N₃O₅, 474.0624 (M+H)⁺; found, 474.0623.

(R)-5-Chloro-N-(1-((2-chloro-4-nitrophenyl)amino)-1-oxo-3-phenylpropan-2-yl)-2-hydroxybenzamide (60). Compound 60 was prepared by a procedure similar to that used to prepare compound 56 starting from 2-chloro-4-nitroanilin, Fmoc-D-Phe-OH and 5-chloro-2-methoxybenzoic acid. The corresponding intermediate (R)-2-amino-N-(2-chloro-4-nitrophenyl)-3-phenylpropanamide 55e was afforded as a yellow solid (869 mg, 94% in two steps). ¹H NMR (300 MHz, CDCl₃) δ 10.66 (s, 1H), 8.77 (d, J=9.3 Hz, 1H), 8.25 (d, J=2.4 Hz, 1H), 8.14 (dd, J=9.0, 2.4 Hz, 1H), 7.39-7.23 (m, 5H), 3.82 (dd, J=9.6, 3.6 Hz, 1H), 3.39 (dd, J=14.1, 3.6 Hz, 1H), 2.83 (dd, J=14.1, 9.6 Hz, 1H), 1.63 (s, 2H). ¹³C NMR (75 MHz, CDCl₃) δ 173.4, 142.9, 140.5, 137.2, 129.3 (2C), 129.0 (2C), 127.3, 124.8, 123.6, 122.9, 119.6, 57.2, 40.5.

Compound 60 was obtained as a yellow solid (300 mg, 72% in two steps). HPLC purity 96.7% (t_{R}=20.47 min). ¹H NMR (300 MHz, CDCl₃) δ 11.71 (s, 1H), 8.61 (d, J=9.3 Hz, 1H), 8.31 (s, 1H), 8.23 (d, J=2.4 Hz, 1H), 8.15 (dd, J=9.0, 2.4 Hz, 1H), 7.41-7.26 (m, 7H), 7.13 (d, J=7.2 Hz, 1H), 6.98-6.88 (m, 1H), 5.08-4.97 (m, 1H), 3.38 (dd, J=13.8, 6.3 Hz, 1H), 3.24 (dd, J=13.8, 8.4 Hz, 1H). ¹³C NMR (75 MHz, CDCl₃) δ 169.4, 169.2, 160.3, 143.6, 139.6, 135.4, 135.1, 129.5 (2C), 129.3 (2C), 128.0, 125.5, 124.9, 124.0, 123.6, 123.0, 120.6, 120.5, 114.4, 56.0, 38.0. HRMS (ESI) calcd for C₂₂H₁₈Cl₁₂N₃O₅, 474.0624 (M+H)⁺; found, 474.0618.

(S)-5-Chloro-N-(1-((2-chloro-4-nitrophenyl)amino)-4-methyl-1-oxopentan-2-yl)-2-hydroxybenzamide (61). Compound 61 was prepared by a procedure similar to that used to prepare compound 56 starting from 2-chloro-4-nitroanilin, Fmoc-L-Leu-OH and 5-chloro-2-methoxybenzoic acid. The corresponding intermediate (S)-2-amino-N-(2-chloro-4-nitrophenyl)-4-methylpentanamide 55f was afforded as a yellow solid (1.5 g, 90% in two steps). ¹H NMR (300 MHz, CDCl3) δ 10.75 (s, 1H), 8.69 (d, J=9.3 Hz, 1H), 8.22 (d, J=2.4 Hz, 1H), 8.09 (dd, J=9.3, 2.4 Hz, 1H), 3.64-3.48 (m, 1H), 1.87-1.75 (m, 2H), 1.68 (s, 2H), 1.54-1.37 (m, 1H), 0.97 (t, J=6.3 Hz, 6H). ¹³C NMR (75 MHz, CDCl₃) δ 174.7, 142.7, 140.7, 124.8, 123.6, 122.8, 119.5, 54.4, 43.8, 25.1, 23.4, 21.3.

Compound 61 was obtained as a pale yellow solid (145 mg, 74% in two steps). HPLC purity 99.6% (t_{R}=19.88 min). ¹H NMR (300 MHz, CDCl₃) δ 11.70 (s, 1H), 8.81 (s, 1H), 8.61 (d, J=9.0 Hz, 1H), 8.29 (d, J=2.4 Hz, 1H), 8.15 (dd, J=9.3, 2.4 Hz, 1H), 7.45-7.32 (m, 2H), 6.93 (d, J=8.7 Hz, 1H), 6.86 (d, J=7.2 Hz, 1H), 4.90-4.78 (m, 1H), 2.03-1.74 (m, 3H), 1.08-0.98 (m, 6H). ¹³C NMR (75 MHz, CDCl₃) δ 170.3, 169.6, 160.3, 143.6, 140.1, 135.1, 125.4, 125.0, 124.0, 123.6, 123.2, 120.8, 120.5, 114.3, 53.1, 40.1, 25.1, 23.0, 22.2. HRMS (ESI) calcd for C₁₉H₂₀Cl₂N₃O₅, 440.0780 (M+H)⁺; found, 440.0774.

5-Chloro-N-((2S,3R)-1-((2-chloro-4-nitrophenyl)amino)-3-methyl-1-oxopentan-2-yl)-2-hydroxybenzamide (62). Compound 62 was prepared by a procedure similar to that used to prepare compound 56 starting from 2-chloro-4-nitroanilin, Fmoc-L-Ile-OH and 5-chloro-2-methoxybenzoic acid. The corresponding intermediate (2S,3R)-2-amino-N-(2-chloro-4-nitrophenyl)-3-methylpentanamide 55g was obtained as a yellow solid (1.3 g, 78% in two steps). ¹H NMR (300 MHz, CDCl₃) δ 10.65 (s, 1H), 8.57 (d, J=9.3 Hz, 1H), 8.05 (d, J=2.4 Hz, 1H), 7.94 (dd, J=9.3, 2.7 Hz, 1H), 3.41 (d, J=3.3 Hz, 1H), 2.09-1.96 (m, 1H), 1.36-1.20 (m, 1H), 1.19-1.00 (m, 1H), 0.93 (d, J=6.9 Hz, 3H), 0.79 (t, J=7.5 Hz, 3H). ¹³C NMR (75 MHz, CDCl₃) δ 173.6, 142.3, 140.3, 124.4, 123.1, 122.5, 119.0, 60.3, 37.9, 23.7, 16.0, 11.8.

Compound 62 was obtained as an off-white solid (170 mg, 72% in two steps). HPLC purity 98.7% (t_{R}=19.38 min). ¹H NMR (300 MHz, CDCl₃) δ 11.75 (s, 1H), 8.65 (d, J=9.0 Hz, 1H), 8.42 (s, 1H), 8.32 (d, J=2.1 Hz, 1H), 8.19 (dd, J=9.0, 2.7 Hz, 1H), 7.44-7.35 (m, 2H), 6.96 (d, J=8.7 Hz, 1H), 6.88 (d, J=7.5 Hz, 1H), 4.66 (t, J=7.5 Hz, 1H), 2.25-2.10 (m, 1H), 1.79-1.62 (m, 1H), 1.45-1.27 (m, 1H), 1.10 (d, J=6.6 Hz, 3H), 1.03 (t, J=7.5 Hz, 3H). ¹³C NMR (75 MHz, CDCl3+CD₃OD) a 170.5, 168.7, 158.5, 143.6, 140.0, 134.2, 127.4, 125.0, 124.2, 123.7, 123.3, 121.4, 119.4, 116.1, 59.2, 36.0, 25.1, 15.8, 10.9. HRMS (ESI) calcd for C₁₉H₂₀Cl₂N₃O₅, 440.0780 (M+H)⁺; found, 440.0773.

(S)-5-Chloro-N-(1-((2-chloro-4-nitrophenyl)amino)-4-(methylthio)-1-oxobutan-2-yl)-2-hydroxybenzamide (63). Compound 63 was prepared by a procedure similar to that used to prepare compound 56 starting from 2-chloro-4-nitroanilin, Fmoc-L-Met-OH and 5-chloro-2-methoxybenzoic acid. The corresponding intermediate (S)-2-amino-N-(2-chloro-4-nitrophenyl)-4-(methylthio)butanamide 55h was obtained as a yellow solid (1.53 g, 87% in two steps). ¹H NMR (300 MHz, CDCl₃) δ 10.73 (s, 1H), 8.73 (d, J=9.0 Hz, 1H), 8.28 (d, J=2.4 Hz, 1H), 8.14 (dd, J=9.0, 2.4 Hz, 1H), 3.82-3.68 (m, 1H), 2.78-2.59 (m, 2H), 2.42-2.27 (m, 1H), 2.13 (s, 3H), 1.97-1.71 (m, 3H). ¹³C NMR (75 MHz, CDCl₃) δ 173.7, 143.0, 140.5, 124.9, 123.7, 123.0, 119.6, 55.0, 33.5, 30.8, 15.4.

Compound 63 was obtained as a grey solid (130 mg, 73% in two steps). HPLC purity 99.1% (t_{R}=19.08 min). ¹H NMR (300 MHz, CDCl₃) δ 11.76 (s, 1H), 9.02 (s, 1H), 8.65 (d, J=9.3 Hz, 1H), 8.31 (d, J=2.4 Hz, 1H), 8.18 (dd, J=9.3, 2.4 Hz, 1H), 7.50-7.36 (m, 3H), 6.97 (d, J=9.0 Hz, 1H), 5.11-5.01 (m, 1H), 2.92-2.79 (m, 1H), 2.77-2.65 (m, 1H), 2.38-2.28 (m, 2H), 2.21 (s, 3H). ¹³C NMR (75 MHz, CDCl₃) δ 169.6, 169.5, 160.5, 143.7, 140.0, 135.2, 125.6, 125.1, 124.0, 123.6, 123.3, 120.8, 120.6, 114.4, 53.7, 30.5, 29.8, 15.5. HRMS (ESI) calcd for C₁₈H₁₈Cl₂N₃O₅S, 458.0344 (M+H)⁺; found, 458.0340.

(S)-tert-Butyl 4-(5-chloro-2-hydroxybenzamido)-5-((2-chloro-4-nitrophenyl)amino)-5-oxopentanoate (64). To a solution of 2-chloro-4-nitroanilin (300 mg, 1.7 mmol) and Fmoc-Glu(O^{t}Bu)-OH (962 mg, 2.3 mmol) in 30 mL of toluene was added PCl₃ (310 mg, 2.3 mmol). The resulting mixture was stirred at 80° C. for 1 h. The mixture was diluted with 150 mL of DCM, washed with water (50 mL), dried (Na₂SO₄) and concentrated to give the intermediate. The intermediate was dissolved in 20 mL of CH₃CN, and piperidine (254 mg, 3.0 mmol) was added. The mixture was stirred at r.t. overnight, and then concentrated. The residue was purified by column chromatography (Hex/EtOAc=5/1 to 3/1) to give (S)-tert-butyl 4-amino-5-((2-chloro-4-nitrophenyl)amino)-5-oxopentanoate 55i (380 mg, 61% in two steps) as yellow oil. ¹H NMR (300 MHz, CDCl₃) δ 10.67 (s, 1H), 8.69 (d, J=9.3 Hz, 1H), 8.23 (d, J=2.7 Hz, 1H), 8.10 (dd, J=9.3, 2.7 Hz, 1H), 3.58 (dd, J=7.8, 4.8 Hz, 1H), 2.46-2.36 (m, 2H), 2.32-2.18 (m, 1H), 1.98-1.66 (m, 3H), 1.42 (s, 9H). ¹³C NMR (75 MHz, CDCl₃) δ 173.6, 172.5, 142.9, 140.5, 124.8, 123.6, 122.9, 119.6, 81.0, 55.6, 32.1, 29.9, 28.1 (3C).

To a solution of amine 55i (100 mg, 0.28 mmol), 5-chloro-2-hydroxybenzoic acid (48 mg, 0.28 mmol) and DMAP (7 mg, 0.056 mmol) in 15 mL of DCM was added EDCI (107 mg, 0.56 mmol) at 0° C. The resulting mixture was stirred at r.t. for 24 h and then concentrated. The residue was purified by preparative TLC to give compound 64 (27 mg, 18%) as a yellow solid. HPLC purity 98.4% (t_{R}=19.94 min). ¹H NMR (300 MHz, CDCl₃) δ 11.94 (s, 1H), 9.32 (s, 1H), 8.62 (d, J=9.3 Hz, 1H), 8.49 (d, J=5.7 Hz, 1H), 8.28 (d, J=2.4 Hz, 1H), 8.14 (dd, J=9.3, 2.4 Hz, 1H), 7.62 (d, J=2.1 Hz, 1H), 7.36 (dd, J=9.0, 2.1 Hz, 1H), 6.94 (d, J=8.7 Hz, 1H), 4.81-4.69 (m, 1H), 2.85-2.71 (m, 1H), 2.58-2.44 (m, 1H), 2.39-2.19 (m, 2H), 1.49 (s, 9H). ¹³C NMR (75 MHz, CDCl3) δ 175.2, 170.3, 169.7, 160.5, 143.5, 140.3, 135.0, 126.0, 125.0, 124.0, 123.5, 123.3, 120.8, 120.3, 114.3, 82.9, 55.0, 32.0, 28.3 (3C), 25.6. HRMS (ESI) calcd for C₂₂H₂₄Cl₂N₃O₇, 512.0991 (M+H)⁺; found, 512.0988.

(S)—N-(1-((3,5-Bis(trifluoromethyl)phenyl)amino)-3-methyl-1-oxobutan-2-yl)-5-chloro-2-hydroxybenzamide (65). Compound 65 was prepared by a procedure similar to that used to prepare compound 56 starting from 3,5-bis(trifluoromethyl)aniline, Fmoc-L-Val-OH and 5-chloro-2-methoxybenzoic acid. The corresponding intermediate (S)-2-amino-N-(3,5-bis(trifluoromethyl)phenyl)-3-methylbutanamide 55j was obtained as pale yellow oil (1.4 g, 94% in two steps). ¹H NMR (300 MHz, CDCl₃) δ 10.01 (s, 1H), 8.12 (s, 2H), 7.56 (s, 1H), 3.40 (d, J=3.5 Hz, 1H), 2.54-2.37 (m, 1H), 1.55 (s, 2H), 1.05 (d, J=6.9 Hz, 3H), 0.87 (d, J=6.9 Hz, 3H). ¹³C NMR (75 MHz, CDCl₃) δ 173.4, 139.4, 132.5 (q, J=33.2 Hz, 2C), 123.3 (q, J=271.1 Hz, 2C), 119.1 (q, J=3.2 Hz, 2C), 117.2 (m), 60.5, 30.8, 19.8, 16.0.

Compound 65 was obtained as a yellow solid (148 mg, 62% in two steps). HPLC purity 99.5% (t_{R}=19.90 min). ¹H NMR (300 MHz, CD₃OD+CDCl₃) δ 10.50 (s, 1H), 8.80 (d, J=8.1 Hz, 1H), 8.15 (s, 2H), 7.89 (d, J=2.1 Hz, 1H), 7.54 (s, 1H), 7.27 (dd, J=8.7, 2.1 Hz, 1H), 6.86 (d, J=9.0 Hz, 1H), 2.33-2.17 (m, 1H), 1.13-1.01 (m, 6H). ¹³C NMR (75 MHz, CD₃OD+CDCl₃) δ 172.0, 167.6, 157.4, 140.5, 133.9, 132.7 (q, J=33.2 Hz, 2C), 129.6, 125.0, 123.8 (q, J=271.6 Hz, 2C), 120.2 (q, J=3.5 Hz, 2C), 119.1, 118.6, 117.6 (m), 60.4, 31.9, 19.6, 18.6. HRMS (ESI) calcd for C₂₀H₁₈ClF₆N₂O₃, 483.0910 (M+H)⁺; found, 483.0907.

(S)-5-Chloro-N-(1-((2-chloro-4-(trifluoromethyl)phenyl)amino)-3-methyl-1-oxobutan-2-yl)-2-hydroxybenzamide (66). Compound 66 was prepared by a procedure similar to that used to prepare compound 56 starting from 2-chloro-4-(trifluoromethyl)aniline, Fmoc-L-Val-OH and 5-chloro-2-methoxybenzoic acid. The corresponding intermediate (S)-2-amino-N-(2-chloro-4-(trifluoromethyl)phenyl)-3-methylbutanamide 55k was obtained as a pale yellow solid (1.3 g, 86% in two steps). ¹H NMR (300 MHz, CDCl3) δ 10.47 (s, 1H), 8.67 (d, J=8.7 Hz, 1H), 7.63 (s, 1H), 7.52 (d, J=8.7 Hz, 1H), 3.45 (d, J=3.3 Hz, 1H), 2.52-2.39 (m, 1H), 1.55 (s, 2H), 1.06 (d, J=6.9 Hz, 3H), 0.89 (d, J=6.9 Hz, 3H). ¹³C NMR (75 MHz, CDCl₃) δ 173.4, 137.9, 126.3 (q, J=3.9 Hz), 126.2 (q, J=33.3 Hz), 125.0 (q, J=3.8 Hz), 123.6 (q, J=270.2 Hz), 123.1, 120.5, 61.0, 30.9, 19.8, 16.1.

Compound 66 was obtained as a pale yellow solid (142 mg, 71%). HPLC purity 99.7% (t_{R}=19.56 min). ¹H NMR (300 MHz, CDCl₃) δ 11.82 (s, 1H), 8.53 (d, J=8.7 Hz, 1H), 8.21 (s, 1H), 7.68 (s, 1H), 7.56 (d, J=9.0 Hz, 1H), 7.44 (d, J=2.1 Hz, 1H), 7.37 (dd, J=9.0, 2.4 Hz, 1H), 7.00-6.91 (m, 2H), 4.66-4.57 (m, 1H), 2.45-2.31 (m, 1H), 1.17-1.09 (m, 6H). ¹³C NMR (75 MHz, CDCl₃) δ 169.4, 169.3, 160.3, 137.0, 134.9, 127.5 (q, J=33.6 Hz), 126.6 (q, J=3.8 Hz), 125.4, 125.2 (q, J=3.7 Hz), 123.9, 123.3 (q, J=270.3 Hz), 123.3, 121.6, 120.4, 114.7, 59.9, 31.2, 19.4, 18.6. HRMS (ESI) calcd for C₁₉H₁₈Cl₂F₃N₂O₃, 449.0647 (M+H)⁺; found, 449.0641.

(S)-5-Chloro-N-(1-((3-fluoro-5-(trifluoromethyl)phenyl)amino)-3-methyl-1-oxobutan-2-yl)-2-hydroxybenzamide (67). Compound 67 was prepared by a procedure similar to that used to prepare compound 56 starting from 3-amino-5-fluorobenzotrifluoride, Fmoc-L-Val-OH and 5-chloro-2-methoxybenzoic acid. The corresponding intermediate (S)-2-amino-N-(3-fluoro-5-(trifluoromethyl)phenyl)-3-methylbutanamide 55l was obtained as pale yellow oil (1.2 g, 89% in two steps). ¹H NMR (300 MHz, CDCl₃) δ 9.87 (s, 1H), 7.81 (d, J=10.5 Hz, 1H), 7.51 (s, 1H), 7.03 (d, J=8.1 Hz, 1H), 3.38 (d, J=3.3 Hz, 1H), 2.52-2.37 (m, 1H), 1.52 (s, 2H), 1.04 (d, J=6.9 Hz, 3H), 0.86 (d, J=6.9 Hz, 3H). ¹³C NMR (75 MHz, CDCl₃) δ 173.2, 163.0 (d, J=245.4 Hz), 140.2 (d, J=11.3 Hz), 132.8 (qd, J=33.3, 9.4 Hz), 123.3 (qd, J=271.0, 3.3 Hz), 111.8 (m), 109.9 (d, J=25.7 Hz), 107.9 (dq, J=24.8, 3.8 Hz), 60.5, 30.8, 19.8, 16.0.

Compound 67 was obtained as an off-white solid (150 mg, 78%). HPLC purity 99.8% (t_{R}=19.42 min). ¹H NMR (300 MHz, CD₃OD+CDCl₃) δ 10.30 (s, 1H), 8.71 (d, J=8.1 Hz, 1H), 7.86 (d, J=2.4 Hz, 1H), 7.72-7.60 (m, 2H), 7.25 (dd, J=8.7, 2.4 Hz, 1H), 7.01 (d, J=8.4 Hz, 1H), 6.84 (d, J=9.0 Hz, 1H), 4.57-4.48 (m, 1H), 2.31-2.15 (m, 1H), 1.09-0.99 (m, 6H). ¹³C NMR (75 MHz, CD₃OD+CDCl₃) δ 171.7, 167.6, 163.1 (d, J=245.0 Hz), 157.4, 141.0 (d, J=11.0 Hz), 133.8, 133.0 (qd, J=33.2, 9.3 Hz), 129.3, 124.9, 123.7 (qd, J=270.5, 3.4 Hz), 119.0, 118.3, 112.7 (m), 110.7 (d, J=26.0 Hz), 108.3 (dq, J=24.8, 3.8 Hz), 60.3, 31.8, 19.6, 18.6. HRMS (ESI) calcd for C₁₉H₁₈ClF₄N₂O₃, 433.0942 (M+H)⁺; found, 433.0934.

(S)-5-Chloro-N-(1-((3,4-difluorophenyl)amino)-3-methyl-1-oxobutan-2-yl)-2-hydroxybenzamide (68). Compound 68 was prepared by a procedure similar to that used to prepare compound 56 starting from 3,4-difluoroaniline, Fmoc-L-Val-OH and 5-chloro-2-methoxybenzoic acid. The corresponding intermediate (S)-2-amino-N-(3,4-difluorophenyl)-3-methylbutanamide 55m was obtained as yellow oil (1.25 g, 69% in two steps). ¹H NMR (300 MHz, CDCl₃) δ 9.55 (s, 1H), 7.74-7.64 (m, 1H), 7.18-6.99 (m, 2H), 3.34 (d, J=3.9 Hz, 1H), 2.50-2.33 (m, 1H), 1.48 (s, 2H), 1.03 (d, J=6.9 Hz, 3H), 0.86 (t, J=6.9 Hz, 3H). ¹³C NMR (75 MHz, CDCl₃) δ 172.8, 150.3 (dd, J=245.1, 13.1 Hz), 146.9 (dd, J=243.2, 12.8 Hz), 134.6 (dd, J=8.8, 3.1 Hz), 117.2 (dd, J=18.1, 1.2 Hz), 115.1 (dd, J=5.8, 3.6 Hz), 109.2 (d, J=21.5 Hz), 60.5, 30.9, 19.8, 16.1.

Compound 68 was obtained as a white solid (200 mg, 76%). HPLC purity 99.6% (t_{R}=18.03 min). ¹H NMR (300 MHz, CD₃OD+CDCl₃) δ 9.99 (s, 1H), 8.66 (d, J=7.8 Hz, 1H), 7.82 (d, J=2.4 Hz, 1H), 7.66-7.54 (m, 1H), 7.26-6.95 (m, 3H), 6.81 (d, J=8.7 Hz, 1H), 4.54-4.44 (m, 1H), 2.30-2.12 (m, 1H), 1.03 (d, J=6.6 Hz, 6H). ¹³C NMR (75 MHz, CD₃OD+CDCl₃) δ 170.7, 167.1, 156.9, 149.7 (dd, J=244.7, 13.1 Hz), 146.8 (dd, J=243.5, 12.8 Hz), 134.3 (dd, J=8.6, 3.2 Hz), 133.2, 128.4, 124.2, 118.5, 117.3, 116.8 (d, J=18.1 Hz), 115.7 (dd, J=5.9, 3.6 Hz), 109.5 (d, J=21.7 Hz), 59.6, 31.1, 18.9, 18.1. HRMS (ESI) calcd for C₁₈H₁₈ClF₂N₂O₃, 383.0974 (M+H)⁺; found, 383.0966.

(S)—N-(1-((3,5-Bis(trifluoromethyl)phenyl)amino)-1-oxo-3-phenylpropan-2-yl)-5-chloro-2-hydroxybenzamide (69). Compound 69 was prepared by a procedure similar to that used to prepare compound 56 starting from 3,5-bis(trifluoromethyl)aniline, Fmoc-L-Phe-OH and 5-chloro-2-methoxybenzoic acid. The corresponding intermediate (S)-2-amino-N-(3,5-bis(trifluoromethyl)phenyl)-3-phenylpropanamide 55n was obtained as a white solid (1.63 g, 99% in two steps). ¹H NMR (300 MHz, CDCl₃) δ 9.89 (s, 1H), 8.11 (s, 2H), 7.60 (s, 1H), 7.38-7.27 (m, 3H), 7.26-7.21 (m, 2H), 3.77 (dd, J=9.3, 3.9 Hz, 1H), 3.37 (dd, J=13.8, 4.0 Hz, 1H), 2.83 (dd, J=13.8, 9.3 Hz, 1H), 1.56 (s, 2H). ¹³C NMR (75 MHz, CDCl₃) δ 173.2, 139.2, 137.2, 132.4 (q, J=33.2 Hz, 2C), 129.4 (2C), 129.1 (2C), 127.3, 123.3 (q, J=271.0 Hz, 2C), 119.2 (q, J=2.9 Hz, 2C), 117.4 (m), 56.7, 40.5.

Compound 69 was obtained as a pale yellow solid (240 mg, 65%). HPLC purity 99.5% (t_{R}=20.17 min). ¹H NMR (300 MHz, CDCl₃+CD₃OD) δ 8.04 (s, 2H), 7.86-7.83 (m, 1H), 7.58 (s, 1H), 7.33-7.20 (m, 6H), 6.87 (d, J=9.0 Hz, 1H), 5.02-4.92 (m, 1H), 3.34-3.14 (m, 2H). ¹³C NMR (75 MHz, CDCl₃+CD₃OD) δ 171.2, 167.5, 157.6, 139.8, 136.4, 133.9, 132.4 (q, J=33.2 Hz, 2C), 129.5 (2C), 128.9 (3C), 127.4, 124.6, 123.5 (q, J=270.8 Hz, 2C), 120.1 (q, J=3.2 Hz, 2C), 119.0, 117.6, 117.6, 56.2, 38.7. HRMS (ESI) calcd for C₂₄H₁₈ClF₆N₂O₃, 531.0910 (M+H)⁺; found, 531.0909.

(S)-5-Chloro-N-(1-((2-chloro-4-nitrophenyl)amino)-3-methyl-1-oxobutan-2-yl)-2-(methylsulfonamido)benzamide (70). To a solution of (S)-2-amino-N-(2-chloro-4-nitrophenyl)-3-methylbutanamide 55c (50 mg, 0.23 mmol), 5-chloro-2-(methylsulfonamido)benzoic acid 23 (63 mg, 0.25 mmol) and DIEA (59 mg, 0.46 mmol) in 10 mL of DCM was added HBTU (175 mg, 0.46 mmol) at 0° C. The resulting mixture was stirred at r.t. overnight, and then concentrated. The residue was purified by preparative TLC (DCM/MeOH) to give compound 70 as pale yellow solid (81 mg, 70%). HPLC purity 98.0% (t_{R}=18.57 min). ¹H NMR (300 MHz, CDCl₃+CD₃OD) δ 8.40 (d, J=9.0 Hz, 1H), 8.28 (d, J=2.4 Hz, 1H), 8.12 (dd, J=9.3, 2.4 Hz, 1H), 7.78 (d, J=2.1 Hz, 1H), 7.60 (d, J=9.0 Hz, 1H), 7.48-7.42 (m, 1H), 4.52 (d, J=8.1 Hz, 1H), 2.99 (s, 3H), 2.42-2.28 (m, 1H), 1.12-1.03 (m, 6H). ¹³C NMR (75 MHz, CDCl₃+CD₃OD) δ 171.1, 168.7, 144.2, 140.6, 137.6, 133.3, 129.6, 128.8, 125.4, 124.9, 123.4, 122.8 (2C), 122.1, 61.1, 39.9, 30.3, 19.7, 19.0. HRMS (ESI) calcd for C₁₉H₂₁Cl₂N₄O₆S, 503.0559 (M+H)⁺; found, 503.0553.

(S)-5-Chloro-N-(1-((2-chloro-4-nitrophenyl)amino)-1-oxo-3-phenylpropan-2-yl)-2-(methylsulfonamido)benzamide (71). Compound 71 was prepared by a procedure similar to that used to prepare compound 70 starting from (S)-2-amino-N-(2-chloro-4-nitrophenyl)-3-phenylpropanamide 55d and 5-chloro-2-(methylsulfonamido)benzoic acid 23. The title compound was obtained as a pale yellow solid (80 mg, 77%). HPLC purity 98.6% (t_{R}=19.21 min). ¹H NMR (300 MHz, CD₃OD) δ 8.42 (d, J=9.3 Hz, 1H), 8.25 (d, J=2.4 Hz, 1H), 8.11 (dd, J=9.0, 2.4 Hz, 1H), 7.63 (d, J=2.1 Hz, 1H), 7.55 (d, J=1.8 Hz, 1H), 7.44 (dd, J=8.7, 2.4 Hz, 1H), 7.32-7.19 (m, 5H), 5.04 (dd, J=8.7, 6.9 Hz, 1H), 3.37 (dd, J=13.8, 6.9 Hz, 1H), 3.17 (dd, J=13.8, 8.7 Hz, 1H), 2.90 (s, 3H). ¹³C NMR (75 MHz, CD₃OD) δ 171.1, 168.9, 144.3, 140.8, 137.7, 137.1, 133.4, 129.8, 129.7 (2C), 129.3 (2C), 129.0, 127.7, 125.5, 124.8, 123.6, 123.1, 122.6, 122.5, 56.8, 39.9, 37.2. HRMS (ESI) calcd for C₂₃H₂₁Cl₂N₄O₆S, 551.0559 (M+H)⁺; found, 551.0557.

5-Chloro-N-(2-((2-chloro-4-nitrophenyl)amino)-2-oxoethyl)-2-(methylsulfonamido)benzamide (72). Compound 72 was prepared by a procedure similar to that used to prepare compound 70 starting from 2-amino-N-(2-chloro-4-nitrophenyl)acetamide 55a and 5-chloro-2-(methylsulfonamido)benzoic acid 23. The title compound was obtained as a pale yellow solid (54 mg, 60%). HPLC purity 98.5% (t_{R}=17.14 min). ¹H NMR (300 MHz, DMSO-d₆) δ 10.74 (s, 1H), 10.03 (s, 1H), 9.42 (t, J=5.1 Hz, 1H), 8.38 (d, J=1.2 Hz, 1H), 8.29-8.19 (m, 2H), 7.97 (d, J=1.8 Hz, 1H), 7.65 (dd, J=9.0, 2.1 Hz, 1H), 7.57 (d, J=9.0 Hz, 1H), 4.26 (d, J=5.4 Hz, 2H), 3.14 (s, 3H). ¹³C NMR (75 MHz, DMSO-d₆) δ 168.3, 167.5, 143.4, 140.8, 137.5, 132.5, 128.3, 127.2, 125.0, 124.5, 123.6, 123.2, 122.0, 121.4, 43.5, 39.9. HRMS (ESI) calcd for C₁₆H₁₅Cl₂N₄O₆S, 461.0089 (M+H)⁺; found, 461.0085.

Plaque Assay. Compounds were tested using low MOI infections (0.06 vp/cell) and at concentrations of 10 μM and in a dose-response assay ranging from 10 to 0.375 μM in a plaque assay. Briefly, 293β5 cells were seeded in 6-well plates at a density of 4×10⁵ cells per well in duplicate for each condition. When cells reached 80-90% confluency, they were infected with HAdV5-GFP (0.06 vp/cell) and rocked for 2 h at 37° C. After the incubation the inoculum was removed, and the cells were washed once with PBS. The cells were then carefully overlaid with 4 mL/well of equal parts of 1.6% (water/vol) Difco Agar Noble (Becton, Dickinson & Co., Sparks, Md.) and 2×EMEM (Minimum Essential Medium Eagle, BioWhittaker) supplemented with 2×penicillin/streptomycin, 2×L-glutamine, and 10% FBS. The mixture also contained the drugs in concentrations ranging from 10 to 0.375 μM. Following incubation for 7 days at 37° C., plates were scanned with a Typhoon FLA 9000 imager (GE Healthcare Life Sciences) and plaques were quantified with ImageJ (Schneider et al., Nat. Methods. 2012, 9, 671-675).

Entry Assay. The anti-HAdV activity was measured in an entry assay using human A549 epithelial cells (3×10⁵ cells/well in coming black wall, clear bottom 96-well plates) infected with HAdV5-GFP (2000 vp/cell) in the presence 50 μM of the candidates and in a dose-response assay. A standard infection curve was generated in parallel by infecting cells in the absence of compounds using serial 2-fold dilutions of virus. All reactions were done in triplicate. Cells, virus, and drugs were incubated for 48 h at 37° C. and 5% CO₂. Infection, as measured by HAdV5-mediated GFP expression, was analyzed using a Typhoon 9410 imager (GE Healthcare Life Sciences) and quantified with ImageQuantTL (GE Healthcare Life Sciences).

Cytotoxicity Assay. The cytotoxicity of the compounds was analyzed by commercial kit AlamarBlue® (Invitrogen, Ref. DAL1025). A549 cells at a density of 5×10³ cells per well in 96-well plates were seeded. Decreasing concentrations of each derivative (200 μM, 150 μM, 100 μM, 80 μM, 60 μM, 40 μM, 30 μM, 20 μM, 10 μM, 5 μM, 2.5 μM, 0 μM) were diluted in 100 μL of Dulbecco's Modified Eagle Medium (DMEM). Cells were then incubated at 37° C. for 48 h following the kit protocol. The cytotoxic concentration 50 (CC₅₀) value was obtained using the statistical package GraphPad Prism. This assay was performed in duplicate.

Virus Yield Reduction. A549 cells (1.5×10⁵ cells/well in a 24-well plate) were incubated 24 h in 500 μL of complete DMEM and they were infected with wild-type HAdV5 (100 vp/cell) when more than 90% of confluency were observed. Infected cells were incubated 48 h at 37° C. in 500 μL of complete DMEM containing 25 μM of either compounds or the same volume of DMSO (positive control). After 48 h, cells were harvested and subjected to three rounds of freeze/thaw. Serial dilutions of clarified lysates were titrated on A549 cells (3×10⁴ cells/well), and TCID₅₀ values were calculated using an end-point dilution method (Reed and Muench, 1938).

Time of Addition Curve Study. The anti-HAdV effect of derivatives at different points of time was measured in a time-curve assay using 293β5 cells (3×10⁵ cells/well in coming black wall, clear bottom 96-well plates) infected with HAdV5-GFP (2.000 vp/cell) in the presence of different concentration of either derivatives (according to their CC₅₀ value) or the same volume of DMSO (positive control). Parallel samples of HAdV-5 were incubated with or without the selected derivatives on ice for 1 h. Virus was then added to 293β5 cells and incubated at 37° C. The derivatives were added at the indicated time points before or during this incubation. After a total of 2 h at 37° C., cells were incubated for an additional 48 h at 37° C. and 5% CO₂ before being analyzed for GFP expression using the Typhoon 9410 imager (GE Healthcare Life Sciences) as above.

Nuclear-Associated HAdV Genomes. The nuclear delivery of HAdV genomes was assessed by real-time PCR following nuclear isolation from infected cells. 1×10⁶ A549 cells in 6-well plates were infected with wild-type HAdV5 at MOI 2,000 vp/cell in the presence of 50 μM of the derivates, or the same volume of DMSO for positive control. Forty-five minutes after infection, A549 cells were trypsinized and collected and then washed twice with PBS. Then, cytoplasmic and nuclear fractions were separated using a hypotonic buffer solution and NP-40 detergent. The cell pellet was resuspended in 500 μL of 1×hypotonic buffer (20 mM Tris-HCl pH 7.4, 10 mM NaCl, 3 mM MgCl₂) and incubated for 15 min at 4° C. Then, 25 μL of NP-40 was added and the samples were vortexed. The homogenates were centrifuged for 10 min at 835 g at 4° C. Following the removal of the cytoplasmic fraction (supernatant), HAdV DNA was isolated from the nuclear fraction (pellet) and from the cytoplasmic fraction using the QIAamp DNA Mini Kit (QIAGEN, Valencia, Calif.).

DNA Quantification by Real-Time PCR. A549 cells (1.5×10⁵ cells/well in a 24-well plate) were incubated 24 h in 500 μL of complete DMEM and they were infected with wild-type HAdV5 (100 vp/cell) when more than 90% of confluency were observed. Infected cells were incubated 24 h at 37° C. in 500 μL of complete DMEM containing 25 μM of either compounds or the same volume of DMSO (positive control). All samples were done in duplicate. After 24 h of incubation at 37° C., DNA was purified from the cell lysate with the QIAamp DNA Mini Kit (QIAGEN, Valencia, Calif.) following the manufacturer's instructions. TaqMan primers and probes for a common region of the HAdV5 were designed with the GenScript Real-Time PCR (TaqMan) Primer Design software (GenScript). Oligonucleotides sequences were: AQ1: 5′-GCCACGGTGGGGTTTCTAAACTT-3′ (SEQ ID NO:1); AQ2: 5′-GCCCCAGTGGTCTTACATGCACAT-3′ (SEQ ID NO:2); Probe: 6-FAM-5′-TGCACCAGACCCGGGCTCAGGTACTCCGA-3′-TAMRA (SEQ ID NO:3). Real-time PCR mixtures consisted of 9.5 μL of the purified DNA, AQ1 and AQ2 at a concentration of 200 nM each and Probe at a concentration of 50 nM in a total volume of 25 μL. The PCR cycling protocol was 95° C. for 3 min followed by 40 cycles of 95° C. for 10 s and 60° C. for 30 s. Human glyceraldehyde-3-phosphate dehydrogenase (GAPDH) gene was used as internal control. Oligonucleotides sequences for GAPDH and conditions were those previously reported by Henke-Gendo et al. (2012).

For quantification, gene fragments from hexon, and GAPDH were cloned into the pGEM-T Easy vector (Promega) and known concentrations of template were used to generate a standard curve in parallel for each experiment. All assays were performed in thermal cycler LightCycler® 96 System (Roche).

Statistical Analyses. One-way ANOVA tests (Dunnet method) were carried out using the GraphPad Prism 6. We considered a statistical significance with a P value under 0.05. This statistical significance was pointed out with asterisk in graphs, and the numbers of them indicate the level of significance (*p≤0.05, **p≤0.01, ***p≤0.001, ****p≤0.0001).

### Example 2

### Substituted N-(4-amino-2-chlorophenyl)-5-chloro-2-Hydroxybenzamide Analogues as Potent Human Adenovirus Inhibitors

General Chemistry Information. All commercially available starting materials and solvents were reagent grade and used without further purification. Reactions were performed under a nitrogen atmosphere in dry glassware with magnetic stirring. Preparative column chromatography was performed using silica gel 60, particle size 0.063-0.200 mm (70-230 mesh, flash). Analytical TLC was carried out employing silica gel 60 F254 plates (Merck, Darmstadt). Visualization of the developed chromatograms was performed with detection by UV (254 nm). NMR spectra were recorded on a Brucker-300 (¹H, 600 and 300 MHz; ¹³C, 150 and 75 MHz) spectrometer. ¹H and ¹³C NMR spectra were recorded with TMS as an internal reference. Chemical shifts were expressed in ppm, and J values were given in Hz. High-resolution mass spectra (HRMS) were obtained from Thermo Fisher LTQ Orbitrap Elite mass spectrometer. Parameters include the following: Nano ESI spray voltage was 1.8 kV; Capillary temperature was 275° C. and the resolution was 60,000; Ionization was achieved by positive mode. Melting points were measured on a Thermo Scientific Electrothermal Digital Melting Point Apparatus and uncorrected. Purities of final compounds were established by analytical HPLC, which was carried out on a Shimadzu HPLC system (model: CBM-20A LC-20AD SPD-20A UV/VIS). HPLC analysis conditions: Waters Bondapak C18 (300×3.9 mm); flow rate 0.5 mL/min; UV detection at 270 and 254 nm; linear gradient from 10% acetonitrile in water to 100% acetonitrile in water in 20 min followed by 30 min of the last-named solvent (0.1% TFA was added into both acetonitrile and water). All biologically evaluated compounds are >95% pure.

N-(4-Amino-2-chlorophenyl)-5-chloro-2-hydroxybenzamide (76). To a solution of niclosamide (500 mg, 1.53 mmol) in 20 mL of MeOH was added 4 mL of saturated NH₄Cl (a.q.). Zinc dust (994 mg, 15.3 mmol) was added into the solution at 0° C. The reaction was stirred at RT for 16 h. TLC indicated that the starting material was gone. 100 mL of MeOH was added to the solution. The Zinc solid was filtered, and the filtrate was concentrated under vacuum. Then 15 mL of MeOH was added, and the resulting mixture was stirred at RT for 20 min. Compound 76 (455 mg, 100%) was filtered as a pale yellow solid. HPLC purity 99.8% (t_{R}=12.54 min). ¹H NMR (300 MHz, DMSO-d₆) δ 12.27 (s, 1H), 10.40 (s, 1H), 8.01 (d, J=2.7 Hz, 1H), 7.66 (d, J=8.7 Hz, 1H), 7.47 (dd, J=8.7, 2.7 Hz, 1H), 7.18 (br s, 1H), 7.04 (d, J=8.7 Hz, 1H), 6.71 (d, J=2.4 Hz, 1H), 6.55 (dd, J=8.7, 2.4 Hz, 1H), 5.37 (s, 2H). ¹³C NMR (75 MHz, DMSO-d₆) δ 163.7, 156.5, 147.5, 133.1, 128.8, 126.6, 126.1, 123.0, 122.6, 119.1, 118.8, 113.4, 112.8. HRMS (ESI) calcd for C₁₃H₁₁Cl₂N₂O₂, 297.0198 (M+H)⁺; found, 297.0189.

N-(4-Acetylamino-2-chlorophenyl)-5-chloro-2-hydroxybenzamide (77). To the mixture of compound 76 (200 mg, 0.67 mmol) in 10 mL of acetone was added Et₃N (341 mg, 3.4 mmol). AcCl (159 mg, 2.0 mmol) was added at 0° C. The resulting mixture was stirred at 50° C. for 2 h. The mixture was concentrated and then the residue was washed with acetone (3 mL). The intermediate 2-((4-acetamido-2-chlorophenyl)carbamoyl)-4-chlorophenyl acetate was filtered as a pale yellow solid (250 mg, 98%). ¹H NMR (600 MHz, CDCl₃) δ 8.65 (s, 1H), 8.45 (d, 1H, J=9.0 Hz), 7.98 (s, 1H), 7.96 (s, 1H), 7.50 (d, 1H, J=8.4 Hz), 7.14-7.17 (m, 3H), 2.37 (s, 3H), 2.17 (s, 3H). ¹³C NMR (150 MHz, DMSO-d6) δ 168.7, 168.6, 162.9, 146.9, 138.3, 131.4, 130.4, 129.9, 129.1, 129.0, 128.8, 128.2, 125.5, 119.3, 117.8, 24.0, 20.8.

To a solution of the intermediate 2-((4-acetamido-2-chlorophenyl)carbamoyl)-4-chlorophenyl acetate (150 mg, 0.4 mmol) in 10 mL of MeOH and 2.5 mL of H₂O was added LiOH (66 mg, 1.6 mmol) at 0° C. The resulting mixture was stirred at RT for 1 h. The mixture was diluted with EtOAc (100 mL) and washed with water (20 mL) and 2 N HCl (5 mL). The organic layer was separated and dried with anhydrous Na₂SO₄. The solution was concentrated to give compound 77 (130 mg, 97%) as a yellow solid. HPLC purity 99.3% (t_{R}=15.97 min). ¹H NMR (600 MHz, DMSO-d₆) δ 12.23 (s, 1H), 10.75 (s, 1H), 10.14 (s, 1H), 8.21 (d, 1H, J=8.4 Hz), 7.99 (s, 1H), 7.96 (s, 1H), 7.50 (d, 1H, J=9.0 Hz), 7.43 (d, 1H, J=9.0 Hz), 7.06 (d, 1H, J=9.0 Hz), 2.05 (s, 3H). ¹³C NMR (150 MHz, DMSO-d₆) δ 168.5, 162.9, 156.1, 136.6, 133.3, 130.0, 129.5, 123.9, 123.5, 123.1, 119.5, 119.2, 119.2, 118.0, 24.0. HRMS (ESI) calcd for C₁₅H₁₃Cl₂N₂O₃, 339.0303 (M+H)⁺; found, 339.0295.

General procedure A for reductive amination reaction: Compound 76 (1.0 eq) and aldehyde (1.5 eq) or ketone (3.0 eq) was suspended in DCE (5 mL/0.1 mmol) and treated with AcOH (2.5 eq). NaBH(OAc)₃ (3 eq) was added in portions at 0° C., and the mixture was stirred at RT overnight. The pH of the mixture was adjusted to 9-10 with NaHCO₃ (aq.) at 0° C. The aqueous phase was extracted with DCM, dried (Na₂SO₄) and concentrated. The residue was purified by column chromatography or recrystallization (MeOH/H₂O) to give the amino products.

General procedure B for reductive amination reaction: Compound 76 (1.0 eq) and aldehyde (1.5 eq) or ketone (3.0 eq) was dissolved in MeOH (7 mL/0.1 mmol), and then AcOH (4.0 eq) was added at 0° C. The mixture was stirred at 0° C. for 30 min, then NaBH₃CN (3.0 eq) was added. The resulting mixture was stirred at RT overnight. The pH of the mixture was adjusted to 9˜10 with NaHCO₃ (aq.) at 0° C. The mixture was extracted with DCM, dried (Na₂SO₄) and concentrated. The residue was purified by column chromatography or recrystallization (MeOH/H₂O) to give the amino products.

5-Chloro-N-(2-chloro-4-(cyclopentylamino)phenyl)-2-hydroxybenzamide (78). Compound 78 (100 mg, 73%) was prepared as a yellow solid according to general procedure B, starting from compound 76 and cyclopentanone. HPLC purity 99.5% (t_{R}=17.54 min). ¹H NMR (300 MHz, CDCl₃) δ 11.97 (s, 1H), 8.07 (s, 1H), 7.89 (d, J=9.0 Hz, 1H), 7.49 (d, J=2.4 Hz, 1H), 7.38 (dd, J=8.7, 2.4 Hz, 1H), 6.97 (d, J=9.0 Hz, 1H), 6.65 (d, J=2.7 Hz, 1H), 6.53 (dd, J=8.7, 2.7 Hz, 1H), 3.82-3.69 (m, 2H), 2.11-1.95 (m, 2H), 1.80-1.60 (m, 4H), 1.53-1.37 (m, 2H). ¹³C NMR (75 MHz, CDCl₃) δ 166.9, 160.5, 146.7, 134.5, 126.7, 125.1, 124.9, 123.8, 122.5, 120.5, 115.8, 112.8, 112.5, 54.8, 33.6 (2C), 24.2 (2C). HRMS (ESI) calcd for C₁₈H₁₉Cl₂N₂O₂, 365.0824 (M+H)⁺; found, 365.0820.

5-Chloro-N-(2-chloro-4-(ethylamino)phenyl)-2-hydroxybenzamide (79). Compound 79 (49 mg, 37%) was prepared as a yellow solid according to general procedure B, starting from compound 76 and acetaldehyde. HPLC purity 98.6% (t_{R}=15.92 min). ¹H NMR (300 MHz, CDCl₃+CD₃OD) δ 7.97 (s, 1H), 7.81 (d, J=8.7 Hz, 1H), 7.29 (dd, J=8.7, 2.4 Hz, 1H), 6.88 (d, J=8.7 Hz, 1H), 6.65 (s, 1H), 6.54 (d, J=9.0 Hz, 1H), 3.08 (q, J=7.2 Hz, 2H), 1.21 (t, J=7.2 Hz, 3H). ¹³C NMR (75 MHz, CDCl₃+CD₃OD) δ 165.3, 156.8, 147.8, 133.6, 129.9, 127.8, 126.0, 125.1, 124.3, 119.6, 118.9, 113.0, 112.3, 38.8, 14.6. HRMS (ESI) calcd for C₁₅H₁₅Cl₂N₂O₂, 325.0511 (M+H)⁺; found, 325.0504.

5-Chloro-N-(2-chloro-4-(propylamino)phenyl)-2-hydroxybenzamide (80). Compound 80 (80 mg, 68%) was prepared as a yellow solid according to general procedure A, starting from compound 76 and propionaldehyde. HPLC purity 99.4% (t_{R}=17.02 min). ¹H NMR (300 MHz, CDCl₃) δ 11.92 (s, 1H), 8.13 (s, 1H), 7.84 (d, J=8.7 Hz, 1H), 7.49 (d, J=2.1 Hz, 1H), 7.36 (dd, J=9.0, 2.4 Hz, 1H), 6.95 (d, J=8.7 Hz, 1H), 6.61 (d, J=2.4 Hz, 1H), 6.50 (dd, J=9.0, 2.4 Hz, 1H), 3.77 (s, 1H), 3.04 (t, J=7.2 Hz, 2H), 1.70-1.54 (m, 2H), 0.99 (t, J=7.2 Hz, 3H). ¹³C NMR (75 MHz, CDCl₃) δ 166.8, 160.2, 147.2, 134.4, 126.9, 125.3, 125.0, 123.8, 122.5, 120.3, 115.8, 112.2, 112.0, 45.8, 22.6, 11.6. HRMS (ESI) calcd for C₁₆H₁₇Cl₂N₂O₂, 339.0667 (M+H)⁺; found, 339.0663.

5-Chloro-N-(2-chloro-4-(isopropylamino)phenyl)-2-hydroxybenzamide (81). Compound 81 (70 mg, 53%) was prepared as a yellow solid according to general procedure B, starting from compound 76 and acetone. HPLC purity 99.7% (t_{R}=16.14 min). ¹H NMR (300 MHz, CDCl₃) δ 11.91 (s, 1H), 8.15 (s, 1H), 7.84 (d, J=8.7 Hz, 1H), 7.50 (d, J=2.4 Hz, 1H), 7.36 (dd, J=9.0, 2.4 Hz, 1H), 6.95 (d, J=8.7 Hz, 1H), 6.60 (d, J=2.7 Hz, 1H), 6.48 (dd, J=9.0, 2.7 Hz, 1H), 3.73-3.43 (m, 2H), 1.20 (d, J=6.3 Hz, 6H). ¹³C NMR (75 MHz, CDCl₃) δ 166.8, 160.2, 146.2, 134.4, 126.9, 125.3, 125.1, 123.8, 122.4, 120.3, 115.9, 112.8, 112.4, 44.5, 22.9 (2C). HRMS (ESI) calcd for C₁₆H₁₇Cl₂N₂O₂, 339.0667 (M+H)⁺; found, 339.0664.

5-Chloro-N-(2-chloro-4-(isobutylamino)phenyl)-2-hydroxybenzamide (82). Compound 82 (52 mg, 42%) was prepared as a yellow solid according to general procedure B, starting from compound 76 and isobutyraldehyde. HPLC purity 99.6% (t_{R}=17.54 min). ¹H NMR (300 MHz, CDCl₃) δ 11.93 (s, 1H), 8.10 (s, 1H), 7.86 (d, J=8.7 Hz, 1H), 7.49 (d, J=2.4 Hz, 1H), 7.37 (dd, J=8.7, 2.4 Hz, 1H), 6.96 (d, J=8.7 Hz, 1H), 6.62 (d, J=2.7 Hz, 1H), 6.52 (dd, J=9.0, 2.7 Hz, 1H), 3.84 (s, 1H), 2.90 (d, J=6.6 Hz, 2H), 1.96-1.80 (m, 1H), 0.99 (d, J=6.6 Hz, 6H). ¹³C NMR (75 MHz, CDCl₃) δ 166.9, 160.3, 147.3, 134.5, 126.9, 125.2, 125.0, 123.8, 122.5, 120.4, 115.8, 112.2, 112.0, 51.8, 28.1, 20.5 (2C). HRMS (ESI) calcd for C₁₇H₁₉Cl₂N₂O₂, 353.0824 (M+H)⁺; found, 353.0821.

5-Chloro-N-(2-chloro-4-(octan-2-ylamino)phenyl)-2-hydroxybenzamide (83). Compound 83 (80 mg, 74%) was prepared as a yellow solid according to general procedure B, starting from compound 76 and 2-octanone. HPLC purity 96.6% (t_{R}=22.46 min). ¹H NMR (300 MHz, CDCl₃) δ 11.96 (s, 1H), 8.09 (s, 1H), 7.87 (d, J=8.7 Hz, 1H), 7.49 (d, J=2.4 Hz, 1H), 7.37 (dd, J=8.7, 2.4 Hz, 1H), 6.97 (d, J=9.0 Hz, 1H), 6.62 (d, J=2.7 Hz, 1H), 6.50 (dd, J=9.0, 2.7 Hz, 1H), 3.90-3.25 (m, 2H), 1.67-1.21 (m, 10H), 1.18 (d, J=6.3 Hz, 3H), 0.95-0.83 (m, 3H). ¹³C NMR (75 MHz, CDCl₃) δ 166.9, 160.4, 146.5, 134.5, 126.9, 125.2, 125.1, 123.8, 122.3, 120.5, 115.8, 112.6, 112.3, 48.8, 37.2, 31.9, 29.4, 26.2, 22.7, 20.8, 14.2. HRMS (ESI) calcd for C₂₁H₂₇Cl₂N₂O₂, 409.1450 (M+H)⁺; found, 409.1449.

5-Chloro-N-(2-chloro-4-(decan-2-ylamino)phenyl)-2-hydroxybenzamide (84). Compound 84 (570 mg, 77%) was prepared as a yellow solid according to general procedure B, starting from compound 76 and 2-decanone. HPLC purity 99.4% (t_{R}=20.76 min) ¹H NMR (300 MHz, CDCl₃) δ 11.90 (s, 1H), 8.21 (s, 1H), 7.82 (d, J=8.7 Hz, 1H), 7.51 (d, J=2.1 Hz, 1H), 7.34 (dd, J=8.7, 1.5 Hz, 1H), 6.94 (d, J=8.7 Hz, 1H), 6.59 (d, J=1.8 Hz, 1H), 6.47 (dd, J=8.7, 1.5 Hz, 1H), 3.60 (s, 1H), 3.45-3.31 (m, 1H), 1.55-1.25 (m, 14H), 1.16 (d, J=6.0 Hz, 3H), 0.94-0.84 (m, 3H). ¹³C NMR (75 MHz, CDCl₃) δ 166.8, 160.0, 146.4, 134.3, 127.0, 125.4, 125.2, 123.8, 122.2, 120.2, 115.9, 112.6, 112.1, 48.7, 37.0, 31.9, 29.7, 29.6, 29.3, 26.1, 22.7, 20.6, 14.2. HRMS (ESI) calcd for C₂₃H₃₁Cl₂N₂O₂, 437.1763 (M+H)⁺; found, 437.1753.

5-Chloro-N-(2-chloro-4-((cyclopropylmethyl)amino)phenyl)-2-hydroxybenzamide (85). Compound 85 (59 mg, 45%) was prepared as a yellow solid according to general procedure A, starting from compound 76 and cyclopropanecarboxaldehyde. HPLC purity 95.1% (t_{R}=16.87 min). ¹H NMR (300 MHz, CDCl₃) δ 11.94 (s, 1H), 8.10 (s, 1H), 7.89 (d, J=9.0 Hz, 1H), 7.49 (d, J=2.1 Hz, 1H), 7.37 (dd, J=8.7, 2.1 Hz, 1H), 6.96 (d, J=9.0 Hz, 1H), 6.64 (d, J=2.7 Hz, 1H), 6.54 (dd, J=8.7, 2.4 Hz, 1H), 3.93 (s, 1H), 2.93 (d, J=6.6 Hz, 2H), 1.14-0.95 (m, 1H), 0.61-0.53 (m, 2H), 0.29-0.21 (m, 2H). ¹³C NMR (75 MHz, CDCl₃) δ 166.9, 160.4 147.1, 134.5, 126.7, 125.2, 124.9, 123.8, 122.8, 120.5, 115.8, 112.4, 112.1, 49.1, 10.8, 3.6 (2C). HRMS (ESI) calcd for C₁₇H₁₇Cl₂N₂O₂, 351.0667 (M+H)⁺; found, 351.0663.

5-Chloro-N-(2-chloro-4-((cyclopentylmethyl)amino)phenyl)-2-hydroxybenzamide (86). Compound 86 (101 mg, 79%) was prepared as a yellow solid according to general procedure A, starting from compound 76 and cyclopentanecarboxaldehyde. HPLC purity 98.0% (t_{R}=20.39 min). ¹H NMR (300 MHz, CDCl₃) δ 11.92 (s, 1H), 8.11 (s, 1H), 7.86 (d, J=8.7 Hz, 1H), 7.49 (d, J=2.4 Hz, 1H), 7.37 (dd, J=9.0, 2.4 Hz, 1H), 6.96 (d, J=9.0 Hz, 1H), 6.63 (d, J=2.7 Hz, 1H), 6.52 (dd, J=9.0, 2.7 Hz, 1H), 3.80 (s, 1H), 2.99 (d, J=7.2 Hz, 2H), 2.20-2.08 (m, 1H), 1.89-1.76 (m, 2H), 1.71-1.45 (m, 4H), 1.32-1.18 (m, 2H). ¹³C NMR (75 MHz, CDCl₃) δ 166.9, 160.3, 147.3, 134.5, 126.8, 125.2, 125.0, 123.8, 122.6, 120.4, 115.8, 112.2, 112.0, 49.4, 39.4, 30.7 (2C), 25.4 (2C). HRMS (ESI) calcd for C₁₉H₂₁Cl₂N₂O₂, 379.0980 (M+H)⁺; found, 379.0977.

5-Chloro-N-(2-chloro-4-((1-methylcyclopentyl) amino)phenyl)-2-hydroxybenzamide (87). To a solution of niclosamide (150 mg, 0.46 mmol) and Fe(acac)₃ (49 mg, 0.14 mmol) in EtOH (4 mL) was added donor olefin 1-methyl-1-cyclopentene (113 mg, 1.38 mmol), and PhSiH₃ (99 mg, 0.92 mmol). The resulting mixture was heated in an oil bath preheated to 60° C. with stirring for 1 h. The reaction mixture was then cooled to room temperature and Zn (595 mg, 9.2 mmol) and 2N HCl (2 ml) was added to the reaction mixture. After stirring at 60° C. for another 1 h, the reaction mixture was cooled to room temperature and filtered through Celite©. After the filter cake was washed with EtOAc, the filtrate was neutralized with sat. NaHCO₃ (aq.) and extracted with EtOAc three times. The combined organic layers were washed with brine, dried over Na₂SO₄, filtered, and concentrated under reduced pressure. The resulting crude product was then purified on SiO₂ to furnish compound 87 (52 mg, 30%) as an off-white solid. HPLC purity 96.8% (t_{R}=21.41 min). ¹H NMR (300 MHz, CDCl₃) δ 11.66 (br s, 1H), 8.12 (s, 1H), 7.84 (d, J=9.0 Hz, 1H), 7.50 (d, J=2.4 Hz, 1H), 7.37 (dd, J=9.0, 2.4 Hz, 1H), 6.96 (d, J=9.0 Hz, 1H), 6.70 (d, J=2.7 Hz, 1H), 6.57 (dd, J=9.0, 2.7 Hz, 1H), 1.98-1.84 (m, 2H), 1.82-1.65 (m, 6H), 1.40 (s, 3H). ¹³C NMR (75 MHz, CDCl₃) δ 166.9, 160.3, 145.6, 134.5, 126.4, 125.2, 124.7, 123.8, 122.4, 120.4, 115.8, 114.5, 114.0, 61.7, 40.7 (2C), 25.9, 24.4 (2C). HRMS (ESI) calcd for C₁₉H₂₁Cl₂N₂O₂, 379.0980 (M+H)⁺; found, 379.0979.

5-Chloro-N-(2-chloro-4-(cyclohexylamino)phenyl)-2-hydroxybenzamide (88). Compound 88 (110 mg, 86%) was prepared as an off-white solid according to general procedure B, starting from compound 76 and cyclohexanone. HPLC purity 99.3% (t_{R}=17.47 min). ¹H NMR (300 MHz, CDCl₃) δ 11.94 (s, 1H), 8.08 (s, 1H), 7.85 (d, J=8.7 Hz, 1H), 7.48 (s, 1H), 7.37 (d, J=7.5 Hz, 1H), 6.96 (d, J=9.0 Hz, 1H), 6.61 (s, 1H), 6.50 (d, J=8.7 Hz, 2.1, 1H), 3.67 (s, 1H), 3.28-3.14 (m, 1H), 2.10-1.96 (m, 2H), 1.84-1.72 (m, 2H), 1.71-1.61 (m, 1H), 1.45-1.31 (m, 2H), 1.29-1.09 (m, 3H). ¹³C NMR (75 MHz, CDCl₃) δ 166.9, 160.4, 146.1, 134.5, 126.8, 125.2, 125.0, 123.8, 122.3, 120.4, 115.8, 112.6, 112.4, 51.9, 33.3 (2C), 25.9, 25.0 (2C). HRMS (ESI) calcd for C₁₉H₂₁Cl₂N₂O₂, 379.0980 (M+H)⁺; found, 379.0970.

5-Chloro-N-(2-chloro-4-(dimethylamino)phenyl)-2-hydroxybenzamide (89). Compound 89 (100 mg, 76%) was prepared as a yellow solid according to general procedure B, starting from compound 76 and paraformaldehyde. HPLC purity 96.1% (t_{R}=18.82 min). ¹H NMR (300 MHz, DMSO-d₆) δ 12.21 (s, 1H), 10.49 (s, 1H), 8.02 (d, J=2.7 Hz, 1H), 7.89 (d, J=9.0 Hz, 1H), 7.48 (dd, J=9.0, 2.7 Hz, 1H), 7.03 (d, J=9.0 Hz, 1H), 6.81 (d, J=3.0 Hz, 1H), 6.73 (dd, J=9.0, 3.0 Hz, 1H), 2.91 (s, 6H). ¹³C NMR (75 MHz, DMSO-d₆) δ 163.5, 156.3, 148.6, 133.1, 129.0, 126.5, 125.5, 123.3, 123.1, 119.1, 119.0, 112.0, 111.3, 40.0 (2C). HRMS (ESI) calcd for C₁₅H₁₅Cl₂N₂O₂, 325.0511 (M+H)⁺; found, 325.0509.

5-Chloro-N-(2-chloro-4-(diethylamino)phenyl)-2-hydroxybenzamide (90). Compound 90 was prepared by a procedure the same as that used to prepare compound 79. The title compound (58 mg, 41%) was obtained as a yellow solid. HPLC purity 99.8% (t_{R}=16.50 min). ¹H NMR (300 MHz, CDCl₃) δ 11.99 (s, 1H), 8.10 (s, 1H), 7.88 (d, J=9.0 Hz, 1H), 7.50 (s, 1H), 7.37 (d, J=9.0 Hz, 1H), 6.96 (d, J=9.0 Hz, 1H), 6.68 (d, J=2.1 Hz, 1H), 6.59 (dd, J=9.0, 2.1 Hz, 1H), 3.33 (q, J=6.9 Hz, 4H), 1.17 (t, J=6.9 Hz, 6H). ¹³C NMR (75 MHz, CDCl₃) δ 166.9, 160.4, 146.5, 134.4, 127.1, 125.2, 125.0, 123.7, 121.1, 120.4, 115.9, 111.7, 110.8, 44.6 (2C), 12.6 (2C). HRMS (ESI) calcd for C₁₇H₁₉Cl₂N₂O₂, 353.0824 (M+H)⁺; found, 353.0824.

5-Chloro-N-(2-chloro-4-(dipropylamino)phenyl)-2-hydroxybenzamide (91). Compound 91 was prepared by a procedure the same as that used to prepare compound 80. The title compound (10 mg, 7%) was obtained as a yellow solid. HPLC purity 95.7% (t_{R}=18.11 min). ¹H NMR (300 MHz, CDCl3) δ 12.02 (s, 1H), 8.05 (s, 1H), 7.89 (d, J=9.0 Hz, 1H), 7.49 (d, J=2.4 Hz, 1H), 7.38 (dd, J=9.0, 2.4 Hz, 1H), 6.97 (d, J=8.7 Hz, 1H), 6.65 (d, J=2.7 Hz, 1H), 6.57 (dd, J=9.0, 2.7 Hz, 1H), 3.23 (d, J=7.5 Hz, 4H), 1.65-1.56 (m, 4H), 0.94 (t, J=7.5 Hz, 6H). ¹³C NMR (75 MHz, CDCl₃) δ 166.8, 160.4, 147.0, 134.4, 127.0, 125.2, 124.9, 123.7, 121.0, 120.5, 115.9, 111.6, 110.8, 53.0 (2C), 20.5 (2C), 11.5 (2C). HRMS (ESI) calcd for C₁₉H₂₃Cl₂N₂O₂, 381.1137 (M+H)⁺; found, 381.1133.

N-(4-(Bis(cyclopropylmethyl)amino)-2-chlorophenyl)-5-chloro-2-hydroxybenzamide (92). Compound 92 was prepared by a procedure the same as that used to prepare compound 85. The title compound (53 mg, 43%) was obtained as a yellow solid. HPLC purity 97.7% (t_{R}=17.75 min). ¹H NMR (300 MHz, CDCl₃) δ 11.97 (s, 1H), 8.15 (s, 1H), 7.91 (d, J=9.3 Hz, 1H), 7.50 (d, J=2.4 Hz, 1H), 7.36 (dd, J=8.7, 2.4 Hz, 1H), 6.95 (d, J=8.7 Hz, 1H), 6.82 (d, J=3.0 Hz, 1H), 6.73 (dd, J=9.3, 3.0 Hz, 1H), 3.25 (d, J=6.0 Hz, 4H), 1.11-0.98 (m, 2H), 0.58-0.50 (m, 4H), 0.26-0.19 (m, 4H). ¹³C NMR (75 MHz, CDCl₃) δ 166.8, 160.3, 147.6, 134.4, 126.8, 125.2, 124.7, 123.7, 121.5, 120.4, 115.9, 112.5, 111.6, 55.1 (2C), 9.4 (2C), 4.0 (4C). HRMS (ESI) calcd for C₂₁H₂₃Cl₂N₂O₂, 405.1137 (M+H)⁺; found, 405.1129.

5-Chloro-N-(2-chloro-4-((1-methylcyclopentyl)((1-methylcyclopentyl)oxy)amino) phenyl)-2-hydroxybenzamide (93). Compound 93 was prepared by a procedure the same as that used to prepare compound 87. The title compound (46 mg, 21%) was obtained as a pale yellow solid. HPLC purity 95.5% (t_{R}=25.71 min). ¹H NMR (300 MHz, CDCl₃) δ 11.78 (s, 1H), 8.38 (s, 1H), 8.20 (d, J=9.0 Hz, 1H), 7.51 (d, J=2.4 Hz, 1H), 7.46 (d, J=2.1 Hz, 1H), 7.40 (dd, J=8.7, 2.4 Hz, 1H), 7.28-7.22 (m, 1H), 6.99 (d, J=9.0 Hz, 1H), 2.10-1.84 (m, 3H), 1.82-1.41 (m, 10H), 1.40-1.19 (m, 3H), 1.17 (s, 3H), 1.05 (s, 3H). ¹³C NMR (75 MHz, CDCl₃) δ 166.9, 160.5, 151.0, 134.9, 129.8, 125.9, 125.2, 124.8, 124.0, 123.2, 121.2, 120.6, 115.8, 90.1, 71.9, 38.0, 37.7 (2C), 36.8, 25.8, 24.2, 24.0, 23.2, 22.9, 20.6. HRMS (ESI) calcd for C₂₅H₃₁Cl₂N₂O₃, 477.1712 (M+H)⁺; found, 477.1711.

N-(4-(Benzylamino)-2-chlorophenyl)-5-chloro-2-hydroxybenzamide (94). Compound 94 (110 mg, 84%) was prepared as a yellow solid according to general procedure A, starting from compound 76 and benzaldehyde. HPLC purity 95.2% (t_{R}=19.59 min). ¹H NMR (300 MHz, CD₃OD+CDCl₃) δ 7.93 (d, J=2.7 Hz, 1H), 7.81 (d, J=8.7 Hz, 1H), 7.34-7.19 (m, 6H), 6.87 (d, J=8.7 Hz, 1H), 6.66 (d, J=2.4 Hz, 1H), 6.54 (dd, J=9.0, 2.7 Hz, 1H), 4.27 (s, 2H). ¹³C NMR (75 MHz, CD₃OD+CDCl₃) δ 165.2, 156.8, 147.1, 139.2, 133.5, 129.3, 128.9 (2C), 127.5 (2C), 127.5, 127.5, 125.8, 124.8, 124.3, 119.1, 118.9, 113.0, 112.2, 48.2. HRMS (ESI) calcd for C₂₀H₁₇Cl₂N₂O₂, 387.0667 (M+H)⁺; found, 387.0659.

5-Chloro-N-(2-chloro-4-((4-fluorobenzyl)amino)phenyl)-2-hydroxybenzamide (95). Compound 95 (82 mg, 39%) was prepared as a white solid according to general procedure B, starting from compound 76 and 4-fluorobenzaldehyde. HPLC purity 99.5% (t_{R}=19.67 min). ¹H NMR (300 MHz, CD₃OD+CDCl₃) δ 7.85 (d, J=2.7 Hz, 1H), 7.80 (d, J=9.0 Hz, 1H), 7.30-7.22 (m, 3H), 7.01-6.93 (m, 2H), 6.86 (d, J=8.7 Hz, 1H), 6.62 (d, J=2.7 Hz, 1H), 6.52 (dd, J=8.7, 2.7 Hz, 1H), 4.22 (s, 2H). ¹³C NMR (75 MHz, CD₃OD+CDCl₃) δ 165.2, 162.0 (d, J=243.5 Hz), 157.1, 146.5, 134.5 (d, J=3.1 Hz), 133.4, 128.8 (d, J=8.0 Hz, 2C), 128.4, 127.2, 125.5, 124.4, 124.0, 118.8, 118.2, 115.4 (d, J=243.5 Hz, 2C), 112.7, 112.0, 47.2. HRMS (ESI) calcd for C₂₀H₁₆Cl₂FN₂O₂, 405.0573 (M+H)⁺; found, 405.0570.

5-Chloro-N-(2-chloro-4-((4-chlorobenzyl)amino)phenyl)-2-hydroxybenzamide (96). Compound 96 (126 mg, 88%) was prepared as a pale yellow solid according to general procedure B, starting from compound 76 and 4-chlorobenzaldehyde. HPLC purity 97.3% (t_{R}=20.01 min). ¹H NMR (300 MHz, DMSO-d₆) δ 12.20 (s, 1H), 10.39 (s, 1H), 7.99 (d, J=2.7 Hz, 1H), 7.70 (d, J=8.7 Hz, 1H), 7.47 (dd, J=9.0, 2.7 Hz, 1H), 7.42-7.34 (m, 4H), 7.01 (d, J=8.7 Hz, 1H), 6.68 (d, J=2.7 Hz, 1H), 6.65-6.54 (m, 2H), 4.29 (d, J=6.0 Hz, 2H). ¹³C NMR (75 MHz, DMSO-d₆) δ 163.7, 156.4, 147.0, 138.8, 133.2, 131.2, 129.0 (2C), 128.9, 128.3 (2C), 126.8, 126.1, 123.0, 123.0, 119.1, 118.8, 111.8, 111.5, 45.5. HRMS (ESI) calcd for C₂₀H₁₆C₃N₂O₂, 421.0277 (M+H)⁺; found, 421.0270.

N-(4-((4-Bromobenzyl)amino)-2-chlorophenyl)-5-chloro-2-hydroxybenzamide (97). Compound 97 (135 mg, 86%) was prepared as a pale yellow solid according to general procedure B, starting from compound 76 and 4-bromobenzaldehyde. HPLC purity 97.0% (t_{R}=20.18 min). ¹H NMR (300 MHz, DMSO-d₆) δ 12.20 (s, 1H), 10.39 (s, 1H), 7.99 (d, J=2.7 Hz, 1H), 7.70 (d, J=8.7 Hz, 1H), 7.56-7.50 (m, 2H), 7.47 (dd, J=8.7, 2.7 Hz, 1H), 7.32 (d, J=8.4 Hz, 2H), 7.01 (d, J=8.7 Hz, 1H), 6.68 (d, J=2.4 Hz, 1H), 6.65-6.54 (m, 2H), 4.27 (d, J=6.0 Hz, 2H). ¹³C NMR (75 MHz, DMSO-d₆) δ 163.7, 156.4, 147.0, 139.2, 133.2, 131.2 (2C), 129.4 (2C), 128.9, 126.8, 126.0, 123.0, 123.0, 119.7, 119.1, 118.8, 111.8, 111.5, 45.6. HRMS (ESI) calcd for C₂₀H₁₆BrCl₂N₂O₂, 464.9772 (M+H)⁺; found, 464.9764.

5-Chloro-N-(2-chloro-4-((4-(trifluoromethyl)benzyl)amino)phenyl)-2-hydroxybenzamide (98). Compound 98 (132 mg, 86%) was prepared as a pale yellow solid according to general procedure B, starting from compound 76 and 4-(trifluoromethyl)benzaldehyde. HPLC purity 95.0% (t_{R}=20.12 min). ¹H NMR (300 MHz, DMSO-d₆) δ 12.20 (s, 1H), 10.39 (s, 1H), 7.99 (d, J=2.7 Hz, 1H), 7.71 (dd, J=8.6, 3.2 Hz, 3H), 7.57 (d, J=8.0 Hz, 2H), 7.47 (dd, J=8.8, 2.8 Hz, 1H), 7.01 (d, J=8.8 Hz, 1H), 6.76-6.66 (m, 2H), 6.59 (dd, J=8.8, 2.6 Hz, 1H), 4.41 (d, J=6.0 Hz, 2H). ¹³C NMR (75 MHz, DMSO-d₆) δ 163.7, 156.4, 146.9, 144.9, 133.2, 128.9, 127.8 (2C), 127.5 (q, J=31.5 Hz), 126.8, 126.1, 125.2 (q, J=3.8 Hz), 124.3 (q, J=31.5 Hz, 2C), 123.1, 123.0, 119.1, 118.8, 111.9, 111.4, 45.8. HRMS (ESI) calcd for C₂₁H₁₆Cl₂F₃N₂O₂, 455.0541 (M+H)⁺; found, 455.0533.

5-Chloro-N-(2-chloro-4-((3-(trifluoromethyl)benzyl)amino)phenyl)-2-hydroxybenzamide (99). Compound 99 (131 mg, 85%) was prepared as a white solid according to general procedure B, starting from compound 76 and 3-(trifluoromethyl)benzaldehyde. HPLC purity 95.6% (t_{R}=20.07 min). ¹H NMR (300 MHz, DMSO-d₆) δ 12.20 (s, 1H), 10.40 (s, 1H), 7.99 (d, J=2.4 Hz, 1H), 7.76-7.53 (m, 5H), 7.47 (dd, J=8.7, 2.7 Hz, 1H), 7.02 (d, J=9.0 Hz, 1H), 6.73 (d, J=2.4 Hz, 1H), 6.67 (t, J=6.0 Hz, 1H), 6.61 (dd, J=8.7, 2.4 Hz, 1H), 4.40 (d, J=6.0 Hz, 2H). ¹³C NMR (75 MHz, CDCl₃) δ 163.7, 156.4, 146.9, 141.4, 133.2, 131.3, 129.4, 129.1 (q, J=31.2 Hz), 128.9, 126.8, 126.1, 124.3 (q, J=270.8 Hz), 123.1, 123.6 (m, 2C), 123.0, 119.1, 118.8, 111.9, 111.5, 45.7. HRMS (ESI) calcd for C₂₁H₁₆Cl₂F₃N₂O₂, 455.0541 (M+H)⁺; found, 455.0534.

5-Chloro-N-(2-chloro-4-((4-methoxybenzyl)amino)phenyl)-2-hydroxybenzamide (100). Compound 100 (55 mg, 39%) was prepared as a yellow solid according to general procedure B, starting from compound 76 and 4-methoxybenzaldehyde. HPLC purity 99.1% (t_{R}=19.07 min). ¹H NMR (300 MHz, CDCl₃) δ 11.93 (s, 1H), 8.07 (s, 1H), 7.91 (d, J=8.7 Hz, 1H), 7.48 (d, J=2.1 Hz, 1H), 7.38 (dd, J=9.0, 2.1 Hz, 1H), 7.27 (d, J=7.8 Hz, 2H), 6.97 (d, J=9.0 Hz, 1H), 6.89 (d, J=8.4 Hz, 2H), 6.68 (d, J=2.4 Hz, 1H), 6.57 (dd, J=8.7, 2.4 Hz, 1H), 4.24 (s, 2H), 4.09 (s, 1H), 3.81 (s, 3H). ¹³C NMR (75 MHz, CDCl₃) δ 166.9, 160.5, 159.2, 146.8, 134.6, 130.5, 128.9 (2C), 126.7, 125.1, 124.9, 123.8, 123.1, 120.5, 115.8, 114.3 (2C), 112.7, 112.3, 55.5, 47.9. HRMS (ESI) calcd for C₂₁H₁₉Cl₂N₂O₃, 417.0773 (M+H)⁺; found, 417.0771.

5-Chloro-N-(2-chloro-4-((4-hydroxybenzyl)amino)phenyl)-2-hydroxybenzamide (101). Compound 101 (70 mg, 51%) was prepared as a yellow solid according to general procedure B, starting from compound 76 and 4-hydroxybenzaldehyde. HPLC purity 96.8% (t_{R}=17.31 min). ¹H NMR (300 MHz, CD₃OD+CDCl₃) δ 7.98 (d, J=2.4 Hz, 1H), 7.77 (d, J=9.0 Hz, 1H), 7.29 (dd, J=8.7, 2.7 Hz, 1H), 7.15 (d, J=8.4 Hz, 2H), 6.88 (d, J=8.7 Hz, 1H), 6.79-6.72 (m, 2H), 6.67 (d, J=2.7 Hz, 1H), 6.55 (dd, J=8.7, 2.4 Hz, 1H), 4.16 (s, 2H). ¹³C NMR (75 MHz, CD₃OD+CDCl₃) δ 165.4, 156.9, 156.7, 147.9, 133.8, 130.7, 130.1, 129.3 (2C), 128.0, 126.2, 125.3, 124.4, 119.8, 119.1, 115.9 (2C), 113.3, 112.5, 48.0. HRMS (ESI) calcd for C₂₀H₁₇Cl₂N₂O₃, 403.0616 (M+H)⁺; found, 403.0610.

5-Chloro-N-(2-chloro-4-((4-(dimethylamino)benzyl)amino)phenyl)-2-hydroxybenzamide (102). Compound 102 (60 mg, 41%) was prepared as a yellow solid according to general procedure B, starting from compound 76 and 4-dimethylaminobenzaldehyde. HPLC purity 98.9% (t_{R}=14.97 min). ¹H NMR (300 MHz, CDCl₃) δ 11.96 (s, 1H), 8.07 (s, 1H), 7.92 (d, J=9.0 Hz, 1H), 7.49 (d, J=2.1 Hz, 1H), 7.38 (dd, J=8.7, 2.1 Hz, 1H), 7.22 (d, J=8.7 Hz, 2H), 6.98 (d, J=9.0 Hz, 1H), 6.76-6.69 (m, 3H), 6.59 (dd, J=9.0, 2.4 Hz, 1H), 4.19 (s, 2H), 4.03 (s, 1H), 2.95 (s, 6H). ¹³C NMR (75 MHz, CDCl₃) δ 166.9, 160.5, 150.4, 147.0, 134.6, 128.8 (2C), 126.6, 126.1, 125.1, 124.8, 123.8, 122.9, 120.5, 115.8, 112.9 (2C), 112.6, 112.2, 48.0, 40.8 (2C). HRMS (ESI) calcd for C₂₂H₂₂Cl₂N₃O₂, 430.1089 (M+H)⁺; found, 430.1082.

5-Chloro-N-(2-chloro-4-((3-chloro-5-fluorobenzyl)amino)phenyl)-2-hydroxybenzamide (103). Compound 103 (125 mg, 84%) was prepared as a pale solid according to general procedure B, starting from compound 76 and 3-chloro-5-fluorobenzaldehyde. HPLC purity 97.7% (t_{R}=20.29 min). ¹H NMR (300 MHz, DMSO-d₆) δ 12.21 (s, 1H), 10.41 (s, 1H), 7.99 (d, J=2.7 Hz, 1H), 7.73 (d, J=8.7 Hz, 1H), 7.47 (dd, J=8.7, 2.7 Hz, 1H), 7.33-7.23 (m, 2H), 7.19 (d, J=9.3 Hz, 1H), 7.02 (d, J=8.7 Hz, 1H), 6.71 (d, J=2.7 Hz, 1H), 6.66 (t, J=6.3 Hz, 1H), 6.60 (dd, J=9.0, 2.4 Hz, 1H), 4.34 (d, J=6.3 Hz, 2H). ¹³C NMR (75 MHz, DMSO-d₆) δ 163.7, 162.2 (d, J=245.9 Hz), 156.4, 146.7, 145.0 (d, J=7.6 Hz), 133.8 (d, J=10.9 Hz), 133.2, 128.9, 126.8, 126.1, 123.3, 123.2 (d, J=2.8 Hz), 123.0, 119.1, 118.8, 114.3 (d, J=25.1 Hz), 112.9 (d, J=21.5 Hz), 112.0, 111.5, 45.3. HRMS (ESI) calcd for C₂₀H₁₅Cl₃FN₂O₂, 439.0183 (M+H)⁺; found, 439.0175.

5-Chloro-N-(2-chloro-4-((3,5-dichlorobenzyl)amino)phenyl)-2-hydroxybenzamide (104). Compound 104 (130 mg, 84%) was prepared as a white solid according to general procedure B, starting from compound 76 and 3,5-dichlorobenzaldehyde. HPLC purity 97.9% (t_{R}=20.95 min). ¹H NMR (300 MHz, DMSO-d₆) δ 12.20 (s, 1H), 10.41 (s, 1H), 7.99 (d, J=2.7 Hz, 1H), 7.73 (d, J=8.7 Hz, 1H), 7.50-7.44 (m, 2H), 7.41 (d, J=1.8 Hz, 2H), 7.02 (d, J=8.7 Hz, 1H), 6.72 (d, J=2.4 Hz, 1H), 6.66 (t, J=6.3 Hz, 1H), 6.60 (dd, J=9.0, 2.7 Hz, 1H), 4.33 (d, J=6.3 Hz, 2H). ¹³C NMR (75 MHz, DMSO-d₆) δ 163.7, 156.4, 146.6, 144.6, 134.0 (2C), 133.2, 128.9, 126.8, 126.4, 126.1, 125.8 (2C), 123.3, 123.0, 119.1, 118.8, 112.0, 111.5, 45.2. HRMS (ESI) calcd for C₂₀H₁₅Cl₄N₂O₂ 454.9888 (M+H)⁺, found 454.9880.

5-Chloro-N-(2-chloro-4-((3-chloro-5-(trifluoromethyl)benzyl)amino)phenyl)-2-hydroxybenzamide (105). Compound 105 (96 mg, 58%) was prepared as a white solid according to general procedure B, starting from compound 76 and 3-chloro-5-(trifluoromethyl)benzaldehyde. HPLC purity 96.9% (t_{R}=20.91 min). ¹H NMR (300 MHz, DMSO-d₆) δ 12.19 (s, 1H), 10.41 (s, 1H), 7.99 (d, J=2.7 Hz, 1H), 7.78-7.69 (m, 4H), 7.47 (dd, J=8.7, 2.7 Hz, 1H), 7.02 (d, J=8.7 Hz, 1H), 6.75 (d, J=2.4 Hz, 1H), 6.69 (t, J=6.3 Hz, 1H), 6.62 (dd, J=9.0, 2.4 Hz, 1H), 4.42 (d, J=6.0 Hz, 2H). ¹³C NMR (75 MHz, DMSO-d₆) δ 163.7, 156.4, 146.6, 144.3, 134.0, 133.2, 131.1, 130.8 (q, J=32.2 Hz), 128.9, 126.8, 126.1, 123.4, 123.4 (q, J=271.1 Hz), 123.6 (q, J=3.8 Hz), 123.1, 122.6 (q, J=3.7 Hz), 119.1, 118.8, 112.0, 111.5, 45.2. HRMS (ESI) calcd for C₂₁H₁₅Cl₃F₃N₂O₂, 489.0151 (M+H)⁺; found, 489.0146.

5-Chloro-N-(2-chloro-4-((3-fluoro-5-(trifluoromethyl)benzyl)amino)phenyl)-2-hydroxybenzamide (106). To a solution of 2-chloro-4-fluoronitrobenzene (300 mg, 1.71 mmol) and 3-fluoro-5-(trifluoromethyl)benzylamine (396 mg, 2.05 mmol) in 5 mL of DMF was added K₂CO₃ (473 mg, 3.42 mmol). The resulting mixture was stirred at 100° C. for 1 h. Then the mixture was cooled to RT and poured into 50 mL of H₂O. The mixture was extracted with EtOAc (2×80 mL), washed with H₂O (3×20 mL) and brine (30 mL), dried (Na₂SO₄) and concentrated. The residue was purified by column chromatography (Hex/EtOAc=6/1 to 3/1) to give 3-chloro-N-(3-fluoro-5-(trifluoromethyl)benzyl)-4-nitroaniline 131b as a yellow solid (450 mg, 87%). ¹H NMR (300 MHz, CDCl₃) δ 7.97 (d, J=9.0 Hz, 1H), 7.39 (s, 1H), 7.32-7.17 (m, 2H), 6.64 (d, J=2.7 Hz, 1H), 6.48 (dd, J=9.3, 2.7 Hz, 1H), 5.05 (t, J=5.4 Hz, 1H), 4.50 (d, J=6.0 Hz, 2H).

To a solution of compound 131b (450 mg, 1.29 mmol) in 10 mL of MeOH was added 5 mL of saturated NH₄Cl (aq.). Zinc dust (838 mg, 12.9 mmol) was added into the solution at 0° C. The reaction was stirred at RT for 16 h. TLC indicated that the starting material was gone. 150 mL of EtOAc was added to the solution. The Zinc solid was filtered, and the filtrate was washed with 30 mL of brine, dried (Na₂SO₄) and concentrated under vacuum. The residue was purified by column chromatography to afford 3-chloro-N¹-(3-fluoro-5-(trifluoromethyl)benzyl)benzene-1,4-diamine 132b (310 mg, 75%) as a yellow solid. ¹H NMR (300 MHz, CDCl₃) δ 7.42 (s, 1H), 7.30-7.19 (m, 2H), 6.65 (d, J=8.4 Hz, 1H), 6.57 (d, J=2.7 Hz, 1H), 6.41 (dd, J=8.7, 2.7 Hz, 1H), 4.30 (s, 2H), 3.96-3.46 (m, 3H).

To a solution of 3-chloro-N¹-(3-fluoro-5-(trifluoromethyl)benzyl)benzene-1,4-diamine (84 mg, 0.26 mmol), 5-chloro-2-methoxybenzoic acid (59 mg, 0.32 mmol) and DMAP (7 mg, 0.05 mmol) in 15 mL of DCM was added EDCI (101 mg, 0.53 mmol) at 0° C. The resulting mixture was stirred at RT overnight, and then concentrated. The residue was purified by column chromatography (Hex/EtOAc=3/1) to afford 5-chloro-N-(2-chloro-4-((3-fluoro-5-(trifluoromethyl)benzyl)amino)phenyl)-2-methoxybenzamide (120 mg, 93%) as a yellow solid. ¹H NMR (300 MHz, CDCl3) δ 10.23 (s, 1H), 8.33 (d, J=8.7 Hz, 1H), 8.20 (d, J=2.7 Hz, 1H), 7.42-7.34 (m, 2H), 7.26-7.17 (m, 2H), 6.92 (d, J=8.7 Hz, 1H), 6.61 (d, J=2.7 Hz, 1H), 6.51 (dd, J=9.0, 2.7 Hz, 1H), 4.43 (br s, 1H), 4.34 (s, 2H), 4.01 (s, 3H).

To a solution of 5-chloro-N-(2-chloro-4-((3-fluoro-5-(trifluoromethyl)benzyl)amino)phenyl)-2-methoxybenzamide (120 mg, 0.25 mmol) in DCM (15 mL) was added BBr₃ (0.74 mL, 0.74 mmol, 1 M in DCM) dropwise at 0° C. The mixture was stirred at 0° C. for 2 h. The mixture was diluted with DCM, washed with H₂O and brine, dried (Na₂SO₄) and concentrated. The residue was purified by preparative TLC to afford compound 106 (68 mg, 58%) as a pale yellow solid. HPLC purity 98.4% (t_{R}=20.33 min). ¹H NMR (300 MHz, CDCl₃+CD₃OD) δ 7.97 (d, J=2.4 Hz, 1H), 7.82 (d, J=8.7 Hz, 1H), 7.41 (s, 1H), 7.31-7.24 (m, 2H), 7.19 (d, J=8.4 Hz, 1H), 6.88 (d, J=8.7 Hz, 1H), 6.63 (d, J=2.7 Hz, 1H), 6.52 (dd, J=8.7, 2.7 Hz, 1H), 4.36 (s, 2H). ¹³C NMR (75 MHz, CDCl₃+CD₃OD) δ 165.2, 163.3 (d, J=247.0 Hz), 156.6, 146.8, 144.6 (d, J=6.8 Hz), 133.6, 133.0 (qd, J=32.9, 8.1 Hz), 129.9, 127.7, 126.0, 125.1, 124.9, 123.8 (qd, J=270.0, 3.0 Hz), 119.8 (quint, J=3.6 Hz), 119.5, 118.9, 117.8 (d, J=21.8 Hz), 113.2, 112.2, 111.7 (dq, J=24.7, 3.8 Hz), 47.3 (d, J=1.1 Hz). HRMS (ESI) calcd for C₂₁H₁₅Cl₂F₄N₂O₂, 473.0447 (M+H)⁺; found, 473.0439.

N-(4-((3,5-Bis(trifluoromethyl)benzyl)amino)-2-chlorophenyl)-5-chloro-2-hydroxybenzamide (107). Compound 107 (119 mg, 67%) was prepared as an off-white solid according to general procedure B, starting from compound 76 and 3,5-bis(trifluoromethyl)benzaldehyde. HPLC purity 98.2% (t_{R}=21.01 min). ¹H NMR (300 MHz, CD₃OD+CDCl₃) δ 7.98 (d, J=2.4 Hz, 1H), 7.88 (s, 2H), 7.82 (d, J=8.7 Hz, 1H), 7.76 (s, 1H), 7.30 (dd, J=8.7, 2.4 Hz, 1H), 6.89 (d, J=8.7 Hz, 1H), 6.68 (d, J=2.1 Hz, 1H), 6.55 (dd, J=8.7, 2.4 Hz, 1H), 4.45 (s, 2H). ¹³C NMR (75 MHz, CD₃OD+CDCl₃) δ 165.5, 156.9, 147.3, 144.0, 133.9, 132.5 (q, J=132.5 Hz, 2C), 130.3, 128.2, 128.1 (q, J=2.7 Hz, 2C), 126.5, 125.4, 125.3, 124.3 (q, J=270.5 Hz, 2C), 121.5 (septet, J=3.8 Hz), 119.9, 119.2, 113.6, 112.3, 47.4. HRMS (ESI) calcd for C₂₂H₁₅Cl₂F₆N₂O₂, 523.0415 (M+H)⁺; found, 523.0413.

5-Chloro-N-(2-chloro-4-((pyridin-4-ylmethyl)amino)phenyl)-2-hydroxybenzamide (108). Compound 108 (54 mg, 41%) was prepared as a yellow solid according to general procedure B, starting from compound 76 and 4-pyridinecarboxaldehyde. HPLC purity 98.3% (t_{R}=16.26 min). ¹H NMR (300 MHz, DMSO-d₆) δ 12.16 (s, 1H), 10.40 (s, 1H), 8.51 (d, J=4.2 Hz, 2H), 7.99 (d, J=2.7 Hz, 1H), 7.72 (d, J=9.0 Hz, 1H), 7.46 (dd, J=8.7, 2.7 Hz, 1H), 7.35 (d, J=5.7 Hz, 2H), 7.01 (d, J=8.7 Hz, 1H), 6.72-6.62 (m, 2H), 6.58 (dd, J=8.7, 2.4 Hz, 1H), 4.35 (d, J=6.3 Hz, 2H). ¹³C NMR (75 MHz, DMSO-d₆) δ 163.7, 156.4, 149.5 (2C), 149.0, 146.9, 133.2, 128.9, 126.8, 126.1, 123.2, 123.0, 122.2 (2C), 119.01, 118.8, 111.9, 111.4, 45.2. HRMS (ESI) calcd for C₁₉H₁₆Cl₂N₃O₂, 388.0620 (M+H)⁺; found, 388.0615.

5-Chloro-N-(2-chloro-4-(((2-chloropyridin-3-yl)methyl)amino)phenyl)-2-hydroxybenzamide (109). Compound 109 (90 mg, 63%) was prepared as a yellow solid according to general procedure B, starting from compound 76 and 2-chloro-3-formylpyridine. HPLC purity 96.0% (t_{R}=18.73 min). ¹H NMR (300 MHz, DMSO-d₆) δ 12.20 (s, 1H), 10.42 (s, 1H), 8.32 (dd, J=4.8, 1.8 Hz, 1H), 7.99 (d, J=2.7 Hz, 1H), 7.84-7.72 (m, 2H), 7.47 (dd, J=8.7, 2.7 Hz, 1H), 7.42 (dd, J=7.5, 4.8 Hz, 1H), 7.02 (d, J=8.7 Hz, 1H), 6.72 (d, J=2.4 Hz, 1H), 6.65 (t, J=6.0 Hz, 1H), 6.59 (dd, J=9.0, 2.4 Hz, 1H), 4.36 (d, J=5.7 Hz, 2H). ¹³C NMR (75 MHz, DMSO-d₆) δ 163.7, 156.4, 149.4, 148.1, 146.6, 137.8, 133.2, 133.2, 128.9, 126.9, 126.1, 123.5, 123.3, 123.0, 119.1, 118.8, 111.9, 111.3, 43.6. HRMS (ESI) calcd for C₁₉H₁₅C₃N₃O₂, 422.0230 (M+H)⁺; found, 422.0228.

5-Chloro-N-(2-chloro-4-(((4-chloropyridin-3-yl)methyl)amino)phenyl)-2-hydroxybenzamide (110). Compound 110 (110 mg, 77%) was prepared as a yellow solid according to general procedure B, starting from compound 76 and 4-chloropyridine-3-carboxaldehyde. HPLC purity 99.1% (t_{R}=18.96 min). ¹H NMR (300 MHz, DMSO-d₆) δ 12.20 (s, 1H), 10.42 (s, 1H), 8.57 (s, 1H), 8.46 (d, J=5.1 Hz, 1H), 8.00 (d, J=2.7 Hz, 1H), 7.76 (d, J=9.0 Hz, 1H), 7.57 (d, J=5.43 Hz, 1H), 7.47 (dd, J=8.7, 2.7 Hz, 1H), 7.02 (d, J=8.7 Hz, 1H), 6.77 (d, J=2.4 Hz, 1H), 6.64 (dd, J=9.0, 2.4 Hz, 1H), 6.55 (t, J=6.0 Hz, 1H), 4.41 (d, J=5.7 Hz, 2H). ¹³C NMR (75 MHz, DMSO-d₆) δ 163.7, 156.4, 150.3, 149.6, 146.6, 142.6, 133.2, 132.2, 128.9, 126.9, 126.1, 124.4, 123.4, 123.0, 119.1, 118.8, 111.9, 111.3, 42.2. HRMS (ESI) calcd for C₁₉H₁₅Cl₃N₃O₂, 422.0230 (M+H)⁺; found, 422.0223.

N-(4-(((6-Bromopyridin-2-yl)methyl)amino)-2-chlorophenyl)-5-chloro-2-hydroxybenzamide (111). Compound 111 (90 mg, 57%) was prepared as a yellow solid according to general procedure B, starting from compound 76 and 6-bromo-2-pyridinecarboxaldehyde. HPLC purity 96.7% (t_{R}=19.06 min). H NMR (300 MHz, DMSO-d₆) δ 12.19 (s, 1H), 10.40 (s, 1H), 7.99 (d, J=2.7 Hz, 1H), 7.77-7.68 (m, 2H), 7.52 (d, J=7.8 Hz, 1H), 7.47 (dd, J=9.0, 2.7 Hz, 1H), 7.39 (d, J=7.5 Hz, 1H), 7.02 (d, J=8.7 Hz, 1H), 6.75-6.67 (m, 2H), 6.59 (dd, J=8.7, 2.4 Hz, 1H), 4.38 (d, J=6.0 Hz, 2H). ¹³C NMR (75 MHz, DMSO-d₆) δ 163.7, 161.4, 156.4, 146.8, 140.8, 140.1, 133.2, 128.9, 126.9, 126.3, 126.1, 123.3, 123.1, 120.5, 119.1, 118.8, 111.9, 111.3, 47.7. HRMS (ESI) calcd for C₁₉H₁₅BrCl₂N₃O₂, 465.9725 (M+H)⁺; found, 465.9724.

5-Chloro-N-(2-chloro-4-(((6-hydroxypyridin-3-yl)methyl)amino)phenyl)-2-hydroxybenzamide (112). Compound 112 (120 mg, 88%) was prepared as a yellow solid according to general procedure B, starting from compound 76 and 6-hydroxynicotinaldehyde. HPLC purity 98.6% (t_{R}=16.79 min). ¹H NMR (300 MHz, DMSO-d₆) δ 12.20 (s, 1H), 11.43 (s, 1H), 10.41 (s, 1H), 8.00 (d, J=2.7 Hz, 1H), 7.74 (d, J=8.7 Hz, 1H), 7.46 (td, J=9.3, 2.4 Hz, 2H), 7.33 (s, 1H), 7.02 (d, J=8.7 Hz, 1H), 6.74 (d, J=2.1 Hz, 1H), 6.62 (dd, J=8.7, 2.1 Hz, 1H), 6.37-6.29 (m, 2H), 4.00 (d, J=4.8 Hz, 2H). ¹³C NMR (75 MHz, DMSO-d₆) δ 163.7, 162.0, 156.4, 146.9, 141.5, 133.2, 128.9, 126.8, 126.0, 123.0, 119.9, 119.1, 118.8, 115.7, 111.9, 111.5, 42.8. HRMS (ESI) calcd for C₁₉H₁₆Cl₂N₃O₃, 404.0569 (M+H)⁺; found, 404.0567.

N-(4-(((5-Bromothiophen-2-yl)methyl)amino)-2-chlorophenyl)-5-chloro-2-hydroxybenzamide (113). Compound 113 (95 mg, 60%) was prepared as a yellow solid according to general procedure B, starting from compound 76 and 5-bromothiophene-2-carboxaldehyde. HPLC purity 99.5% (t_{R}=20.16 min). ¹H NMR (300 MHz, DMSO-d₆) δ 12.20 (s, 1H), 10.42 (s, 1H), 8.00 (d, J=2.7 Hz, 1H), 7.75 (d, J=8.7 Hz, 1H), 7.47 (dd, J=8.7, 2.7 Hz, 1H), 7.07 (d, J=3.6 Hz, 1H), 7.02 (d, J=8.7 Hz, 1H), 6.92 (d, J=3.9 Hz, 1H), 6.77 (d, J=2.4 Hz, 1H), 6.68-6.57 (m, 2H), 4.44 (d, J=5.7 Hz, 2H). ¹³C NMR (75 MHz, DMSO-d₆) δ 163.6, 156.4, 146.5, 146.2, 133.2, 130.0, 128.9, 126.7, 125.9, 125.8, 123.5, 123.1, 119.1, 118.9, 112.2, 111.8, 109.3, 42.0. HRMS (ESI) calcd for C₁₈H₁₄Cl₂N₂O₂S, 470.9336 (M+H)⁺; found, 470.9334.

N-(4-(((1H-Indazol-6-yl)methyl)amino)-2-chlorophenyl)-5-chloro-2-hydroxybenzamide (114). Compound 114 (122 mg, 84%) was prepared as a yellow solid according to general procedure B, starting from compound 76 and 1H-indazole-6-carboxaldehyde. HPLC purity 97.2% (t_{R}=18.05 min). ¹H NMR (300 MHz, DMSO-d₆) δ 12.95 (s, 1H), 12.20 (s, 1H), 10.39 (s, 1H), 8.05-7.95 (m, 2H), 7.71 (dd, J=8.7, 2.7 Hz, 2H), 7.52-7.41 (m, 2H), 7.12 (d, J=8.1 Hz, 1H), 7.01 (d, J=8.7 Hz, 1H), 6.75-6.58 (m, 3H), 4.43 (d, J=5.7 Hz, 2H). ¹³C NMR (75 MHz, DMSO-d₆) δ 163.7, 156.4, 147.3, 140.2, 137.8, 133.3, 133.2, 128.9, 126.8, 126.1, 123.0, 122.8, 121.9, 120.4, 120.2, 119.1, 118.8, 111.8, 111.4, 107.7, 46.4. HRMS (ESI) calcd for C₂₁H₁₇Cl₂N₄O₂, 427.0729 (M+H)⁺; found, 427.0720.

5-Chloro-N-(2-chloro-4-((2-methyl-1-phenylpropan-2-yl)((2-methyl-1-phenylpropan-2-yl)oxy)amino)phenyl)-2-hydroxybenzamide (115). Compound 115 was prepared by a procedure similar to that used to prepare compound 87, starting from niclosamide and 2-methyl-3-phenyl-1-propene. The title compound (115 mg, 43%) was obtained as a yellow solid. HPLC purity 97.1% (t_{R}=21.59 min). ¹H NMR (300 MHz, CDCl3) δ 8.38 (s, 1H), 7.91 (d, J=9.0 Hz, 1H), 7.55 (d, J=2.4 Hz, 1H), 7.36-7.16 (m, 10H), 7.08 (dd, J=7.5, 1.5 Hz, 2H), 6.93 (d, J=8.7 Hz, 1H), 6.79 (d, J=2.7 Hz, 1H), 6.65 (dd, J=9.0, 2.7 Hz, 1H), 2.93 (s, 2H), 2.74 (s, 2H), 1.30 (s, 6H), 1.21 (s, 6H). ¹³C NMR (75 MHz, CDCl3) δ 166.6, 159.9, 145.3, 137.8, 137.7, 134.3, 130.6 (2C), 130.5 (2C), 128.3 (2C), 128.1 (2C), 126.6, 126.5, 126.3, 125.7, 124.7, 123.9, 123.5, 120.2, 116.1, 116.0, 115.4, 71.0, 54.4, 49.8, 46.2, 29.2 (2C), 28.4 (2C). HRMS (ESI) calcd for C₃₃H₃₅Cl₂N₂O₃ 577.2025 (M+H)⁺, found 577.2017.

5-Chloro-N-(2-chloro-4-((4-hydroxy-2-methylbutan-2-yl)amino)phenyl)-2-hydroxybenzamide (118). Compound 118 was prepared by a procedure similar to that used to prepare compound 87, starting from niclosamide and 3-methyl-3-buten-1-ol. The title compound (44 mg, 58%) was obtained as a yellow solid. HPLC purity 95.6% (t_{R}=15.89 min). ¹H NMR (300 MHz, CDCl₃) δ 8.66 (s, 1H), 7.93 (d, J=8.7 Hz, 1H), 7.61 (s, 1H), 7.35 (d, J=8.4 Hz, 1H), 7.00-6.89 (m, 2H), 6.75 (dd, J=8.7, 1.8 Hz, 1H), 5.85 (br s, 2H), 3.88 (t, J=6.0 Hz, 2H), 1.89 (t, J=6.0 Hz, 2H), 1.31 (s, 6H). ¹³C NMR (75 MHz, CDCl₃) δ 166.6, 159.6, 143.7, 134.4, 126.1, 125.9, 125.9, 124.3, 124.1, 120.2, 119.1, 118.3, 116.3, 59.9, 55.2, 42.7, 28.1 (2C). HRMS (ESI) calcd for C₁₈H₂₁Cl₂N₂O₃, 383.0929 (M+H)⁺; found, 383.0920.

5-Chloro-N-(2-chloro-4-((4-hydroxybutan-2-yl)amino)phenyl)-2-hydroxybenzamide (119). Compound 119 was prepared by a procedure similar to that used to prepare compound 87, starting from niclosamide and 3-buten-1-ol. The title compound (20 mg, 27%) was obtained as a yellow solid. HPLC purity 95.1% (t_{R}=15.65 min). ¹H NMR (300 MHz, CDCl₃) δ 8.28 (s, 1H), 7.85 (d, J=8.7 Hz, 1H), 7.54 (d, J=2.4 Hz, 1H), 7.37 (dd, J=9.0, 2.4 Hz, 1H), 6.96 (d, J=8.7 Hz, 1H), 6.68 (d, J=2.7 Hz, 1H), 6.55 (dd, J=9.0, 2.7 Hz, 1H), 3.87-3.73 (m, 2H), 3.70-3.61 (m, 1H), 1.80-1.72 (m, 2H), 1.21 (d, J=6.3 Hz, 3H). ¹³C NMR (75 MHz, CDCl₃) δ 166.9, 160.2, 146.2, 134.5, 127.0, 125.5, 125.2, 123.9, 123.1, 120.4, 116.0, 113.4, 112.9, 60.6, 47.5, 39.3, 21.0. HRMS (ESI) calcd for C₁₇H₁₉Cl₂N₂O₃, 369.0773 (M+H)⁺; found, 369.0767.

5-Chloro-N-(2-chloro-4-((3-hydroxypropyl)amino)phenyl)-2-hydroxybenzamide (120). To a solution of 3-((tetrahydro-2H-pyran-2-yl)oxy)propanal (260 mg, 1.64 mmol) and compound 76 (250 mg, 1.10 mmol) in 10 mL of MeOH was added AcOH (197 mg, 3.30 mmol) at 0° C. The mixture was stirred at 0° C. for 30 min, then NaBH₃CN (226 mg, 3.30 mmol) was added. The resulting mixture was stirred at RT overnight. The pH of the mixture was adjusted to 9-10 with NaHCO₃ (aq.) at 0° C. The mixture was extracted with DCM (2×70 mL), dried (Na₂SO₄) and concentrated. The residue was purified by column chromatography to give the product as a mixture. The mixture was dissolved in 20 mL of MeOH, and p-TsOH (30 mg, 0.17 mmol) was added. The resulting mixture was stirred at RT for 12 h and then concentrated. The residue was purified by column chromatography (DCM/MeOH=20/1) to afford compound 120 (86 mg, 22% in two steps) as a yellow solid. HPLC purity 95.5% (t_{R}=16.81 min). ¹H NMR (300 MHz, CD₃OD) δ 8.00 (d, J=2.1 Hz, 1H), 7.76 (d, J=8.7 Hz, 1H), 7.37 (dd, J=8.7, 2.4 Hz, 1H), 6.95 (d, J=8.7 Hz, 1H), 6.71 (d, J=2.1 Hz, 1H), 6.59 (dd, J=8.7, 2.1 Hz, 1H), 3.68 (t, J=6.3 Hz, 2H), 3.17 (t, J=6.9 Hz, 2H), 1.90-1.76 (m, 2H). ¹³C NMR (75 MHz, CD₃OD) δ 166.2, 157.7, 149.3, 134.3, 130.4, 128.8, 127.1, 125.7, 124.5, 120.3, 119.7, 113.3, 112.6, 60.8, 41.6, 33.0. HRMS (ESI) calcd for C₁₆H₁₇Cl₂N₂O₃, 355.0616 (M+H)⁺; found, 355.0608.

N-(4-(Bis(3-hydroxypropyl)amino)-2-chlorophenyl)-5-chloro-2-hydroxybenzamide (121). Compound 121 was prepared by a procedure the same as that used to prepare compound 120. The title compound (34 mg, 8% in two steps) was obtained as a yellow solid. HPLC purity 95.8% (t_{R}=16.79 min). ¹H NMR (300 MHz, CD₃OD+CDCl₃) δ 8.00 (d, J=2.7 Hz, 1H), 7.85 (d, J=9.0 Hz, 1H), 7.37 (dd, J=9.0, 2.7 Hz, 1H), 6.95 (d, J=8.7 Hz, 1H), 6.83 (d, J=3.0 Hz, 1H), 6.72 (dd, J=9.0, 2.7 Hz, 1H), 3.63 (t, J=6.0 Hz, 4H), 3.43 (t, J=7.2 Hz, 4H), 1.87-1.73 (m, 4H). ¹³C NMR (75 MHz, CD₃OD) δ 165.9, 157.6, 148.1, 134.3, 130.5, 128.8, 126.9, 125.7, 124.0, 120.4, 119.7, 113.2, 112.1, 60.5 (2C), 48.1 (2C), 31.0 (2C). HRMS (ESI) calcd for C₁₉H₂₃Cl₂N₂O₄, 413.1035 (M+H)⁺; found, 413.1028.

tert-Butyl (2-((3-chloro-4-(5-chloro-2-hydroxybenzamido)phenyl)amino)ethyl)carbamate (122). Compound 122 (100 mg, 67%) was prepared as a yellow solid according to general procedure B, starting from compound 76 and N-Boc-2-aminoacetaldehyde. HPLC purity 98.2% (t_{R}=17.72 min). ¹H NMR (300 MHz, CDCl₃) δ 11.85 (s, 1H), 8.32 (s, 1H), 7.83 (d, J=8.7 Hz, 1H), 7.55 (d, J=2.1 Hz, 1H), 7.36 (dd, J=8.7, 2.4 Hz, 1H), 6.95 (d, J=8.7 Hz, 1H), 6.61 (d, J=2.4 Hz, 1H), 6.51 (dd, J=8.7, 2.7 Hz, 1H), 4.92 (s, 1H), 4.35 (br s, 1H), 3.39-3.29 (m, 2H), 3.23-3.15 (m, 2H), 1.45 (s, 9H). ¹³C NMR (75 MHz, CDCl3) δ 166.8, 160.0, 156.8, 146.8, 134.4, 127.1, 125.6, 125.3, 123.9, 123.1, 120.3, 116.0, 112.4, 112.0, 80.1, 44.7, 40.1, 28.5 (3C). HRMS (ESI) calcd for C₂₀H₂₄Cl₂N₃O₄, 440.1144 (M+H)⁺; found, 440.1146.

N-(4-((2-Aminoethyl)amino)-2-chlorophenyl)-5-chloro-2-hydroxybenzamide hydrochloride (123). To a solution of compound 122 (40 mg, 0.091 mmol) in 5 mL of DCM was added 1 mL of TFA at 0° C. The resulting mixture was stirred at 0° C. for 2 h, and concentrated. Then the residue was dissolved in 5 mL of MeOH and 1 mL of ammonium hydroxide was added. The mixture was stirred at RT for 10 min, and then concentrated. The residue was purified through column chromatography on reverse phase C-18 silica gel (eluent, H₂O (1% HCl)/CH₃CN=2/1 to 1/1) to afford compound 123 (28 mg, 78%) as a yellow solid. HPLC purity 99.4% (t_{R}=14.84 min). ¹H NMR (300 MHz, CD₃OD) δ 7.96 (d, J=2.4 Hz, 1H), 7.81 (d, J=9.0 Hz, 1H), 7.42 (dd, J=8.7, 2.7 Hz, 1H), 7.02 (d, J=8.7 Hz, 1H), 6.87 (d, J=2.4 Hz, 1H), 6.73 (dd, J=8.7, 2.4 Hz, 1H), 3.47 (t, J=6.0 Hz, 2H), 3.19 (t, J=6.0 Hz, 2H). ¹³C NMR (75 MHz, CD₃OD) δ 165.9, 156.9, 147.6, 134.6, 130.5, 128.7, 127.0, 125.8, 125.6, 120.2, 119.7, 114.2, 113.2, 41.8, 39.6. HRMS (ESI) calcd for C₁₅H₁₆Cl₂N₃O₂, 340.0620 (M-Cl)⁺; found, 340.0610.

tert-Butyl 4-((3-chloro-4-(5-chloro-2-hydroxybenzamido)phenyl)amino)piperidine-1-carboxylate (124). Compound 124 (305 mg, 72%) was prepared as an off-white solid according to general procedure B, starting from compound 76 and 1-tert-butoxycarbonylpiperidin-4-one. HPLC purity 99.5% (t_{R}=18.48 min). ¹H NMR (300 MHz, DMSO-d₆) δ 11.53 (br s, 1H), 7.99-7.85 (m, 2H), 7.32 (dd, J=8.7, 2.7 Hz, 1H), 6.94 (d, J=8.7 Hz, 1H), 6.72 (d, J=2.1 Hz, 1H), 6.59 (dd, J=9.0, 2.1 Hz, 1H), 5.71 (d, J=8.1 Hz, 1H), 3.92-3.80 (m, 2H), 3.48-3.36 (m, 1H), 3.00-2.84 (m, 2H), 1.98-1.78 (m, 2H), 1.40 (s, 9H), 1.29-1.14 (m, 2H). ¹³C NMR (75 MHz, DMSO-d₆) δ 163.7, 159.7, 153.9, 145.5, 132.5, 128.8, 125.6, 125.1, 123.8, 120.4, 120.0, 119.3, 112.0, 111.5, 78.5, 48.6, 42.2 (2C), 31.5 (2C), 28.1 (3C). HRMS (ESI) calcd for C₂₃H₂₈Cl₂N₃O₄, 480.1457 (M+H)⁺; found, 480.1450.

5-Chloro-N-(2-chloro-4-(piperidin-4-ylamino)phenyl)-2-hydroxybenzamide hydrochloride (125). To a solution of compound 124 (245 mg, 0.51 mmol) in 5 mL of MeOH was added 4 M HCl (5 mL) at 0° C. The resulting mixture was stirred at RT for 48 h. Then most of MeOH was evaporated and compound 125 (185 mg, 87%) was isolated as a pale yellow solid by filtration. HPLC purity 99.8% (t_{R}=15.40 min). ¹H NMR (300 MHz, DMSO-d₆) δ 12.22 (s, 1H), 10.42 (s, 1H), 9.10-8.80 (m, 2H), 8.01 (d, J=2.4 Hz, 1H), 7.80 (d, J=9.0 Hz, 1H), 7.46 (dd, J=8.7, 2.4 Hz, 1H), 7.09 (d, J=9.0 Hz, 1H), 6.86 (d, J=2.4 Hz, 1H), 6.71 (dd, J=8.7, 2.1 Hz, 1H), 3.64-3.50 (m, 1H), 3.36-3.22 (m, 2H), 3.08-2.90 (m, 2H), 2.13-1.97 (m, 2H), 1.74-1.56 (m, 2H). ¹³C NMR (75 MHz, DMSO-d₆) δ 163.5, 156.3, 144.6, 133.1, 129.0, 126.5, 125.7, 124.1, 123.1, 119.1, 119.0, 113.1, 112.6, 47.1, 41.9 (2C), 27.9 (2C). HRMS (ESI) calcd for C₁₈H₂₀Cl₂N₃O₂, 380.0933 (M-Cl)⁺; found, 380.0922.

tert-Butyl 4-(((3-chloro-4-(5-chloro-2-hydroxybenzamido)phenyl)amino)methyl)piperidine-1-carboxylate (126). Compound 126 (120 mg, 72%) was prepared as a yellow solid according to general procedure B, starting from compound 76 and N-Boc-4-piperidinecarboxaldehyde. HPLC purity 97.0% (t_{R}=20.32 min). ¹H NMR (300 MHz, DMSO-d₆) δ 12.22 (s, 1H), 10.39 (s, 1H), 8.01 (d, J=2.7 Hz, 1H), 7.70 (d, J=8.7 Hz, 1H), 7.47 (dd, J=8.7, 2.7 Hz, 1H), 7.02 (d, J=9.0 Hz, 1H), 6.70 (d, J=2.4 Hz, 1H), 6.58 (dd, J=8.7, 2.4 Hz, 1H), 6.00 (t, J=5.7 Hz, 1H), 4.02-3.88 (m, 2H), 2.91 (d, J=5.4 Hz, 2H), 2.79-2.58 (m, 2H), 1.79-1.63 (m, 3H), 1.39 (s, 9H), 1.14-0.95 (m, 2H). ¹³C NMR (75 MHz, DMSO-d₆) δ 163.8, 156.5, 153.8, 147.7, 133.1, 128.8, 127.0, 126.2, 123.0, 122.3, 119.1, 118.8, 111.3, 110.9, 78.4, 48.3 (2C), 43.2, 35.0 (2C), 29.6, 28.1 (3C). HRMS (ESI) calcd for C₂₄H₃₀Cl₂N₃O₄, 494.1613 (M+H)⁺; found, 494.1602.

5-Chloro-N-(2-chloro-4-((piperidin-4-ylmethyl)amino)phenyl)-2-hydroxybenzamide (127). To a solution of compound 126 (60 mg, 0.12 mmol) in 5 mL of DCM was added 1 mL of TFA at 0° C. The resulting mixture was stirred at 0° C. for 2 h, and concentrated. Then the residue was dissolved in 5 mL of MeOH and 1 mL of ammonium hydroxide was added. The mixture was stirred at RT for 10 min. The yellow solid was isolated by filtration to afford compound 127 (38 mg, 79%). HPLC purity 96.3% (t_{R}=15.61 min). ¹H NMR (300 MHz, DMSO-d₆) δ 13.83 (s, 1H), 8.12 (d, J=9.0 Hz, 1H), 7.69 (d, J=3.0 Hz, 1H), 7.00 (dd, J=8.7, 3.0 Hz, 1H), 6.63 (d, J=1.5 Hz, 1H), 6.57-6.45 (m, 2H), 5.74 (t, J=5.1 Hz, 1H), 3.35-3.08 (m, 3H), 2.96-2.71 (m, 4H), 1.94-1.70 (m, 3H), 1.37-1.16 (m, 2H). ¹³C NMR (75 MHz, DMSO-d₆) δ 167.1, 164.8, 145.5, 131.5, 128.4, 125.9, 124.2, 124.1, 122.2, 119.4, 114.2, 111.4, 111.1, 48.2, 43.4 (2C), 33.2, 27.3 (2C). HRMS (ESI) calcd for C₁₉H₂₂Cl₂N₃O₂, 394.1089 (M+H)⁺; found, 394.1085.

5-Chloro-N-(2-chloro-4-(((1-methylpiperidin-4-yl)methyl)amino)phenyl)-2-hydroxybenzamide (128). Compound 128 (85 mg, 61%) was prepared as a white solid according to general procedure B, starting from compound 76 and N-methyl-piperidine-4-carboxaldehyde. HPLC purity 96.0% (t_{R}=16.02 min). ¹H NMR (300 MHz, DMSO-d₆) δ 7.96 (d, J=8.4 Hz, 1H), 7.88 (s, 1H), 7.35-7.21 (m, 1H), 6.90 (d, J=8.7 Hz, 1H), 6.67 (s, 1H), 6.56 (d, J=9.0 Hz, 1H), 5.87 (s, 1H), 3.04-2.82 (m, 4H), 2.35 (s, 3H), 2.27-2.12 (m, 2H), 1.85-1.70 (m, 2H), 1.57 (s, 1H), 1.34-1.14 (m, 2H). ¹³C NMR (75 MHz, DMSO-d₆) δ 163.8, 160.8, 146.7, 132.4, 128.8, 125.4, 124.9, 123.9, 120.3, 119.5, 119.4, 111.4, 110.9, 54.4 (2C), 48.3, 44.9, 33.6, 28.9 (2C). HRMS (ESI) calcd for C₂₀H₂₄Cl₂N₃O₂, 408.1246 (M+H)⁺; found, 408.1236.

5-Chloro-N-(2-chloro-4-((2-morpholinoethyl)amino)phenyl)-2-hydroxybenzamide (129). To a solution of 2-chloro-4-fluoronitrobenzene (400 mg, 2.28 mmol) and 2-morpholinoethanamine (355 mg, 2.73 mmol) in 10 mL of DMF was added K₂CO₃ (630 mg, 4.56 mmol). The resulting mixture was stirred at 100° C. for 1 h. Then the mixture was cooled to RT and poured into 50 mL of H₂O. Yellow precipitate was isolated by filtration and dried to afford 3-chloro-N-(2-morpholinoethyl)-4-nitroaniline 131c (570 mg, 87%). ¹H NMR (300 MHz, CDCl₃) δ 7.95 (d, J=9.0 Hz, 1H), 6.57 (d, J=2.7 Hz, 1H), 6.44 (dd, J=9.0, 2.7 Hz, 1H), 5.25 (t, J=4.5 Hz, 1H), 3.71 (t, J=4.5 Hz, 4H), 3.26-3.13 (m, 2H), 2.68-2.58 (m, 2H), 2.52-2.40 (m, 4H). ¹³C NMR (75 MHz, CDCl₃) δ 152.4, 136.0, 130.8, 129.0, 113.7, 110.3, 66.9 (2C), 56.3, 53.2 (2C), 39.1.

To a solution of compound 131c (570 mg, 1.99 mmol) in 20 mL of MeOH was added 10 mL of saturated NH₄Cl (aq.). Zinc dust (648 mg, 9.97 mmol) was added to the solution at 0° C. The reaction was stirred at RT for 16 h. TLC indicated that the starting material was gone. 200 mL of EtOAc was added to the solution. The Zinc solid was filtered, and the filtrate was washed with 30 mL of brine, dried (Na₂SO₄) and concentrated under vacuum. The residue was purified by column chromatography to afford 3-chloro-N¹-(2-morpholinoethyl)benzene-1,4-diamine 132c (450 mg, 88%) as a yellow solid.

To a solution of compound 132c (88 mg, 0.34 mmol), 5-chloro-2-methoxybenzoic acid (71 mg, 0.38 mmol) and DMAP (10 mg, 0.08 mmol) in 10 mL of DCM was added EDCI (130 mg, 0.68 mmol) at 0° C. The resulting mixture was stirred at RT overnight, and then concentrated. The residue was purified by column chromatography (DCM/MeOH=100/1 to 30/1) to afford the intermediate 5-chloro-N-(2-chloro-4-((2-morpholinoethyl)amino)phenyl)-2-methoxybenzamide (130 mg, 89%) as a yellow solid. ¹H NMR (300 MHz CDCl₃) δ 10.24 (s, 1H), 8.36 (d, J=8.7 Hz, 1H), 8.26 (d, J=2.7 Hz, 1H), 7.41 (dd, J=8.7, 2.7 Hz, 1H), 6.96 (d, J=8.7 Hz, 1H), 6.67 (d, J=2.7 Hz, 1H), 6.58 (dd, J=9.0, 2.7 Hz, 1H), 4.34 (br s, 1H), 4.05 (s, 3H), 3.72 (t, J=4.5 Hz, 4H), 3.14 (t, J=6.0 Hz, 2H), 2.62 (t, J=6.0 Hz, 2H), 2.47 (t, J=4.5 Hz, 4H). ¹³C NMR (75 MHz, CDCl₃) δ 161.4, 155.9, 145.8, 132.7, 132.2, 127.0, 126.0, 124.5, 123.6, 123.4, 113.1, 112.7, 112.4, 67.1 (2C), 57.1, 56.7, 53.5 (2C), 40.1.

To a solution of the intermediate 5-chloro-N-(2-chloro-4-((2-morpholinoethyl)amino)phenyl)-2-methoxybenzamide (130 mg, 0.38 mmol) in DCM (15 mL) was added BBr₃ (1.53 mL, 1.53 mmol, 1 M in DCM) dropwise at 0° C. The mixture was stirred at RT for 2 h. The mixture was diluted with DCM, washed with H₂O and brine, dried (Na₂SO₄) and concentrated. The residue was purified by preparative TLC developed by 6% MeOH in DCM to afford compound 129 (97 mg, 77%) as a yellow solid. HPLC purity 98.8% (t_{R}=15.54 min). ¹H NMR (300 MHz, CDCl₃) δ 8.30 (s, 1H), 7.86 (d, J=8.7 Hz, 1H), 7.54 (d, J=2.4 Hz, 1H), 7.36 (dd, J=9.0, 2.4 Hz, 1H), 6.95 (d, J=9.0 Hz, 1H), 6.66 (d, J=2.4 Hz, 1H), 6.55 (dd, J=8.7, 2.4 Hz, 1H), 3.73 (t, J=4.5 Hz, 4H), 3.15 (t, J=6.0 Hz, 2H), 2.64 (t, J=6.0 Hz, 2H), 2.49 (t, J=4.5 Hz, 4H). ¹³C NMR (75 MHz, CDCl₃) δ 166.8, 160.0, 147.0, 134.4, 127.0, 125.6, 125.2, 123.8, 122.9, 120.3, 116.0, 112.6, 112.1, 66.9 (2C), 56.9, 53.3 (2C), 39.8. HRMS (ESI) calcd for C₁₉H₂₂Cl₂N₃O₃, 410.1038 (M+H)⁺; found, 410.1028.

N-(2-Chloro-4-(cyclopentylamino)phenyl)-2-hydroxybenzamide (135). To a solution of 2-chloro-4-fluoronitrobenzene (760 mg, 4.3 mmol) and cyclopentylamine (442 mg, 5.2 mmol) in 10 mL of DMF was added K₂CO₃ (1.2 g, 8.6 mmol). The resulting mixture was stirred at 100° C. for 1 h. Then the mixture was cooled to RT and poured into 50 mL of H₂O. The mixture was extracted with EtOAc (2×120 mL), washed with H₂O (3×30 mL) and brine (30 mL), dried (Na₂SO₄) and concentrated. The residue was purified by column chromatography (Hex/EtOAc=6/1 to 3/1) to afford 3-chloro-N-cyclopentyl-4-nitroaniline 131a (1.01 g, 96%) as yellow oil. ¹H NMR (300 MHz, CDCl₃) δ 7.97 (d, J=9.3 Hz, 1H), 6.57 (d, J=2.7 Hz, 1H), 6.42 (dd, J=9.13, 2.7 Hz, 1H), 4.59 (d, J=4.8 Hz, 1H), 3.87-3.75 (m, 1H), 2.15-1.97 (m, 2H), 1.82-1.57 (m, 4H), 1.58-1.41 (m, 2H). ¹³C NMR (75 MHz, CDCl₃) δ 152.2, 135.8, 130.9, 129.1, 113.9, 110.6, 54.5, 33.4 (2C), 24.0 (2C).

To a solution of compound 131a (1.01 g, 4.2 mmol) in 20 mL of MeOH was added 10 mL of saturated NH₄Cl (aq.). Zinc dust (1.36 g, 21.0 mmol) was added into the solution at 0° C. The reaction was stirred at RT for 16 h. TLC indicated that the starting material was gone. 200 mL of EtOAc was added to the solution. The Zinc solid was filtered, and the filtrate was washed with 30 mL of brine, dried (Na₂SO₄) and concentrated under vacuum. The residue was purified by column chromatography to afford 3-chloro-N¹-cyclopentylbenzene-1,4-diamine 132a (800 mg, 90%) as brown oil. ¹H NMR (300 MHz, Chloroform-d) δ 6.64 (d, J=8.7 Hz, 1H), 6.58 (d, J=2.7 Hz, 1H), 6.41 (dd, J=8.7, 2.7 Hz, 1H), 3.75-3.46 (m, 3H), 3.30 (s, 1H), 2.07-1.89 (m, 2H), 1.79-1.51 (m, 4H), 1.51-1.33 (m, 2H). ¹³C NMR (75 MHz, CDCl₃) δ 141.8, 134.0, 120.8, 117.7, 114.3, 114.2, 55.6, 33.6 (C), 24.2 (2C).

To a solution of 2-acetoxybenzoic acid (163 mg, 0.90 mmol) and DIEA (85 mg, 0.66 mmol) in 20 mL of DCM was added HBTU (686 mg, 1.80 mmol) at 0° C. The resulting mixture was stirred at 0° C. for 30 min, then compound 132a (153 mg, 0.73 mmol) was added. The mixture was stirred at RT for 2 h, and then concentrated. The residue was purified by column chromatography (Hex/EtOAc=6/1 to 3/1) to afford the intermediate. The intermediate was dissolved in 10 mL of MeOH, and NaOH (145 mg, 3.62 mmol) in 8 mL of H₂O was added at 0° C. The resulting mixture was stirred at RT for 1 h. Then the PH of mixture was adjusted to 6-7 with 2M HCl (aq.). The mixture was extracted with EA (2×80 mL), dried (Na₂SO₄), and concentrated. The residue was purified by column chromatography (Hex/EtOAc=8/1 to 5/1) to afford compound 63 (85 mg, 35% in two steps) as a grey solid. HPLC purity 99.7% (t_{R}=19.01 min). ¹H NMR (300 MHz, CDCl₃) δ 12.07 (s, 1H), 8.20 (s, 1H), 7.94 (d, J=9.0 Hz, 1H), 7.54 (dd, J=8.1, 1.5 Hz, 1H), 7.48-7.39 (m, 1H), 7.05-6.99 (m, 1H), 6.96-6.89 (m, 1H), 6.63 (d, J=2.4 Hz, 1H), 6.53 (dd, J=9.0, 2.7 Hz, 1H), 3.86-3.66 (m, 2H), 2.10-1.95 (m, 2H), 1.80-1.55 (m, 4H), 1.52-1.38 (m, 2H). ¹³C NMR (75 MHz, CDCl₃) δ 167.9, 161.8, 146.4, 134.6, 126.4, 125.5, 124.7, 122.9, 119.1, 118.9, 114.8, 112.7, 112.5, 54.8, 33.5 (2C), 24.1 (2C). HRMS (ESI) calcd for C₁₈H₂₀ClN₂O₂, 331.1213 (M+H)⁺; found, 331.1204.

N-(2-Chloro-4-(cyclopentylamino)phenyl)-2-hydroxy-5-methylbenzamide (136). To a solution of compound 132a (127 mg, 0.60 mmol) and 2-methoxy-5-methylbenzoic acid (100 mg, 0.60 mmol) in 20 mL of DCM was added DIPEA (156 mg, 1.20 mmol) and HBTU (388 mg, 1.20 mmol) successively at 0° C. The resulting mixture was stirred at RT overnight, and then concentrated. The residue was purified by column chromatography (Hex/EtOAc=5/1) to afford the intermediate N-(2-chloro-4-(cyclopentylamino)phenyl)-2-methoxy-5-methylbenzamide (190 mg, 88%) as yellow oil. ¹H NMR (300 MHz, CDCl₃) δ 10.32 (s, 1H), 8.38 (d, J=8.7 Hz, 1H), 8.09 (d, J=2.1 Hz, 1H), 7.23 (dd, J=8.1, 2.1 Hz, 1H), 6.87 (d, J=8.4 Hz, 1H), 6.63 (d, J=2.7 Hz, 1H), 6.53 (dd, J=9.0, 2.7 Hz, 1H), 3.98 (s, 3H), 3.78-3.67 (m, 2H), 2.33 (s, 3H), 2.06-1.93 (m, 2H), 1.77-1.53 (m, 4H), 1.50-1.37 (m, 2H). ¹³C NMR (75 MHz, CDCl₃) δ 162.8, 155.3, 145.1, 133.5, 132.5, 130.7, 125.8, 124.3, 123.4, 121.3, 112.8, 112.5, 111.4, 56.2, 54.8, 33.4 (2C), 24.1 (2C), 20.4.

To a solution of the intermediate N-(2-chloro-4-(cyclopentylamino)phenyl)-2-methoxy-5-methylbenzamide (190 mg, 0.51 mmol) in DCM (20 mL) was added BBr₃ (2.5 mL, 2.5 mmol, 1 M in DCM) dropwise at 0° C. The mixture was stirred at RT for 2 h. The mixture was diluted with DCM, washed with H₂O and brine, dried (Na₂SO₄) and concentrated. The residue was purified by preparative TLC to afford compound 136 (170 mg, 96%) as a yellow solid. HPLC purity 96.4% (t_{R}=19.65 min). ¹H NMR (300 MHz, CDCl₃) δ 11.83 (s, 1H), 8.14 (s, 1H), 7.94 (d, J=8.7 Hz, 1H), 7.29 (s, 1H), 7.28-7.22 (m, 1H), 6.93 (d, J=8.4 Hz, 1H), 6.65 (d, J=2.4 Hz, 1H), 6.54 (dd, J=9.0, 2.7 Hz, 1H), 3.82-3.66 (m, 2H), 2.34 (s, 3H), 2.10-1.96 (m, 2H), 1.80-1.59 (m, 4H), 1.53-1.40 (m, 2H). ¹³C NMR (75 MHz, CD₃OD+CDCl₃) δ 167.1, 156.5, 147.3, 134.9, 129.6, 129.3, 127.9, 126.1, 124.2, 117.4, 117.2, 113.4, 112.7, 55.1, 33.5 (2C), 24.4 (2C), 20.6. HRMS (ESI) calcd for C₁₉H₂₂ClN₂O₂, 345.1370 (M+H)⁺; found, 345.1364.

3-Chloro-N-(2-chloro-4-(cyclopentylamino)phenyl)benzamide (137). To a solution of compound 132a (97 mg, 0.43 mmol), 3-chlorobenzoic acid (67 mg, 0.43 mmol) in 20 mL of DCM was added DIEA (111 mg, 0.86 mmol) and HBTU (326 mg, 0.86 mmol) successively at 0° C. The resulting mixture was stirred at RT overnight. The mixture was concentrated and purified by column chromatography (Hex/EtOAc=6/1 to 3/1) to give compound 137 (145 mg, 96%) as a purple solid. HPLC purity 99.2% (t_{R}=19.58 min). ¹H NMR (300 MHz, CDCl₃) δ 8.09-7.99 (m, 2H), 7.87 (t, J=1.5 Hz, 1H), 7.73 (d, J=7.5 Hz, 1H), 7.53-7.47 (m, 1H), 7.40 (t, J=7.8 Hz, 1H), 6.62 (d, J=2.7 Hz, 1H), 6.51 (dd, J=9.0, 2.4 Hz, 1H), 4.02 (s, 1H), 3.79-3.66 (m, 1H), 2.09-1.93 (m, 2H), 1.78-1.54 (m, 4H), 1.52-1.35 (m, 2H). ¹³C NMR (75 MHz, CDCl₃) δ 163.9, 146.1, 136.8, 135.1, 131.9, 130.1, 127.6, 125.7, 125.0, 124.1, 123.9, 112.7, 112.6, 54.8, 33.5 (2C), 24.1 (2C). HRMS (ESI) calcd for C₁₈H₁₉Cl₂N₂O, 349.0874 (M+H)⁺; found, 349.0874.

5-Chloro-N-(2-chloro-4-(cyclopentylamino)phenyl)-2-methoxybenzamide (138). Compound 138 was prepared by a procedure similar to that used to prepare compound 137, starting from compound 132a and 5-chloro-2-methoxybenzoic acid. The title compound (80 mg, 89%) was obtained as a yellow solid. HPLC purity 98.1% (t_{R}=21.77 min). ¹H NMR (300 MHz, CDCl3) δ 10.21 (s, 1H), 8.33 (d, J=9.0 Hz, 1H), 8.25 (d, J=2.7 Hz, 1H), 7.38 (dd, J=8.7, 2.7 Hz, 1H), 6.93 (d, J=8.7 Hz, 1H), 6.62 (d, J=2.4 Hz, 1H), 6.52 (dd, J=9.0, 2.7 Hz, 1H), 4.02 (s, 3H), 3.79-3.62 (m, 2H), 2.10-1.93 (m, 2H), 1.78-1.54 (m, 4H), 1.50-1.37 (m, 2H). ¹³C NMR (75 MHz, CDCl3) δ 161.3, 155.8, 145.4, 132.6, 132.1, 126.9, 125.5, 124.5, 123.5, 123.3, 113.0, 112.8, 112.6, 56.7, 54.9, 33.6 (2C), 24.1 (2C). HRMS (ESI) calcd for C₁₉H₂₁Cl₂N₂O₂, 379.0980 (M+H)⁺; found, 379.0970.

5-Chloro-N-(2-chloro-4-(cyclopentylamino)phenyl)-2-((1-methylpiperidin-4-yl)oxy)benzamide (139). To a solution of compound 132a (70 mg, 0.33 mmol), 5-chloro-2-((1-methylpiperidin-4-yl)oxy)benzoic acid (90 mg, 0.33 mmol) in 20 mL of DCM was added DIEA (85 mg, 0.66 mmol) and HBTU (250 mg, 0.66 mmol) successively at 0° C. The resulting mixture was stirred at RT overnight and then concentrated. The residue was purified by column chromatography (DCM/MeOH=10:1 to DCM/MeOH (7 M NH₃ in MeOH)=10:1) to afford compound 139 (150 mg, 98%) as a yellow solid. HPLC purity 99.4% (t_{R}=15.54 min). ¹H NMR (300 MHz, CDCl₃) δ 9.74 (s, 1H), 8.20 (d, J=2.7 Hz, 1H), 8.06 (d, J=9.0 Hz, 1H), 7.33 (d, J=8.7, 2.7 Hz, 1H), 6.94 (d, J=9.0 Hz, 1H), 6.59 (d, J=2.7 Hz, 1H), 6.50 (dd, J=9.0, 2.7 Hz, 1H), 4.50-4.37 (m, 1H), 3.82-3.60 (m, 2H), 2.79-2.66 (m, 2H), 2.23 (s, 3H), 2.21-1.88 (m, 8H), 1.76-1.52 (m, 4H), 1.50-1.34 (m, 2H). ¹³C NMR (75 MHz, CDCl₃) δ 161.9, 153.9, 145.8, 132.4, 132.2, 126.8, 125.6, 124.8, 124.6, 124.5, 115.7, 112.7, 112.4, 76.1, 54.7, 53.2 (2C), 46.0, 33.46 (2C), 31.2 (2C), 24.06 (2C). HRMS (ESI) calcd for C₂₄H₃₀Cl₂N₃O₂, 462.1715 (M+H)⁺; found, 462.1708.

5-Chloro-N-(2-chloro-4-(cyclopentylamino)phenyl)-2-(methylsulfonamido)benzamide (140). Compound 140 was prepared by a procedure similar to that used to prepare compound 137, starting from compound 132a and 5-chloro-2-(methylsulfonamido)benzoic acid. The title compound (90 mg, 85%) was obtained as a pale yellow solid. HPLC purity 99.4% (t_{R}=19.42 min). ¹H NMR (300 MHz, CDCl₃) δ 10.25 (s, 1H), 7.98 (s, 1H), 7.88 (d, J=8.7 Hz, 1H), 7.76 (d, J=8.7 Hz, 1H), 7.64 (d, J=2.4 Hz, 1H), 7.50 (dd, J=8.7, 2.4 Hz, 1H), 6.65 (d, J=2.7 Hz, 1H), 6.54 (dd, J=8.7, 2.7 Hz, 1H), 3.84-3.68 (m, 2H), 3.03 (s, 3H), 2.1-1.96 (m, 2H), 1.80-1.58 (m, 4H), 1.53-1.39 (m, 2H). ¹³C NMR (75 MHz, CDCl₃) δ 165.2, 147.0, 138.0, 133.1, 129.1, 127.1, 127.1, 125.1, 122.5, 122.3, 121.8, 112.6, 112.3, 54.7, 40.1, 33.5 (2C), 24.1 (2C). HRMS (ESI) calcd for C₁₉H₂₂Cl₂N₃O₃S, 442.0759 (M+H)⁺; found, 442.0751.

The amino intermediates 134a-g were prepared by a procedure similar to that used to prepare compound 76, starting from known nitro derivatives 133a-g.

N-(4-Aminophenyl)-5-chloro-2-hydroxybenzamide 134a. 165 mg, 57%. Yellow solid. ¹H NMR (300 MHz, CDCl3) δ 12.06 (s, 1H), 7.71 (s, 1H), 7.46 (d, J=2.1 Hz, 1H), 7.37 (dd, J=9.0, 2.4 Hz, 1H), 7.31 (d, J=8.7 Hz, 2H), 6.97 (d, J=9.0 Hz, 1H), 6.71 (d, J=8.7 Hz, 2H), 3.71 (s, 2H).

N-(4-Amino-2-fluorophenyl)-5-chloro-2-hydroxybenzamide 134b. 67 mg, 82%. Yellow solid. ¹H NMR (300 MHz, CDCl₃) δ 11.90 (s, 1H), 7.85-7.74 (m, 2H), 7.48 (d, J=2.4 Hz, 1H), 7.39 (dd, J=9.0, 2.4 Hz, 1H), 6.98 (d, J=8.7 Hz, 1H), 6.54-6.45 (m, 2H), 3.80 (s, 2H).

N-(4-Amino-3-chlorophenyl)-5-chloro-2-hydroxybenzamide 134c. 63 mg, 78%. Yellow solid. ¹H NMR (300 MHz, CDCl₃) δ 11.91 (s, 1H), 7.69 (s, 1H), 7.56 (d, J=2.4 Hz, 1H), 7.45 (d, J=2.4 Hz, 1H), 7.38 (dd, J=9.0, 2.4 Hz, 1H), 7.20 (dd, J=8.7, 2.4 Hz, 1H), 6.98 (d, J=9.0 Hz, 1H), 6.79 (d, J=8.7 Hz, 1H), 4.08 (s, 2H).

N-(5-Amino-2-fluorophenyl)-5-chloro-2-hydroxybenzamide 134d. 66 mg, 81%. Yellow solid. ¹H NMR (300 MHz, CDCl₃) δ 11.72 (s, 1H), 8.03 (s, 1H), 7.70 (dd, J=6.6, 2.7 Hz, 1H), 7.48 (d, J=2.4 Hz, 1H), 7.41 (dd, J=8.7, 2.4 Hz, 1H), 7.03-6.91 (m, 2H), 6.46-6.39 (m, 1H), 3.66 (s, 2H).

N-(4-Amino-2-chlorobenzyl)-5-chloro-2-hydroxybenzamide 134e. 90 mg, 76%. Yellow solid. ¹H NMR (300 MHz, CDCl₃) δ 12.20 (br s, 1H), 7.33-7.27 (m, 2H), 7.16 (d, J=8.1 Hz, 1H), 6.94-6.86 (m, 1H), 6.74-6.64 (m, 2H), 6.51 (dd, J=8.1, 2.4 Hz, 1H), 4.56 (d, J=5.7 Hz, 2H), 3.79 (s, 2H).

N-(4-Amino-2-chlorophenyl)-2-(5-chloro-2-hydroxyphenyl)acetamide 134f. 180 mg, 99%. Yellow solid. ¹H NMR (300 MHz, CD₃OD) δ 7.40 (dd, J=8.7, 2.7 Hz, 1H), 7.26-7.19 (m, 1H), 7.13-7.03 (m, 1H), 6.81 (dd, J=8.7, 2.7 Hz, 1H), 6.76-6.69 (m, 1H), 6.61-6.53 (m, 1H), 3.70-3.63 (m, 2H).

(S)—N-(1-((4-Amino-2-chlorophenyl)amino)-3-methyl-1-oxobutan-2-yl)-5-chloro-2-hydroxybenzamide 134g. 127 mg, 80%. Yellow solid. ¹H NMR (300 MHz, CD₃OD) δ 7.94 (d, J=2.4 Hz, 1H), 7.36 (dd, J=9.0, 2.4 Hz, 1H), 7.21 (d, J=8.7 Hz, 1H), 6.93 (d, J=8.7 Hz, 1H), 6.76 (d, J=2.4 Hz, 1H), 6.60 (dd, J=8.7, 2.4 Hz, 1H), 4.64 (d, J=6.6 Hz, 1H), 2.39-2.25 (m, 1H), 1.15-1.05 (m, 6H).

Compounds 141-147 were prepared according to general procedure B, starting from the amino intermediates 132a-g and cyclopentanone.

5-Chloro-N-(4-(cyclopentylamino)phenyl)-2-hydroxybenzamide (141). 65 mg, 57%. Yellow solid. HPLC purity 99.2% (t_{R}=20.29 min). ¹H NMR (300 MHz, CDCl3) δ 12.12 (s, 1H), 7.84 (s, 1H), 7.47 (d, J=2.1 Hz, 1H), 7.34 (dd, J=9.0, 2.1 Hz, 1H), 7.26 (d, J=8.7 Hz, 2H), 6.94 (d, J=8.7 Hz, 1H), 6.57 (d, J=8.7 Hz, 2H), 3.84-3.50 (m, 2H), 2.10-1.91 (m, 2H), 1.80-1.56 (m, 4H), 1.54-1.37 (m, 2H). ¹³C NMR (75 MHz, CDCl3) δ 167.3, 160.3, 146.4, 134.2, 125.6, 125.3, 123.9 (2C), 123.5, 120.3, 115.9, 113.4 (2C), 54.9, 33.6 (2C), 24.2 (2C). HRMS (ESI) calcd for C₁₈H₂₀ClN₂O₂, 331.1213 (M+H)⁺; found, 331.1203.

5-Chloro-N-(4-(cyclopentylamino)-2-fluorophenyl)-2-hydroxybenzamide (142). 80 mg, 95%. Yellow solid. HPLC purity 95.7% (t_{R}=19.53 min). ¹H NMR (300 MHz, CDCl₃) δ 7.87 (s, 1H), 7.67 (t, J=8.7 Hz, 1H), 7.48 (d, J=1.8 Hz, 1H), 7.35 (dd, J=8.7, 1.8 Hz, 1H), 6.95 (d, J=9.0 Hz, 1H), 6.41-6.29 (m, 2H), 3.77-3.65 (m, 1H), 2.09-1.93 (m, 2H), 1.81-1.54 (m, 4H), 1.52-1.38 (m, 2H). ¹³C NMR (75 MHz, CDCl₃) δ 167.1, 160.2, 155.7 (d, J=241.6 Hz), 147.5 (d, J=10.5 Hz), 134.4, 125.4 (2C), 123.7, 120.3, 115.7, 113.1 (d, J=11.6 Hz), 109.0 (d, J=2.5 Hz), 99.6 (d, J=23.3 Hz), 54.8, 33.5 (2C), 24.1 (2C). HRMS (ESI) calcd for C₁₈H₁₉ClFN₂O₂, 349.1119 (M+H)⁺; found, 349.1111.

5-Chloro-N-(3-chloro-4-(cyclopentylamino)phenyl)-2-hydroxybenzamide (143). 78 mg, 91%. Yellow solid. HPLC purity 95.0% (t_{R}=21.36 min). ¹H NMR (300 MHz, CDCl₃) δ 11.98 (s, 1H), 7.76 (s, 1H), 7.51 (d, J=2.1 Hz, 1H), 7.46 (d, J=2.1 Hz, 1H), 7.37 (dd, J=8.7, 2.1 Hz, 1H), 7.22 (dd, J=8.7, 2.1 Hz, 1H), 6.96 (d, J=9.0 Hz, 1H), 6.67 (d, J=9.0 Hz, 1H), 4.28 (s, 1H), 3.80 (s, 1H), 2.11-1.98 (m, 2H), 1.84-1.72 (m, 2H), 1.72-1.62 (m, 2H), 1.58-1.46 (m, 2H). ¹³C NMR (75 MHz, CDCl₃) δ 167.3, 160.4, 142.3, 134.4, 125.2, 125.2, 123.7, 123.7, 122.2, 120.5, 118.9, 115.6, 111.7, 54.7, 33.6 (2C), 24.2 (2C). HRMS (ESI) calcd for C₁₈H₁₉Cl₂N₂O₂, 365.0824 (M+H)⁺; found, 365.0815.

5-Chloro-N-(5-(cyclopentylamino)-2-fluorophenyl)-2-hydroxybenzamide (144). 68 mg, 83%. Yellow solid. HPLC purity 97.7% (t_{R}=20.13 min). ¹H NMR (300 MHz, CDCl3) δ 8.11 (s, 1H), 7.56 (dd, J=6.6, 2.7 Hz, 1H), 7.49 (d, J=2.4 Hz, 1H), 7.38 (dd, J=9.0, 2.4 Hz, 1H), 7.00-6.89 (m, 2H), 6.35-6.27 (m, 1H), 3.80-3.66 (m, 1H), 2.10-1.96 (m, 2H), 1.81-1.57 (m, 4H), 1.52-1.38 (m, 2H). ¹³C NMR (75 MHz, CDCl₃) δ 166.9 (d, J=20.9 Hz), 160.2, 146.0 (d, J=231.8 Hz), 145.0 (d, J=1.7 Hz), 134.7, 125.4, 125.4 (d, J=10.1 Hz), 123.9, 120.4, 115.7 (d, J=24.4 Hz), 115.2, 109.2 (d, J=6.8 Hz), 106.9, 55.2, 33.6 (2C), 24.2 (2C). HRMS (ESI) calcd for C₁₈H₁₉ClFN₂O₂, 349.1119 (M+H)⁺; found, 349.1108.

5-Chloro-N-(2-chloro-4-(cyclopentylamino)benzyl)-2-hydroxybenzamide (145). 74 mg, 67%. Pale yellow solid. HPLC purity 99.2% (t_{R}=20.56 min). ¹H NMR (300 MHz, CDCl₃) δ 12.27 (s, 1H), 7.36-7.23 (m, 2H), 7.17 (d, J=8.4 Hz, 1H), 6.91 (d, J=9.3 Hz, 1H), 6.64-6.54 (m, 2H), 6.45 (dd, J=8.4, 2.4 Hz, 1H), 4.56 (d, J=5.4 Hz, 2H), 3.85-3.68 (m, 2H), 2.08-1.95 (m, 2H), 1.78-1.56 (m, 4H), 1.51-1.37 (m, 2H). ¹³C NMR (75 MHz, CDCl₃) δ 168.6, 160.2, 149.1, 134.9, 134.1, 131.8, 125.2, 123.4, 121.9, 120.1, 115.4, 113.3, 112.0, 54.6, 41.7, 33.5 (2C), 24.1 (2C). HRMS (ESI) calcd for C₁₉H₂₁Cl₂N₂O₂, 379.0980 (M+H)⁺; found, 379.0971.

2-(5-Chloro-2-hydroxyphenyl)-N-(2-chloro-4-(cyclopentylamino)phenyl)acetamide (146). 72 mg, 84%. Grey solid. HPLC purity 99.3% (t_{R}=17.47 min). ¹H NMR (300 MHz, CD₃OD+CDCl₃) δ 7.55 (d, J=8.7 Hz, 1H), 7.18 (s, 1H), 7.06 (d, J=8.4 Hz, 1H), 6.79 (d, J=8.7 Hz, 1H), 6.57 (s, 1H), 6.47 (d, J=8.7 Hz, 1H), 3.74-3.54 (m, 3H), 2.01-1.86 (m, 2H), 1.74-1.51 (m, 4H), 1.49-1.33 (m, 2H). ¹³C NMR (75 MHz, CD₃OD+CDCl₃) δ 171.5, 154.5, 147.6, 131.1, 129.0, 127.6, 126.0, 125.0, 124.1, 124.1, 117.2, 113.4, 112.8, 55.2, 39.6, 33.6 (2C), 24.5 (2C). HRMS (ESI) calcd for C₁₉H₂₁Cl₂N₂O₂, 379.0980 (M+H)⁺; found, 379.0970.

(S)-5-chloro-N-(1-((2-chloro-4-(cyclopentylamino)phenyl)amino)-3-methyl-1-oxobutan-2-yl)-2-hydroxybenzamide (147). 48 mg, 98%. Yellow solid. HPLC purity 99.4% (t_{R}=17.19 min). ¹H NMR (300 MHz, CDCl₃) δ 11.91 (s, 1H), 7.84-7.72 (m, 2H), 7.66-7.48 (m, 2H), 7.32-7.24 (m, 1H), 6.87 (d, J=9.0 Hz, 1H), 6.58 (d, J=2.1 Hz, 1H), 6.45 (dd, J=8.7, 2.1 Hz, 1H), 4.57 (t, J=7.5 Hz, 1H), 3.95-3.58 (m, 2H), 2.40-2.25 (m, 1H), 2.07-1.93 (m, 2H), 1.77-1.56 (m, 4H), 1.49-1.36 (m, 2H), 1.10 (d, J=6.6 Hz, 6H). ¹³C NMR (75 MHz, CDCl3) δ 169.4, 168.7, 159.6, 146.5, 134.3, 126.3, 126.2, 124.8, 123.8, 123.1, 119.9, 115.5, 112.8, 112.4, 59.7, 54.8, 33.5 (2C), 31.6, 24.1 (2C), 19.4, 18.7. HRMS (ESI) calcd for C₂₃H₂₈Cl₂N₃O₃, 464.1508 (M+H)⁺; found, 464.1499.

Plaque Assay. Compounds were tested using low MOI infections (0.06 vp/cell) and at concentrations of 10 μM and in a dose-response assay ranging from 10 to 0.375 μM in a plaque assay. Briefly, 293β5 cells were seeded in 6-well plates at a density of 4×10⁵ cells per well in duplicate for each condition. When cells reached 80-90% confluency, they were infected with HAdV5-GFP (0.06 vp/cell) and rocked for 2 h at 37° C. After the incubation the inoculum was removed, and the cells were washed once with PBS. The cells were then carefully overlaid with 4 mL/well of equal parts of 1.6% (water/vol) Difco Agar Noble (Becton, Dickinson & Co., Sparks, Md.) and 2×EMEM (Minimum Essential Medium Eagle, BioWhittaker) supplemented with 2×penicillin/streptomycin, 2×L-glutamine, and 10% FBS. The mixture also contained the drugs in concentrations ranging from 10 to 0.375 μM. Following incubation for 7 days at 37° C., plates were scanned with a Typhoon FLA 9000 imager (GE Healthcare Life Sciences) and plaques were quantified with ImageJ (Schneider et al., Nat. Methods. 2012, 9, 671-675).

Entry Assay. The anti-HAdV activity was measured in an entry assay using human A549 epithelial cells (3×10⁵ cells/well in coming black wall, clear bottom 96-well plates) infected with HAdV5-GFP (2000 vp/cell) in the presence 50 μM of the candidates and in a dose-response assay. A standard infection curve was generated in parallel by infecting cells in the absence of compounds using serial 2-fold dilutions of virus. All reactions were done in triplicate. Cells, virus, and drugs were incubated for 48 h at 37° C. and 5% CO₂. Infection, as measured by HAdV5-mediated GFP expression, was analyzed using a Typhoon 9410 imager (GE Healthcare Life Sciences) and quantified with ImageQuantTL (GE Healthcare Life Sciences).

Cytotoxicity Assay. The cytotoxicity of the compounds was analyzed by commercial kit AlamarBlue® (Invitrogen, Ref. DAL1025). A549 cells at a density of 5×10³ cells per well in 96-well plates were seeded. Decreasing concentrations of each derivative (200 μM, 150 μM, 100 μM, 80 μM, 60 μM, 40 μM, 30 μM, 20 μM, 10 μM, 5 μM, 2.5 μM, 0 μM) were diluted in 100 μL of Dulbecco's Modified Eagle Medium (DMEM). Cells were then incubated at 37° C. for 48 h following the kit protocol. The cytotoxic concentration 50 (CC₅₀) value was obtained using the statistical package GraphPad Prism. This assay was performed in duplicate.

Virus Yield Reduction. A549 cells (1.5×10⁵ cells/well in a 24-well plate) were incubated 24 h in 500 μL of complete DMEM and they were infected with wild-type HAdV5 (100 vp/cell) when more than 90% of confluency were observed. Infected cells were incubated 48 h at 37° C. in 500 μL of complete DMEM containing 10-fold IC₅₀ concentration obtained in the plaque assay of the compounds or the same volume of DMSO (positive control). After 48 h, cells were harvested and subjected to three rounds of freeze/thaw. Serial dilutions of clarified lysates were titrated on A549 cells (3×10⁴ cells/well), and TCID₅₀ values were calculated using an end-point dilution method (Reed and Muench, 1938).

Time of Addition Assay. The anti-HAdV effect of derivatives at different points of time was measured in a time-curve assay using 293β5 cells (3×10⁵ cells/well in coming black wall, clear bottom 96-well plates) infected with HAdV5-GFP (2.000 vp/cell) in the presence of 10-fold the IC₅₀ concentration obtained in the plaque assay for each compound or the same volume of DMSO (positive control). Parallel samples of HAdV-5 were incubated with or without the selected derivatives on ice for 1 h. Virus was then added to 293β5 cells and incubated at 37° C. The derivatives were added at the indicated time points before or during this incubation. After a total of 2 h at 37° C., cells were incubated for an additional 48 h at 37° C. and 5% CO₂ before being analyzed for GFP expression using the Typhoon 9410 imager (GE Healthcare Life Sciences) as above.

Nuclear-Associated HAdV Genomes. The nuclear delivery of HAdV genomes was assessed by real-time PCR following nuclear isolation from infected cells. 1×10⁶ A549 cells in 6-well plates were infected with wild-type HAdV5 at MOI 2,000 vp/cell in the presence of 10-fold IC₅₀ concentration obtained in the plaque assay of the compounds, or the same volume of DMSO for positive control. Forty-five minutes after infection, A549 cells were trypsinized and collected and then washed twice with PBS. Then, cytoplasmic and nuclear fractions were separated using a hypotonic buffer solution and NP-40 detergent. The cell pellet was resuspended in 500 μL of 1×hypotonic buffer (20 mM Tris-HCl pH 7.4, 10 mM NaCl, 3 mM MgCl₂) and incubated for 15 min at 4° C. Then, 25 μL of NP-40 was added and the samples were vortexed. The homogenates were centrifuged for 10 min at 835 g at 4° C. Following the removal of the cytoplasmic fraction (supernatant), HAdV DNA was isolated from the nuclear fraction (pellet) and from the cytoplasmic fraction using the E.Z.N.A.® Tissue DNA Kit (Omega Bio-tek, Norcross, Ga.).

HAdV DNA Quantification by Real-Time PCR. A549 cells (1.5×10⁵ cells/well in a 24-well plate) were incubated 24 h in 500 μL of complete DMEM and they were infected with wild-type HAdV5 (100 vp/cell) when more than 90% of confluency were observed. Infected cells were incubated 24 h at 37° C. in 500 μL of complete DMEM containing 10-fold IC₅₀ concentration obtained in the plaque assay of the compounds or the same volume of DMSO (positive control). All samples were done in duplicate. After 24 h of incubation at 37° C., DNA was purified from the cell lysate with the E.Z.N.A.® Tissue DNA Kit (Omega Bio-tek, Norcross, Ga.) following the manufacturer's instructions. TaqMan primers and probes for a common region of the HAdV5 were designed with the GenScript Real-Time PCR (TaqMan) Primer Design software (GenScript). Oligonucleotides sequences were: AQ1: 5′-GCCACGGTGGGGTTTCTAAACTT-3′ (SEQ ID NO: 1); AQ2: 5′-GCCCCAGTGGTCTTACATGCACAT-3′ (SEQ ID NO: 2); Probe: 6-FAM-5′-TGCACCAGACCCGGGCTCAGGTACTCCGA-3′-TAMRA (SEQ ID NO: 3). Real-time PCR mixtures consisted of 9.5 μL of the purified DNA, AQ1 and AQ2 at a concentration of 200 nM each and Probe at a concentration of 50 nM in a total volume of 25 μL. The PCR cycling protocol was 95° C. for 3 min followed by 40 cycles of 95° C. for 10 s and 60° C. for 30 s. Human glyceraldehyde-3-phosphate dehydrogenase (GAPDH) gene was used as internal control. Oligonucleotides sequences for GAPDH and conditions were those previously reported by Henke-Gendo et al. (2012).

For quantification, gene fragments from hexon, and GAPDH were cloned into the pGEM-T Easy vector (Promega) and known concentrations of template were used to generate a standard curve in parallel for each experiment. All assays were performed in thermal cycler LightCycler® 96 System (Roche).

HCMV DNA Quantification by Real-Time PCR. To test the anti-HCMV activity of these anthelmintic drugs, HFF cells (ATCC® SCRC-1041™) were seeded in a 24-well plate (1×10⁶ cells/plate), infected with HCMV (MOI of 0.05 vp/cell) and incubated in complete DMEM in the presence of 10-fold IC₅₀ concentration obtained in the plaque assay of the compounds or the same volume of DMSO in triplicate. Then, cells were incubated for 72 h at 37° C. and 5% CO₂ and HCMV DNA was purified from the cell lysate using the QIAamp DNA Mini Kit (Qiagen, Valencia, Calif.) following the manufacturer's instructions. Real-time PCR primers, mixtures and protocols were the same as previously reported (Sanchez-Cespedes et al., J. Med. Chem. 2016, 59, 5432-5448).

Maximum tolerated dose (MTD). Three female Syrian hamsters per group (weight 80-100 gr) were immunosuppressed with a dose of 140 mg/kg of cyclophosphamide 7 days before beginning with the treatments and then twice weekly at a dose of 100 mg/kg. Before dosing, compounds were diluted in 10% DMSO, 10% Cremophor EL and 80% sodium chloride physiological solution. Hamsters were inoculated with 1 ml volume of appropriate suspension for the selected doses for compounds 74, 75, 87 and niclosamide along 14 days. At day 14 animals were sacrificed with a lethal dose of thiopental. All inoculations were done via intraperitoneal.

Statistical Analyses. One-way ANOVA tests (Dunnet method) were carried out using the GraphPad Prism 6. We considered a statistical significance with a P value under 0.05. This statistical significance was pointed out with asterisk in graphs, and the numbers of them indicate the level of significance (*p≤0.05, **p≤0.01, ***p≤0.001, ****p≤0.0001).

### Example 3

### 5-chloro-N-(cyclohexylmethyl)-2-hydroxybenzamide (JMX0493), a Potent Inhibitor of Adenovirus Scape from the Endosome

All commercially available starting materials and solvents were reagent grade and used without further purification. Reactions were performed under a nitrogen atmosphere in dry glassware with magnetic stirring. Preparative column chromatography was performed using silica gel 60, particle size 0.063-0.200 mm (70-230 mesh, flash). Analytical TLC was carried out employing silica gel 60 F254 plates (Merck, Darmstadt). Visualization of the developed chromatograms was performed with detection by UV (254 nm). NMR spectra were recorded on a Brucker-300 (¹H, 600 and 300 MHz; ¹³C, 150 and 75 MHz) spectrometer. ¹H and ¹³C NMR spectra were recorded with TMS as an internal reference. Chemical shifts were expressed in ppm, and J values were given in Hz. High-resolution mass spectra (HRMS) were obtained from Thermo Fisher LTQ Orbitrap Elite mass spectrometer. Parameters include the following: Nano ESI spray voltage was 1.8 kV; Capillary temperature was 275° C. and the resolution was 60,000; Ionization was achieved by positive mode. Melting points were measured on a Thermo Scientific Electrothermal Digital Melting Point Apparatus and uncorrected. Purities of final compounds were established by analytical HPLC, which was carried out on a Shimadzu HPLC system (model: CBM-20A LC-20AD SPD-20A UV/VIS). HPLC analysis conditions: Waters μBondapak C18 (300×3.9 mm); flow rate 0.5 mL/min; UV detection at 270 and 254 nm; linear gradient from 10% acetonitrile in water to 100% acetonitrile in water in 20 min followed by 30 min of the last-named solvent (0.1% TFA was added into both acetonitrile and water). All biologically evaluated compounds are >95% pure.

General procedure A. Methyl 5-chloro-2-hydroxybenzoate (1.0 eq) was dissolved in methanol (10 mL/0.5 mmol) followed by addition of different amine (3.0 eq). The resulting mixture was stirred at r.t. ˜80° C. for 48˜96 h, and then concentrated. The residue was purified by preparative TLC to afford the final amide products.

5-Chloro-N-cyclopentyl-2-hydroxybenzamide (156). Compound 156 (116 mg, 90%) was prepared as a beige solid according to general procedure A (60° C., 48 h), starting from methyl 5-chloro-2-hydroxybenzoate and cyclopentylamine. HPLC purity 99.9% (t_{R}=18.07 min). ¹H NMR (300 MHz, CDCl₃) δ 12.34 (s, 1H), 7.37-7.26 (m, 2H), 6.90 (d, J=8.7 Hz, 1H), 6.39 (d, J=4.2 Hz, 1H), 4.45-4.27 (m, 1H), 2.21-1.98 (m, 2H), 1.83-1.45 (m, 6H). ¹³C NMR (75 MHz, CDCl₃) δ 168.6, 160.0, 133.9, 125.2, 123.3, 120.1, 115.5, 51.8, 33.1 (2C), 23.9 (2C). HRMS (ESI) calcd for C₁₂H₁₅ClNO₂, 240.0791 (M+H)⁺; found, 240.0785.

5-Chloro-N-cyclohexyl-2-hydroxybenzamide (157). Compound 157 (71 mg, 51%) was prepared as a beige solid according to general procedure A (60° C., 48 h), starting from methyl 5-chloro-2-hydroxybenzoate and cyclohexylamine. HPLC purity 99.9% (t_{R}=18.85 min). ¹H NMR (300 MHz, CDCl₃) δ 12.36 (s, 1H), 7.35-7.27 (m, 2H), 6.90 (d, J=8.4 Hz, 1H), 6.21 (d, J=5.4 Hz, 1H), 4.00-3.86 (m, 1H), 2.06-1.94 (m, 2H), 1.83-1.60 (m, 3H), 1.48-1.13 (m, 5H). ¹³C NMR (75 MHz, CDCl₃) δ 168.1, 160.2, 134.0, 125.1, 123.3, 120.2, 115.6, 49.0, 33.0 (2C), 25.5, 25.0 (2C). HRMS (ESI) calcd for C₁₃H₁₇ClNO₂, 254.0948 (M+H)⁺; found, 254.0941.

5-Chloro-N-cycloheptyl-2-hydroxybenzamide (158). Compound 158 (75 mg, 52%) was prepared as an off-white solid according to general procedure A (60° C., 48 h), starting from methyl 5-chloro-2-hydroxybenzoate and cycloheptylamine. HPLC purity 99.5% (t_{R}=19.58 min). ¹H NMR (300 MHz, CDCl₃) δ 12.36 (s, 1H), 7.34-7.27 (m, 2H), 6.90 (d, J=9.0 Hz, 1H), 6.27 (d, J=5.7 Hz, 1H), 4.20-4.03 (m, 1H), 2.08-1.93 (m, 2H), 1.74-1.47 (m, 10H). ¹³C NMR (75 MHz, CDCl₃) δ 167.8, 160.2, 133.9, 125.1, 123.3, 120.2, 115.6, 51.2, 35.1 (2C), 28.0 (2C), 24.2 (2C). HRMS (ESI) calcd for C₁₄H₁₉ClNO₂, 268.1104 (M+H)⁺; found, 268.1097.

5-Chloro-2-hydroxy-N-(tetrahydro-2H-pyran-4-yl)benzamide (159). Compound 159 (30 mg, 22%) was prepared as a light-yellow solid according to general procedure A (80° C., 96 h), starting from methyl 5-chloro-2-hydroxybenzoate and 4-aminotetrahydropyran. HPLC purity 99.9% (t_{R}=15.48 min). ¹H NMR (300 MHz, CDCl₃) δ 12.18 (s, 1H), 7.38-7.29 (m, 2H), 6.98-6.88 (m, 1H), 6.21 (d, J=6.6 Hz, 1H), 4.26-4.12 (m, 1H), 4.07-3.96 (m, 2H), 3.59-3.46 (m, 2H), 2.05-1.91 (m, 2H), 1.69-1.52 (m, 2H). ¹³C NMR (75 MHz, CDCl₃) δ 168.4, 160.3, 134.3, 125.1, 123.4, 120.3, 115.3, 66.8 (2C), 46.5, 33.1 (2C). HRMS (ESI) calcd for C₁₂H₁₅ClNO₃, 256.0740 (M+H)⁺; found, 256.0734.

tert-Butyl 4-(5-chloro-2-hydroxybenzamido)piperidine-1-carboxylate (160). Compound 160 (24 mg, 13%) was prepared as an off-white solid according to general procedure A (80° C., 96 h), starting from methyl 5-chloro-2-hydroxybenzoate and 4-aminopiperidine-1-carboxylic acid tert-butyl ester. HPLC purity 98.8% (t_{R}=18.51 min). 11H NMR (300 MHz, CDCl₃) δ 12.21 (s, 1H), 7.43-7.23 (m, 2H), 6.96-6.88 (m, 1H), 6.44 (d, J=6.6 Hz, 1H), 4.20-4.02 (m, 3H), 2.97-2.80 (m, 2H), 2.05-1.94 (m, 2H), 1.52-1.40 (m, 11H). ¹³C NMR (75 MHz, CDCl₃) δ 168.5, 160.3, 154.9, 134.2, 125.3, 123.4, 120.3, 115.4, 80.1, 47.5, 42.9 (2C), 32.0 (2C), 28.6 (3C). HRMS (ESI) calcd for C₁₇H₂₄ClN₂O₄, 355.1425 (M+H)⁺; found, 355.1417.

5-Chloro-N-(cyclohexylmethyl)-2-hydroxybenzamide (161). To a solution of cyclohexanemethylamine (200 mg, 1.77 mmol), 5-chlorosalicylic acid (244 mg, 1.41 mmol) and DMAP (18 mg, 0.14 mmol) in 20 mL of DCM was added EDCI (509 mg, 2.66 mmol) at 0° C. The resulting mixture was stirred at r.t. for 24 h and then concentrated. The residue was purified by column chromatography (Hex/EtOAc=10/1) to afford compound 161 (240 mg, 63%) as a light-yellow solid. HPLC purity 99.9% (t_{R}=19.68 min). 1H NMR (300 MHz, CDCl₃) δ 12.22 (brs, 1H), 7.36-7.28 (m, 2H), 6.92 (d, J=8.4 Hz, 1H), 6.43 (s, 1H), 3.28 (t, J=6.3 Hz, 2H), 1.82-1.50 (m, 6H), 1.34-1.10 (m, 3H), 1.06-0.90 (m, 2H). ¹³C NMR (75 MHz, CDCl₃) δ 169.0, 160.2, 134.0, 125.0, 123.4, 120.2, 115.5, 46.1, 38.0, 31.0 (2C), 26.4, 25.9 (2C). HRMS (ESI) calcd for C₁₄H₁₉ClNO₂, 268.1104 (M+H)⁺; found, 268.1101.

(S)-5-chloro-N-(1-cyclohexylethyl)-2-hydroxybenzamide (162). Compound 162 was prepared by a procedure similar to that used to prepare compound 161 starting from 5-chlorosalicylic acid and (S)-1-cyclohexylethanamine. The title compound was obtained (138 mg, 39%) as an off-white solid. HPLC purity 99.6% (t_{R}=20.25 min). ¹H NMR (300 MHz, CDCl3) δ 12.36 (s, 1H), 7.33 (dd, J=8.7, 2.4 Hz, 1H), 7.29 (d, J=2.4 Hz, 1H), 6.93 (d, J=8.7 Hz, 1H), 6.03 (d, J=7.5 Hz, 1H), 4.12-3.98 (m, 1H), 1.85-1.61 (m, 5H), 1.50-1.38 (m, 1H), 1.32-0.95 (m, 8H). ¹³C NMR (75 MHz, CDCl₃) δ 168.3, 160.4, 134.0, 124.8, 123.3, 120.3, 115.6, 50.1, 43.2, 29.4, 29.2, 26.4, 26.2, 26.2, 18.0. HRMS (ESI) calcd for C₁₅H₂₁ClNO₂, 282.1261 (M+H)⁺; found, 282.1257.

5-Chloro-2-hydroxy-N-((tetrahydro-2H-pyran-4-yl)methyl)benzamide (163). Compound 163 (117 mg, 80%) was prepared as a light-yellow solid according to general procedure A (r.t., 48 h), starting from methyl 5-chloro-2-hydroxybenzoate and 4-(aminomethyl)tetrahydropyran. HPLC purity 99.8% (t_{R}=16.12 min). ¹H NMR (300 MHz, CDCl₃) δ 12.23 (s, 1H), 7.45 (d, J=2.4 Hz, 1H), 7.31 (dd, J=9.0, 2.4 Hz, 1H), 7.06 (s, 1H), 6.91 (d, J=9.0 Hz, 1H), 4.05-3.94 (m, 2H), 3.49-3.27 (m, 4H), 1.99-1.81 (m, 1H), 1.73-1.61 (m, 2H), 1.47-1.29 (m, 2H). ¹³C NMR (75 MHz, CDCl₃) δ 169.1, 159.8, 134.0, 125.5, 123.5, 120.0, 115.6, 67.6 (2C), 45.4, 35.2, 30.6 (2C). HRMS (ESI) calcd for C₁₃H₁₇ClNO₃, 270.0897 (M+H)⁺; found, 270.0890.

tert-Butyl 4-((5-chloro-2-hydroxybenzamido)methyl)piperidine-1-carboxylate (164). Compound 164 (74 mg, 37%) was prepared as an off-white solid according to general procedure A (r.t., 48 h), starting from methyl 5-chloro-2-hydroxybenzoate and 4-(aminomethyl)-1-N-Boc-piperidine. HPLC purity 99.8% (t_{R}=18.83 min). ¹H NMR (300 MHz, CDCl₃) δ 12.23 (s, 1H), 7.44 (d, J=2.1 Hz, 1H), 7.30 (dd, J=8.7, 2.1 Hz, 1H), 7.08-6.86 (m, 2H), 4.20-4.02 (m, 2H), 3.32 (s, 2H), 2.79-2.59 (m, 2H), 1.87-1.67 (m, 3H), 1.45 (s, 9H), 1.25-1.09 (m, 2H). ¹³C NMR (75 MHz, CDCl₃) δ 169.2, 160.1, 155.0, 134.1, 125.5, 123.5, 120.1, 115.6, 79.8, 45.2, 43.7 (2C), 36.4, 30.0 (2C), 28.6 (3C). HRMS (ESI) calcd for C₁₈H₂₆ClN₂O₄, 369.1581 (M+H)⁺; found, 369.1572.

5-Chloro-N-(2-cyclohexylethyl)-2-hydroxybenzamide (165). Compound 165 (69 mg, 46%) was prepared as an off-white solid according to general procedure A (r.t., 48 h), starting from methyl 5-chloro-2-hydroxybenzoate and 2-cyclohexylethylamine. HPLC purity 99.9% (t_{R}=20.07 min). ¹H NMR (300 MHz, CDCl3) δ 12.33 (s, 1H), 7.38-7.27 (m, 2H), 6.91 (d, J=8.7 Hz, 1H), 6.37 (s, 1H), 3.51-3.39 (m, 2H), 1.80-1.60 (m, 5H), 1.56-1.44 (m, 2H), 1.39-1.14 (m, 4H), 1.02-0.84 (m, 2H). ¹³C NMR (75 MHz, CDCl₃) δ 169.0, 160.1, 134.0, 125.1, 123.4, 120.2, 115.5, 37.9, 36.9, 35.5, 33.2 (2C), 26.5, 26.2 (2C). HRMS (ESI) calcd for C₁₅H₂₁ClNO₂, 282.1261 (M+H)⁺; found, 282.1253.

tert-Butyl 4-(2-(5-chloro-2-hydroxybenzamido)ethyl)piperazine-1-carboxylate (166). Compound 166 (88 mg, 43%) was prepared as an off-white solid according to general procedure A (r.t., 48 h), starting from methyl 5-chloro-2-hydroxybenzoate and 4-N-(2-aminoethyl)-1-N-Boc-piperazine. HPLC purity 98.4% (t_{R}=15.08 min). ¹H NMR (300 MHz, CDCl₃) δ 7.35-7.25 (m, 2H), 7.15 (s, 1H), 6.89 (d, J=8.7 Hz, 1H), 3.56-3.39 (m, 6H), 2.59 (t, J=6.0 Hz, 2H), 2.49-2.38 (m, 4H), 1.44 (s, 9H). ¹³C NMR (75 MHz, CDCl3) δ 168.9, 160.1, 154.8, 134.0, 125.4, 123.3, 120.1, 115.5, 80.0, 56.3, 52.8 (2C), 43.8 (2C), 36.1, 28.5 (3C). HRMS (ESI) calcd for C₁₈H₂₇ClN₃O₄, 384.1690 (M+H)⁺; found, 384.1680.

5-Chloro-2-hydroxy-N-(2-(4-methylpiperazin-1-yl)ethyl)benzamide (167). Compound 167 (62 mg, 37%) was prepared as a pale solid according to general procedure A (r.t., 48 h), starting from methyl 5-chloro-2-hydroxybenzoate and 2-(4-methylpiperazin-1-yl)ethylamine. HPLC purity 95.0% (t_{R}=11.25 min). ¹H NMR (300 MHz, CDCl₃) δ 9.45 (br s, 1H, 7.34 (d, J=2.4 Hz, 1H), 7.29 (dd, J=9.0, 2.7 Hz, 1H), 7.20 (s, 1H), 6.88 (d, J=9.0 Hz, 1H), 3.53-3.44 (m, 2H), 2.70-2.33 (m, 10H), 2.29 (s, 3H). ¹³C NMR (75 MHz, CDCl₃) δ 168.7, 160.0, 133.9, 125.6, 123.3, 120.0, 115.7, 56.0, 55.2 (2C), 52.8 (2C), 46.1, 36.0. HRMS (ESI) calcd for C₁₄H₂₁ClN₃O₂, 298.1322 (M+H)⁺; found, 298.1314.

5-Chloro-2-hydroxy-N-(2-morpholinoethyl)benzamide (168). Compound 168 (60 mg, 39%) was prepared as a grey solid according to general procedure A (r.t., 96 h), starting from methyl 5-chloro-2-hydroxybenzoate and 4-(2-aminoethyl)morpholine. HPLC purity 99.6% (t_{R}=11.66 min). ¹H NMR (300 MHz, CDCl₃) δ 7.35-7.28 (m, 2H), 7.03 (s, 1H), 6.94-6.88 (m, 1H), 3.74 (t, J=4.5 Hz, 4H), 3.51 (t, J=5.7 Hz, 2H), 2.61 (t, J=6.0 Hz, 2H), 2.51 (t, J=4.5 Hz, 4H). ¹³C NMR (75 MHz, CDCl₃) δ 168.9, 160.2, 134.1, 125.3, 123.3, 120.2, 115.4, 67.1 (2C), 56.5, 53.4 (2C), 35.8. HRMS (ESI) calcd for C₁₃H₁₈ClN₂O₃, 285.1006 (M+H)⁺; found, 285.0999.

5-Chloro-N-hexyl-2-hydroxybenzamide (169). Compound 169 (107 mg, 78%) was prepared as a white solid according to general procedure A (r.t., 48 h), starting from methyl 5-chloro-2-hydroxybenzoate and hexylamine. HPLC purity 98.0% (t_{R}=19.72 min). ¹H NMR (300 MHz, CDCl₃) δ 12.33 (s, 1H), 7.35 (d, J=2.4 Hz, 1H), 7.30 (dd, J=8.7, 2.4 Hz, 1H), 6.90 (d, J=8.7 Hz, 1H), 6.52 (s, 1H), 3.46-3.36 (s, 2H), 1.67-1.53 (m, 2H), 1.42-1.20 (m, 6H), 0.92-0.82 (m, 3H). ¹³C NMR (75 MHz, CDCl₃) δ 169.0, 160.0, 134.0, 125.2, 123.4, 120.1, 115.5, 40.1, 31.5, 29.4, 26.7, 22.6, 14.1. HRMS (ESI) calcd for C₁₃H₁₉ClNO₂, 256.1104 (M+H)⁺; found, 256.1098.

tert-Butyl (4-(5-chloro-2-hydroxybenzamido)butyl)carbamate (170). Compound 170 (56 mg, 30%) was prepared as a white solid according to general procedure A (r.t., 48 h), starting from methyl 5-chloro-2-hydroxybenzoate and N-Boc-1,4-butanediamine. HPLC purity 98.5% (t_{R}=17.75 min). ¹H NMR (300 MHz, CDCl₃) δ 12.53 (s, 1H), 7.63 (s, 1H), 7.53 (s, 1H), 7.29 (dd, J=8.7, 2.4 Hz, 1H), 6.90 (d, J=8.7 Hz, 1H), 4.77 (s, 1H), 3.48 (q, J=6.0 Hz, 2H), 3.16 (q, J=6.3 Hz, 2H), 1.72-1.53 (m, 4H), 1.45 (s, 9H). ¹³C NMR (75 MHz, CDCl₃) δ 169.3, 160.2, 156.9, 133.9, 125.9, 123.4, 119.9, 115.6, 79.9, 39.9, 39.8, 28.6, 28.6 (3C), 25.2. HRMS (ESI) calcd for C₁₆H₂₄ClN₂O₄, 343.1425 (M+H)⁺; found, 343.1417.

5-Chloro-2-hydroxyphenyl)(piperidin-1-yl)methanone (171). To a solution of 5-chloro-2-methoxybenzoic acid (210 mg, 1.13 mmol), piperidine (80 mg, 0.94 mmol) and DMAP (28 mg, 0.23 mmol) in DCM (20 mL) was added EDCI (433 mg, 2.26 mmol) at 0° C. The resulting mixture was stirred at r.t. for 12 h and concentrated. The residue was purified by column chromatography to afford the amide intermediate (5-chloro-2-methoxyphenyl)(piperidin-1-yl)methanone (230 mg, 96%) as colorless oil. ¹H NMR (300 MHz, CDCl₃) δ 7.25-7.09 (m, 2H), 6.77 (d, J=8.7 Hz, 1H), 3.74 (s, 3H), 3.69-3.52 (m, 2H), 3.19-3.03 (m, 2H), 1.69-1.31 (m, 6H). ¹³C NMR (75 MHz, CDCl₃) δ 165.9, 153.8, 129.7, 127.8, 127.5, 125.7, 112.2, 55.8, 47.9, 42.5, 26.3, 25.5, 24.5.

The amide intermediate (5-chloro-2-methoxyphenyl)(piperidin-1-yl)methanone (230 mg, 0.91 mmol) was dissolved in DCM (50 mL), and then BBr₃ (4.53 mL, 4.53 mmol, 1 M in DCM) was added at 0° C. The mixture was stirred at r.t. for 2 h. The mixture was diluted with DCM, washed with H₂O and brine, dried (Na₂SO₄) and concentrated. The residue was purified by preparative TLC to afford compound 171 (203 mg, 93%) as a white solid. HPLC purity 95.1% (t_{R}=15.13 min). ¹H NMR (300 MHz, CDCl₃) δ 9.57 (s, 1H), 7.26 (dd, J=8.7, 2.7 Hz, 1H), 7.19 (d, J=2.7 Hz, 1H), 6.93 (d, J=8.7 Hz, 1H), 3.67-3.60 (m, 4H), 1.77-1.61 (m, 6H). ¹³C NMR (75 MHz, CDCl₃) δ 169.4, 157.5, 132.3, 127.8, 123.4, 119.6, 118.9, 47.0 (2C), 26.2 (2C), 24.6. HRMS (ESI) calcd for C₁₂H₁₅ClNO₂, 240.0791 (M+H)⁺; found, 240.0786.

1-(4-(5-Chloro-2-hydroxybenzoyl)piperazin-1-yl)ethanone (172). Compound 172 was prepared by a procedure similar to that used to prepare compound 171 starting from 5-chlorosalicylic acid and 1-acetylpiperazine. The title compound was obtained (139 mg, 91% in two steps) as a yellow solid. HPLC purity 97.2% (t_{R}=12.32 min). ¹H NMR (300 MHz, CDCl3) δ 9.51 (s, 1H), 7.23-7.14 (m, 2H), 6.86 (d, J=8.7 Hz, 1H), 3.72-3.48 (m, 8H), 2.11 (s, 3H). ¹³C NMR (75 MHz, CDCl₃) δ 169.6, 168.9, 155.0, 132.0, 127.9, 124.2, 120.8, 118.8, 46.1, 45.3, 44.8, 41.5, 21.4. HRMS (ESI) calcd for C₁₃H₁₆ClN₂O₃, 283.0849 (M+H)⁺; found, 283.0843.

(5-Chloro-2-hydroxyphenyl)(4-methylpiperazin-1-yl)methanone (173). Compound 173 (39 mg, 28%) was prepared as a pale yellow solid solid according to general procedure A (60° C., 96 h), starting from methyl 5-chloro-2-hydroxybenzoate and 1-methylpiperazine. HPLC purity 98.3% (t_{R}=10.32 min). ¹H NMR (300 MHz, CDCl₃) δ 7.23 (dd, J=8.7, 2.7 Hz, 1H), 7.17 (d, J=2.7 Hz, 1H), 6.87 (d, J=8.7 Hz, 1H), 3.69 (t, J=5.1 Hz, 4H), 2.45 (t, J=5.1 Hz, 4H), 2.32 (s, 3H). ¹³C NMR (75 MHz, CDCl₃) δ 169.2, 156.7, 132.2, 127.8 (2C), 123.7, 119.4, 55.0 (2C), 46.0, 45.5 (2C). HRMS (ESI) calcd for C₁₂H₁₆ClN₂O₂, 255.0900 (M+H)⁺; found, 255.0895.

Plaque Assay. Compounds were tested using low MOI infections (0.06 vp/cell) and at concentrations of 10 μM and in a dose-response assay ranging from 10 to 0.3 μM in a plaque assay. Briefly, 293β5 cells were seeded in 6-well plates at a density of 4×10⁵ cells per well in duplicate for each condition. When cells reached 80-90% confluency, they were infected with HAdV5-GFP (0.06 vp/cell) and rocked for 2 h at 37° C. After the incubation the inoculum was removed, and the cells were washed once with PBS. The cells were then carefully overlaid with 4 mL/well of equal parts of 1.6% (water/vol) Difco Agar Noble (Becton, Dickinson & Co., Sparks, Md.) and 2×EMEM (Minimum Essential Medium Eagle, BioWhittaker) supplemented with 2×penicillin/streptomycin, 2×L-glutamine, and 10% FBS. The mixture also contained the compounds in concentrations ranging from 10 to 0.3 μM. Following incubation for 7 days at 37° C., plates were scanned with a Typhoon FLA 9000 imager (GE Healthcare Life Sciences) and plaques were quantified with ImageJ (Schneider et al., Nat. Methods. 2012, 9, 671-675).

Entry Assay. The anti-HAdV activity was measured in an entry assay using human A549 epithelial cells (3×10⁵ cells/well in coming black wall, clear bottom 96-well plates) infected with HAdV5-GFP (2000 vp/cell) in the presence 50 μM of the candidates and in a dose-response assay. A standard infection curve was generated in parallel by infecting cells in the absence of compounds using serial 2-fold dilutions of virus. All reactions were done in triplicate. Cells, virus, and drugs were incubated for 48 h at 37° C. and 5% CO₂. Infection, as measured by HAdV5-mediated GFP expression, was analyzed using a Typhoon 9410 imager (GE Healthcare Life Sciences) and quantified with ImageQuantTL (GE Healthcare Life Sciences).

Cytotoxicity Assay. The cytotoxicity of the compounds was analyzed by commercial kit AlamarBlue® (Invitrogen, Ref. DAL1025). A549 cells at a density of 5×10³ cells per well in 96-well plates were seeded. Decreasing concentrations of each derivative (200 μM, 150 μM, 100 μM, 80 μM, 60 μM, 40 μM, 30 μM, 20 μM, 10 μM, 5 μM, 2.5 μM, 0 μM) were diluted in 100 μL of Dulbecco's Modified Eagle Medium (DMEM). Cells were then incubated at 37° C. for 48 h following the kit protocol. The cytotoxic concentration 50 (CC₅₀) value was obtained using the statistical package GraphPad Prism. This assay was performed in duplicate.

Virus Yield Reduction. A549 cells (1.5×10⁵ cells/well in a 24-well plate) were incubated 24 h in 500 μL of complete DMEM and they were infected with wild-type HAdV5 (100 vp/cell) when more than 90% of confluency were observed. Infected cells were incubated 48 h at 37° C. in 500 μL of complete DMEM containing 10-fold IC₅₀ concentration obtained in the plaque assay of either compounds or the same volume of DMSO (positive control). After 48 h, cells were harvested and subjected to three rounds of freeze/thaw. Serial dilutions of clarified lysates were titrated on A549 cells (3×10⁴ cells/well), and TCID₅₀ values were calculated using an end-point dilution method (Reed and Muench, 1938).

Nuclear-Associated HAdV Genomes. The nuclear delivery of HAdV genomes was assessed by real-time PCR following nuclear isolation from infected cells. 1×10⁶ A549 cells in 6-well plates were infected with wild-type HAdV5 at MOI 2,000 vp/cell in the presence of 10-fold IC₅₀ concentration obtained in the plaque assay of the compounds, or the same volume of DMSO for positive control. Forty-five minutes after infection, A549 cells were trypsinized and collected and then washed twice with PBS. Then, cytoplasmic and nuclear fractions were separated using a hypotonic buffer solution and NP-40 detergent. The cell pellet was resuspended in 500 μL of 1×hypotonic buffer (20 mM Tris-HCl pH 7.4, 10 mM NaCl, 3 mM MgCl₂) and incubated for 15 min at 4° C. Then, 25 μL of NP-40 was added and the samples were vortexed. The homogenates were centrifuged for 10 min at 835 g at 4° C. Following the removal of the cytoplasmic fraction (supernatant), HAdV DNA was isolated from the nuclear fraction (pellet) and from the cytoplasmic fraction using the E.Z.N.A.® Tissue DNA Kit (Omega Bio-tek, Norcross, Ga.).

DNA Quantification by Real-Time PCR. A549 cells (1.5×10⁵ cells/well in a 24-well plate) were incubated 24 h in 500 μL of complete DMEM and they were infected with wild-type HAdV5 (100 vp/cell) when more than 90% of confluency were observed. Infected cells were incubated 24 h at 37° C. in 500 μL of complete DMEM containing 10-fold IC₅₀ concentration obtained in the plaque assay of either compounds or the same volume of DMSO (positive control). All samples were done in duplicate. After 24 h of incubation at 37° C., DNA was purified from the cell lysate with the E.Z.N.A.® Tissue DNA Kit (Omega Bio-tek, Norcross, Ga.) following the manufacturer's instructions. TaqMan primers and probes for a common region of the HAdV5 were designed with the GenScript Real-Time PCR (TaqMan) Primer Design software (GenScript). Oligonucleotides sequences were: AQ1: 5′-GCCACGGTGGGGTTTCTAAACTT-3′ (SEQ ID NO:1); AQ2: 5′-GCCCCAGTGGTCTTACATGCACAT-3′ (SEQ ID NO:2); Probe: 6-FAM-5′-TGCACCAGACCCGGGCTCAGGTACTCCGA-3′-TAMRA (SEQ ID NO:3). Real-time PCR mixtures consisted of 9.5 μL of the purified DNA, AQ1 and AQ2 at a concentration of 200 nM each and Probe at a concentration of 50 nM in a total volume of 25 μL. The PCR cycling protocol was 95° C. for 3 min followed by 40 cycles of 95° C. for 10 s and 60° C. for 30 s. Human glyceraldehyde-3-phosphate dehydrogenase (GAPDH) gene was used as internal control. Oligonucleotides sequences for GAPDH and conditions were those previously reported by Henke-Gendo et al. (2012).

For quantification, gene fragments from hexon, and GAPDH were cloned into the pGEM-T Easy vector (Promega) and known concentrations of template were used to generate a standard curve in parallel for each experiment. All assays were performed in thermal cycler LightCycler® 96 System (Roche).

Time of Addition Assay. The anti-HAdV effect of compounds 153 and 161 at different points was measured in a time-curve assay using 293β5 cells (3×10⁵ cells/well in coming black wall, clear bottom 96-well plates) infected with HAdV5-GFP (2.000 vp/cell) in the presence of 5 μM of compound 153 and 0.78 μM for derivative 161. Parallel samples of HAdV-5 were incubated with or without the selected compounds on ice for 1 h. Virus was then added to 293β5 cells and incubated at 37° C. Compounds 153 and 161 were added at the indicated time points before or during this incubation. After a total of 2 h at 37° C., cells were incubated for an additional 48 h at 37° C. and 5% CO₂ before being analyzed for GFP expression using the Typhoon 9410 imager (GE Healthcare Life Sciences) as above.

HadV-mediated Endosome Disruption. A549 cells (˜20,000 cells/well) were incubated in DMEM without cysteine or methionine supplemented with 10% dialyzed FBS [DMEM(−)] in black 96-well plates for 1 h prior to infection. Three-fold serial dilutions (0.45 ng to 1000 ng) of HAdV5, or AdV2ts1 were preincubated with cells in the presence of 5 μM niclosamide, compound 161 or the same volume of DMSO (negative control) for one hour. The medium was then removed and replaced with 50 μl DMEM(−) containing 0.1 mg/ml α-sarcin (Santa Cruz Biotechnology, Dallas, Tex., USA) and the virus and drug mixtures. AFter 2 hours at 37° C., the Click-iT HPG Alexa Fluor 488 Protein Synthesis Assay Kits (Invitrogen) was used to analyse protein synthesis according to the manufacturer's instructions. The incorporation of the amino acid analog of methionine L-homopropargylglycine (HPG) containing Alexa Fluor 488 azide was measured using a Typhoon 9410 imager (GE Healthcare Life Sciences) and calculated subtracting the background level of the control well containing L-homopropargylglycine (HPG) and α-sarcin but not virus (100% incorporation).

Statistical Analyses. One-way ANOVA tests (Dunnet method) were carried out using the GraphPad Prism 6. We considered a statistical significance with a P value under 0.05. This statistical significance was pointed out with asterisk in graphs, and the numbers of them indicate the level of significance (*p≤0.05, **p≤0.01, ***p≤0.001, ****p≤0.0001).

Abbreviations—HAdVs, human adenoviruses; SAR, structure-activity relationship; allo-HSCT, allogenic hematopoietic stem cell transplant; SOT, solid-organ transplant; AIDS, acquired immune deficiency syndrome; BCV, brincidofovir; CAP, community-acquired pneumonia; CMV, cytomegalovirus; HCMV, human cytomegalovirus; HTS, high-throughput screening; HPMPA, 9-(3-hydroxy-2-phosphonyl-methoxy-propyl)-adenine; HIV, human immunodeficiency virus; EC₅₀, half maximal effective concentration; IC₅₀, half maximal inhibitory concentration; CC₅₀, cytotoxicity concentration 50%; ED₅₀, median effective dose; TCID₅₀, median tissue culture infective dose; MTD, maximum tolerated dose; p.i., post-infection; qPCR, quantitative real-time PCR; TLC, thin layer chromatography; UV, ultraviolet; TMS, tetramethylsilane; THP, tetrahydropyran; HRMS, high-resolution mass spectrometry; HPLC, high-performance liquid chromatography; HBTU, 2-(1H-benzotriazole-1-yl)-1,1,3,3-tetramethyluronium hexafluorophosphate; DIEA, N,N-diisopropylethylamine; DCM, dichloromethane; DMAP, 4-(dimethylamino)pyridine; DMSO, dimethyl sulfoxide; EDCI, 1-(3-dimethylaminopropyl)-3-ethylcarbodiimide hydrochloride; EtOAc, ethyl acetate.

1. A compound according to Formula I: 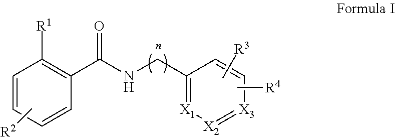
wherein R¹ is chosen from OH, —OR⁵—NHSO₂R⁵ or —NHCOR⁵, wherein R⁵ is a straight chained, or branched alkyl; R² is chosen from H, halogen, CN, NO₂, amino, or alkyl; R³ and R⁴ are independently chosen from H, halogen, CN, NO₂, CF₃, mono-substituted amino, di-substituted amino, or R³ and R⁴ taken together with other atoms form 5-membered or 6-membered fused ring; X₁, X₂, and X₃ are independently chosen from CH and N; and n is 0, 1, or 2.

2. The compound of claim 1, R¹ is OH.

3. The compound of claim 2, R¹ form an ester prodrugs.

4. A compound according to Formula Ia: 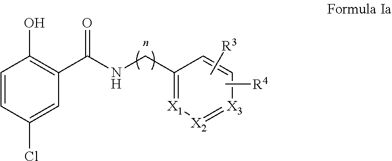
wherein R³ and R⁴ are independently chosen from H, halogen, CN, NO₂, CF₃, monosubstituted amino, di-substituted amino, or R³ and R⁴ with other atoms form a 5-membered or 6-membered fused ring; X₁, X₂, and X₃ are independently chosen from CH and N; and n is 0, 1, or 2.

5. A compound according to Formula Ib: 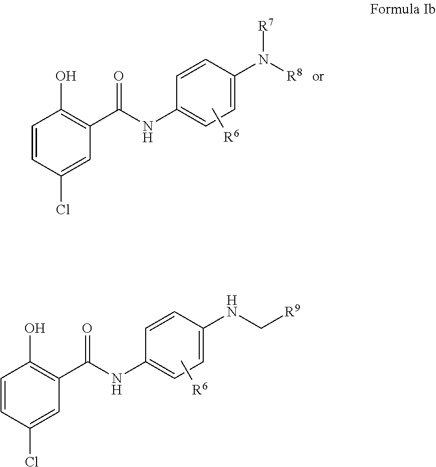
wherein R⁶ is chosen from H or halogen; R⁷ and R⁸ are independently chosen from H, alkyl, aryl, arylalkyl, cycloalkyl, alkoxy, heteroalkyl, hydroxyalkyl, or R⁷ and R⁵ form with other atoms a 5-membered or 6-membered fused ring; and R⁹ is an unsubstituted or substituted aryl, or heteroaryl.

6. A compound according to Formula II: 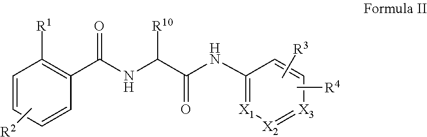
wherein R¹ is chosen from —OH, —OR⁵—NHSO₂R⁵ or —NHCOR⁵, wherein R⁵ is a straight chained, or branched alkyl; R² is H, halogen, CN, NO₂, amino, or alkyl; R³ and R⁴ are independently chosen from H, halogen, CN, NO₂, CF₃, mono-substituted amino, di-substituted amino, or R³ and R⁴, with other atoms, form a 5-membered or 6-membered fused ring; R¹⁰ is H, alkyl, cycloalkyl, heteroalkyl, aryl, heteroaryl, arylalkyl, or heteroarylalkyl, wherein the heteroalkyl includes an ester bond, an amide bond, a carbamate, a sulfur or an oxygen; and X₁, X₂, and X₃ are independently CH or N.

7. The compound of claim 6, wherein R¹ forms an ester prodrug.

8. A compound according to Formula III: 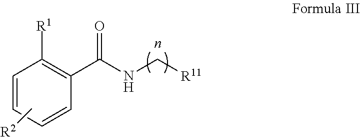
wherein R¹ is chosen from OH, —OR⁵, —NHSO₂R⁵ or —NHCOR⁵ wherein R⁵ is a straight chained or branched alkyl; R² is chosen from H, halogen, CN, NO₂, amino, or alkyl; R¹¹ is chosen from alkyl, heteroalkyl, cycloalkyl, or heterocycle, wherein the heteroalkyl and/or the heterocycle include an ester bond, an amide bond, a carbamate or an oxygen; and n is 0, 1, 2, 3, 4, 5, or 6.

9. The compound of claim 8, wherein R¹ forms an ester prodrug.

10. A compound of any one of claims 1-9, wherein the compound is 5-Chloro-N-(2-fluoro-4-nitrophenyl)-2-hydroxybenzamide (11), 5-Chloro-2-hydroxy-N-(4-nitrophenyl)benzamide (13), 5-Chloro-N-(2-chloro-4-(trifluoromethyl)phenyl)-2-hydroxybenzamide (14), 5-Chloro-N-(3-fluoro-5-(trifluoromethyl)phenyl)-2-hydroxybenzamide (17), N-(3-Fluoro-5-(trifluoromethyl)phenyl)-2-hydroxybenzamide (18), N-(3-Fluoro-5-(trifluoromethyl)phenyl)-2-hydroxy-5-methylbenzamide (19), 4-Chloro-N-(3-fluoro-5-(trifluoromethyl)phenyl)-2-hydroxybenzamide (20), 5-Chloro-N-(2,4-dichlorobenzyl)-2-hydroxybenzamide (32), 5-Chloro-N-(3-fluoro-4-(trifluoromethyl)benzyl)-2-hydroxybenzamide (36), 5-Chloro-N-(2-fluoro-4-(trifluoromethyl)benzyl)-2-hydroxybenzamide (37), (S)-5-Chloro-N-(1-((2-chloro-4-nitrophenyl)amino)-3-methyl-1-oxobutan-2-yl)-2-hydroxybenzamide (58), (R)-5-Chloro-N-(1-((2-chloro-4-nitrophenyl)amino)-1-oxo-3-phenylpropan-2-yl)-2-hydroxybenzamide (60), 5-Chloro-N-((2S,3R)-1-((2-chloro-4-nitrophenyl)amino)-3-methyl-1-oxopentan-2-yl)-2-hydroxybenzamide (62), (S)-tert-Butyl 4-(5-chloro-2-hydroxybenzamido)-5-((2-chloro-4-nitrophenyl)amino)-5-oxopentanoate (64), (S)—N-(1-((3,5-Bis(trifluoromethyl)phenyl)amino)-3-methyl-1-oxobutan-2-yl)-5-chloro-2-hydroxybenzamide (65), (S)-5-Chloro-N-(1-((3-fluoro-5-(trifluoromethyl)phenyl)amino)-3-methyl-1-oxobutan-2-yl)-2-hydroxybenzamide (67), or (S)-5-Chloro-N-(1-((2-chloro-4-nitrophenyl)amino)-3-methyl-1-oxobutan-2-yl)-2-(methylsulfonamido)benzamide (70).

11. A method of treating a viral infection comprising administering to a subject a compound of claim 1, 4, 5, 6, 8 or 10.
